SEQUENCE LISTING (1 sequence)

SEQ ID NO: 1 - protein, 26 aa, Artificial Sequence
  source: /note=“Description of Artificial Sequence Synthetic peptide”
  MOD_RES (1)..(1): Beta-Ala
  MOD_RES (2)..(11): Sarcosine
  MOD_RES (14)..(14): D-Ala
  MOD_RES (17)..(17): 1Nal
  MOD_RES (18)..(18): D-Ala
  MOD_RES (25)..(25): tBuGly
XXXXXXXXXXXACXNEXXCEDFYDXC

(19) United States
(12) Patent Application Publication
(10) Pub. No.: US 2020/0283482 A1
(43) Pub. Date: Sep. 10, 2020

(54) BICYCLIC PEPTIDE LIGAND PRR-A CONJUGATES AND USES THEREOF

(71) Applicant: BicycleRD Limited, Cambridge (GB)

(72) Inventors: Nicholas Keen, Carlisle, MA (US); Kevin McDonnell, Lexington, MA (US); Peter U. Park, Lincoln, MA (US); Gemma Elizabeth Mudd, Cambridge (GB); Gabriela Ivanova-Berndt, Cambridge (GB)

(21) Appl. No.: 16/639,183

(22) PCT Filed: Aug. 14, 2018
(86) PCT No.: PCT/GB2018/052310

§ 371 Date: Feb. 14, 2020

Related U.S. Application Data
(60) Provisional application No. 62/545,415, filed on Aug. 14, 2017.
(60) Provisional application No. 62/562,954, filed on Sep. 25, 2017.

(51) Int. Cl. C07K 7/50; A61K 47/65; A61K 47/64; C07D 401/10; C07D 471/04; C07D 487/04; A61P 35/00

(52) U.S. Cl. CPC C07K 7/50; A61K 47/65; A61K 47/64; A61K 38/00; C07D 471/04; C07D 487/04; A61P 35/00; C07D 401/10

(57) ABSTRACT

The present invention provides compounds, compositions thereof, and methods of using the same.

### TECHNICAL FIELD OF THE INVENTION

The present invention relates to polypeptides which are covalently bound to molecular scaffolds such that two or more peptide loops are subtended between attachment points to the scaffold and further linked to pattern recognition receptor agonist (PRR-A). In particular, the invention describes bicyclic peptide ligands useful for selectively delivering the linked PRR-A to cancer cells. The invention also describes peptides which are high affinity binders of membrane type 1 metalloprotease (MT1-MMP). The invention also includes pharmaceutical compositions comprising said peptide ligands and to the use of said peptide ligands in preventing, suppressing or treating cancer.

### BACKGROUND OF THE INVENTION

The human immune response is mediated through two parallel immune components. The innate immunes system responds to pathogens and abnormal cells through multiple cell types including dendritic cells, macrophages, neutrophils, and natural killer cells and represents a first line of defense in mammals. The adaptive immune response system responds to pathogens and abnormal cells through the T cell and B cell systems, neutralizing these components with T-cell receptors and antibodies respectively.

The innate immune system relies on specialized receptors known as pattern recognition receptors (PRRs), which recognize specific pathogen-associated molecular patterns (PAMPs), which are common to microbes but not to mammals, and damage-associated molecular patterns (DAMPs), which are molecules released by the host's own tissue (e.g. from dying cells) or by the environment (e.g. toxins). Upon detection of PAMPs and DAMPs, certain PRRs trigger an inflammatory response that leads to efficient destruction of the invading pathogens.

Toll-like receptors (TLRs) in the innate immune system are transmembrane pattern recognition receptors capable of recognizing generic pathogen-associated molecular patterns (PAMPs) and danger-associated molecular patterns (DAMPs). Their activation induces intracellular signaling pathways that result in production of inflammatory cytokines as well as type I interferons (IFNs). The action of vaccines is due, in part, to the activation of the TLR system. There are 10 known functional TLRs in humans and 12 in mice; TLRs 1-9 are conserved in both species. Each TLR is associated with the recognition of specific PAMPs, and the response that ensues upon their activation is dependent upon the particular pathogen and the immune cell subtype involved. TLR-mediated recognition of its cognate PAMPs can occur at the plasma membrane or at endosomal and/or endolysosomal membranes. TLR1, 2, 4, 5, 6 and 11 are primarily, although not exclusively, expressed on the plasma membrane of immune cells. These TLRs recognize a variety of unique microbial membrane components like lipids, lipoproteins and proteins. Conversely, TLR 3, 7, 8 and 9 are expressed on intracellular vesicular membranes and are commonly involved in recognition of nucleic acids.

Agonists of the TLRs would be immune system enhancers and have been proposed to be useful in the treatment of cancer and infectious diseases. Antagonists, on the other hand, are thought to have a therapeutic role in suppressing overactive immune responses, as occurs in chronic inflammatory and autoimmune diseases. Preclinical and clinical evidence demonstrates that the release of TLR-activating DAMPs by dying cancer cells contributes to the elicitation of therapeutically relevant anticancer immune responses. TLR ligands have therefore gained substantial attention as targeted agents that are designed to activate innate adaptive immune responses in the host. Bacillus Calmette-Guérin (BCG) is an attenuated variant of Mycobacterium bovis that is licensed as a standalone therapeutic intervention for the treatment of non-invasive transitional cell carcinomas of the bladder. Three other TLR agonists are approved for use in oncological indications: (1) picibanil, a lyophilized preparation of Streptococcus pyogenes that is approved in Japan for the treatment of various carcinomas; (2) monophosphoryl lipid A (MPL), a derivative of Salmonella minnesota LPS that is employed as immunological adjuvant in a peptide-based vaccine specific for cervical carcinoma-associated strains of human papillomavirus (i.e., HPV-16 and HPV-18); and (3) imiquimod, an imidazoquinoline derivative that is used for the topical treatment of actinic keratosis, superficial basal cell carcinoma, and external genital/perianal warts (Condylomata acuminata).

TLR2, TLR4, TLR7, TLR8, and TLR9 have been the targets for small molecule drug discovery efforts. Several synthetic small molecule and oligonucleotide ligands have been extensively evaluated in preclinical and clinical studies. TLR7 and 8 agonists include imidazoquinolines, purine-like molecules and benzodiazepine structures. TLR2 and 4 antagonists have included lipopeptide and liposaccharide mimetics while TLR9 agonists are derived from unmethylated CpG motifs in ssDNA and include various oligodeoxnucleotides (ODN) constructs.

As various immunomodulatory features of the tumor microenvironment have been identified, it has become increasingly clear that selective triggering of TLRs at the site of a tumor can have both direct and indirect therapeutic benefits. A major concern with therapeutic use of any TLR agonist, which has to date highly limited their clinical use, is that systemic TLR activation can be fatal, with toxic shock caused by cytokine syndrome or cytokine storms. Recent efforts have therefore focused on reducing and eliminating this systemic toxicity. Typical prodrug and antedrug formulations have had limited success in imparting tolerability to TLR 7 agonists. Antedrugs are active compounds that are metabolically inactivated before entering systemic circulation. An alternative approach is to limit drug availability and localize inflammation by covalent conjugation to macromolecular scaffolds such as peptides, proteins and polymers, which may limit systemic cytokine levels but can induce high levels of inflammation in the target tumor or diseased tissue.

NOD-Like Receptors (NLRs) constitute a family of intracellular pattern recognition receptors (PRRs), in humans there are 22 known NLRs. Their primary role is to recognize cytoplasmic pathogen-associated molecular patterns (PAMPs) and/or endogenous danger signal, inducing immune responses.

NLRs are characterized by a tripartite-domain organization with a conserved nucleotide binding oligomerization domain (NOD) and leucine-rich repeats (LRRs).

The inflammasome is a large multiprotein complex which plays a key role in innate immunity by participating in the production of the pro-inflammatory cytokines interleukin-1β (IL-1β) and IL-18. These related cytokines cause a wide variety of biological effects associated with infection, inflammation and autoimmune processes.

They are both produced as inactive precursors, pro-IL-1β and pro-IL-18, and share a common maturation mechanism that requires caspase-1. Caspase-1 itself is synthesized as a zymogen, pro-caspase-1, that undergoes autocatalytic processing resulting in two subunits that form the active caspase-1. Activation of caspase-1 occurs within the inflammasome following its assembly.

The best characterized inflammasome is the NLRP3 (also known as NALP3 and cryopyrin) inflammasome. It comprises the NLR protein NLRP3, the adapter ASC and pro-caspase-1. The general consensus is that maturation and release of IL-1 requires two distinct signals: the first signal leads to synthesis of pro-IL-1β and other components of the inflammasome, such as NLRP3 itself; the second signal results in the assembly of the NLRP3 inflammasome, caspase-1 activation and IL-1β secretion.

As various immunomodulatory features of the tumor microenvironment have been identified, it has become increasingly clear that selective triggering of NLRs at the site of a tumor can have both direct and indirect therapeutic benefits.

Another PAMP protein family are RIG-I-like receptors (RLRs), which include RIG-I and MDA5, that detect viral double-stranded RNA in the cytoplasm. RIG-I recognizes short RNA ligands with 5′-triphosphate caps. MDA5 recognizes long kilobase-scale genomic RNA and replication intermediates. Ligand binding induces conformational changes and oligomerization of RLRs that activate the signaling partner MAVS on the mitochondrial and peroxisomal membranes. This signaling process is under tight regulation, dependent on post-translational modifications of RIG-I and MDA5, and on regulatory proteins including unanchored ubiquitin chains and a third RLR, LGP2.

In attempts to discover effective cellular targets for cancer therapy, researchers have sought to identify transmembrane or otherwise tumor-associated polypeptides that are specifically expressed on the surface of one or more particular type(s) of cancer cell as compared to on one or more normal non-cancerous cell(s). Often, such tumor-associated polypeptides are more abundantly expressed on the surface of the cancer cells as compared to on the surface of the non-cancerous cells. The identification of such tumor-associated cell surface antigen polypeptides, i.e. tumor-associated antigens (TAA), has given rise to the ability to specifically target cancer cells for destruction.

A proprietary phage display and cyclic peptide technology (Bicycle® technology) can be utilized to identify high affinity binding peptides to TAA.

TAA include, but are not limited to: 5T4, AOC3, ALK, AXL, C242, CA-125, CCL11, CCR 5, CD2, CD3, CD4, CD5, CD15, CA15-3, CD18, CD19, CA19-9, CD20, CD22, CD23, CD25, CD28, CD30, CD31, CD33, CD37, CD38, CD40, CD41, CD44, CD44 v6, CD51, CD52, CD54, CD56, CD62E, CD62P, CD62L, CD70, CD74, CD79-B, CD80, CD125, CD138, CD141, CD147, CD152, CD154, CD326, CEA, CTLA-4, CXCR2, EGFR, ErbB2, ErbB3, EpCAM, EphA2, EphB2, EphB4, FGFR (i.e. FGFR1, FGFR2, FGFR3, FGFR4), FLT3, folate receptor, FAP, GD2, GD3, GPNMB, HGF, HER2, ICAM, IGF-1 receptor, VEGFR1, TRPV1, CFTR, gpNMB, CA9, Cripto, c-KIT, c-MET, ACE, APP, adrenergic receptor-beta2, Claudine 3, Mesothelin, MUC1, RON, ROR1, PD-1, PD-L1, PD-L2, B7-H3, B7-B4, IL-2 receptor, IL-4 receptor, IL-13 receptor, integrins (including α₄, αᵥβ₃, αᵥβ₅, αᵥβ₆, α₁β₄, α₄β₁, α₄β₇, α₅β₁, α₆β₄, α_{IIb}β₃ integrins), IFN-α, IFN-γ, IgE, IGF-1 receptor, IL-1, IL-12, IL-23, IL-13, IL-22, IL-4, IL-5, IL-6, interferon receptor, ITGB2 (CD18), LFA-1 (CD11a), L-selectin (CD62L), mucin, MUC1, myostatin, NCA-90, NGF, PDGFRα, phosphatidylserine, prostatic carcinoma cell, RANKL, Rhesus factor, SLAMF7, sphingosine-1-phosphate, TAG-72, T-cell receptor, tenascin C, TGF-1, TGF-β2, TGF-β, TNF-α, TRAIL-R1, TRAIL-R2, tumor antigen CTAA16.88, VEGFA, VEGFR2, vimentin, and the like.

Additionally, Bicycle® technology can be utilized to identify high affinity binding peptides to one or more tumor-associated antigens or cell-surface receptors selected from (1)-(36):

(1) BMPR1B (bone morphogenetic protein receptor-type IB, Genbank accession no. NM.sub.--001203);

(2) E16 (LAT1, SLC7A5, Genbank accession no. NM.sub.--003486);

(3) STEAP1 (six transmembrane epithelial antigen of prostate, Genbank accession no. NM.sub.--012449);

(4) 0772P (CA125, MUC16, Genbank accession no. AF361486);

(5) MPF (MPF, MSLN, SMR, megakaryocyte potentiating factor, mesothelin, Genbank accession no. NM.sub.-005823);

(6) Napi3b (NAPI-3B, NPTIIb, SLC34 Å2, solute carrier family 34 (sodium phosphate), member 2, type II sodium-dependent phosphate transporter 3b, Genbank accession no. NM.sub.--006424);

(7) Sema 5b (FLJ10372, KIAA1445, Mm.42015, SEMA5B, SEMAG, Semaphorin 5b Hlog, sema domain, seven thrombospondin repeats (type 1 and type 1-like), transmembrane domain (TM) and short cytoplasmic domain, (semaphorin) 5B, Genbank accession no. AB040878);

(8) PSCA hlg (2700050C12Rik, C530008016Rik, RIKEN cDNA 2700050C12, RIKEN cDNA 2700050C12 gene, Genbank accession no. AY358628);

(9) ETBR (Endothelin type B receptor, Genbank accession no. AY275463);

(10) MSG783 (RNF124, hypothetical protein FLJ20315, Genbank accession no. NM.sub.--017763);

(11) STEAP2 (HGNC.sub.--8639, IPCA-1, PCANAP1, STAMPI, STEAP2, STMP, prostate cancer associated gene 1, prostate cancer associated protein 1, six transmembrane epithelial antigen of prostate 2, six transmembrane prostate protein, Genbank accession no. AF455138);

(12) TrpM4 (BR22450, FLJ20041, TRPM4, TRPM4B, transient receptor potential cation channel, subfamily M, member 4, Genbank accession no. NM.sub.--017636);

(13) CRIPTO (CR, CR1, CRGF, CRIPTO, TDGF1, teratocarcinoma-derived growth factor, Genbank accession no. NP.sub.--003203 or NM.sub.--003212);

(14) CD21 (CR2 (Complement receptor 2) or C3DR(C3d/Epstein Barr virus receptor) or Hs.73792 Genbank accession no. M26004);

(15) CD79b (CD79B, CD79.beta., IGb (immunoglobulin-associated beta), B29, Genbank accession no. NM.sub.--000626);

(16) FcRH2 (IFGP4, IRTA4, SPAP1A (SH2 domain containing phosphatase anchor protein 1a), SPAP1B, SPAP1C, Genbank accession no. NM.sub.--030764);

(17) HER2 (Genbank accession no. M1730);

(18) NCA (Genbank accession no. M18728);

(19) MDP (Genbank accession no. BC017023);

(20) IL20Ra (Genbank accession no. AF184971);

(21) Brevican (Genbank accession no. AF229053;

(22) EphB2R (Genbank accession no. NM.sub.--004442);

(23) ASLG659 (Genbank accession no. AX092328);

(24) PSCA (Genbank accession no. AJ297436);

(25) GEDA (Genbank accession no. AY260763;

(26) BAFF--R (B cell-activating factor receptor, BLyS receptor 3, BR3, NP.sub.--443177.1);

(27) CD22 (B-cell receptor CD22-B isoform, NP.sub.--001762.1);

(28) CD79a (CD79A, CD79.alpha., immunoglobulin-associated alpha, a B cell-specific protein that covalently interacts with Ig beta (CD79B) and forms a complex on the surface with Ig M molecules, transduces a signal involved in B-cell differentiation, Genbank accession No. NP.sub.--001774.1);

(29) CXCR5 (Burkitt's lymphoma receptor 1, a G protein-coupled receptor that is activated by the CXCL13 chemokine, functions in lymphocyte migration and humoral defense, plays a role in HIV-2 infection and perhaps development of AIDS, lymphoma, myeloma, and leukemia, Genbank accession No. NP.sub.--001707.1);

(30) HLA-DOB (Beta subunit of MEC class II molecule (Ia antigen) that binds peptides and presents them to CD4+ T lymphocytes, Genbank accession No. NP.sub.--002111.1);

(31) P2X5 (Purinergic receptor P2X ligand-gated ion channel 5, an ion channel gated by extracellular ATP, may be involved in synaptic transmission and neurogenesis, deficiency may contribute to the pathophysiology of idiopathic detrusor instability, Genbank accession No. NP.sub.--002552.2);

(32) CD72 (B-cell differentiation antigen CD72, Lyb-2, Genbank accession No. NP.sub.--001773.1);

(33) LY64 (Lymphocyte antigen 64 (RP105), type I membrane protein of the leucine rich repeat (LRR) family, regulates B-cell activation and apoptosis, loss of function is associated with increased disease activity in patients with systemic lupus erythematosis, Genbank accession No. NP.sub.--005573.1);

(34) FcRH1 (Fc receptor-like protein 1, a putative receptor for the immunoglobulin Fc domain that contains C2 type Ig-like and ITAM domains, may have a role in B-lymphocyte differentiation, Genbank accession No. NP.sub.--443170.1);

(35) IRTA2 (Immunoglobulin superfamily receptor translocation associated 2, a putative immunoreceptor with possible roles in B cell development and lymphomagenesis; deregulation of the gene by translocation occurs in some B cell malignancies, Genbank accession No. NP.sub.--112571.1); and

(36) TENB2 (putative transmembrane proteoglycan, related to the EGF/heregulin family of growth factors and follistatin, Genbank accession No. AF 179274.

Additionally, the proprietary phage display and cyclic peptide technology (Bicycle® technology) can be utilized to identify high affinity binding peptides to the following markers and/or targets on immune cells:

Dendritic cells (DC)—

Myeloid/conventional DC markers and/or targets such as CD1a, CD1c (BDCA1), CD123, CD141 (BDCA3), CD205, and CD209;

Plasmacytoid DC markers and/or targets such as CD85g, CD289, CD303 (BDCA2), CD304 (BDCA4), TLR7, TLR8, and TLR9;

Markers and/or targets on Langherhans cells such as CD1a, CD207, and CD324; Markers and/or targets on macrophages such as CD11b, CD11c, CD14, CD68, CD80, and CD163;

Markers and/or targets on M1 Macrophages such as CD68, CD86, CD282, and CD284;

Markers and/or targets on M2 Macrophages such as CD163, CD220R, and CD206; and

Markers and/or targets on Tumor-Associated Macrophages such as CD81, CD106, and Dectin-1.

Transmembrane proteins which are overexpressed in cancer cells provide a potential means for selectively targeting cancer cells. One such transmembrane protein is membrane type 1-matrix metalloproteinase (MT1-MMP).

MT1-MMP is a transmembrane metalloprotease that plays a major role in the extracellular matrix remodelling, directly by degrading several of its components and indirectly by activating pro-MMP2. MT1-MMP is crucial for tumor angiogenesis (Sounni et al (2002) FASEB J. 16(6), 555-564) and is over-expressed on a variety of solid tumors.

Accordingly, there remains a high unmet need in developing PRR-A agents that selectively target cancer cells by means of a covalently linked high affinity Bicycle peptide binder to one or more tumor-associated antigens or cell-surface receptors for the treatment of cancer.

### SUMMARY OF THE INVENTION

It has now been found that compounds of this invention, and pharmaceutically acceptable compositions thereof, are effective as immunostimulatory agents. Such compounds have the general formula I:

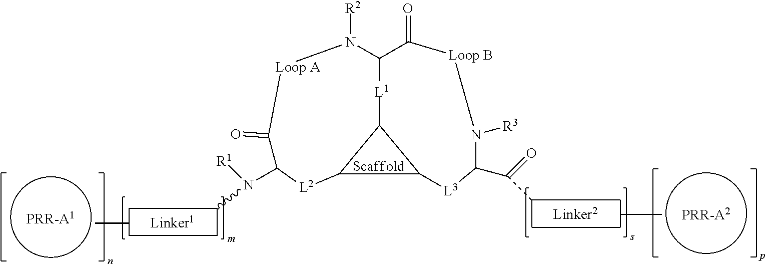

or a pharmaceutically acceptable salt thereof, wherein each variable is as defined and described herein.

Compounds of the present invention, and pharmaceutically acceptable compositions thereof, are useful for treating a variety of diseases, disorders or conditions. Such diseases, disorders, or conditions include those described herein.

### BRIEF DESCRIPTION OF THE FIGURES

FIG. 1 depicts the body weight changes after administering I-8 to female C57BL/6J mice bearing B16F10 xenograft. Data points represent group mean body weight. Error bars represent standard error of the mean (SEM).

FIG. 2 depicts depicts the tumor volume trace after administering I-8 to female C57BL/6J mice bearing B16F10 xenograft. Data points represent group mean body weight. Error bars represent standard error of the mean (SEM).

FIG. 3 depicts the body weight changes after administering I-8 alone or in combination with aPD-1 to female C57BL/6J mice bearing B16F10 xenograft. Data points represent group mean body weight. Error bars represent standard error of the mean (SEM).

FIG. 4 depicts the tumor volume trace after administering I-8 alone or in combination with aPD-1 to female C57BL/6J mice bearing B16F10 xenograft. Data points represent group mean body weight. Error bars represent standard error of the mean (SEM).

FIG. 5 depicts assay dose response curves for I-7, I-8, I-9 and resiquimod in the human TLR7 receptor activation assay using HEK293 reporter cell lines engineered to express TLR7 receptors.

FIG. 6 depicts assay dose response curves for I-7, I-8, I-9 and resiquimod in the human TLR8 receptor activation assay using HEK293 reporter cell lines engineered to express TLR8 receptors.

FIG. 7 depicts plasma concentration of compound I-8 and released payload after IV dosing at 3 mg/kg in CD-1 Mice.

FIG. 8 depicts plasma concentration of compound I-7 and released payload after IV dosing at 3 mg/kg in CD-1 Mice.

FIG. 9 depicts plasma concentration of compound I-22 and released payload after IV dosing at 3 mg/kg in CD-1 Mice.

FIG. 10 depicts plasma concentration of compound I-24 and released payload after IV dosing at 3 mg/kg in CD-1 Mice.

FIG. 11 depicts plasma concentration of compound I-27 and released payload after IV dosing at 3 mg/kg in CD-1 Mice.

FIG. 12 depicts plasma concentration of compound I-29 and released payload after IV dosing at 3 mg/kg in CD-1 Mice.

FIG. 13 depicts plasma concentration of compound I-33 and released payload after IV dosing at 3 mg/kg in CD-1 Mice.

FIG. 14 depicts plasma concentration of compound I-30 and released payload after IV dosing at 3 mg/kg in CD-1 Mice.

FIG. 15 depicts plasma concentration of I-7 and released payload after IV infusion dosing of I-7 at 1 mg/kg in Cynomolgus monkey.

FIG. 16 depicts plasma concentration of I-22 and released payload after IV infusion dosing of I-22 at 1 mg/kg in Cynomolgus monkey.

FIG. 17 depicts tumor, spleen and plasma cytokine levels 1 h after IT dosing in B16F10 bearing C57BL/6 mice with 1 mg of conjugate I-7 or I-22, or 0.1 mg payload R848.

FIG. 18 depicts tumor, spleen and plasma cytokine levels 1 h after IT dosing in B16F10 bearing C57BL/6 mice with 1 mg of conjugate I-24, or I-29, or I-31, or 0.1 mg payload R848.

FIG. 19 depicts tumor, spleen and plasma cytokine levels 1 h after IT dosing in B16F10 bearing C57BL/6 mice with 1 mg of conjugate I-33 or 0.1 mg payload Gardiquimod.

FIG. 20 depicts serum cytokine formation 1 h after IV dosing in C57BL/6 mice of 20 mg/kg conjugate or 2 mg/kg payload.

FIG. 21 depicts serum cytokine formation 1 h after IV dosing in C57BL/6 mice of 20 mg/kg conjugate.

FIG. 22 depicts tumor, spleen and plasma cytokine levels 1 h after IV dosing in B16F10 bearing C57BL/6 mice of 20 mg/kg conjugate I-7 or I-22, or 2 mg/kg payload R848.

FIG. 23 depicts treatment of mice bearing B16.F10 tumours by IV dosing of I-7 or I-22 at 20 or 60 mg/kg tiw.

FIG. 24 depicts treatment of mice bearing MC38 tumors by IV dosing of I-7 or I-22 at 20 mg/kg tiw.

FIG. 25 depicts treatment of mice bearing CT26 tumors by IV dosing of I-7 or I-22 at 20 mg/kg tiw.

FIG. 26 depicts treatment of mice bearing CT26 tumours by IV dosing of I-7 or I-22 at 20 mg/kg tiw in combination with IP dosing of anti-PD1 mAb at 10 mg/kg biw.

### DETAILED DESCRIPTION OF CERTAIN EMBODIMENTS

### 1. General Description of Certain Embodiments of the Invention

A proprietary phage display and cyclic peptide technology (Bicycle technology) was utilized to identify high affinity binding peptides to the membrane type 1-matrix metalloproteinase (MT1-MMP/MMP14). MT1-MMP (MT1) is a cell surface membrane protease normally involved in tissue remodeling which has been found to be over-expressed in many solid tumors. Overexpression of MT1 has been linked to cancer invasiveness and poor prognosis. While attempts to target the proteolytic activity of MT1 and other MMPs in cancer were unsuccessful in clinical trials largely due to toxicity caused by insufficient selectivity, MT1-MMP remains an attractive cancer target for targeted cytotoxic delivery approaches.

Diverse selection phage libraries containing 10¹¹ to 10¹³ unique peptide sequences which are post-translationally cyclized with thiol-reactive scaffolds were used to identify small (1.5-2 kDa) constrained bicyclic peptides binders (Bicycles) to the hemopexin domain of MT1. Initial binders were subject to affinity maturation by directed screens and stabilization by chemical optimization.

A bicyclic constrained peptide binder (Bicycle) was identified that binds to the hemopexin domain of MT1 with an apparent Kd of approximately 2 nM. The Bicycle peptide (N241) binds with similar affinity to the entire ectodomain of the protease but shows no binding to the catalytic domain. N241 also shows no binding toward any of the closely related MMP family members tested (MMP15, MMP16, MMP24, MMP1, Pro-MMP1, MMP2). Characterization of the pharmacologic effect of N241 on MT1 in vitro shows that the peptide has no direct impact on the catalytic activity of the protease, nor related MMP catalytic activity (MMP1, MMP2 and MMP9) nor cell migration or invasion. However, binding of fluorescently-tagged N241 to MT1 on HT1080 fibrosarcoma cells results in the rapid internalization and subsequent lysosomal localization of the compound. In addition, ¹⁷⁷Lu-loaded N241 demonstrates rapid tumor localization when injected IV into mice bearing MT1-positive tumor xenografts, with levels as high as 15-20% injected dose per gram of tumor in less than 60 minutes. In contrast, a non-binding Bicycle peptide shows no tumor localization. Bicycle Drug Conjugates (BDCs) with a variety of linkers and detectable moieties were prepared which retained binding to MT1. The activity of select BDCs was demonstrated in MT1-positive human tumor cell xenografts in mice as described in WO 2016/067035, which is hereby incorporated in its entirety by reference. These properties suggest that N241 may be a good delivery vehicle for PRR-A targeting MT1-positive tumor cells.

MT1-MMP is naturally involved in tissue remodeling, however overexpression of the cell-surface protease has been tied to tumor aggressiveness and invasiveness, as well as poor patient prognosis for many cancer indications. The Bicycle binder for MT1-MMP (N241) was identified using a proprietary phage display peptide technology consisting of highly diverse phage libraries of linear amino acid sequences constrained into two loops by a central chemical scaffold. While binding with similar affinity and specificity to that observed with monoclonal antibodies, the small size of a Bicycle peptide (1.5-2 kDa) aids in its rapid extravasation and tumor penetration making it an ideal format for the targeted delivery of PRR-A for treating cancer.

A series of Bicycle-Linker-(PRR-A) conjugates were prepared, with varying spacer format to adjust the presentation of the Bicycle for evaluation of their ability to target tumors in an MT1-positive tumor xenograft model.

It is believed that the Bicycle PRR-A conjugates (BPCs) of the present invention may show selective targeting of tumor cells in human tumor xenograft models of fibrosarcomas. Without wishing to be bound by any particular theory, it is believed that the small size of the BPC may offer a significant advantage to other targeted imaging approaches such as antibody-detectable moiety conjugates due to rapid extravasation and improved tumor penetration.

In certain aspects, the present invention provides a method of treating certain cancers in a subject, comprising administering to the subject an effective amount of a PRR-A conjugate comprising a high affinity binder of MT1-MMP, or a pharmaceutically acceptable salt or composition thereof.

In some embodiments, peptide sequences are treated with molecular scaffold reagents to form compounds of the present invention.

In certain embodiments, the present invention provides a compound of formula I:

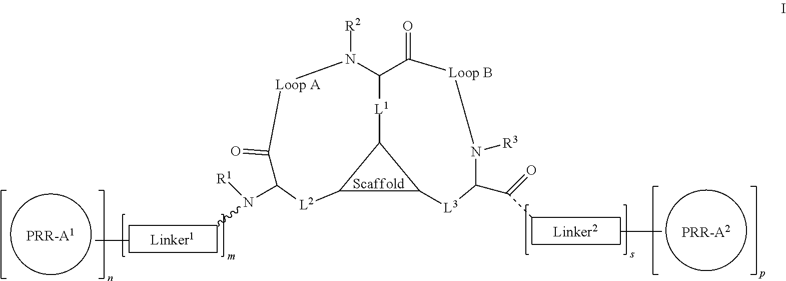

or a pharmaceutically acceptable salt thereof, wherein:
each of L¹, L², and L³ is independently a covalent bond or a C₁₋₈ bivalent hydrocarbon chain wherein one, two or three methylene units of the chain are optionally and independently replaced by —S—, —N(R)—, —O—, —C(O)—, —OC(O)—, —C(O)O—, —C(O)N(R)—, —N(R)C(O)—, —S(O)—, —S(O)₂— or —N(R)CH₂C(O)—;
each of R is independently hydrogen or C₁₋₄ alkyl;
each of m, n, s, and p is independently 0 or 1;
each of q and r is independently 1, 2, 3, 4, 5, 6, 7, 8, 9, 10, 11, 12, 13, 14 or 15;
R¹ is R or —C(O)R;
each of R⁴ and R⁶ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
each of R^{4′} and R^{6′} is independently hydrogen or methyl;
each of R², R³, R⁵, and R⁷ is independently hydrogen, or C₁₋₄ aliphatic, or:
   an R⁵ group and its adjacent R⁴ group are optionally taken together with their intervening atoms to form a 4-8 membered saturated or partially unsaturated monocyclic heterocyclic ring having 1-2 heteroatoms independently selected from nitrogen, oxygen, or sulfur; or
   an R⁷ group and its adjacent R⁶ group are optionally taken together with their intervening atoms to form a 4-8 membered saturated or partially unsaturated monocyclic heterocyclic ring having 1-2 heteroatoms independently selected from nitrogen, oxygen, or sulfur;
Scaffold is a trivalent group that connects and orients a cyclic peptide;
Loop A is a bivalent natural or unnatural amino acid residue or peptide attached to the amino acid residue linked to L² and the amino acid residue linked to L¹, wherein Loop A comprises

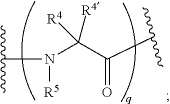

Loop B is a bivalent natural or unnatural amino acid residue or peptide attached to the amino acid residue linked to L¹ and the amino acid residue linked to L³, wherein Loop B comprises

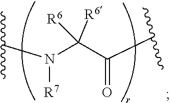

[symbol] indicates the site of attachment to the N-terminus of the Bicycle;
[symbol] indicates the site of attachment to the C-terminus of the Bicycle;
PRR-A¹ is a pattern recognition receptor agonist;
PRR-A² is a pattern recognition receptor agonist;
Linker¹ is hydrogen or a bivalent moiety that connects the N-terminus of the Bicycle with PRR-A¹, wherein when n is 0, Linker¹ is hydrogen;
Linker² is —NH₂ or a bivalent moiety that connects the C-terminus of the Bicycle with PRR-A², wherein when p is 0, Linker² is —NH₂; and
Ring A is selected from the group consisting of 18-crown-6, 1,7,13-triaza-18-crown-6, and a 3-12-membered saturated, partially unsaturated, bridged bicyclic, bridged tricyclic, propellane, or aromatic ring optionally substituted with 0-3 oxo, methyl, ethyl or spiroethylene groups and having 0-6 heteroatoms independently selected from nitrogen, oxygen, or sulfur.

### 2. Compounds and Definitions

### Peptide Ligands

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75′ Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

Cyclic peptides are able to bind with high affinity and target specificity to protein targets and hence are an attractive molecule class for the development of therapeutics. In fact, several cyclic peptides are already successfully used in the clinic, as for example the antibacterial peptide vancomycin, the immunosuppressant drug cyclosporine or the anti-cancer drug octreotide (Driggers et al. (2008), Nat Rev Drug Discov 7 (7), 608-24). Good binding properties result from a relatively large interaction surface formed between the peptide and the target as well as the reduced conformational flexibility of the cyclic structures. Typically, macrocycles bind to surfaces of several hundred square angstrom, as for example the cyclic peptide CXCR4 antagonist CVX15 (400 Å2; Wu et al. (2007), Science 330, 1066-71), a cyclic peptide with the Arg-Gly-Asp motif binding to integrin αVβ3 (355 Å2) (Xiong et al. (2002), Science 296 (5565), 151-5) or the cyclic peptide inhibitor upain-1 binding to urokinase-type plasminogen activator (603 Å2; Zhao et al. (2007), J Struct Biol 160 (1), 1-10).

Due to their cyclic configuration, peptide macrocycles are less flexible than linear peptides, leading to a smaller loss of entropy upon binding to targets and resulting in a higher binding affinity. The reduced flexibility also leads to locking target-specific conformations, increasing binding specificity compared to linear peptides. This effect has been exemplified by a potent and selective inhibitor of matrix metalloproteinase 8, MMP-8) which lost its selectivity over other MMPs when its ring was opened (Cherney et al. (1998), J Med Chem 41 (11), 1749-51). The favorable binding properties achieved through macrocyclization are even more pronounced in multicyclic peptides having more than one peptide ring as for example in vancomycin, nisin and actinomycin.

Different research teams have previously tethered polypeptides with cysteine residues to a synthetic molecular structure (Kemp and McNamara (1985), J. Org. Chem; Timmerman et al. (2005), ChemBioChem). Meloen and co-workers had used tris(bromomethyl)benzene and related molecules for rapid and quantitative cyclisation of multiple peptide loops onto synthetic scaffolds for structural mimicry of protein surfaces (Timmerman et al. (2005), ChemBioChem). Methods for the generation of candidate drug compounds wherein said compounds are generated by linking cysteine containing polypeptides to a molecular scaffold as for example tris(bromomethyl)benzene are disclosed in WO 2004/077062 and WO 2006/078161.

Phage display-based combinatorial approaches have been developed to generate and screen large libraries of bicyclic peptides to targets of interest (Heinis et al. (2009), Nat Chem Biol 5 (7), 502-7 and WO2009/098450). Briefly, combinatorial libraries of linear peptides containing three cysteine residues and two regions of six random amino acids (Cys-(Xaa)6-Cys-(Xaa)6-Cys) were displayed on phage and cyclised by covalently linking the cysteine side chains to a small molecule (tris-(bromomethyl)benzene).

A peptide ligand, as referred to herein, refers to a peptide covalently bound to a molecular scaffold. Typically, such peptides comprise two or more reactive groups (e.g. cysteine residues) which are capable of forming covalent bonds to the scaffold, and a sequence subtended between said reactive groups which is referred to as the loop sequence, since it forms a loop when the peptide is bound to the scaffold. In the present case, the peptides comprise at least three cysteine residues and form at least two loops on the scaffold. One of ordinary skill in the art will recognize that other amino acid residues capable of forming covalent bonds to the scaffold can be used (e.g. lysine, Dap or serine) to form bicyclic peptides of the present invention.

### Advantages of the Peptide Ligands

Certain bicyclic peptides of the present invention have a number of advantageous properties which enable them to be considered as suitable drug-like molecules for injection, inhalation, nasal, ocular, oral or topical administration. Without being bound by any particular theory, such advantageous properties may include:

Species cross-reactivity. This is a typical requirement for preclinical pharmacodynamics and pharmacokinetic evaluation;

Protease stability. Bicyclic peptide ligands should ideally demonstrate stability to plasma proteases, epithelial (“membrane-anchored”) proteases, gastric and intestinal proteases, lung surface proteases, intracellular proteases and the like. Protease stability should be maintained between different species such that a bicycle lead candidate can be developed in animal models as well as administered with confidence to humans;

Desirable solubility profile. This is a function of the proportion of charged and hydrophilic versus hydrophobic residues and intra/inter-molecular H-bonding, which is important for formulation and absorption purposes;

An optimal plasma half-life in the circulation. Depending upon the clinical indication and treatment regimen, it may be required to develop a bicyclic peptide for short exposure in an acute illness management setting, or develop a bicyclic peptide with enhanced retention in the circulation, and is therefore optimal for the management of more chronic disease states. Other factors driving the desirable plasma half-life are requirements of sustained exposure for maximal therapeutic efficiency versus the accompanying toxicology due to sustained exposure of the agent; and

Selectivity. Certain peptide ligands of the invention demonstrate good selectivity over other metalloproteases.

### Pattern Recognition Receptor Agonists

As mentioned above and described herein, PRR-A is a pattern recognition receptor agonist. Toll-like receptors (TLRs) in the innate immune system are transmembrane pattern recognition receptors, whereas NOD-like receptor pyrin domain containing 3 (NLRP3) receptors in the innate immune system are intracellular pattern recognition receptors. It has also been found that certain toll-like receptor (TLR) agonists are also NOD-like receptor pyrin domain containing 3 (NLRP3) agonists. VTX-2337 (motolimod), a selective toll-like receptor 8 (TLR8) agonist, stimulates the release of mature IL-1β and IL-18 from monocytic cells through coordinated actions on both TLR8 and NLRP3 (Dietsch et al. (2016) PLoS ONE 11(2) e0148764). Additionally, imiquimod, a TLR7 agonist and CL097, a TLR7/8 agonist, activate NLRP3 to trigger apoptosis-associated speck-like protein containing a CARD (ASC) oligomerization, IL-1 secretion and pyroptosis (Groβ et al. (2016) Immunity 45, 761-773). Accordingly, in some embodiments, a PRR-A may be both a TLR and NLRP3 agonist.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

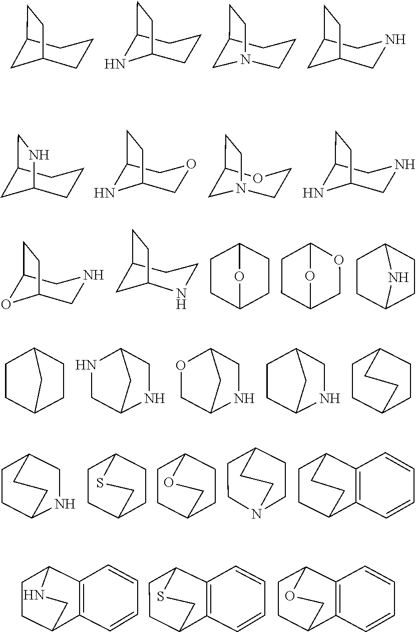

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C_{1_6}) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

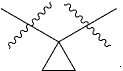 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄ CH(OR^{∘})₂; —(CH₂)₀₋₄ SR^{∘}; —(CH₂)₀₋₄ Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘}C(S)R^{∘}; —N(R^{∘}C(NR^{∘})N(R^{∘})₂; —(CH₂)₀₋₄N(R^{∘})C(O)NR^{∘}₂; —N(R^{∘})C(S)NR^{∘}₂; —(CH₂)₀₋₄N(R^{∘}C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄ SR—, —SC(S)SR^{∘}; —(CH₂)₀₋₄ SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄ SSR^{∘}; —(CH₂)₀₋₄ S(O)₂R^{∘}; —(CH₂)₀₋₄ S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}2; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘)}₂; —SiR^{∘}₃; —(C₁₋₄ straight or branched alkylene)O—N(R^{∘})₂; or —(C₁₋₄ straight or branched)alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, (haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂ SR^{●}, —(CH₂)₀₋₂ SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of Rt are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention. In certain embodiments, a provided compound comprises one or more deuterium atoms.

As used herein, the term “inhibitor” is defined as a compound that binds to and/or inhibits MT1-MMP with measurable affinity. In certain embodiments, an inhibitor has an ICso and/or binding constant of less than about 50 μM, less than about 1 μM, less than about 500 nM, less than about 100 nM, less than about 10 nM, or less than about 1 nM.

A compound of the present invention may be tethered to a PRR-A. It will be appreciated that such compounds are useful as therapeutic agents. One of ordinary skill in the art will recognize that a PRR-A may be attached to a provided compound via a suitable substituent. As used herein, the term “suitable substituent” refers to a moiety that is capable of covalent attachment to a PRR-A. Such moieties are well known to one of ordinary skill in the art and include groups containing, e.g., a carboxylate moiety, an amino moiety, a thiol moiety, or a hydroxyl moiety, to name but a few. It will be appreciated that such moieties may be directly attached to a provided compound or via a tethering group, such as a bivalent saturated or unsaturated hydrocarbon chain. In some embodiments, such moieties may be attached via click chemistry. In some embodiments, such moieties may be attached via a 1,3-cycloaddition of an azide with an alkyne, optionally in the presence of a copper catalyst. Methods of using click chemistry are known in the art and include those described by Rostovtsev et al., Angew. Chem. Int. Ed. 2002, 41, 2596-99 and Sun et al., Bioconjugate Chem., 2006, 17, 52-57.

As used herein, the term “detectable moiety” is used interchangeably with the term “label” and relates to any moiety capable of being detected, e.g., primary labels and secondary labels. Primary labels, such as radioisotopes (e.g., tritium, ²²⁵Ac, ²²⁷Ac, ²⁴¹Am, ⁷²As, ⁷⁴As, ²¹¹At, ¹⁹⁸Au, ¹¹B, ⁷Be, ²¹²Bi, ²¹³Bi, ⁷⁵Br, ⁷⁷Br, ¹¹C, ¹⁴C, ⁴⁸Ca, ¹⁰⁹Cd, ¹³⁹Ce, ¹⁴¹Ce, ²⁵²Cf, ⁵⁵Co, ⁵⁷Co, ⁶⁰Co, ⁵¹Cr, ¹³⁰Cs, ¹³¹Cs, ¹³⁷Cs, ⁶¹Cu, ⁶²Cu, ⁶⁴Cu, ⁶⁷Cu, ¹⁶⁵Dy, ¹⁵²Eu, ¹⁵⁵Eu, ¹⁸F, ⁵⁵Fe, ⁵⁹Fe, ⁶⁴Ga, ⁶⁷Ga, ⁶⁸Ga, ¹⁵³Gd, ⁶⁸Ge, ¹²²I, ¹²³I, ¹²⁴I, ¹²⁵I, ¹³¹I, ¹³²I, ¹¹¹In, ^{115m}In, ^{191m}Ir, ¹⁹²Ir, ^{81m}Kr, ¹⁷⁷Lu, ⁵¹Mn, ⁵²Mn, ⁹⁹Mo, ¹³N, ⁹⁵Nb, ¹⁵O, ¹⁹¹Os, ¹⁹⁴Os, ³²P, ³³P, ²⁰³Pb, ²¹²Pb, ¹⁰³Pd, ¹⁰⁹Pd, ²³⁸Pu, ²²³Ra, ²²⁶Ra, ⁸²Rb, ¹⁸⁶Re, ¹⁸⁸Re, ¹⁰⁵Rh, ⁹⁷Ru, ¹⁰³Ru, ³⁵S, ⁴⁶Sc, ⁴⁷Sc, ⁷²Se, ⁷⁵Se, ²⁸Si, ¹⁴⁵Sm, ¹⁵³Sm, ^{117m}Sn, ⁸⁵Sr, ⁸⁹Sr, ⁹⁰Sr, ¹⁷⁸Ta, ¹⁷⁹Ta, ¹⁸²Ta, ¹⁴⁹Tb, ⁹⁶Tc, ^{99m}Tc, ²²⁸Th, ²²⁹Th, ²⁰¹Tl, ¹⁷⁰Tm, ¹⁷¹Tm, ¹⁸⁸W, ¹²⁷Xe, ¹³³Xe, ⁸⁸Y, ⁹⁰Y ⁹¹Y, ¹⁶⁹Yb, ⁶²Zn, ⁶⁵Zn, ⁸⁹Zr or ⁹⁵Zr, wherein a superscripted m denotes a meta-state), mass-tags, and fluorescent labels are signal generating reporter groups which can be detected without further modifications. Detectable moieties also include luminescent and phosphorescent groups.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The terms “fluorescent label”, “fluorescent dye”, and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5, Cy7, Cy7.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAIVIRA), Texas Red, Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzypoxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The term “quantum dot” as used herein refers to any moiety that is a highly luminescent semiconductor nanocrystal (e.g. zincsulfide-capped cadmium selenide). The synthesis and utility of these quantum dots is described in U.S. Pat. Nos. 6,326,144, 6,468,808, 7,192,785, 7,151,047, and in the scientific literature (see: Chan and Nie (1998) Science 281(5385) 2016-2018).

The terms “measurable affinity” and “measurably inhibit,” as used herein, means a measurable change in MT1-MMP activity between a sample comprising a compound of the present invention, or composition thereof, and MT1-MMP, and an equivalent sample comprising MT1-MMP, in the absence of said compound, or composition thereof.

### 3. Description of Exemplary Embodiments

As described above, in certain embodiments, the present invention provides a compound of formula I:

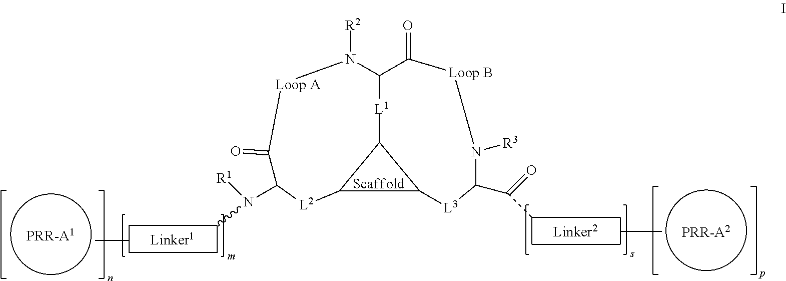

or a pharmaceutically acceptable salt thereof, wherein:
each of L¹, L², and L³ is independently a covalent bond or a C₁₋₈ bivalent hydrocarbon chain wherein one, two or three methylene units of the chain are optionally and independently replaced by —S—, —N(R)—, —O—, —C(O)—, —OC(O)—, —C(O)O—, —C(O)N(R)—, —N(R)C(O)—, —S(O)—, —S(O)₂— or —N(R)CH₂C(O)—;
each of R is independently hydrogen or C₁₋₄ alkyl;
each of m, n, s, and p is independently 0 or 1;
each of q and r is independently 1, 2, 3, 4, 5, 6, 7, 8, 9, 10, 11, 12, 13, 14 or 15;
R¹ is R or —C(O)R;
each of R⁴ and R⁶ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
each of R^{4′} and R^{6′} is independently hydrogen or methyl;
each of R², R³, R⁵, and R⁷ is independently hydrogen, or C₁₋₄ aliphatic, or:
   an R⁵ group and its adjacent R⁴ group are optionally taken together with their intervening atoms to form a 4-8 membered saturated or partially unsaturated monocyclic heterocyclic ring having 1-2 heteroatoms independently selected from nitrogen, oxygen, or sulfur; or
   an R⁷ group and its adjacent R⁶ group are optionally taken together with their intervening atoms to form a 4-8 membered saturated or partially unsaturated monocyclic heterocyclic ring having 1-2 heteroatoms independently selected from nitrogen, oxygen, or sulfur;
Scaffold is a trivalent group that connects and orients a cyclic peptide;
Loop A is a bivalent natural or unnatural amino acid residue or peptide attached to the amino acid residue linked to L² and the amino acid residue linked to L¹, wherein Loop A comprises

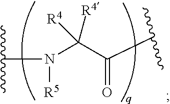

Loop B is a bivalent natural or unnatural amino acid residue or peptide attached to the amino acid residue linked to L¹ and the amino acid residue linked to L³, wherein Loop B comprises

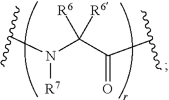

[symbol] indicates the site of attachment to the N-terminus of the Bicycle;
[symbol] indicates the site of attachment to the C-terminus of the Bicycle;
PRR-A¹ is a pattern recognition receptor agonist;
PRR-A² is a pattern recognition receptor agonist;
Linker¹ is hydrogen or a bivalent moiety that connects the N-terminus of the Bicycle with PRR-A¹, wherein when n is 0, Linker¹ is hydrogen;
Linker² is —NH₂ or a bivalent moiety that connects the C-terminus of the Bicycle with PRR-A², wherein when p is 0, Linker² is —NH₂; and
Ring A is selected from the group consisting of 18-crown-6, 1,7,13-triaza-18-crown-6, and a 3-12-membered saturated, partially unsaturated, bridged bicyclic, bridged tricyclic, propellane, or aromatic ring optionally substituted with 0-3 oxo, methyl, ethyl or spiroethylene groups and having 0-6 heteroatoms independently selected from nitrogen, oxygen, or sulfur.

As defined above and described herein, each of L¹, L², and L³ is a covalent bond or a C₁₋₈ bivalent hydrocarbon chain wherein one, two or three methylene units of the chain are optionally and independently replaced by —S—, —N(R)—, —O—, —C(O)—, —OC(O)—, —C(O)O—, —C(O)N(R)—, —N(R)C(O)—, —S(O)—, —S(O)₂— or —N(R)CH₂C(O)—.

In some embodiments, each of L¹, L², and L³ is a covalent bond. In some embodiments, each of L¹, L², and L³ is —CH₂S—. In some embodiments, each of L¹, L², and L³ is —CH₂NH—. In some embodiments, each of L¹, L², and L³ is —CH₂O—. In some embodiments, each of L¹, L², and L³ is —CH₂CH₂O—. In some embodiments, each of L¹, L², and L³ is —CH₂CH₂CH₂CH₂NH—. In some embodiments, each of L¹, L², and L³ is —CH₂N(CH₃)—. In some embodiments, each of L¹, L², and L³ is —CH₂CH₂CH₂CH₂N(CH₃)—.

In some embodiments, L¹ is a covalent bond. In some embodiments, L¹ is —CH₂S—. In some embodiments, L¹ is —CH₂O—. In some embodiments, L¹ is —CH₂CH₂O—. In some embodiments, L¹ is —CH₂NH—. In some embodiments, L¹ is —CH₂CH₂CH₂CH₂NH—. In some embodiments, L¹ is —CH₂N(CH₃)—. In some embodiments, L¹ is —CH₂CH₂CH₂CH₂N(CH₃)—. In some embodiments, L¹ is —CH₂SCH₂—. In some embodiments, L¹ is —CH₂OCH₂—. In some embodiments, L¹ is —CH₂CH₂OCH₂—. In some embodiments, L¹ is —CH₂NHCH₂—. In some embodiments, L¹ is —CH₂N(CH₃)CH₂—. In some embodiments, L¹ is —CH₂CH₂CH₂CH₂NHCH₂—. In some embodiments, L¹ is —CH₂CH₂CH₂CH₂N(CH₃)CH₂—. In some embodiments, L¹ is —CH₂SCH₂C(O)NH—. In some embodiments, L¹ is —CH₂OCH₂C(O)NH—. In some embodiments, L¹ is —CH₂CH₂OCH₂C(O)NH—. In some embodiments, L¹ is —CH₂NHCH₂C(O)NH—. In some embodiments, L¹ is —CH₂N(CH₃)CH₂C(O)NH—. In some embodiments, L¹ is —CH₂CH₂CH₂CH₂NHCH₂C(O)NH—. In some embodiments, L¹ is CH₂CH₂CH₂CH₂N(CH₃)CH₂C(O)NH—. In some embodiments, L¹ is —CH₂SCH₂C(O)—. In some embodiments, L¹ is —CH₂OCH₂C(O)—. In some embodiments, L¹ is —CH₂CH₂OCH₂C(O)—. In some embodiments, L¹ is —CH₂NHCH₂C(O)—. In some embodiments, L¹ is —CH₂N(CH₃)CH₂C(O)—. In some embodiments, L¹ is —CH₂CH₂CH₂CH₂NHCH₂C(O)—. In some embodiments, L¹ is —CH₂CH₂CH₂CH₂N(CH₃)CH₂C(O)—. In some embodiments, L¹ is —CH₂SCH₂CH₂C(O)NH—. In some embodiments, L¹ is —CH₂OCH₂CH₂C(O)NH—. In some embodiments, L¹ is —CH₂CH₂OCH₂CH₂C(O)NH—. In some embodiments, L¹ is —CH₂NHCH₂CH₂C(O)NH—. In some embodiments, L¹ is —CH₂N(CH₃)CH₂CH₂C(O)NH—. In some embodiments, L¹ is —CH₂CH₂CH₂CH₂NHCH₂CH₂C(O)NH—. In some embodiments, L¹ is —CH₂CH₂CH₂CH₂N(CH₃)CH₂CH₂C(O)NH—. In some embodiments, L¹ is —CH₂SCH₂CH₂C(O)—. In some embodiments, L¹ is —CH₂OCH₂CH₂C(O)—. In some embodiments, L¹ is —CH₂CH₂OCH₂CH₂C(O)—. In some embodiments, L¹ is —CH₂NHCH₂CH₂C(O)—. In some embodiments, L¹ is —CH₂N(CH₃)CH₂CH₂C(O)—. In some embodiments, L¹ is —CH₂CH₂CH₂CH₂NHCH₂CH₂C(O)—. In some embodiments, L¹ is CH₂CH₂CH₂CH₂N(CH₃)CH₂CH₂C(O)—. In some embodiments, L¹ is selected from those depicted in Table 1, below.

In some embodiments, L² is a covalent bond. In some embodiments, L² is —CH₂S—. In some embodiments, L² is —CH₂O—. In some embodiments, L² is —CH₂CH₂O—. In some embodiments, L² is —CH₂NH—. In some embodiments, L² is —CH₂CH₂CH₂CH₂NH—. In some embodiments, L² is —CH₂N(CH₃)—. In some embodiments, L² is —CH₂CH₂CH₂CH₂N(CH₃)—. In some embodiments, L² is —CH₂SCH₂—. In some embodiments, L² is —CH₂OCH₂—. In some embodiments, L² is —CH₂CH₂OCH₂—. In some embodiments, L² is —CH₂NHCH₂—. In some embodiments, L² is —CH₂N(CH₃)CH₂—. In some embodiments, L² is —CH₂CH₂CH₂CH₂NHCH₂—. In some embodiments, L² is —CH₂CH₂CH₂CH₂N(CH₃)CH₂—. In some embodiments, L² is —CH₂SCH₂C(O)NH—. In some embodiments, L² is —CH₂OCH₂C(O)NH—. In some embodiments, L² is —CH₂CH₂OCH₂C(O)NH—. In some embodiments, L² is —CH₂NHCH₂C(O)NH—. In some embodiments, L² is —CH₂N(CH₃)CH₂C(O)NH—. In some embodiments, L² is —CH₂CH₂CH₂CH₂NHCH₂C(O)NH—. In some embodiments, L² is CH₂CH₂CH₂CH₂N(CH₃)CH₂C(O)NH—. In some embodiments, L² is —CH₂SCH₂C(O)—. In some embodiments, L² is —CH₂OCH₂C(O)—. In some embodiments, L² is —CH₂CH₂OCH₂C(O)—. In some embodiments, L² is —CH₂NHCH₂C(O)—. In some embodiments, L² is —CH₂N(CH₃)CH₂C(O)—. In some embodiments, L² is —CH₂CH₂CH₂CH₂NHCH₂C(O)—. In some embodiments, L² is —CH₂CH₂CH₂CH₂N(CH₃)CH₂C(O)—. In some embodiments, L² is —CH₂SCH₂CH₂C(O)NH—. In some embodiments, L² is —CH₂OCH₂CH₂C(O)NH—. In some embodiments, L² is —CH₂CH₂OCH₂CH₂C(O)NH—. In some embodiments, L² is —CH₂NHCH₂CH₂C(O)NH—. In some embodiments, L² is —CH₂N(CH₃)CH₂CH₂C(O)NH—. In some embodiments, L² is —CH₂CH₂CH₂CH₂NHCH₂CH₂C(O)NH—. In some embodiments, L² is —CH₂CH₂CH₂CH₂N(CH₃)CH₂CH₂C(O)NH—. In some embodiments, L² is —CH₂SCH₂CH₂C(O)—. In some embodiments, L² is —CH₂OCH₂CH₂C(O)—. In some embodiments, L² is —CH₂CH₂OCH₂CH₂C(O)—. In some embodiments, L² is —CH₂NHCH₂CH₂C(O)—. In some embodiments, L² is —CH₂N(CH₃)CH₂CH₂C(O)—. In some embodiments, L² is —CH₂CH₂CH₂CH₂NHCH₂CH₂C(O)—. In some embodiments, L² is CH₂CH₂CH₂CH₂N(CH₃)CH₂CH₂C(O)—. In some embodiments, L² is selected from those depicted in Table 1, below.

In some embodiments, L³ is a covalent bond. In some embodiments, L³ is —CH₂S—. In some embodiments, L³ is —CH₂O—. In some embodiments, L³ is —CH₂CH₂O—. In some embodiments, L³ is —CH₂NH—. In some embodiments, L³ is —CH₂CH₂CH₂CH₂NH—. In some embodiments, L³ is —CH₂N(CH₃)—. In some embodiments, L³ is —CH₂CH₂CH₂CH₂N(CH₃)—. In some embodiments, L³ is —CH₂SCH₂—. In some embodiments, L³ is —CH₂OCH₂—. In some embodiments, L³ is —CH₂CH₂OCH₂—. In some embodiments, L³ is —CH₂NHCH₂—. In some embodiments, L³ is —CH₂N(CH₃)CH₂—. In some embodiments, L³ is —CH₂CH₂CH₂CH₂NHCH₂—. In some embodiments, L³ is —CH₂CH₂CH₂CH₂N(CH₃)CH₂—. In some embodiments, L³ is —CH₂SCH₂C(O)NH—. In some embodiments, L³ is —CH₂OCH₂C(O)NH—. In some embodiments, L³ is —CH₂CH₂OCH₂C(O)NH—. In some embodiments, L³ is —CH₂NHCH₂C(O)NH—. In some embodiments, L³ is —CH₂N(CH₃)CH₂C(O)NH—. In some embodiments, L³ is —CH₂CH₂CH₂CH₂NHCH₂C(O)NH—. In some embodiments, L³ is CH₂CH₂CH₂CH₂N(CH₃)CH₂C(O)NH—. In some embodiments, L³ is —CH₂SCH₂C(O)—. In some embodiments, L³ is —CH₂OCH₂C(O)—. In some embodiments, L³ is —CH₂CH₂OCH₂C(O)—. In some embodiments, L³ is —CH₂NHCH₂C(O)—. In some embodiments, L³ is —CH₂N(CH₃)CH₂C(O)—. In some embodiments, L³ is —CH₂CH₂CH₂CH₂NHCH₂C(O)—. In some embodiments, L³ is —CH₂CH₂CH₂CH₂N(CH₃)CH₂C(O)—. In some embodiments, L³ is —CH₂SCH₂CH₂C(O)NH—. In some embodiments, L³ is —CH₂OCH₂CH₂C(O)NH—. In some embodiments, L³ is —CH₂CH₂OCH₂CH₂C(O)NH—. In some embodiments, L³ is —CH₂NHCH₂CH₂C(O)NH—. In some embodiments, L³ is —CH₂N(CH₃)CH₂CH₂C(O)NH—. In some embodiments, L³ is —CH₂CH₂CH₂CH₂NHCH₂CH₂C(O)NH—. In some embodiments, L³ is —CH₂CH₂CH₂CH₂N(CH₃)CH₂CH₂C(O)NH—. In some embodiments, L³ is —CH₂SCH₂CH₂C(O)—. In some embodiments, L³ is —CH₂OCH₂CH₂C(O)—. In some embodiments, L³ is —CH₂CH₂OCH₂CH₂C(O)—. In some embodiments, L³ is —CH₂NHCH₂CH₂C(O)—. In some embodiments, L³ is —CH₂N(CH₃)CH₂CH₂C(O)—. In some embodiments, L³ is —CH₂CH₂CH₂CH₂NHCH₂CH₂C(O)—. In some embodiments, L³ is CH₂CH₂CH₂CH₂N(CH₃)CH₂CH₂C(O)—. In some embodiments, L³ is selected from those depicted in Table 1, below.

As defined above and described herein, each of R is independently hydrogen or C₁₋₄ alkyl.

In some embodiments, R is hydrogen. In some embodiments, R is C₁₋₄ alkyl.

In some embodiments, R is methyl. In some embodiments, R is ethyl. In some embodiments, R is n-propyl. In some embodiments, R is isopropyl. In some embodiments, R is n-butyl. In some embodiments, R is isobutyl. In some embodiments, R is tert-butyl.

In some embodiments, R is selected from those depicted in Table 1, below.

As defined above and described herein, each of m, n, s, and p is independently 0 or 1.

In some embodiments, m is 0. In some embodiments, m is 1. In some embodiments, m is selected from those depicted in Table 1, below.

In some embodiments, n is 0. In some embodiments, n is 1. In some embodiments, n is selected from those depicted in Table 1, below.

In some embodiments, s is 0. In some embodiments, s is 1. In some embodiments, s is selected from those depicted in Table 1, below.

In some embodiments, p is 0. In some embodiments, p is 1. In some embodiments, p is selected from those depicted in Table 1, below.

As defined above and described herein, each of q and r is independently 1, 2, 3, 4, 5, 6, 7, 8, 9, 10, 11, 12, 13, 14 or 15.

In some embodiments, q is 1. In some embodiments, q is 2. In some embodiments, q is 3. In some embodiments, q is 4. In some embodiments, q is 5. In some embodiments, q is 6. In some embodiments, q is 7. In some embodiments, q is 8. In some embodiments, q is 9. In some embodiments, q is 10. In some embodiments, q is 11. In some embodiments, q is 12. In some embodiments, q is 13. In some embodiments, q is 14. In some embodiments, q is 15. In some embodiments, q is selected from those depicted in Table 1, below.

In some embodiments, r is 1. In some embodiments, r is 2. In some embodiments, r is 3. In some embodiments, r is 4. In some embodiments, r is 5. In some embodiments, r is 6. In some embodiments, r is 7. In some embodiments, r is 8. In some embodiments, r is 9. In some embodiments, r is 10. In some embodiments, r is 11. In some embodiments, r is 12. In some embodiments, r is 13. In some embodiments, r is 14. In some embodiments, r is 15. In some embodiments, r is selected from those depicted in Table 1, below.

As defined above and described herein, R¹ is R or —C(O)R.

In some embodiments, R¹ is R. In some embodiments, R¹ is —C(O)R.

In some embodiments, R¹ is hydrogen. In some embodiments, R¹ is methyl. In some embodiments, R¹ is ethyl. In some embodiments, R¹ is n-propyl. In some embodiments, R¹ is isopropyl. In some embodiments, R¹ is n-butyl. In some embodiments, R¹ is isobutyl. In some embodiments, R¹ is tert-butyl.

In some embodiments, R¹ is —C(O)CH₃. In some embodiments, R¹ is —C(O)CH₂CH₃. In some embodiments, R¹ is —C(O)CH₂CH₂CH₃. In some embodiments, R¹ is —C(O)CH(CH₃)₂. In some embodiments, R¹ is —C(O)CH₂CH₂CH₂CH₃. In some embodiments, R¹ is —C(O)CH₂CH(CH₃)₂. In some embodiments, R¹ is —C(O)C(CH₃)₃. In some embodiments, R¹ is selected from those depicted in Table 1, below.

As defined above and described herein, each of R⁴ and R⁶ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur.

In some embodiments, R⁴ is hydrogen. In some embodiments, R⁴ is an optionally substituted C₁₋₆ aliphatic. In some embodiments, R⁴ is an optionally substituted 3-8 membered saturated or partially unsaturated monocyclic carbocyclic ring. In some embodiments, R⁴ is an optionally substituted phenyl. In some embodiments, R⁴ is an optionally substituted 8-10 membered bicyclic aromatic carbocyclic ring. In some embodiments, R⁴ is an optionally substituted 4-8 membered saturated or partially unsaturated monocyclic heterocyclic ring having 1-2 heteroatoms independently selected from nitrogen, oxygen, or sulfur. In some embodiments, R⁴ is an optionally substituted 5-6 membered monocyclic heteroaromatic ring having 1-4 heteroatoms independently selected from nitrogen, oxygen, or sulfur. In some embodiments, R⁴ is an optionally substituted 8-10 membered bicyclic heteroaromatic ring having 1-5 heteroatoms independently selected from nitrogen, oxygen, or sulfur.

In some embodiments, R⁴ is methyl. In some embodiments, R⁴ is

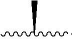

In some embodiments, R⁴ is

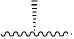

In some embodiments, R⁴ is

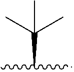

In some embodiments, R⁴ is

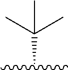

In some embodiments, R⁴ is

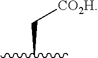

In some embodiments, R⁴ is

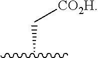

In some embodiments, R⁴ is

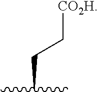

In some embodiments, R⁴ is

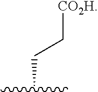

In some embodiments, R⁴ is

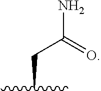

In some embodiments, R⁴ is

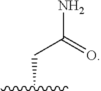

In some embodiments, R⁴ is

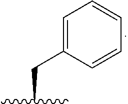

In some embodiments, R⁴ is

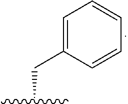

In some embodiments, R⁴ is

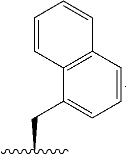

In some embodiments, R⁴ is

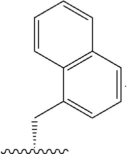

In some embodiments, R⁴ is

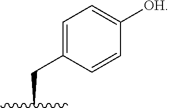

In some embodiments, R⁴ is

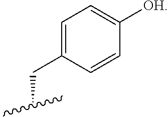

In some embodiments, R⁴ is selected from those depicted in Table 1, below.

In some embodiments, R⁶ is hydrogen. In some embodiments, R⁶ is an optionally substituted C₁₋₆ aliphatic. In some embodiments, R⁶ is an optionally substituted 3-8 membered saturated or partially unsaturated monocyclic carbocyclic ring. In some embodiments, R⁶ is an optionally substituted phenyl. In some embodiments, R⁶ is an optionally substituted 8-10 membered bicyclic aromatic carbocyclic ring. In some embodiments, R⁶ is an optionally substituted 4-8 membered saturated or partially unsaturated monocyclic heterocyclic ring having 1-2 heteroatoms independently selected from nitrogen, oxygen, or sulfur. In some embodiments, R⁶ is an optionally substituted 5-6 membered monocyclic heteroaromatic ring having 1-4 heteroatoms independently selected from nitrogen, oxygen, or sulfur. In some embodiments, R⁶ is an optionally substituted 8-10 membered bicyclic heteroaromatic ring having 1-5 heteroatoms independently selected from nitrogen, oxygen, or sulfur.

In some embodiments, R⁶ is methyl. In some embodiments, R⁶ is

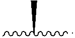

In some embodiments, R⁶ is

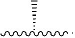

In some embodiments, R⁶ is

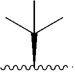

In some embodiments, R⁶ is

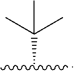

In some embodiments, R⁶ is

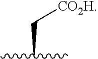

In some embodiments, R⁶ is

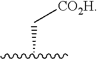

In some embodiments, R⁶ is

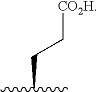

In some embodiments, R⁶ is

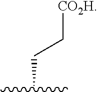

In some embodiments, R⁶ is

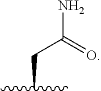

In some embodiments, R⁶ is

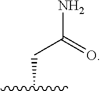

In some embodiments, R⁶ is

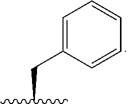

In some embodiments, R⁶ is

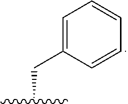

In some embodiments, R⁶ is

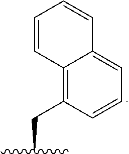

In some embodiments, R⁶ is

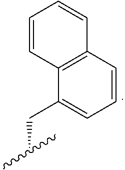

In some embodiments, R⁶ is

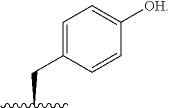

In some embodiments, R⁶ is

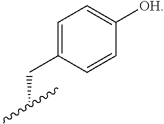

In some embodiments, R⁶ is selected from those depicted in Table 1, below.

As defined above and described herein, each of R^{4′} and R^{6′} is independently hydrogen or methyl.

In some embodiments, R^{4′} is hydrogen. In some embodiments, R^{4′} is methyl.

In some embodiments, R^{4′} is selected from those depicted in Table 1, below.

In some embodiments, R^{6′} is hydrogen. In some embodiments, R^{6′} is methyl.

In some embodiments, R^{6′} is selected from those depicted in Table 1, below.

As defined above and described herein, each of R², R³, R⁵, and R⁷ is independently hydrogen, or C₁₋₄ aliphatic, or: an R⁵ group and its adjacent R⁴ group are optionally taken together with their intervening atoms to form a 4-8 membered saturated or partially unsaturated monocyclic heterocyclic ring having 1-2 heteroatoms independently selected from nitrogen, oxygen, or sulfur; or an R⁷ group and its adjacent R⁶ group are optionally taken together with their intervening atoms to form a 4-8 membered saturated or partially unsaturated monocyclic heterocyclic ring having 1-2 heteroatoms independently selected from nitrogen, oxygen, or sulfur.

In some embodiments, R² is hydrogen. In some embodiments, R² is C₁₋₄ aliphatic. In some embodiments, R² is methyl. In some embodiments, R² is ethyl. In some embodiments, R² is n-propyl. In some embodiments, R² is isopropyl. In some embodiments, R² is n-butyl. In some embodiments, R² is isobutyl. In some embodiments, R² is tert-butyl.

In some embodiments, R² is selected from those depicted in Table 1, below.

In some embodiments, R³ is hydrogen. In some embodiments, R³ is C₁₋₄ aliphatic. In some embodiments, R³ is methyl. In some embodiments, R³ is ethyl. In some embodiments, R³ is n-propyl. In some embodiments, R³ is isopropyl. In some embodiments, R³ is n-butyl. In some embodiments, R³ is isobutyl. In some embodiments, R³ is tert-butyl.

In some embodiments, R³ is selected from those depicted in Table 1, below.

In some embodiments, R⁵ is hydrogen. In some embodiments, R⁵ is C₁₋₄ aliphatic. In some embodiments, R⁵ is methyl. In some embodiments, R⁵ is ethyl. In some embodiments, R⁵ is n-propyl. In some embodiments, R⁵ is isopropyl. In some embodiments, R⁵ is n-butyl. In some embodiments, R⁵ is isobutyl. In some embodiments, R⁵ is tert-butyl.

In some embodiments, an R⁵ group and its adjacent R⁴ group are taken together with their intervening atoms to form

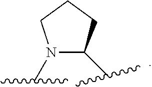 [SMILES: *[C@@H]1CCCN1*]

In some embodiments, an R⁵ group and its adjacent R⁴ group are taken together with their intervening atoms to form

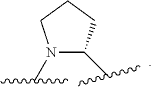 [SMILES: *[C@H]1CCCN1*]

In some embodiments, R⁵ is selected from those depicted in Table 1, below.

In some embodiments, R⁷ is hydrogen. In some embodiments, R⁷ is C₁₋₄ aliphatic. In some embodiments, R⁷ is methyl. In some embodiments, R⁷ is ethyl. In some embodiments, R⁷ is n-propyl. In some embodiments, R⁷ is isopropyl. In some embodiments, R⁷ is n-butyl. In some embodiments, R⁷ is isobutyl. In some embodiments, R⁷ is tert-butyl.

In some embodiments, an R⁷ group and its adjacent R⁶ group are taken together with their intervening atoms to form

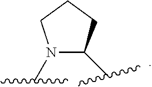 [SMILES: *[C@@H]1CCCN1*]

In some embodiments, an R⁷ group and its adjacent R⁶ group are taken together with their intervening atoms to form

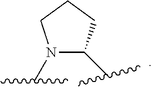 [SMILES: *[C@H]1CCCN1*]

In some embodiments, R⁷ is selected from those depicted in Table 1, below.

As defined above and described herein, Scaffold is a trivalent group that connects and orients a cyclic peptide.

In some embodiments, Scaffold is

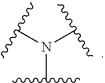 [SMILES: *N(*)*]

In some embodiments, Scaffold is

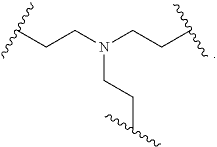 [SMILES: *CCN(CC*)CC*]

In some embodiments, Scaffold is

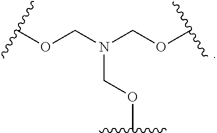 [SMILES: *OCN(CO*)CO*]

In some embodiments, Scaffold is

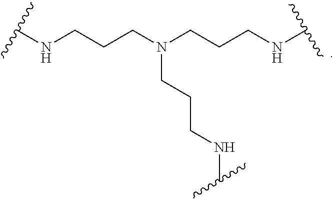 [SMILES: *NCCCN(CCCN*)CCCN*]

In some embodiments, Scaffold is

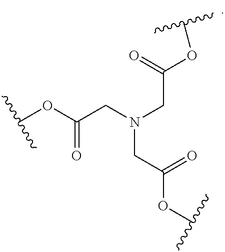 [SMILES: *OC(=O)CN(CC(=O)O*)CC(=O)O*]

In some embodiments, Scaffold is

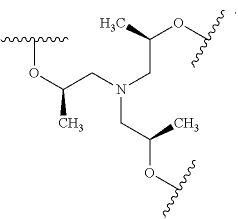 [SMILES: *O[C@H](C)CN(C[C@@H](C)O*)C[C@@H](C)O*]

In some embodiments, Scaffold is

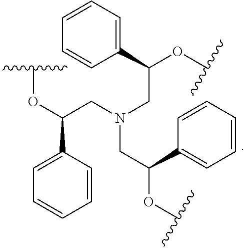 [SMILES: *O[C@@H](CN(C[C@H](O*)c1ccccc1)C[C@H](O*)c1ccccc1)c1ccccc1]

In some embodiments, Scaffold is

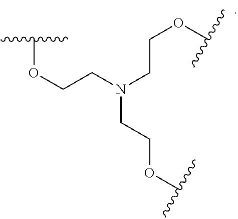 [SMILES: *OCCN(CCO*)CCO*]

In some embodiments, Scaffold is

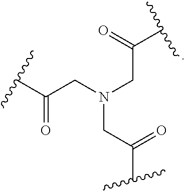 [SMILES: *C(=O)CN(CC(*)=O)CC(*)=O]

In some embodiments, Scaffold is

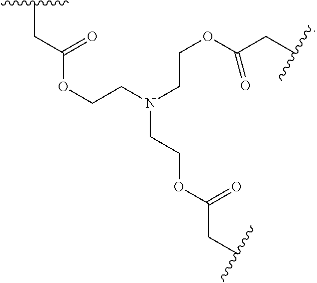 [SMILES: *CC(=O)OCCN(CCOC(=O)C*)CCOC(=O)C*]

In some embodiments, Scaffold is

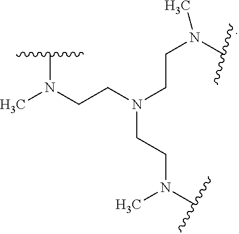 [SMILES: *N(C)CCN(CCN(*)C)CCN(*)C]

In some embodiments, Scaffold is

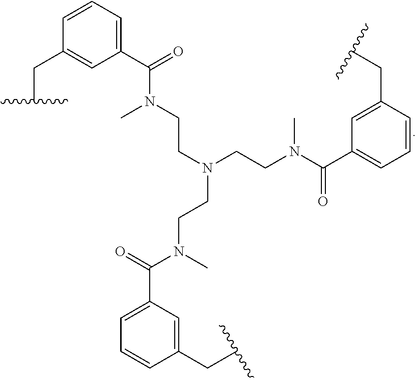 [SMILES: *Cc1cccc(C(=O)N(C)CCN(CCN(C)C(=O)c2cccc(C*)c2)CCN(C)C(=O)c2cccc(C*)c2)c1]

In some embodiments, Scaffold is

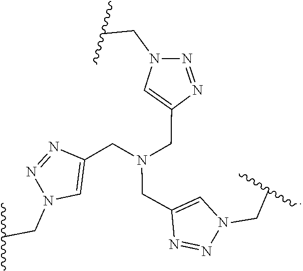

In some embodiments, Scaffold is

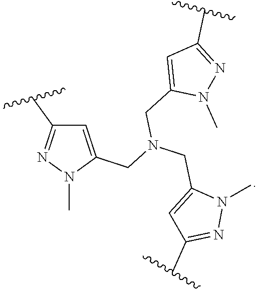 [SMILES: *c1cc(CN(Cc2cc(*)nn2C)Cc2cc(*)nn2C)n(C)n1]

In some embodiments, Scaffold is

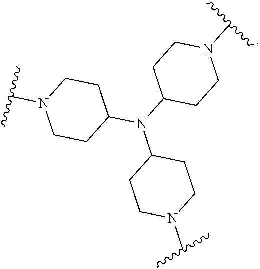 [SMILES: *N1CCC(N(C2CCN(*)CC2)C2CCN(*)CC2)CC1]

In some embodiments, Scaffold is

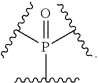 [SMILES: *P(*)(*)=O]

In some embodiments, Scaffold is

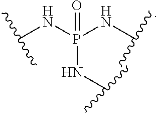 [SMILES: *NP(=O)(N*)N*]

In some embodiments, Scaffold is

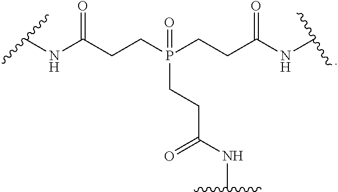 [SMILES: *NC(=O)CCP(=O)(CCC(=O)N*)CCC(=O)N*]

In some embodiments, Scaffold is

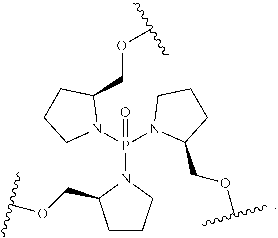 [SMILES: *OC[C@@H]1CCCN1P(=O)(N1CCC[C@H]1CO*)N1CCC[C@H]1CO*]

In some embodiments, Scaffold is

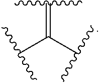 [SMILES: *C(*)=*]

In some embodiments, Scaffold is

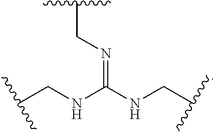 [SMILES: *CN=C(NC*)NC*]

In some embodiments, Scaffold is

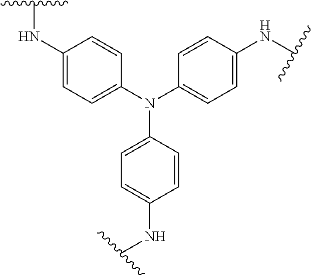 [SMILES: *Nc1ccc(N(c2ccc(N*)cc2)c2ccc(N*)cc2)cc1]

In some embodiments, Scaffold is

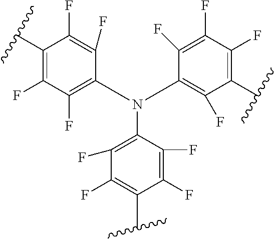 [SMILES: *c1c(F)c(F)c(N(c2c(F)c(*)c(F)c(F)c2F)c2c(F)c(F)c(*)c(F)c2F)c(F)c1F]

In some embodiments, Scaffold is

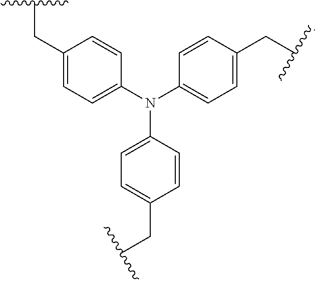 [SMILES: *Cc1ccc(N(c2ccc(C*)cc2)c2ccc(C*)cc2)cc1]

In some embodiments, Scaffold is

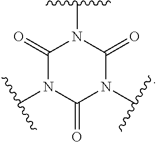 [SMILES: *n1c(=O)n(*)c(=O)n(*)c1=O]

In some embodiments, Scaffold is

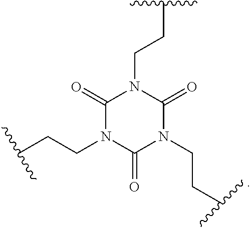 [SMILES: *CCn1c(=O)n(CC*)c(=O)n(CC*)c1=O]

In some embodiments, Scaffold is

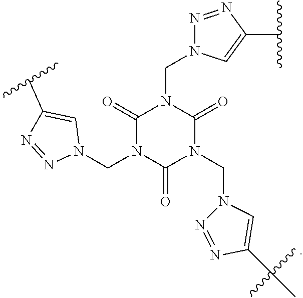 [SMILES: *c1cn(Cn2c(=O)n(Cn3cc(*)nn3)c(=O)n(Cn3cc(*)nn3)c2=O)nn1]

In some embodiments, Scaffold is

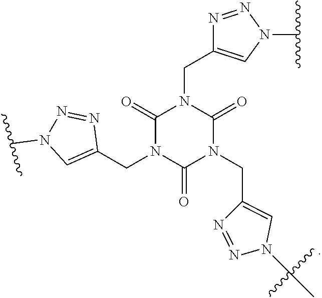 [SMILES: *n1cc(Cn2c(=O)n(Cc3cn(*)nn3)c(=O)n(Cc3cn(*)nn3)c2=O)nn1]

In some embodiments, Scaffold is

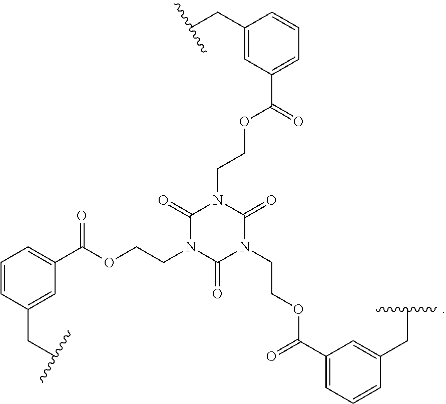 [SMILES: *Cc1cccc(C(=O)OCCn2c(=O)n(CCOC(=O)c3cccc(C*)c3)c(=O)n(CCOC(=O)c3cccc(C*)c3)c2=O)c1]

In some embodiments, Scaffold is

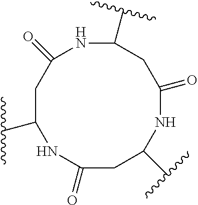 [SMILES: *C1CC(=O)NC(*)CC(=O)NC(*)CC(=O)N1]

In some embodiments, Scaffold is

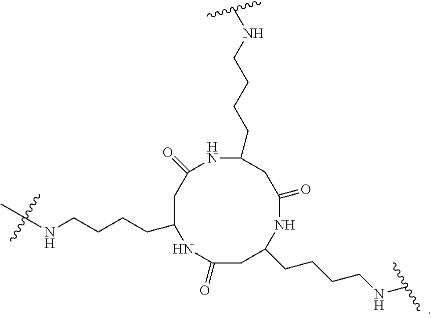 [SMILES: *NCCCCC1CC(=O)NC(CCCCN*)CC(=O)NC(CCCCN*)CC(=O)N1]

In some embodiments, Scaffold is

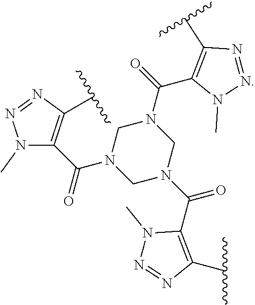 [SMILES: *c1nnn(C)c1C(=O)N1CN(C(=O)c2c(*)nnn2C)CN(C(=O)c2c(*)nnn2C)C1]

In some embodiments, Scaffold is

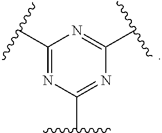 [SMILES: *c1nc(*)nc(*)n1]

In some embodiments, Scaffold is

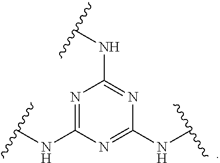 [SMILES: *Nc1nc(N*)nc(N*)n1]

In some embodiments, Scaffold is

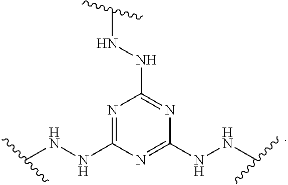 [SMILES: *NNc1nc(NN*)nc(NN*)n1]

In some embodiments, Scaffold is

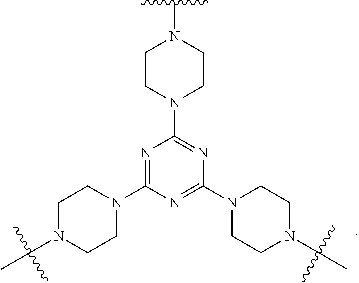 [SMILES: *N1CCN(c2nc(N3CCN(*)CC3)nc(N3CCN(*)CC3)n2)CC1]

In some embodiments, Scaffold is

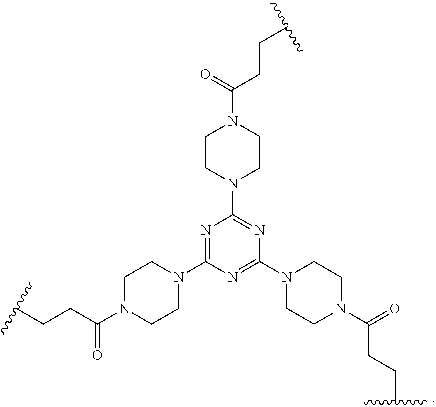 [SMILES: *CCC(=O)N1CCN(c2nc(N3CCN(C(=O)CC*)CC3)nc(N3CCN(C(=O)CC*)CC3)n2)CC1]

In some embodiments, Scaffold is

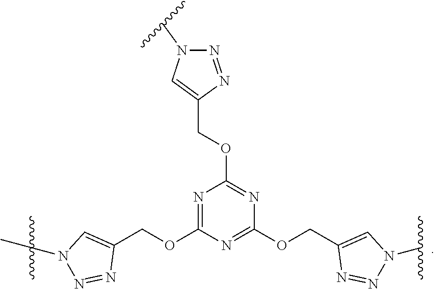 [SMILES: *n1cc(COc2nc(OCc3cn(*)nn3)nc(OCc3cn(*)nn3)n2)nn1]

In some embodiments, Scaffold is

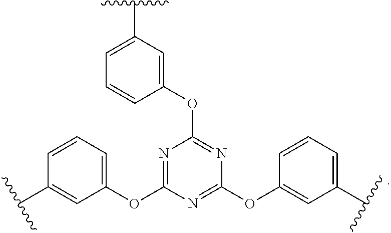 [SMILES: *c1cccc(Oc2nc(Oc3cccc(*)c3)nc(Oc3cccc(*)c3)n2)c1]

In some embodiments, Scaffold is

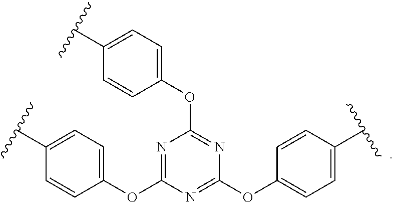 [SMILES: *c1ccc(Oc2nc(Oc3ccc(*)cc3)nc(Oc3ccc(*)cc3)n2)cc1]

In some embodiments, Scaffold is

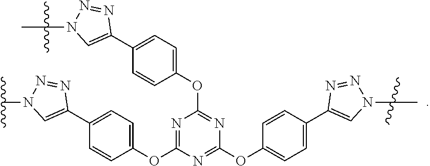 [SMILES: *n1cc(-c2ccc(Oc3nc(Oc4ccc(-c5cn(*)nn5)cc4)nc(Oc4ccc(-c5cn(*)nn5)cc4)n3)cc2)nn1]

In some embodiments, Scaffold is

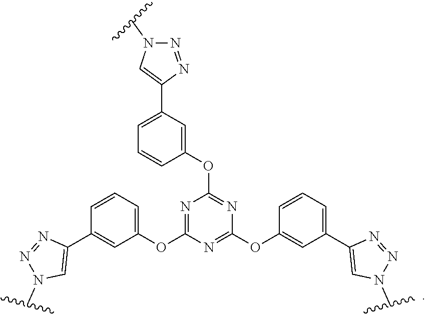 [SMILES: *n1cc(-c2cccc(Oc3nc(Oc4cccc(-c5cn(*)nn5)c4)nc(Oc4cccc(-c5cn(*)nn5)c4)n3)c2)nn1]

In some embodiments, Scaffold is

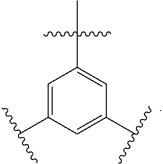 [SMILES: *c1cc(*)cc(*)c1]

In some embodiments, Scaffold is

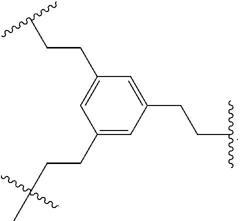 [SMILES: *CCc1cc(CC*)cc(CC*)c1]

In some embodiments, Scaffold is

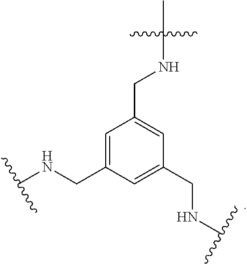 [SMILES: *NCc1cc(CN*)cc(CN*)c1]

In some embodiments, Scaffold is

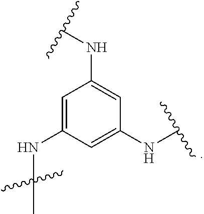 [SMILES: *Nc1cc(N*)cc(N*)c1]

In some embodiments, Scaffold is

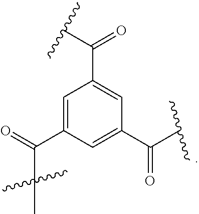 [SMILES: *C(=O)c1cc(C(*)=O)cc(C(*)=O)c1]

In some embodiments, Scaffold is

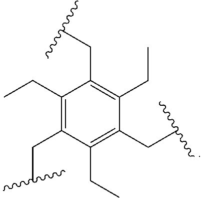 [SMILES: *Cc1c(CC)c(C*)c(CC)c(C*)c1CC]

In some embodiments, Scaffold is

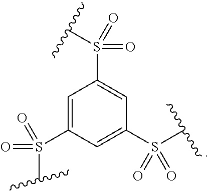 [SMILES: *S(=O)(=O)c1cc(S(*)(=O)=O)cc(S(*)(=O)=O)c1]

In some embodiments, Scaffold is

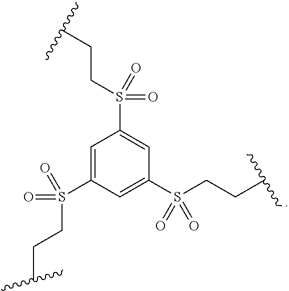 [SMILES: *CCS(=O)(=O)c1cc(S(=O)(=O)CC*)cc(S(=O)(=O)CC*)c1]

In some embodiments, Scaffold is

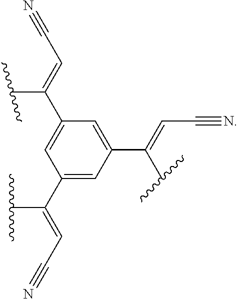 [SMILES: */C(=C\C#N)c1cc(/C(*)=C/C#N)cc(/C(*)=C/C#N)c1]

In some embodiments, Scaffold is

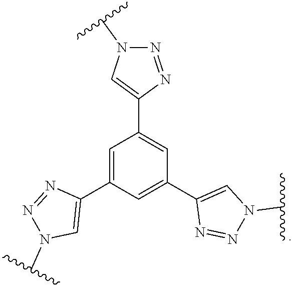 [SMILES: *n1cc(-c2cc(-c3cn(*)nn3)cc(-c3cn(*)nn3)c2)nn1]

In some embodiments, Scaffold is

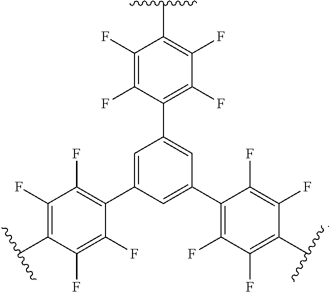 [SMILES: *c1c(F)c(F)c(-c2cc(-c3c(F)c(F)c(*)c(F)c3F)cc(-c3c(F)c(F)c(*)c(F)c3F)c2)c(F)c1F]

In some embodiments, Scaffold is

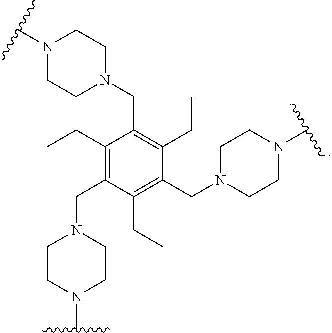

In some embodiments, Scaffold is

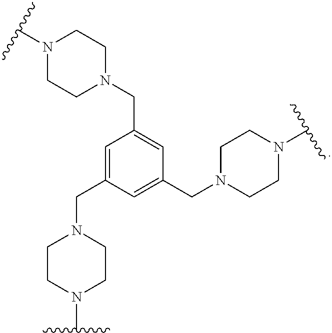

In some embodiments, Scaffold is

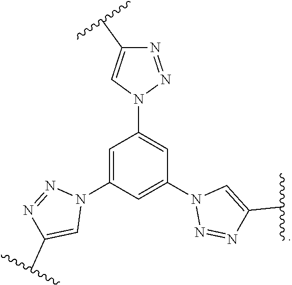 [SMILES: *c1cn(-c2cc(-n3cc(*)nn3)cc(-n3cc(*)nn3)c2)nn1]

In some embodiments, Scaffold is

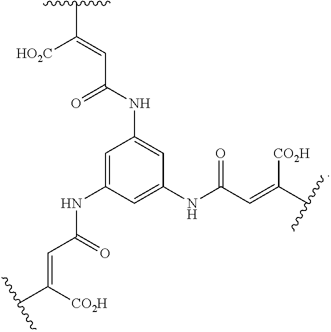 [SMILES: */C(=C/C(=O)Nc1cc(NC(=O)/C=C(/*)C(=O)O)cc(NC(=O)/C=C(/*)C(=O)O)c1)C(=O)O]

In some embodiments, Scaffold is

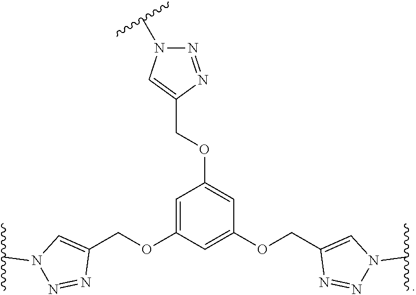 [SMILES: *n1cc(COc2cc(OCc3cn(*)nn3)cc(OCc3cn(*)nn3)c2)nn1]

In some embodiments, Scaffold is

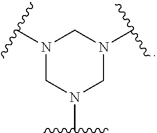 [SMILES: *N1CN(*)CN(*)C1]

In some embodiments, Scaffold is

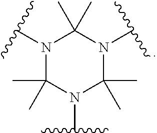

In some embodiments, Scaffold is

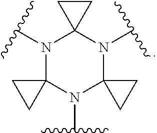 [SMILES: *N1C2(CC2)N(*)C2(CC2)N(*)C12CC2]

In some embodiments, Scaffold is

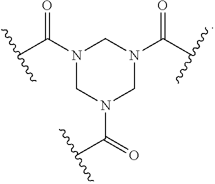 [SMILES: *C(=O)N1CN(C(*)=O)CN(C(*)=O)C1]

In some embodiments, Scaffold is

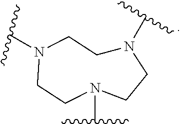 [SMILES: *N1CCN(*)CCN(*)CC1]

In some embodiments, Scaffold is

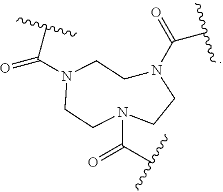 [SMILES: *C(=O)N1CCN(C(*)=O)CCN(C(*)=O)CC1]

In some embodiments, Scaffold is

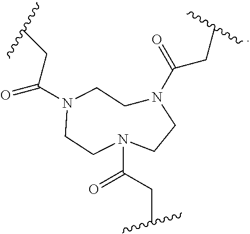 [SMILES: *CC(=O)N1CCN(C(=O)C*)CCN(C(=O)C*)CC1]

In some embodiments, Scaffold is

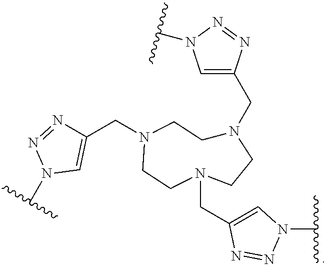 [SMILES: *n1cc(CN2CCN(Cc3cn(*)nn3)CCN(Cc3cn(*)nn3)CC2)nn1]

In some embodiments, Scaffold is

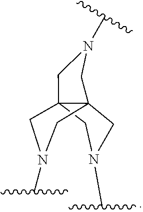 [SMILES: *N1CC23CN(*)CC2(C1)CN(*)C3]

In some embodiments, Scaffold is

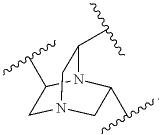 [SMILES: *C1CN2CC(*)N1C(*)C2]

In some embodiments, Scaffold is

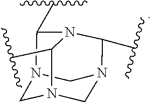 [SMILES: *C1N2CN3CN(C2)C(*)N1C3*]

In some embodiments, Scaffold is

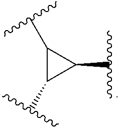 [SMILES: *C1[C@H](*)[C@H]1*]

In some embodiments, Scaffold is

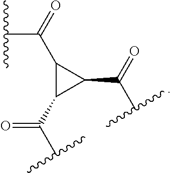 [SMILES: *C(=O)C1[C@H](C(*)=O)[C@H]1C(*)=O]

In some embodiments, Scaffold is

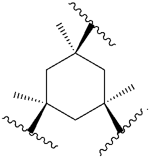

In some embodiments, Scaffold is

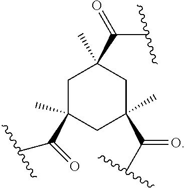 [SMILES: *C(=O)[C@]1(C)C[C@](C)(C(*)=O)C[C@](C)(C(*)=O)C1]

In some embodiments, Scaffold is

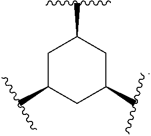 [SMILES: *[C@H]1C[C@@H](*)C[C@@H](*)C1]

In some embodiments, Scaffold is

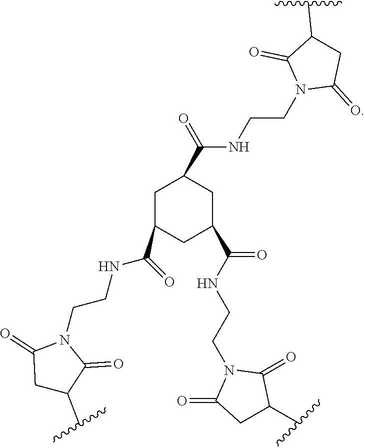 [SMILES: *C1CC(=O)N(CCNC(=O)[C@H]2C[C@@H](C(=O)NCCN3C(=O)CC(*)C3=O)C[C@@H](C(=O)NCCN3C(=O)CC(*)C3=O)C2)C1=O]

In some embodiments, Scaffold is

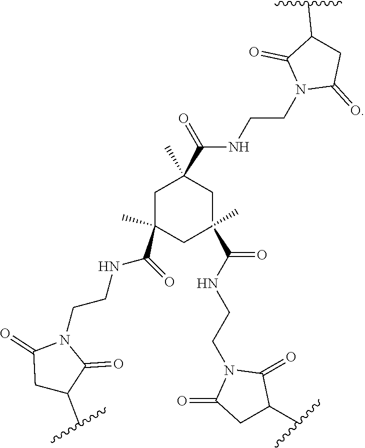

In some embodiments, Scaffold is

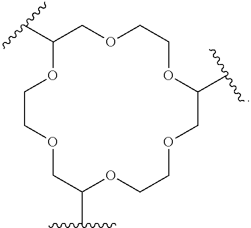 [SMILES: *C1COCCOC(*)COCCOC(*)COCCO1]

In some embodiments, Scaffold is

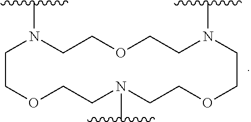 [SMILES: *N1CCOCCN(*)CCOCCN(*)CCOCC1]

In some embodiments, Scaffold is selected from those depicted in Table 1, below

As defined above and described herein, Loop A is a bivalent natural or unnatural amino acid residue or peptide attached to the amino acid residue linked to L² and the amino acid residue linked to L¹, wherein Loop A comprises

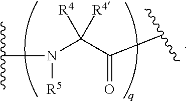

In some embodiments, Loop A is a bivalent natural amino acid residue attached to the amino acid residue linked to L² and the amino acid residue linked to L¹, wherein Loop A comprises

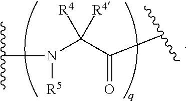

In some embodiments, Loop A is a bivalent unnatural amino acid residue attached to the amino acid residue linked to L² and the amino acid residue linked to L¹, wherein Loop A comprises

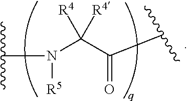

In some embodiments, Loop A is a bivalent peptide attached to the amino acid residue linked to L² and the amino acid residue linked to L¹, wherein Loop A comprises

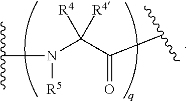

In some embodiments, Loop A is

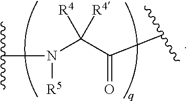

In some embodiments, Loop A is

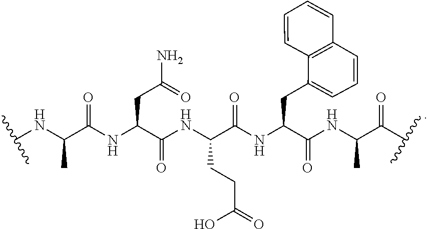 [SMILES: *N[C@H](C)C(=O)N[C@@H](CC(N)=O)C(=O)N[C@@H](CCC(=O)O)C(=O)N[C@@H](Cc1cccc2ccccc12)C(=O)N[C@H](C)C(*)=O]

As defined above and described herein, Loop B is a bivalent natural or unnatural amino acid residue or peptide attached to the amino acid residue linked to L¹ and the amino acid residue linked to L³, wherein Loop B comprises

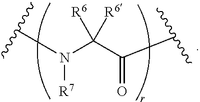

In some embodiments, Loop B is a bivalent natural amino acid residue attached to the amino acid residue linked to L¹ and the amino acid residue linked to L³, wherein Loop B comprises

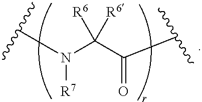

In some embodiments, Loop B is a bivalent unnatural amino acid residue attached to the amino acid residue linked to L¹ and the amino acid residue linked to L³, wherein Loop B comprises

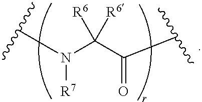

In some embodiments, Loop B is a bivalent peptide attached to the amino acid residue linked to L¹ and the amino acid residue linked to L³, wherein Loop B comprise

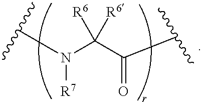

In some embodiments, Loop B is

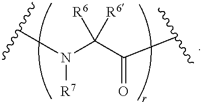

In some embodiments, Loop B is

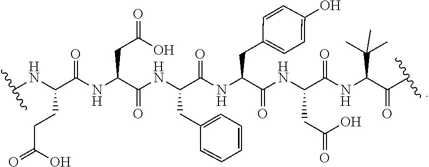 [SMILES: *N[C@@H](CCC(=O)O)C(=O)N[C@@H](CC(=O)O)C(=O)N[C@@H](Cc1ccccc1)C(=O)N[C@@H](Cc1ccc(O)cc1)C(=O)N[C@@H](CC(=O)O)C(=O)N[C@H](C(*)=O)C(C)(C)C]

In some embodiments, Loop A comprises 1-15 amino acid residues and Loop B comprises 1-15 amino acid residues.

In some embodiments, Loop A comprises 5 amino acid residues and Loop B comprises 5 amino acid residues. In some embodiments, Loop A comprises 6 amino acid residues and Loop B comprises 5 amino acid residues. In some embodiments, Loop A comprises 2 amino acid residues and Loop B comprises 7 amino acid residues. In some embodiments, Loop A comprises 3 amino acid residues and Loop B comprises 7 amino acid residues. In some embodiments, Loop A comprises 3 amino acid residues and Loop B comprises 9 amino acid residues. In some embodiments, Loop A comprises 3 amino acid residues and Loop B comprises 6 amino acid residues. In some embodiments, Loop A comprises 2 amino acid residues and Loop B comprises 6 amino acid residues. In some embodiments, Loop A comprises 6 amino acid residues and Loop B comprises 5 amino acid residues.

In some embodiments, Loop A is selected from those depicted in Table 1, below.

In some embodiments, Loop B is selected from those depicted in Table 1, below.

As defined above and described herein, [symbol] indicates the site of attachment to the N-terminus of the Bicycle.

As defined above and described herein, [symbol] indicates the site of attachment to the C-terminus of the Bicycle.

As defined above and described herein, PRR-A¹ is a pattern recognition receptor agonist.

In some embodiments, PRR-A¹ is a pattern recognition receptor agonist.

In some embodiments, PRR-A¹ is a toll-like receptor (TLR) agonist. In some embodiments, PRR-A¹ is a NOD-like receptor pyrin domain containing 3 (NLRP3) agonist. In some embodiments, PRR-A¹ is a both a TLR and NLRP3 agonist.

One of ordinary skill in the art will appreciate that a variety of PRR-A are amenable to achieve the effects of the present invention.

In some embodiments, PRR-A¹ can be connected at any available position. In some embodiments, PRR-A¹ can be connected at any available —OH, —C(O)OH, —SH, —NH₂, or —NHCH₃.

In some embodiments, PRR-A¹ is Motolimod (VTX-2337), both a TLR8 agonist and a NLRP3 agonist:

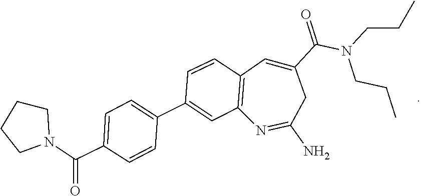 [SMILES: CCCN(CCC)C(=O)C1=Cc2ccc(-c3ccc(C(=O)N4CCCC4)cc3)cc2N=C(N)C1]

In some embodiments, PRR-A¹ is Resiquimod (R848), both a TLR7/8 agonist and a NLRP3 agonist:

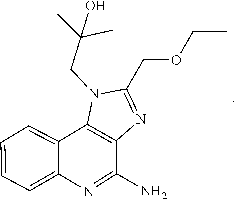 [SMILES: CCOCc1nc2c(N)nc3ccccc3c2n1CC(C)(C)O]

In some embodiments, PRR-A¹ is Vesatolimod (GS-9620), a TLR7 agonist:

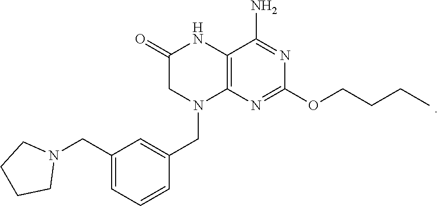 [SMILES: CCCCOc1nc(N)c2c(n1)N(Cc1cccc(CN3CCCC3)c1)CC(=O)N2]

In some embodiments, PRR-A¹ is Gardiquimod:

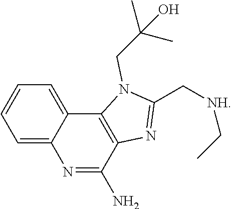 [SMILES: CCNCc1nc2c(N)nc3ccccc3c2n1CC(C)(C)O]

n some embodiments, PRR-A¹ is

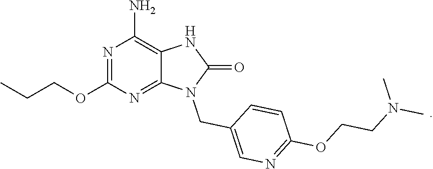 [SMILES: CCCOc1nc(N)c2[nH]c(=O)n(Cc3ccc(OCCN(C)C)nc3)c2n1]

In some embodiments, a portion of PRR-A¹ is replaced with a bioisosteric replacement. Such bioisosteric replacements are described in but not limited to those found in Patani and LaVoie (Chem. Rev. 1996, 96, 3147-3176).

As used herein, depiction of brackets around any PRR-A¹

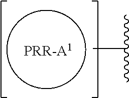

means that the

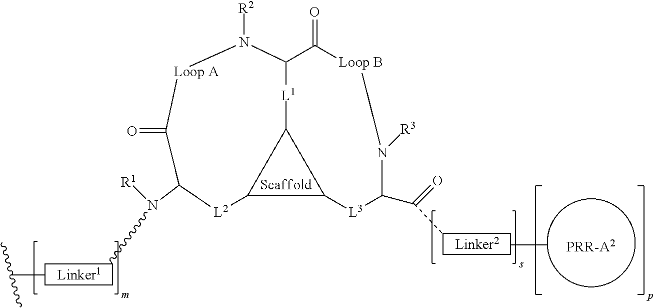

moiety is covalently attached to said PRR-A¹ at any available modifiable carbon, nitrogen, oxygen, or sulfur atom. For purposes of clarity and by way of example, such available modifiable carbon, nitrogen, oxygen, or sulfur atoms in the following PRR-A¹ compound structure are depicted below, wherein each wavy bond defines the point of attachment to said

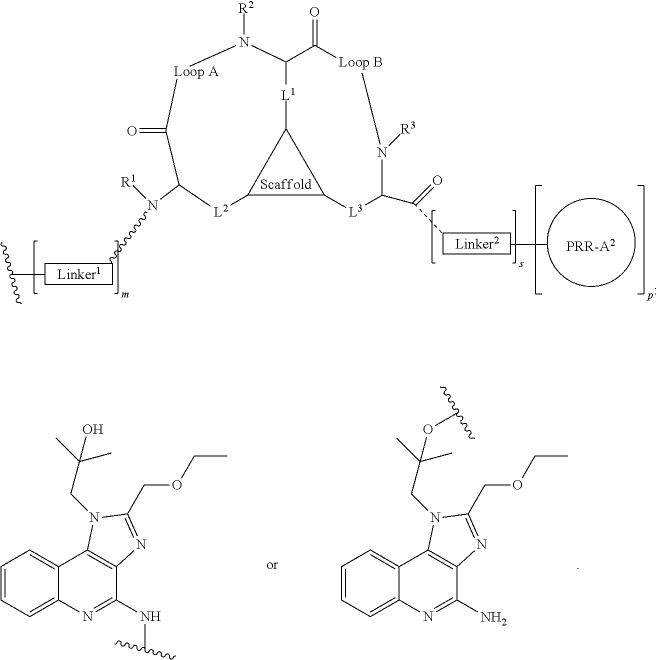

In some embodiments, PRR-A¹ is attached to an amino acid residue in Loop A, Loop B, or the amino acid residues attached to L¹, L² or L³, provided that the site of attachment does not abrogate the binding of the Bicycle portion of the compound with the target.

In some embodiments, PRR-A¹ is attached to L¹, L² or L³, provided that the site of attachment does not abrogate the binding of the Bicycle portion of the compound with the target.

In some embodiments, PRR-A¹ is attached to Scaffold, provided that the site of attachment does not abrogate the binding of the Bicycle portion of the compound with the target.

In some embodiments, PRR-A¹ is selected from those depicted in Table 1, below.

As defined above and described herein, PRR-A² is a pattern recognition receptor agonist.

In some embodiments, PRR-A² is a pattern recognition receptor agonist.

In some embodiments, PRR-A² is a toll-like receptor (TLR) agonist. In some embodiments, PRR-A² is a NOD-like receptor pyrin domain containing 3 (NLRP3) agonist. In some embodiments, PRR-A² is a both a TLR and NLRP3 agonist.

One of ordinary skill in the art will appreciate that a variety of PRR-A are amenable to achieve the effects of the present invention.

In some embodiments, PRR-A² can be connected at any available position. In some embodiments, PRR-A² can be connected at any available —OH, —C(O)OH, —SH, —NH₂, or —NHCH₃.

In some embodiments, PRR-A² is Motolimod (VTX-2337), both a TLR8 agonist and a NLRP3 agonist:

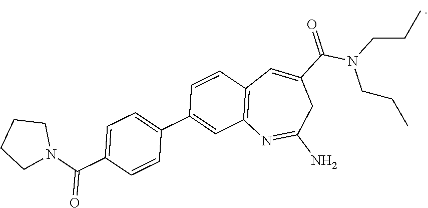 [SMILES: CCCN(CCC)C(=O)C1=Cc2ccc(-c3ccc(C(=O)N4CCCC4)cc3)cc2N=C(N)C1]

In some embodiments, PRR-A² is Resiquimod (R848), both a TLR7/8 agonist and a NLRP3 agonist:

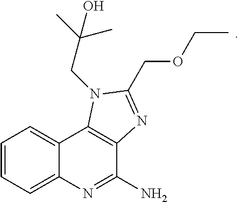 [SMILES: CCOCc1nc2c(N)nc3ccccc3c2n1CC(C)(C)O]

In some embodiments, PRR-A² is Vesatolimod (GS-9620), a TLR7 agonist:

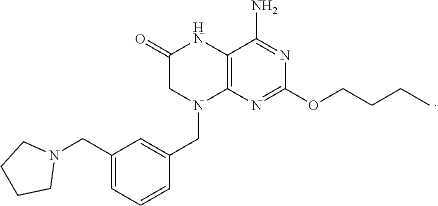 [SMILES: CCCCOc1nc(N)c2c(n1)N(Cc1cccc(CN3CCCC3)c1)CC(=O)N2]

In some embodiments, PRR-A² is Gardiquimod:

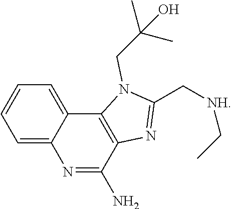 [SMILES: CCNCc1nc2c(N)nc3ccccc3c2n1CC(C)(C)O]

In some embodiments, PRR-A² is

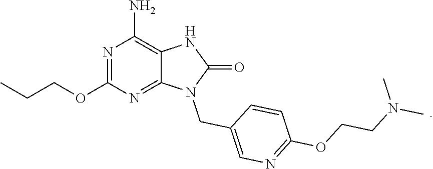 [SMILES: CCCOc1nc(N)c2[nH]c(=O)n(Cc3ccc(OCCN(C)C)nc3)c2n1]

In some embodiments, a portion of PRR-A² is replaced with a bioisosteric replacement. Such bioisosteric replacements are described in but not limited to those found in Patani and LaVoie (Chem. Rev. 1996, 96, 3147-3176).

As used herein, depiction of brackets around any PRR-A²

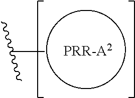

means that the

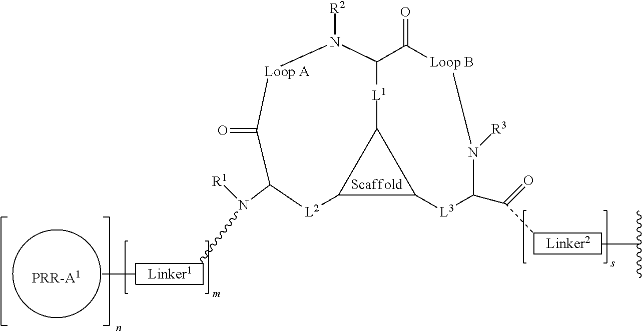

moiety is covalently attached to said PRR-A² at any available modifiable carbon, nitrogen, oxygen, or sulfur atom. For purposes of clarity and by way of example, such available modifiable carbon, nitrogen, oxygen, or sulfur atoms in the following PRR-A² compound structure are depicted below, wherein each wavy bond defines the point of attachment to said

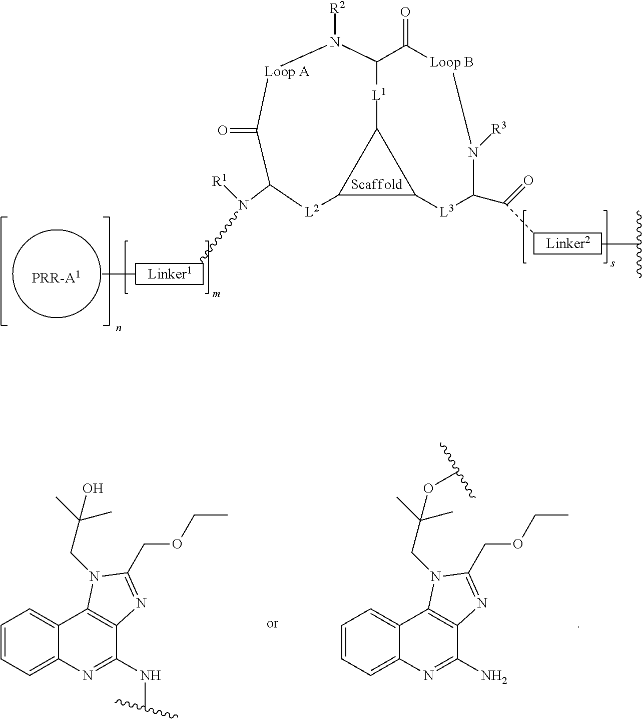

In some embodiments, PRR-A² is attached to an amino acid residue in Loop A, Loop B, or the amino acid residues attached to L¹, L² or L³, provided that the site of attachment does not abrogate the binding of the Bicycle portion of the compound with the target.

In some embodiments, PRR-A² is attached to L¹, L² or L³, provided that the site of attachment does not abrogate the binding of the Bicycle portion of the compound with the target.

In some embodiments, PRR-A² is attached to Scaffold, provided that the site of attachment does not abrogate the binding of the Bicycle portion of the compound with the target.

In some embodiments, PRR-A² is selected from those depicted in Table 1, below.

As defined above and described herein, Linker¹ is hydrogen or a bivalent moiety that connects the N-terminus of the Bicycle with PRR-A¹, wherein when n is 0, Linker¹ is hydrogen.

In some embodiments, Linker¹ is hydrogen, wherein n is 0. In some embodiments, Linker¹ is a bivalent moiety that connects the N-terminus of the Bicycle with PRR-A¹.

In some embodiments, Linker¹ is a covalent bond. In some embodiments, Linker¹ is

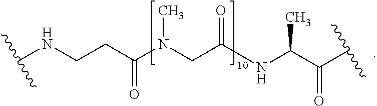

In some embodiments, Linker¹ is

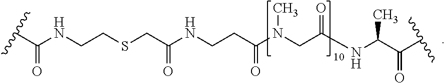

In some embodiments, Linker¹ is

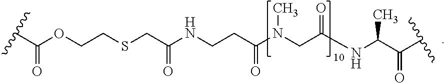

In some embodiments, Linker¹ is

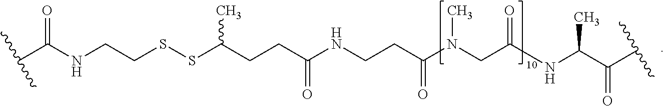

In some embodiments, Linker¹ is

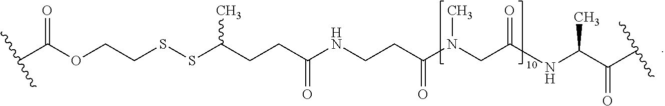

In some embodiments, Linker¹ is

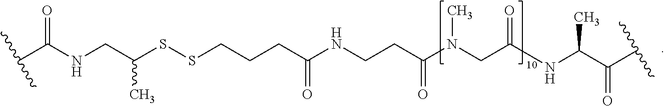

In some embodiments, Linker¹ is

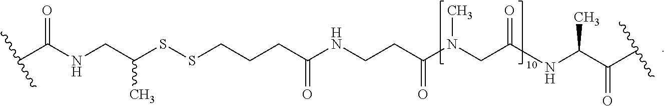

In some embodiments, Linker¹ is

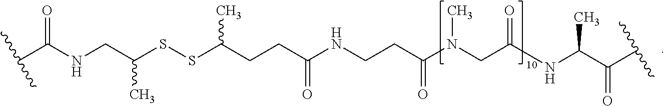

In some embodiments, Linker¹ is

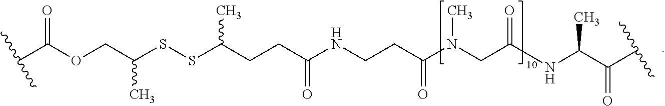

In some embodiments, Linker¹ is

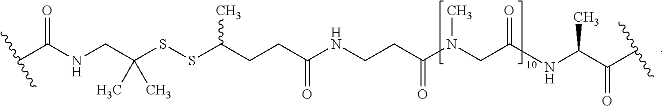

In some embodiments, Linker¹ is

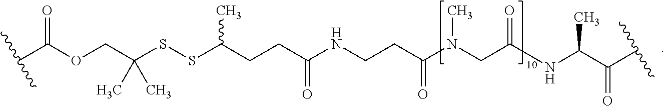

In some embodiments, Linker¹ is

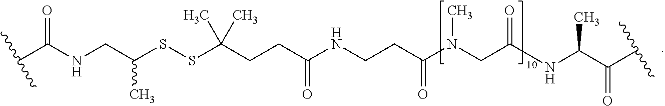

In some embodiments, Linker¹ is

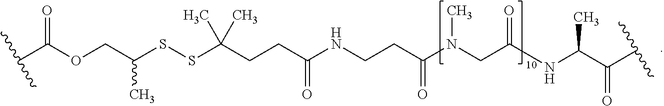

In some embodiments, Linker¹ is

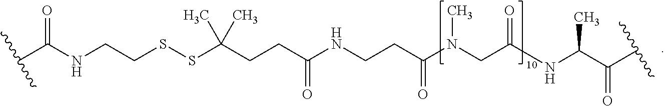

In some embodiments, Linker¹ is

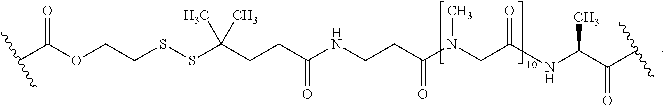

In some embodiments, Linker¹ is

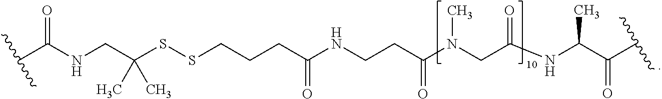

In some embodiments, Linker¹ is

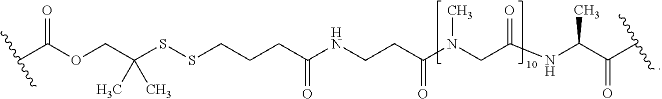

In some embodiments, Linker¹ is

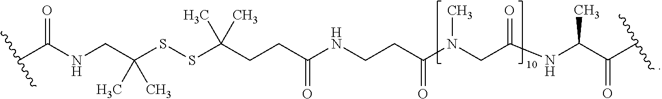

In some embodiments, Linker¹ is

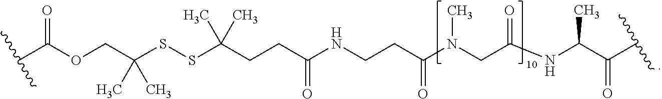

In some embodiments, Linker¹ is

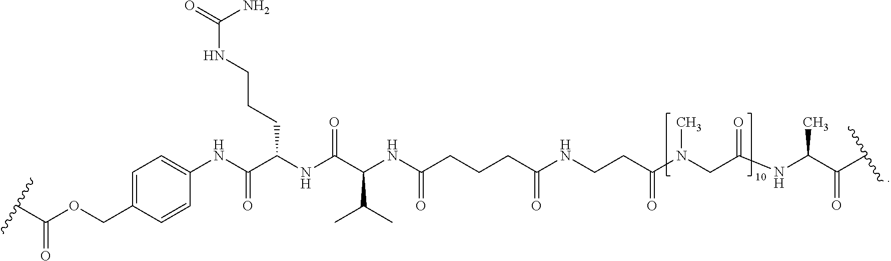

In some embodiments, Linker¹ is

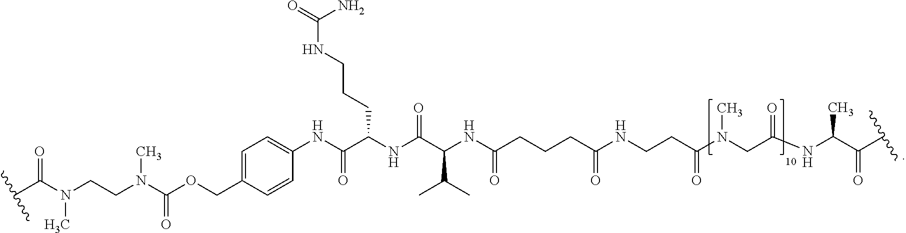

In some embodiments, Linker¹ is

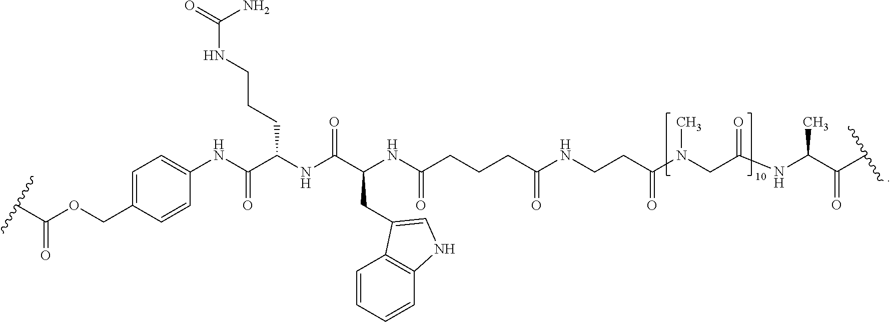

In some embodiments, Linker¹ is

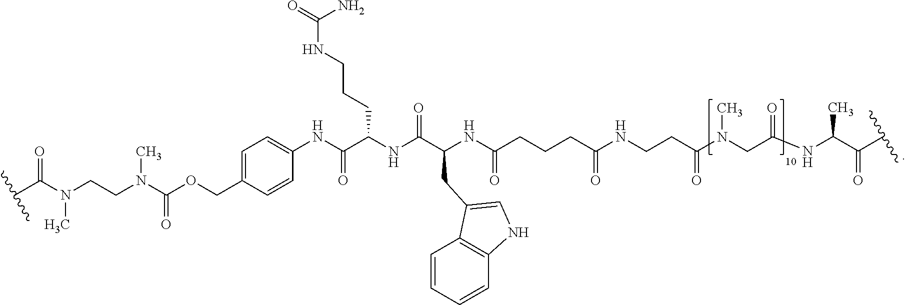

In some embodiments, Linker¹ is

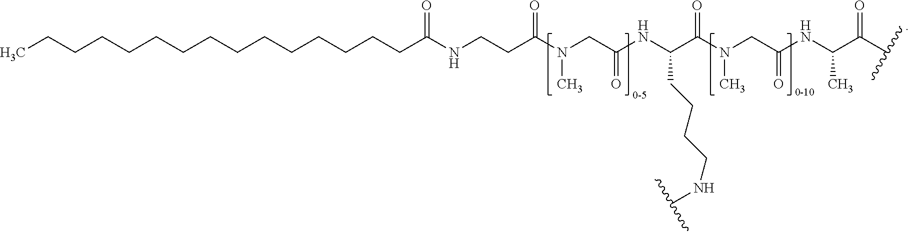

In some embodiments, Linker¹ is

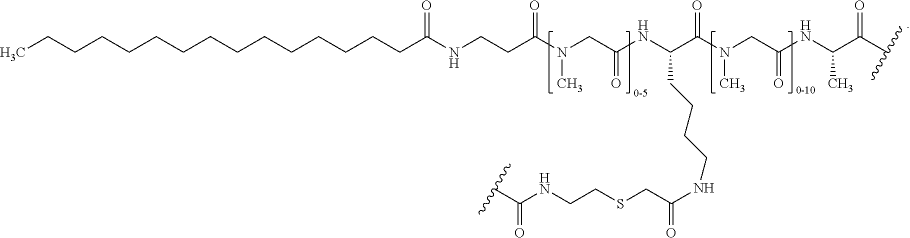

In some embodiments, Linker¹ is

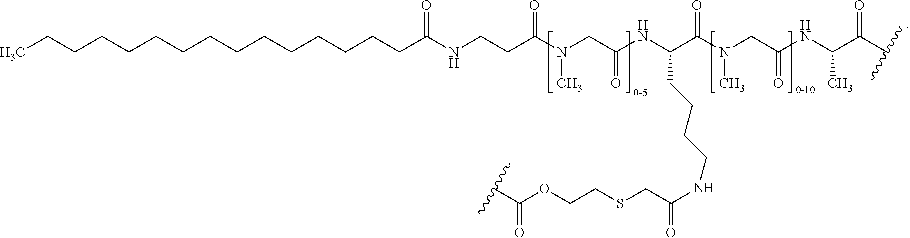

In some embodiments, Linker¹ is

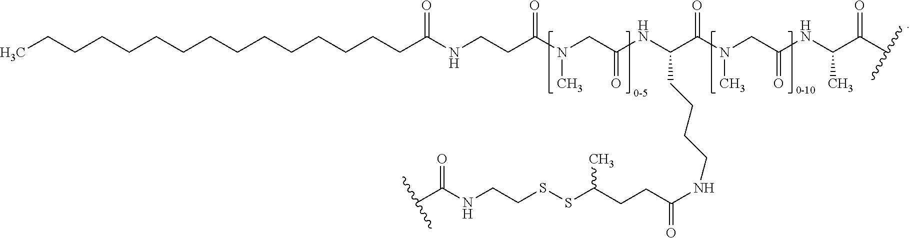

In some embodiments, Linker¹ is

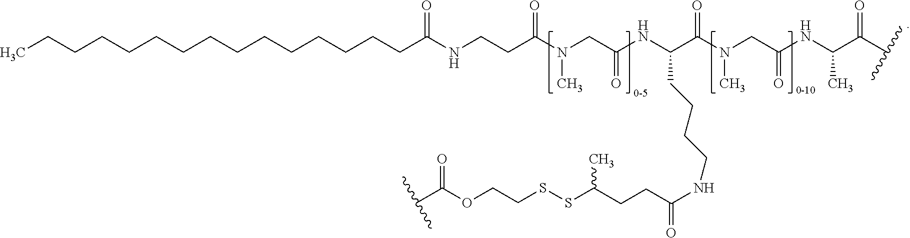

In some embodiments, Linker¹ is

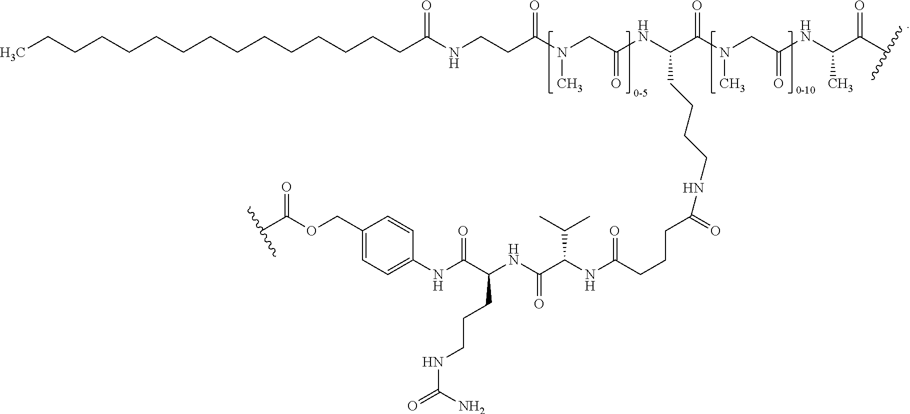

In some embodiments, Linker¹ is

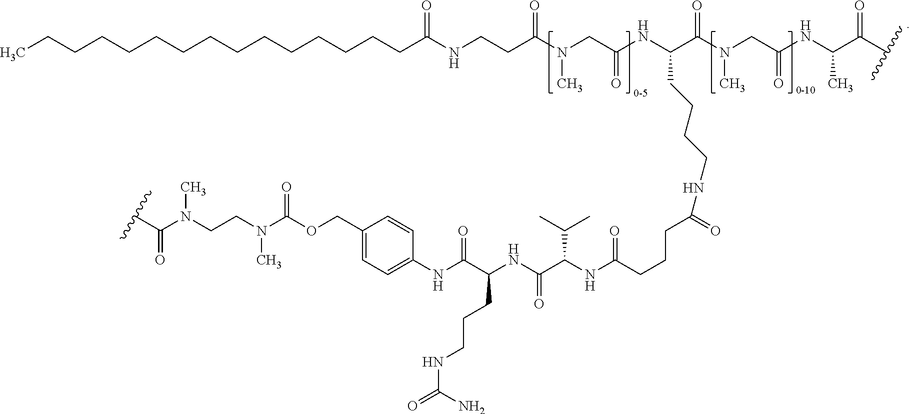

In some embodiments, Linker¹ is selected from the following:

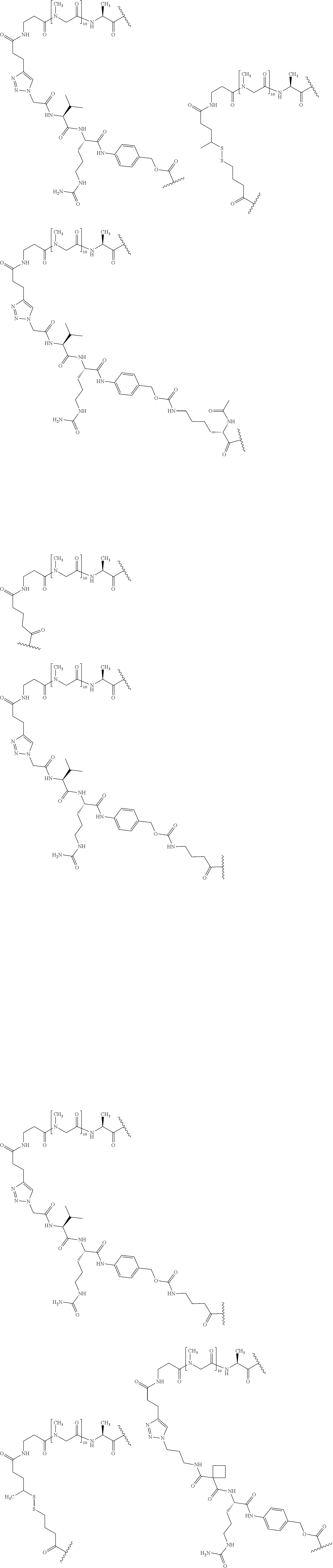

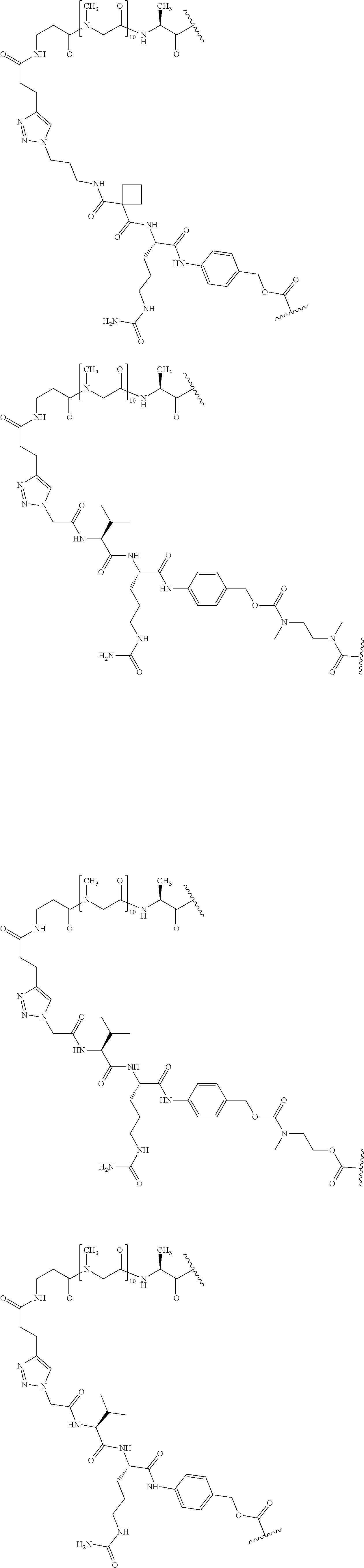

In some embodiments, Linker¹ is selected from those depicted in Table 1, below.

As defined above and described herein, Linker² is —NH₂ or a bivalent moiety that connects the C-terminus of the Bicycle with PRR-A², wherein when p is 0, Linker² is —NH₂.

In some embodiments, Linker² is —NH₂, wherein p is 0. In some embodiments, Linker² is a bivalent moiety that connects the C-terminus of the Bicycle with PRR-A².

In some embodiments, Linker² is a covalent bond. In some embodiments, Linker² is

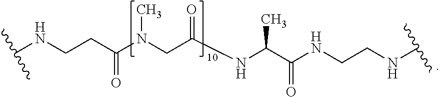

In some embodiments, Linker² is

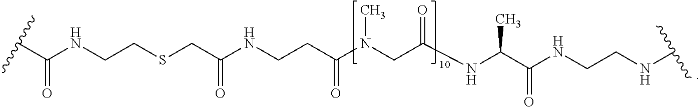

In some embodiments, Linker² is

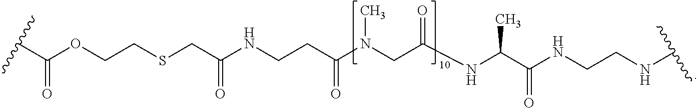

In some embodiments, Linker² is

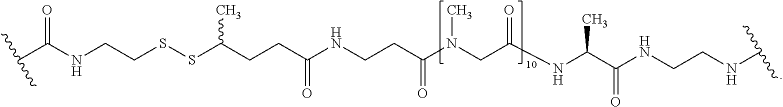

In some embodiments, Linker² is

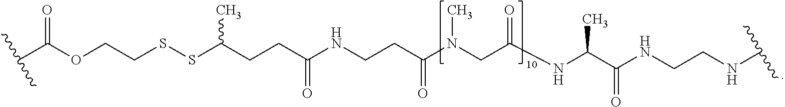

In some embodiments, Linker² is

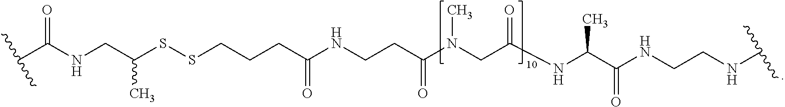

In some embodiments, Linker² is

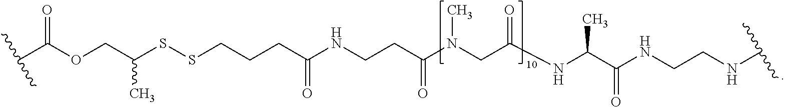

In some embodiments, Linker² is

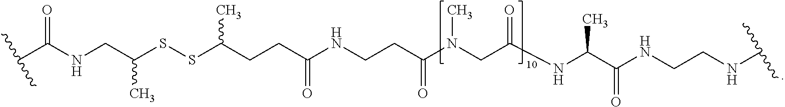

In some embodiments, Linker² is

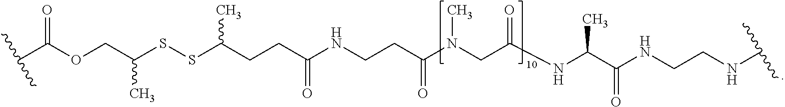

In some embodiments, Linker² is

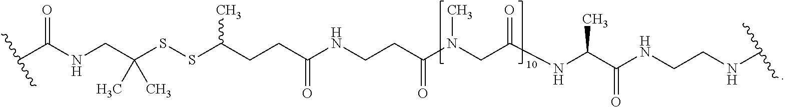

In some embodiments, Linker² is

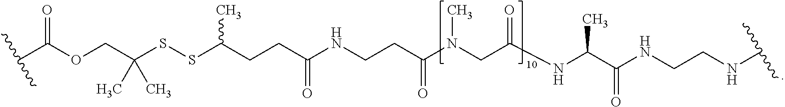

In some embodiments, Linker² is

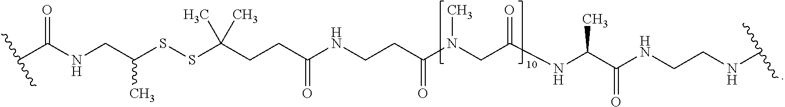

In some embodiments, Linker² is

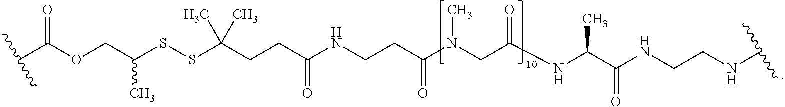

In some embodiments, Linker² is

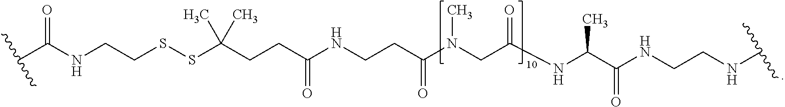

In some embodiments, Linker² is

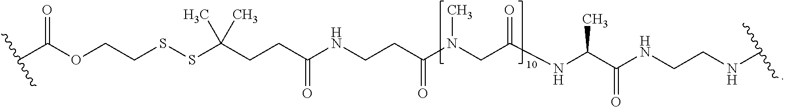

In some embodiments, Linker² is

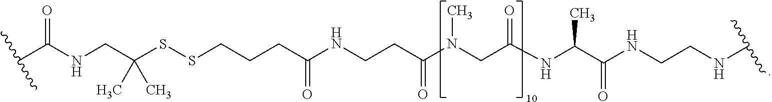

In some embodiments, Linker² is

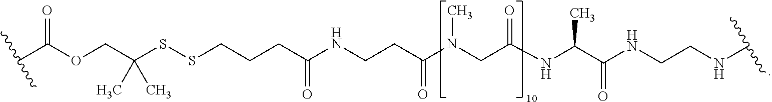

In some embodiments, Linker² is

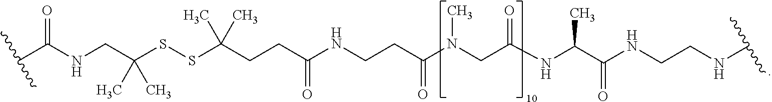

In some embodiments, Linker² is

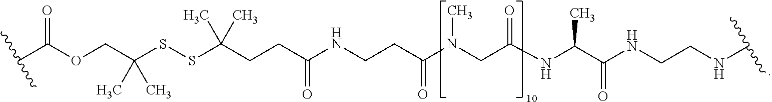

In some embodiments, Linker² is

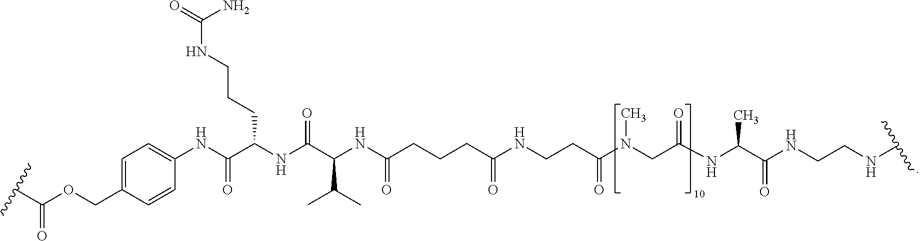

In some embodiments, Linker² is

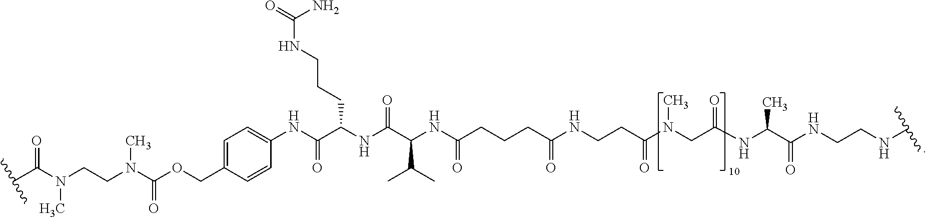

In some embodiments, Linker² is

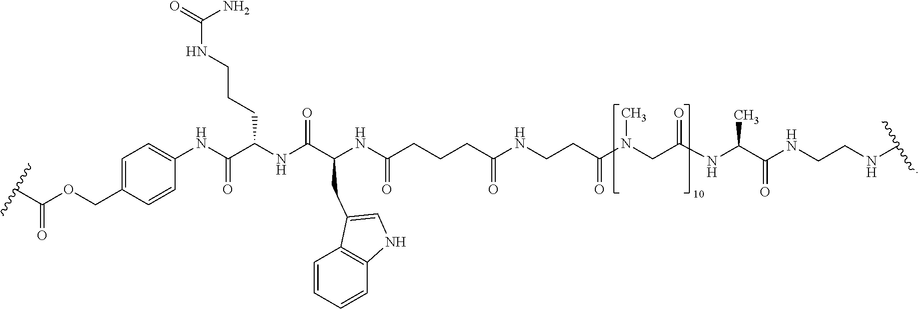

In some embodiments, Linker² is

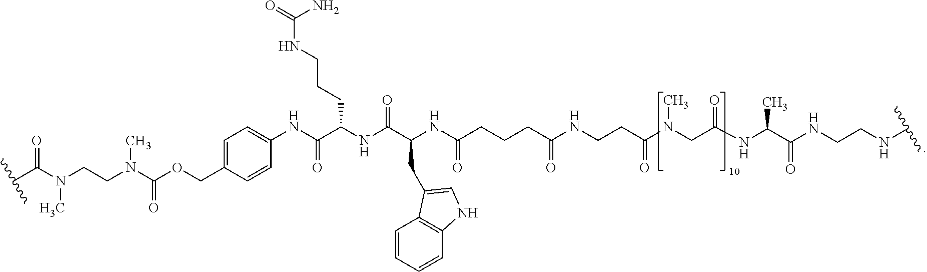

In some embodiments, Linker² is

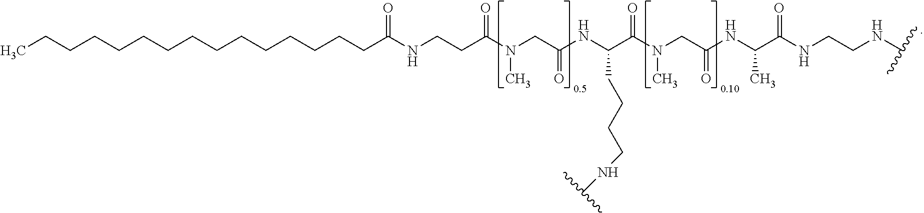

In some embodiments, Linker² is

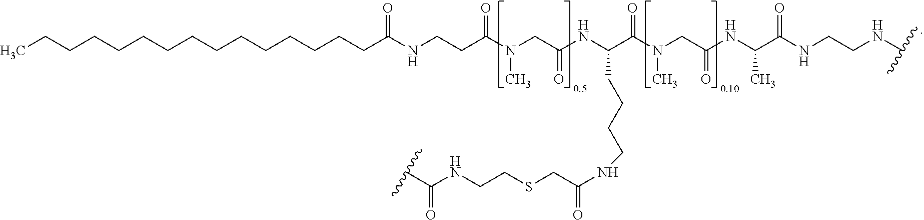

In some embodiments, Linker² is

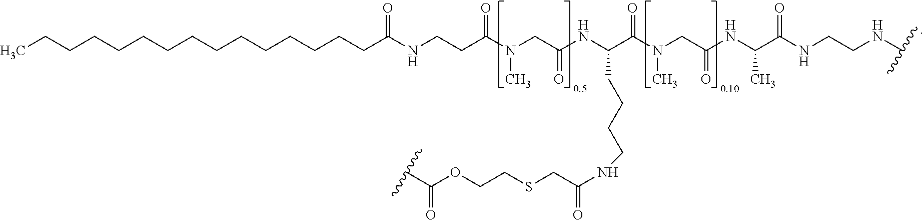

In some embodiments, Linker² is

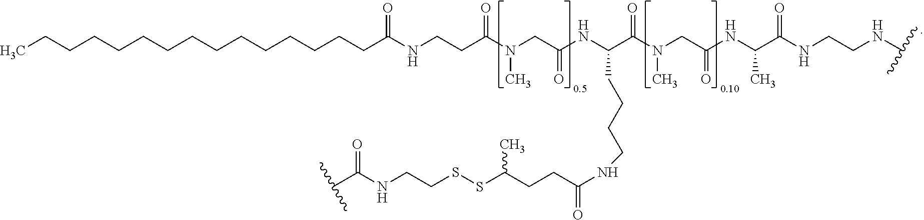

In some embodiments, Linker² is

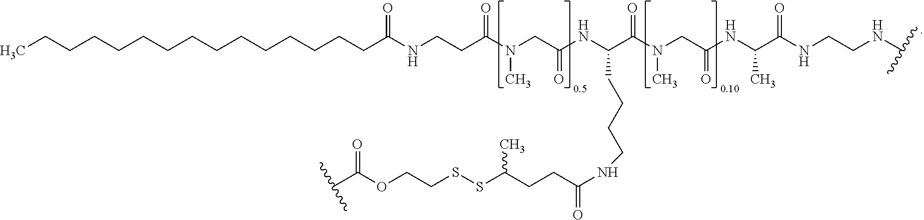

In some embodiments, Linker² is

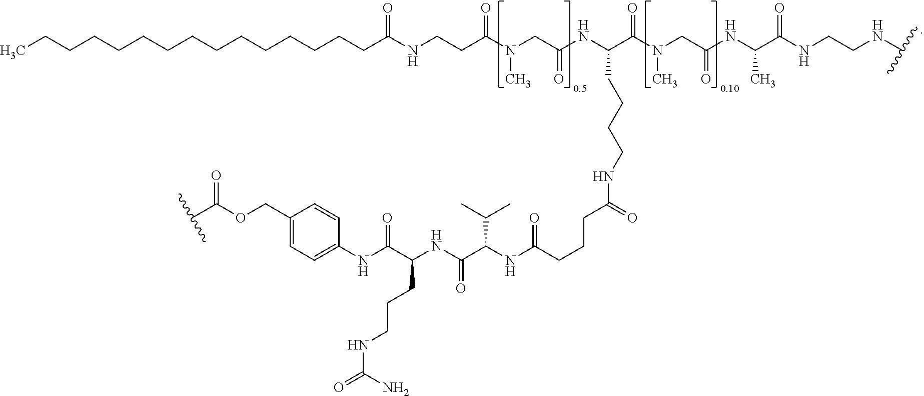

In some embodiments, Linker² is

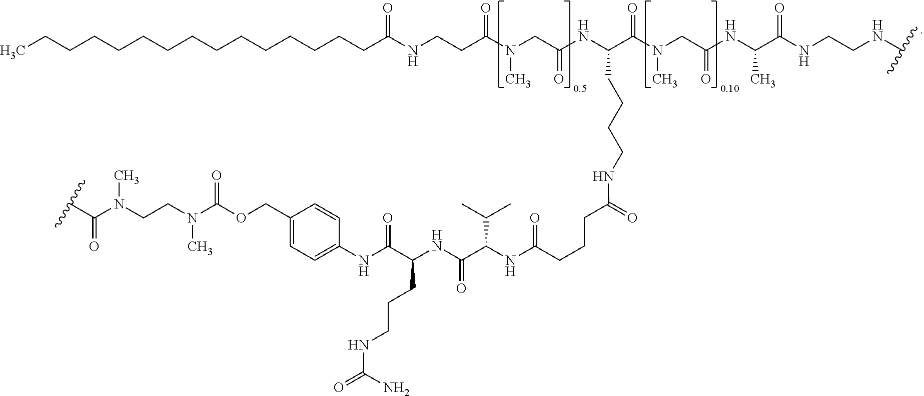

In some embodiments, Linker² is selected from those depicted in Table 1, below.

As defined above and described herein, Ring A is selected from the group consisting of 18-crown-6, 1,7,13-triaza-18-crown-6, and a 3-12-membered saturated, partially unsaturated, bridged bicyclic, bridged tricyclic, propellane, or aromatic ring optionally substituted with 0-3 oxo, methyl, ethyl or spiroethylene groups and having 0-6 heteroatoms independently selected from nitrogen, oxygen, or sulfur.

In some embodiments, Ring A is 18-crown-6. In some embodiments, Ring A is 1,7,13-triaza-18-crown-6. In some embodiments, Ring A is a 3-12-membered saturated, partially unsaturated, bridged bicyclic, bridged tricyclic, propellane, or aromatic ring optionally substituted with 0-3 oxo, methyl, ethyl or spiroethylene groups and having 0-6 heteroatoms independently selected from nitrogen, oxygen, or sulfur.

In some embodiments, Ring A is

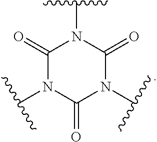 [SMILES: *n1c(=O)n(*)c(=O)n(*)c1=O]

In some embodiments, Ring A is

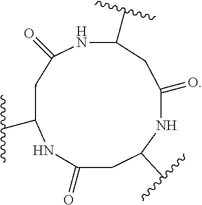 [SMILES: *C1CC(=O)NC(*)CC(=O)NC(*)CC(=O)N1]

In some embodiments, Ring A is

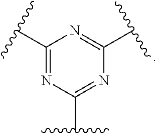 [SMILES: *c1nc(*)nc(*)n1]

In some embodiments, Ring A is

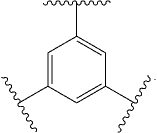 [SMILES: *c1cc(*)cc(*)c1]

In some embodiments, Ring A is

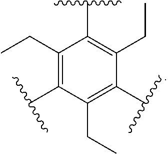 [SMILES: *c1c(CC)c(*)c(CC)c(*)c1CC]

In some embodiments, Ring A is

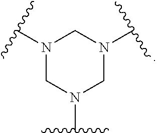 [SMILES: *N1CN(*)CN(*)C1]

In some embodiments, Ring A is

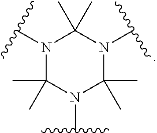

In some embodiments, Ring A is

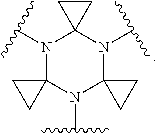 [SMILES: *N1C2(CC2)N(*)C2(CC2)N(*)C12CC2]

In some embodiments, Ring A is

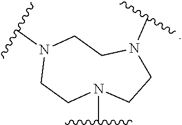 [SMILES: *N1CCN(*)CCN(*)CC1]

In some embodiments, Ring A is

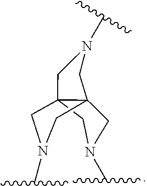 [SMILES: *N1CC23CN(*)CC2(C1)CN(*)C3]

In some embodiments, Ring A is

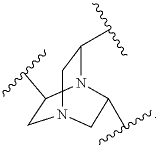 [SMILES: *C1CN2CC(*)N1C(*)C2]

In some embodiments, Ring A is

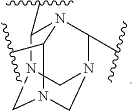 [SMILES: *C1N2CN3CN(C2)C(*)N1C3*]

In some embodiments, Ring A is

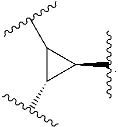 [SMILES: *C1[C@H](*)[C@H]1*]

In some embodiments, Ring A is

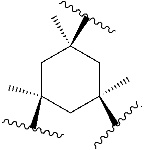

In some embodiments, Ring A is

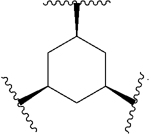 [SMILES: *[C@H]1C[C@@H](*)C[C@@H](*)C1]

In some embodiments, Ring A is

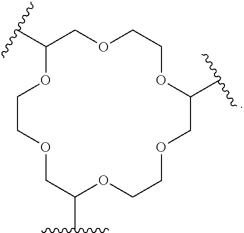 [SMILES: *C1COCCOC(*)COCCOC(*)COCCO1]

In some embodiments, Ring A is

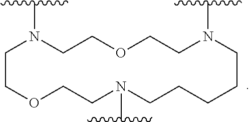 [SMILES: *N1CCCCCN(*)CCOCCN(*)CCOCC1]

In some embodiments, Ring A is selected from those depicted in Table 1, below.

In certain embodiments, the present invention provides a Bicycle of formula I, wherein Scaffold is Ring A, thereby forming a Bicycle of formula I-a:

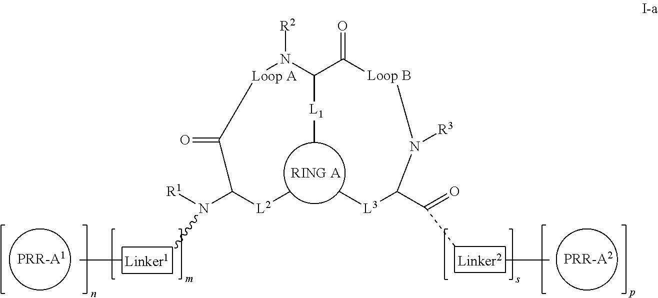

or a pharmaceutically acceptable salt thereof, wherein each of Loop A, Loop B, Ring A, L¹, L², L³, Linker¹, Linker², PRR-A¹, PRR-A², R¹, R², R³, m, n, s and p is as defined above and described in embodiments herein, both singly and in combination.

In certain embodiments, the present invention provides a Bicycle of formula I, wherein Loop A is

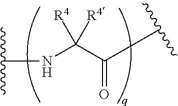

### and Loop B is

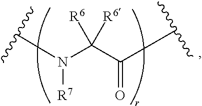

thereby forming a Bicycle of formula II:

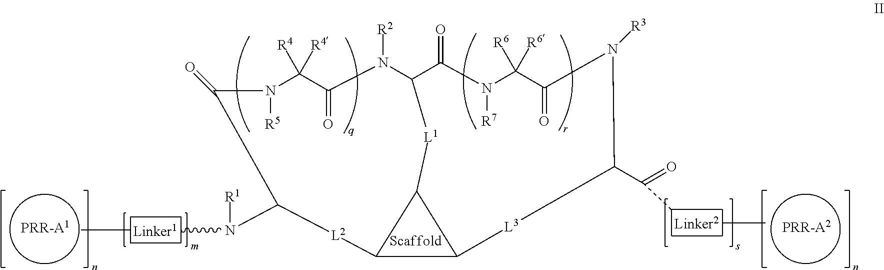

or a pharmaceutically acceptable salt thereof, wherein each of L¹, L², L³, Scaffold, R¹, R², R³, R⁴, R^{4′}, R⁵, R⁶, R^{6′}, R⁷, Linker¹, Linker², PRR-A¹, PRR-A², m, n, s, p, q and r is as defined above and described in embodiments herein, both singly and in combination.

In certain embodiments, the present invention provides a Bicycle of formula II, wherein o is 1, p is 0, Linker² is —NH₂, and R¹ is hydrogen, thereby forming a Bicycle of formula II-a:

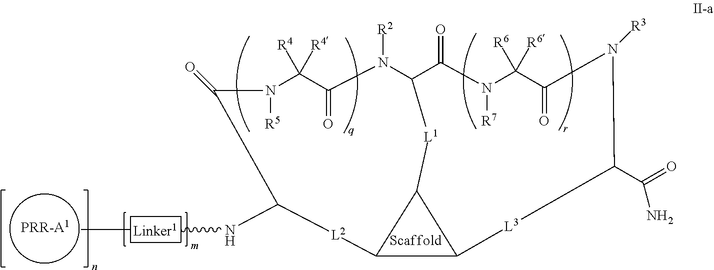

or a pharmaceutically acceptable salt thereof, wherein each of L¹, L², L³, Scaffold, R², R³, R⁴, R^{4′}, R⁵, R⁶, R^{6′}, R⁷, Linker¹, PRR-A¹, m, n, q and r is as defined above and described in embodiments herein, both singly and in combination.

In certain embodiments, the present invention provides a Bicycle of formula II, wherein n is 0 and Linker¹ is hydrogen, thereby forming a Bicycle of formula II-b:

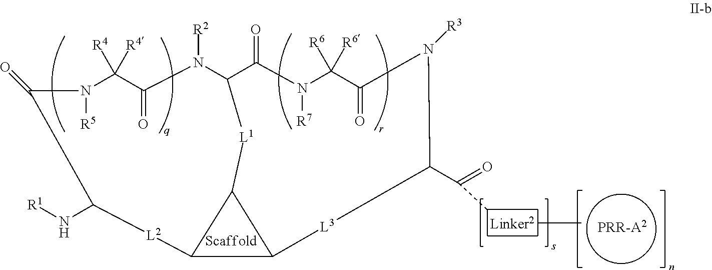

or a pharmaceutically acceptable salt thereof, wherein each of L¹, L², L³, Scaffold, R¹, R², R³, R⁴, R^{4′}, R⁵, R⁶, R^{6′}, R⁷, Linker², PRR-A², s, p, q and r is as defined above and described in embodiments herein, both singly and in combination.

In certain embodiments, a compound of the invention is of formula III:

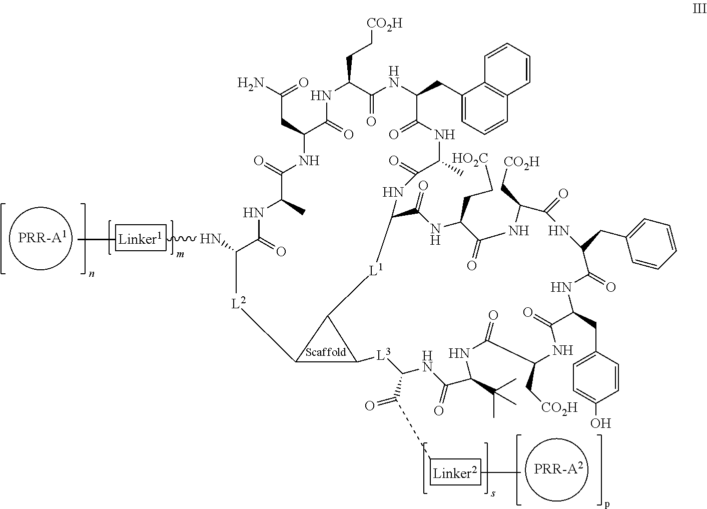

or a pharmaceutically acceptable salt thereof, wherein each of L¹, L², L³, Scaffold, Linker¹, Linker², PRR-A¹, PRR-A², s, p, n, and m is as defined above and described in embodiments herein, both singly and in combination.

In certain embodiments, a compound of the invention is of formula III-a:

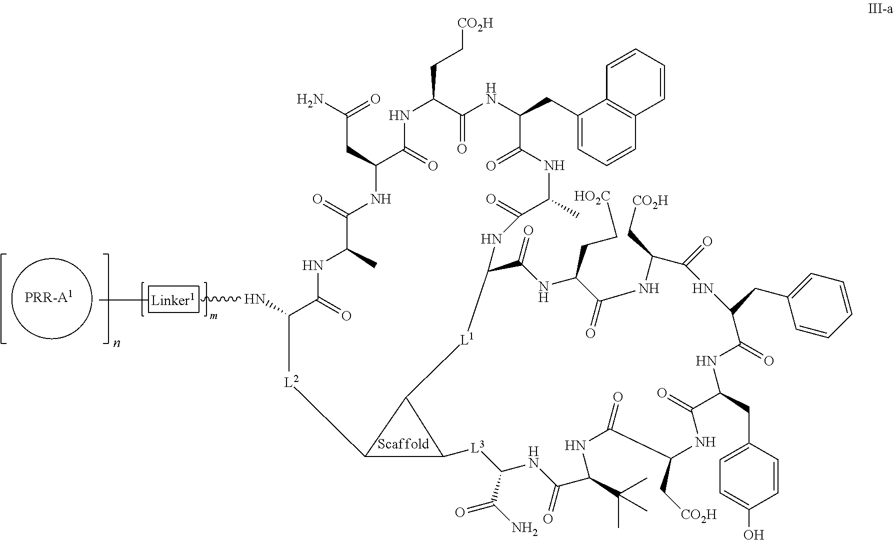

or a pharmaceutically acceptable salt thereof, wherein each of L¹, L², L³, Scaffold, Linker¹, PRR-A¹, n, and m is as defined above and described in embodiments herein, both singly and in combination.

In certain embodiments, a compound of the invention is of formula III-b:

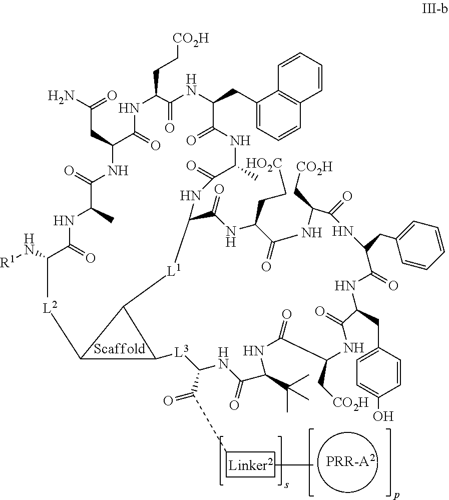

or a pharmaceutically acceptable salt thereof, wherein each of R¹, L¹, L², L³, Scaffold, Linker², PRR-A², s, and p is as defined above and described in embodiments herein, both singly and in combination.

In certain embodiments, a compound of the invention is of formula IV:

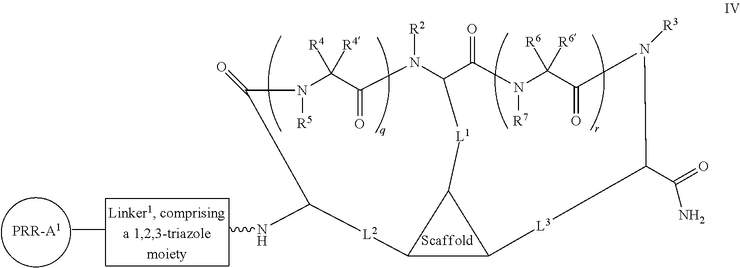

or a pharmaceutically acceptable salt thereof, wherein each of L¹, L², L³, Scaffold, R², R³, R⁴, R^{4′}, R⁵, R⁶, R^{6′}, R⁷, Linker¹, PRR-A¹, q and r is as defined above and below and in classes and subclasses as described herein.

In certain embodiments, a compound of the invention is of formula V:

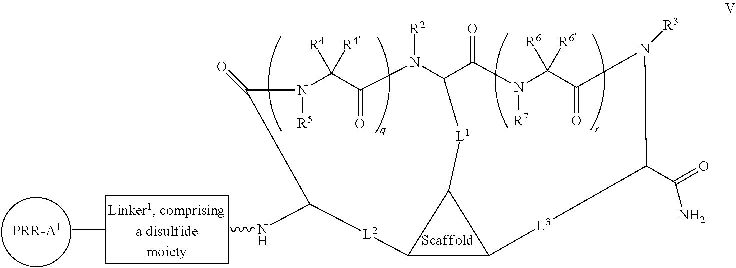

or a pharmaceutically acceptable salt thereof, wherein each of L¹, L², L³, Scaffold, R², R³, R⁴, R^{4′}, R⁵, R⁶, R^{6′}, R⁷, Linker¹, PRR-A¹, q and r is as defined above and below and in classes and subclasses as described herein.

Exemplary compounds of the invention are set forth in Table 1, below.

TABLE 1
Exemplary compounds

| | |
|---|---|
| 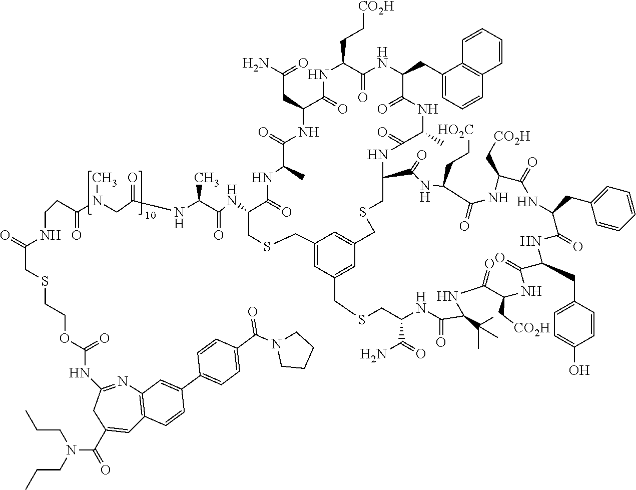 | I-1 |
| 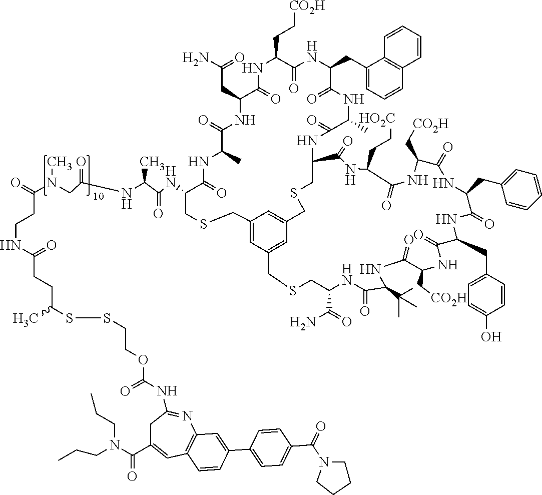 | I-2 |
| 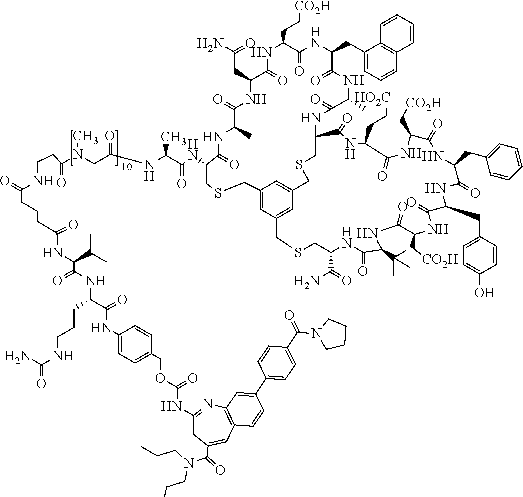 | I-3 |
| 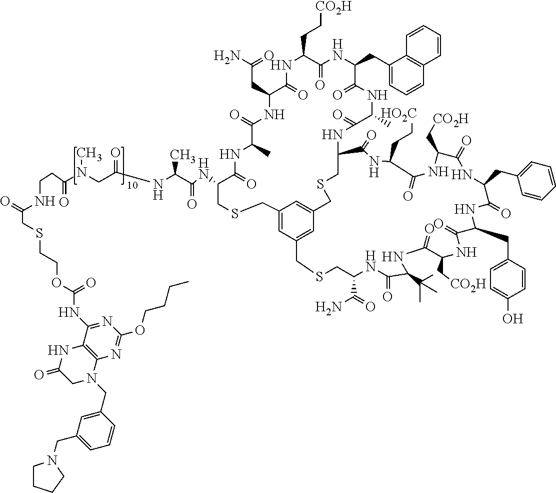 | I-4 |
| 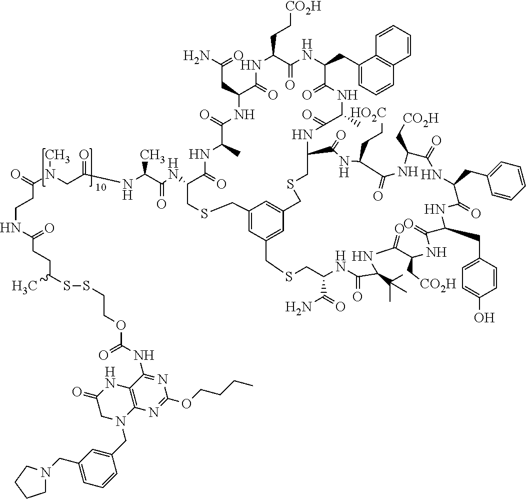 | I-5 |
| 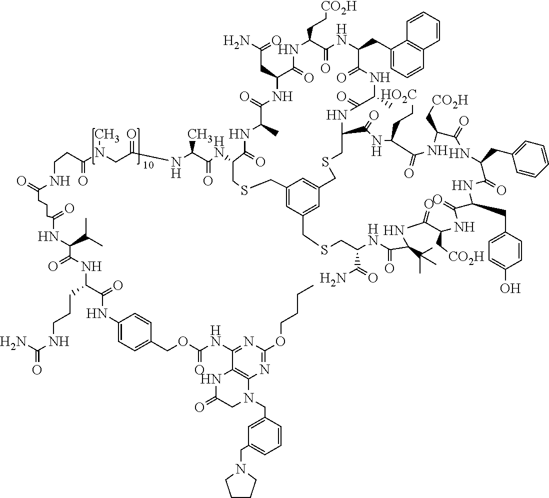 | I-6 |
| 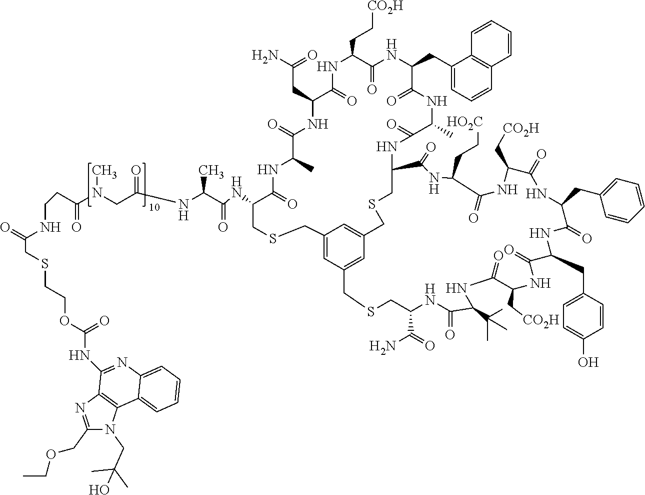 | I-7 |
| 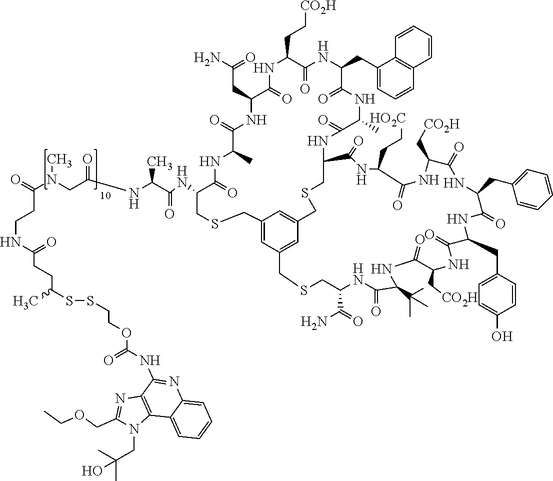 | I-8 |
| 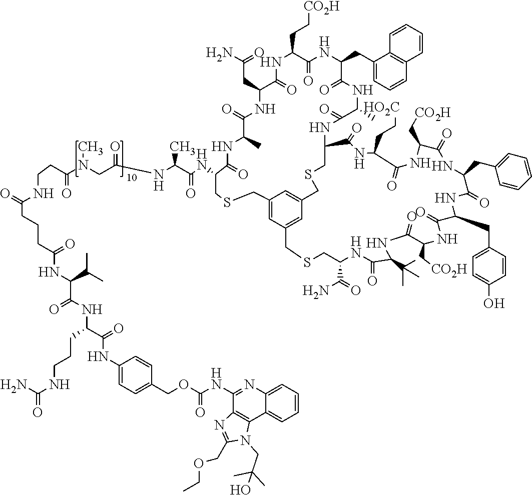 | I-9 |
| 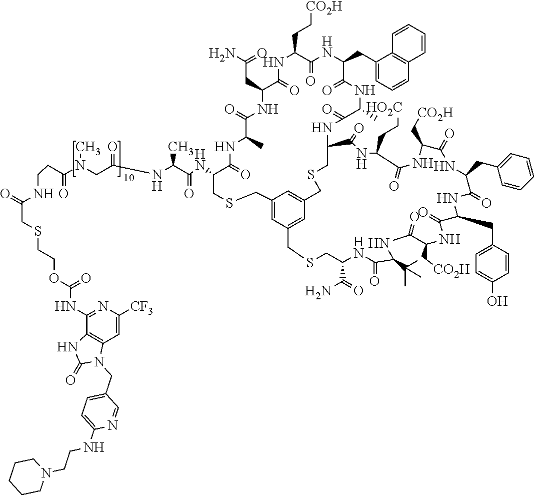 | I-10 |
| 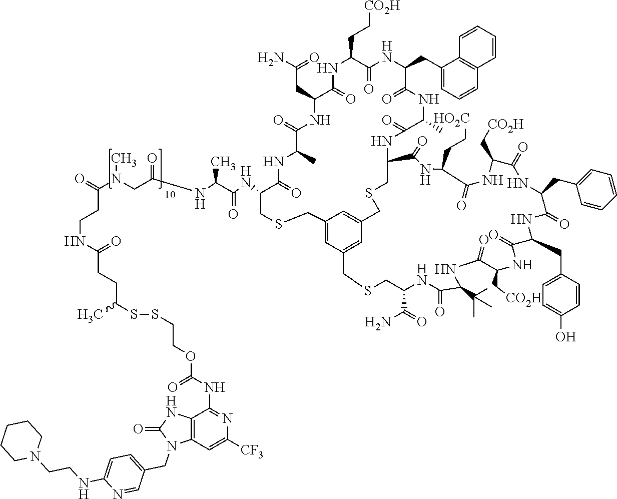 | I-11 |
| 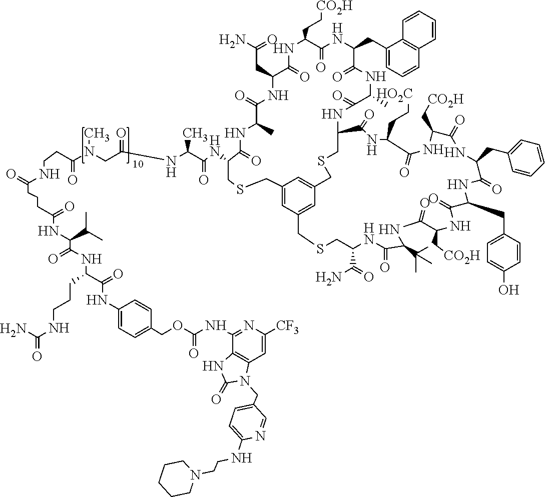 | I-12 |
| 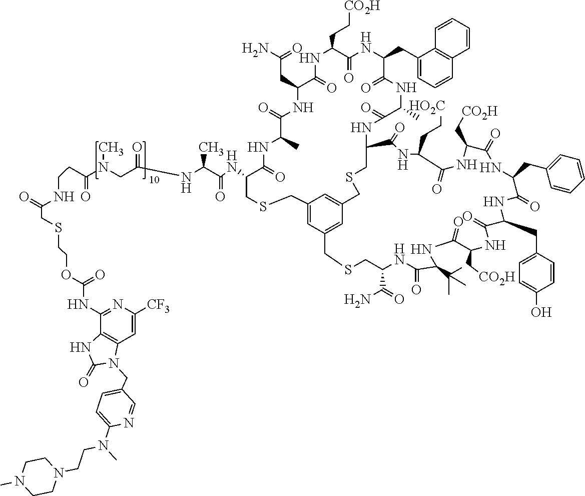 | I-13 |
| 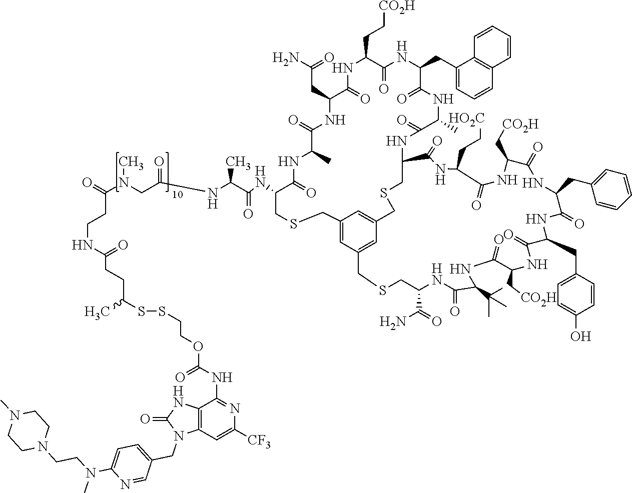 | I-14 |
| 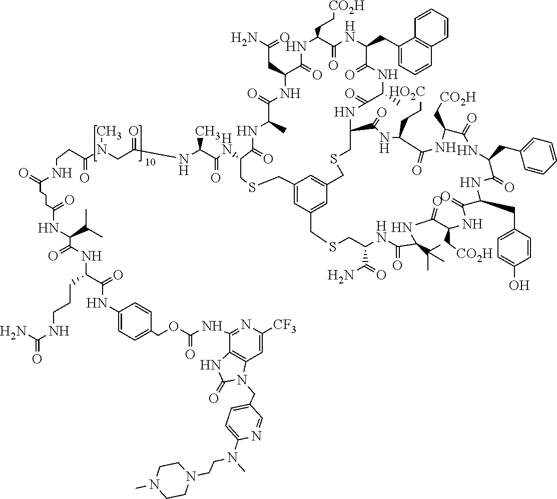 | I-15 |
| 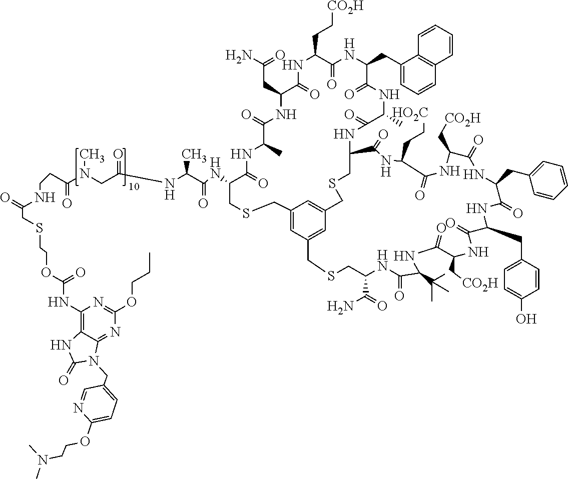 | I-16 |
| 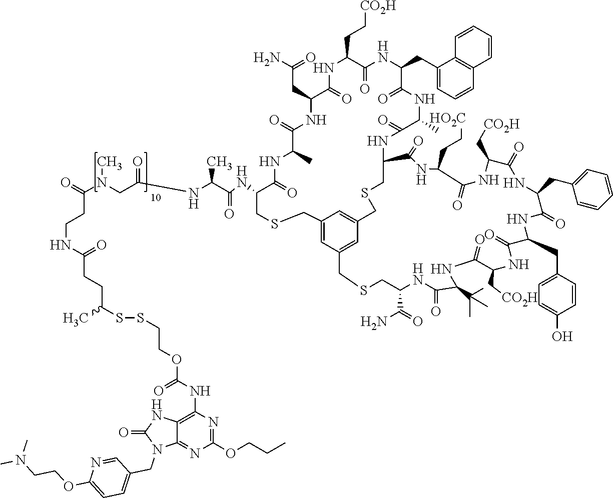 | I-17 |
| 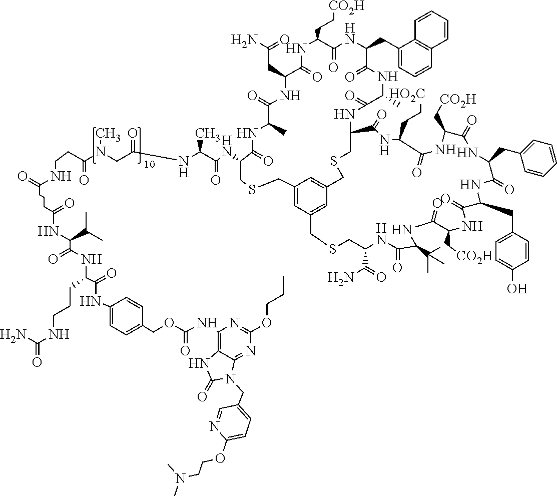 | I-18 |
| 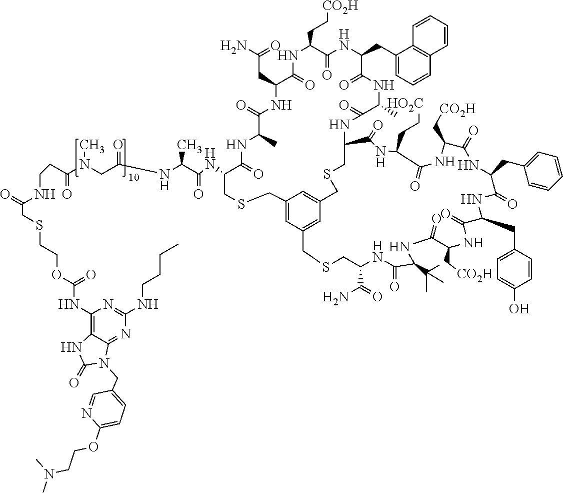 | I-19 |
| 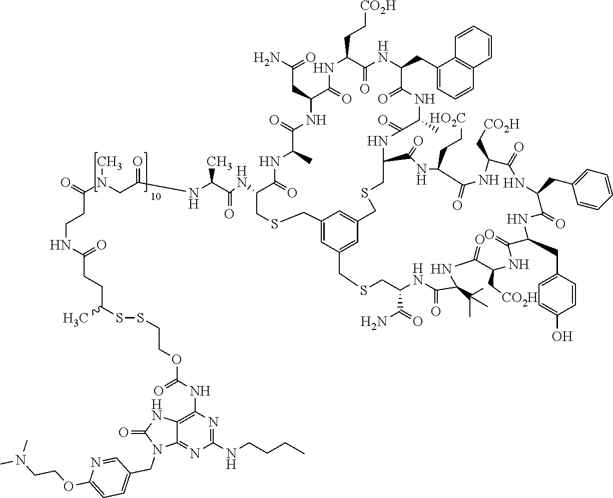 | I-20 |
| 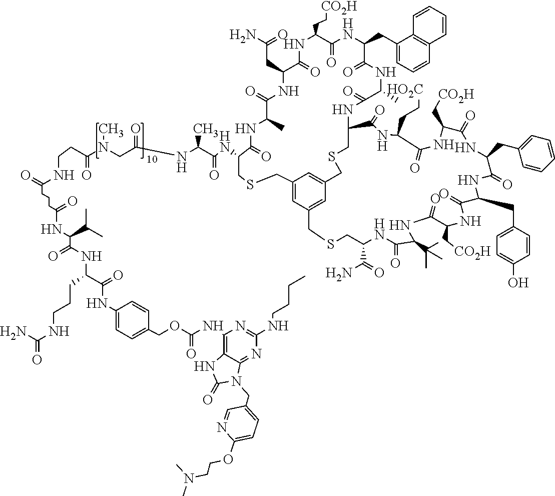 | I-21 |
| 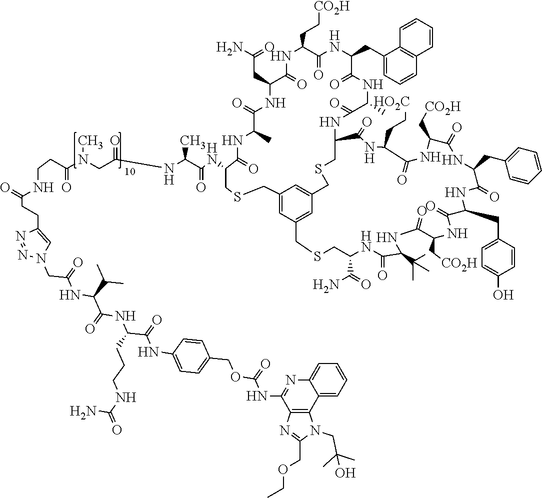 | I-22 |
| 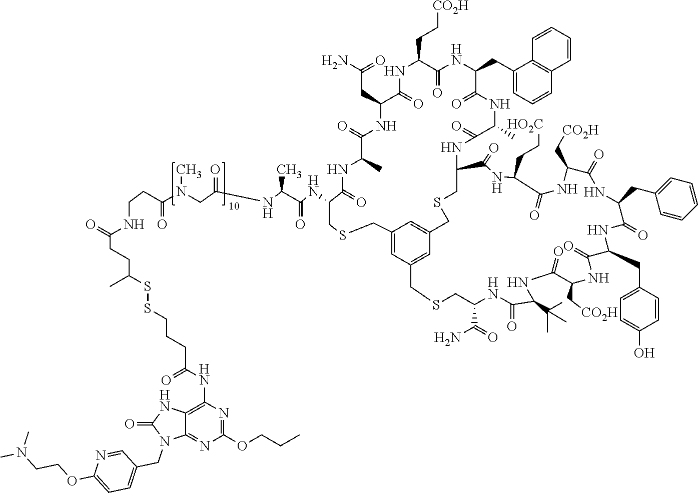 | I-23 |
| 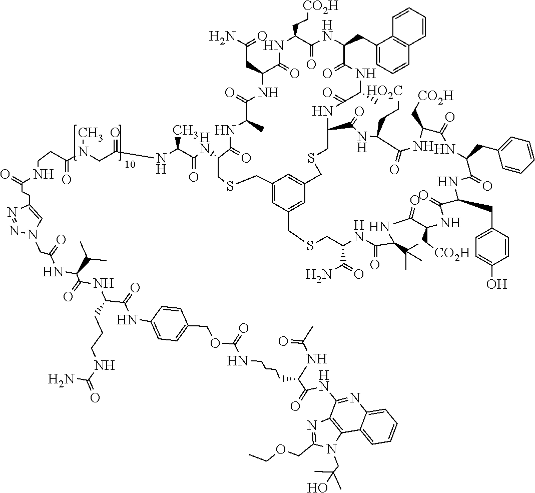 | I-24 |
| 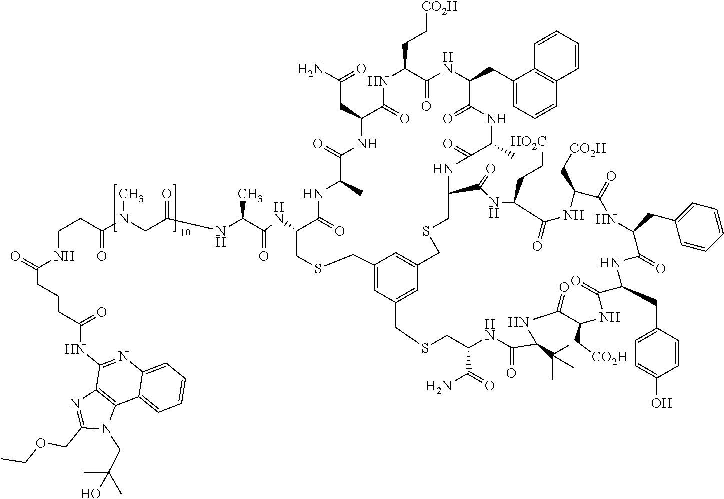 | I-25 |
| 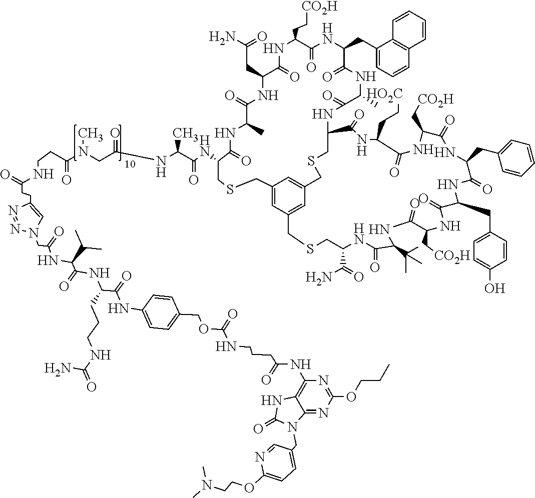 | I-26 |
| 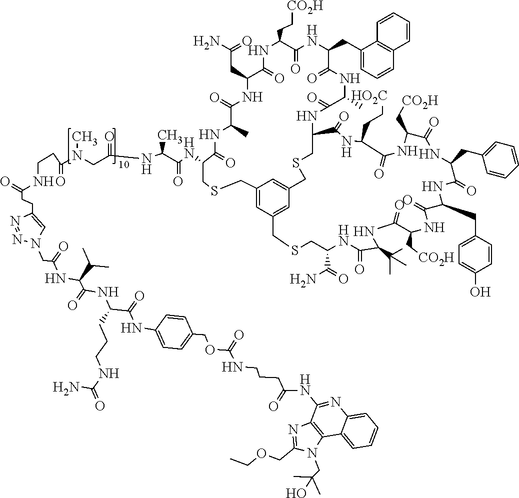 | I-27 |
| 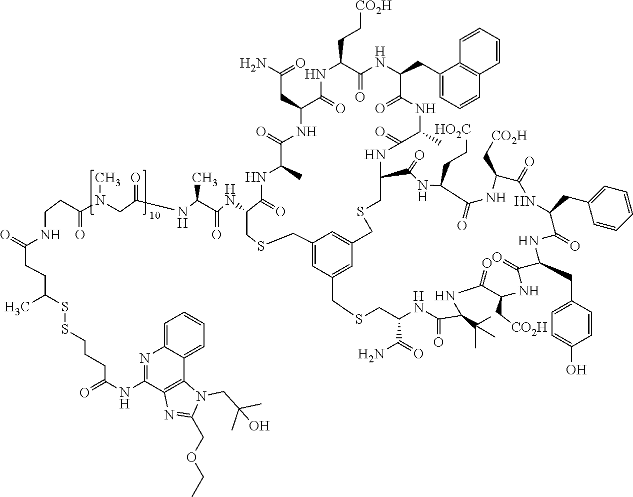 | I-28 |
| 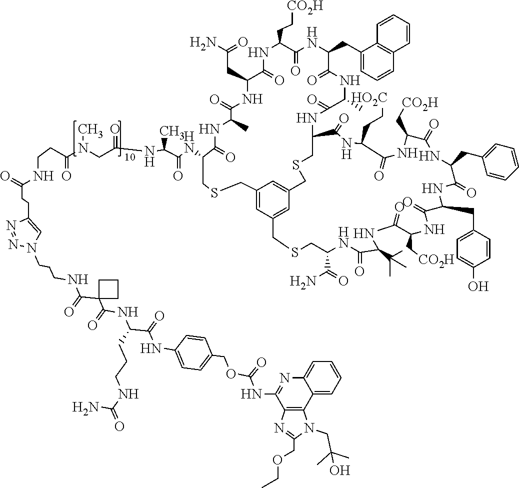 | I-29 |
| 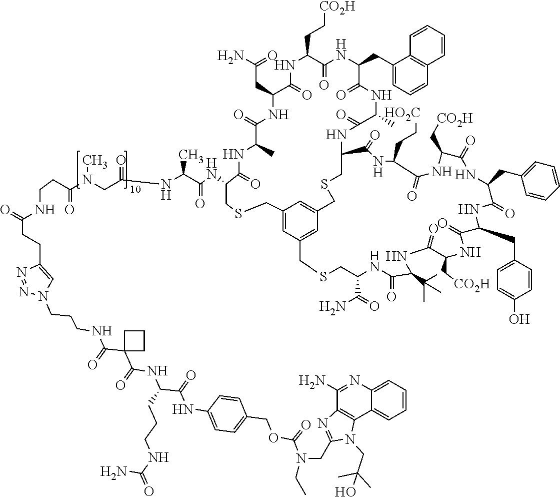 | I-30 |
| 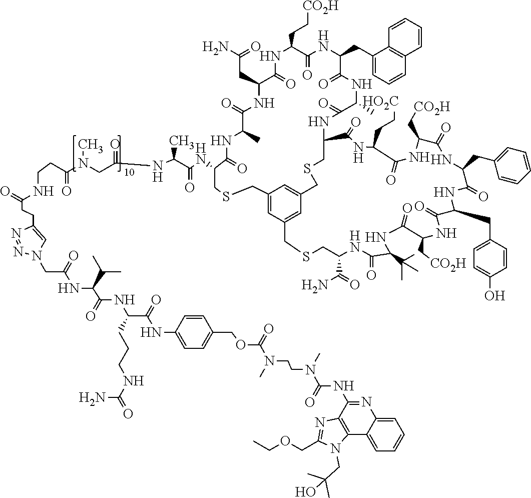 | I-31 |
| 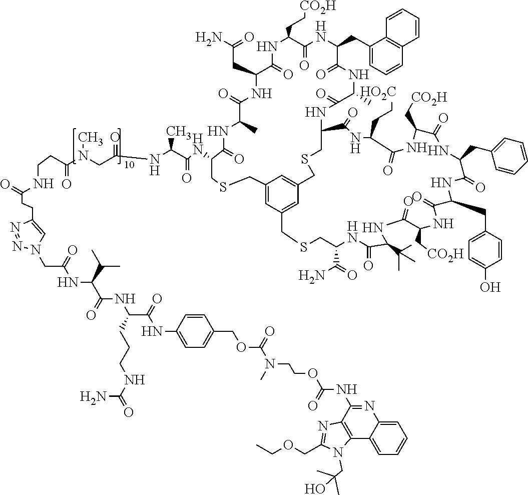 | I-32 |
| 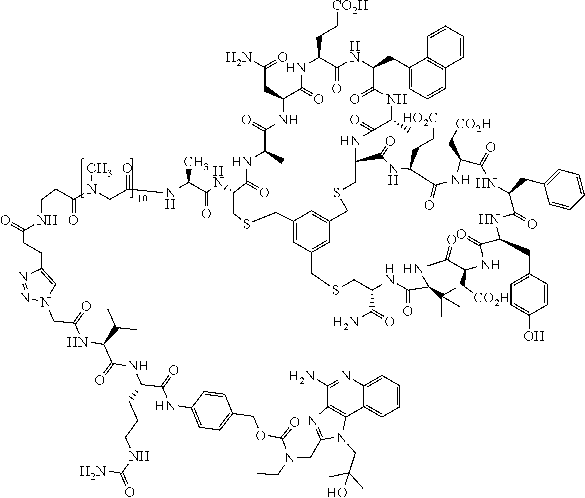 | I-33 |

In some embodiments, the present invention provides a compound set forth in Table 1, above, or a pharmaceutically acceptable salt thereof.

### 4. General Methods of Providing the Present Compounds

The compounds of this invention may be prepared or isolated in general by synthetic and/or semi-synthetic methods known to those skilled in the art for analogous compounds and by methods described in detail in the Examples, herein.

The compounds of this invention may be prepared by treating a peptide with a molecular scaffold reagent. The molecular scaffold reagent comprises the Scaffold and reactive functionality such as leaving groups (“LG”) or Michael acceptors (“MA”), that allow the peptide to form covalent bonds with the molecular scaffold via displacement of the leaving group or addition to the Michael acceptor group followed by subsequent protonation of the addition complex.

Compounds of the present invention are formed by treating peptides with various molecular scaffold reagents to form a Bicycle intermediate which is then coupled to PRR-A using standard amide formation methodology.

One such peptide is peptide 1 (17-69-07-N241), which has the following amino acid sequence:
βAla-Sar10-A-C(D-Ala)NE(1Nal)(D-Ala)CEDFYD(tBuGLy)C (SEQ ID NO:1)

The bicyclic peptide formed by treating 17-69-07-N241 with the molecular scaffold reagent 1,3,5-tris(bromomethyl)benzene (“TBMB”) as described in WO 2016/067035 affords an MT1-MMP binder with a K_{d} of 1.2 nM.

In the Schemes below, where a particular Michael acceptor (“MA”), leaving group (“LG”), or transformation condition is depicted, one of ordinary skill in the art will appreciate that other Michael acceptors, leaving groups, and transformation conditions are also suitable and are contemplated. Such acceptors, groups and transformations are described in detail in March's Advanced Organic Chemistry: Reactions, Mechanisms, and Structure, M. B. Smith and J. March, 5^{th} Edition, John Wiley & Sons, 2001, Comprehensive Organic Transformations, R. C. Larock, 2^{nd} Edition, John Wiley & Sons, 1999, and Protecting Groups in Organic Synthesis, T. W. Greene and P. G. M. Wuts, 3^{rd} edition, John Wiley & Sons, 1999, the entirety of each of which is hereby incorporated herein by reference.

As used herein, the phrase “leaving group” (LG) includes, but is not limited to, halogens (e.g. fluoride, chloride, bromide, iodide), sulfonates (e.g. mesylate, tosylate, benzenesulfonate, brosylate, nosylate, triflate), diazonium, and the like.

As used herein, the phrase “activated ester” (AE) includes, but is not limited to, isocyanates, isothiocyanates, acyl halides (e.g. acyl fluoride, acyl chloride, acyl bromide, acyl iodide), N-succinimidyl esters, uronium esters (e.g. 1-hydroxy-7azabenzotriazole, —OAt), and the like. Additionally, an AE can be prepared from a corresponding PRR-A-AE precursor acid in situ by treatment with coupling reagents known in the art such as, but not limited to DCC, DIC, EDC, HATU, HBTU, HCTU, PyBOP, PyAOP, PyBrOP, BOP, BOP-Cl, DEPBT, T3P, TATU, TBTU, TNTU, TOTU, TPTU, TSTU, or TDBTU.

The synthesis of the Linker-PRR-A conjugate is convergent in that one Linker can be converted to another Linker of the invention by treatment with PRR-A-AE which may comprise parts of the Linker in addition to the activated ester portion.

For purposes of clarity and by way of example, such PRR-A-AE precursor acids are as depicted below:

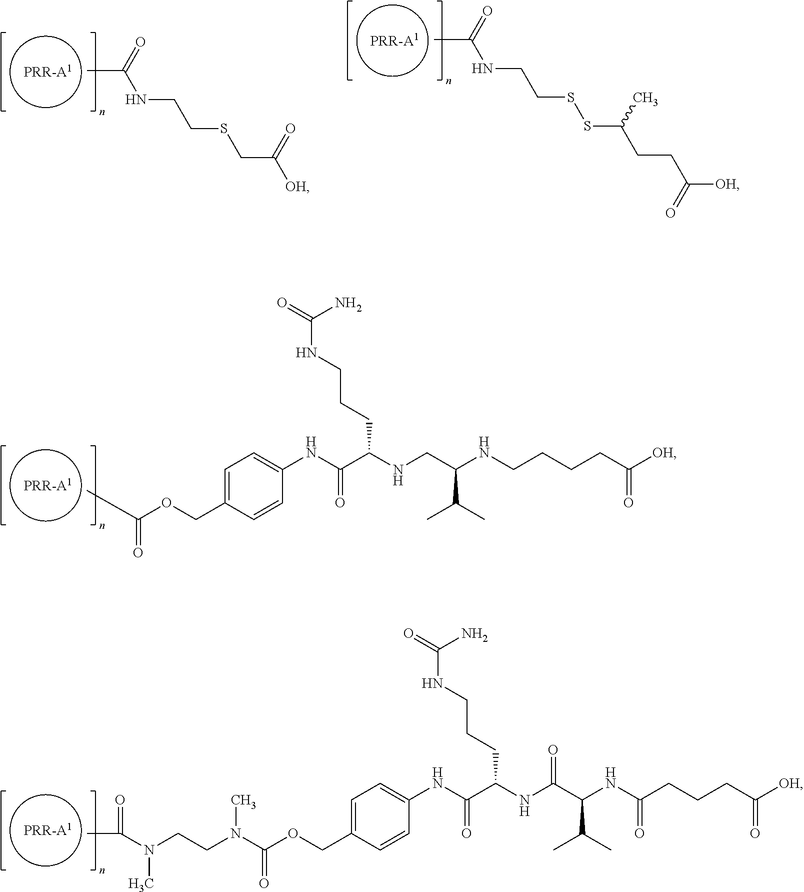

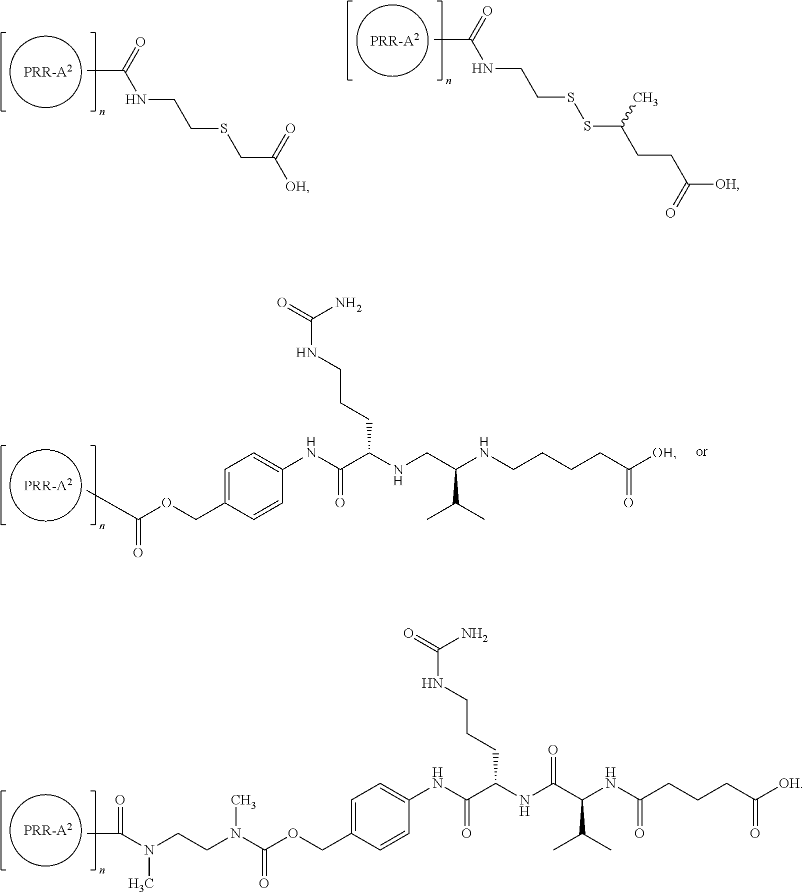

In certain embodiments, compounds of the present invention of formula I are generally prepared according to Scheme I set forth below:

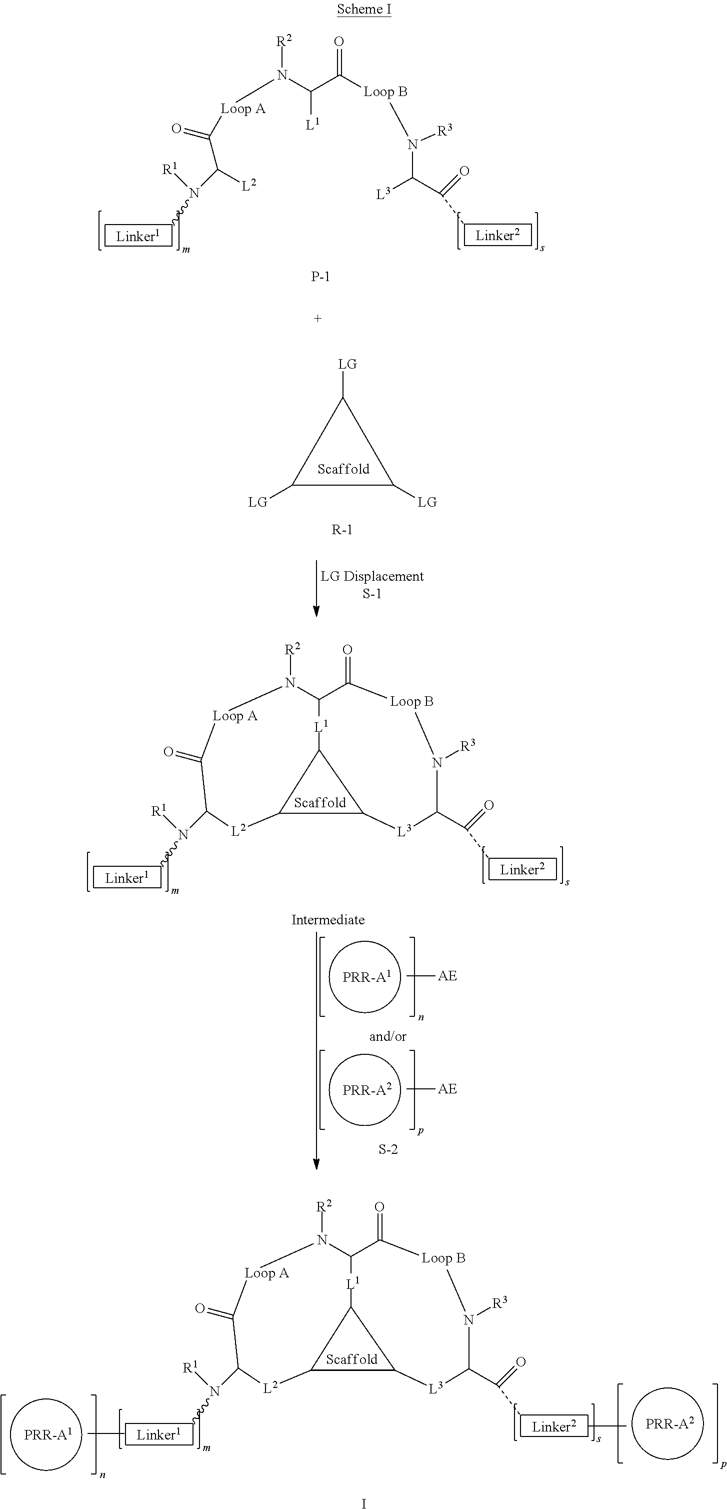

In Scheme I above, each of LG, L¹, L², L³, Scaffold, Linker¹, Linker², R¹, R², R³, Loop A, Loop B, PRR-A¹, PRR-A², AE, m, n, s and p is as defined above and below and in classes and subclasses as described herein.

In one aspect, the present invention provides methods for preparing compounds of formula I according to the steps depicted in Scheme I, above. In some embodiments, step S-1 comprises contacting the scaffold reagent R-1 with a peptide P-1 to displace the leaving group LG, thereby forming an intermediate which is further treated with an activated ester of PRR-A in step S-2 to afford a compound of formula I. In some embodiments, LG is a halogen. In some embodiments, LG is chlorine. In some embodiments, LG is a sulfonate. In some embodiments, AE is a N-succinimidyl ester. In some embodiments, a base is added to promote the displacement. In some embodiments, the base is ammonium carbonate. In some embodiments, the base is an amine. In some embodiments, the base is N,N-diisopropylethylamine.

In certain embodiments, step S-1 comprises contacting a compound of formula P-1 with a compound of the formula

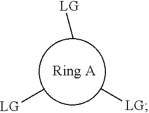

wherein
LG and Ring A are defined above and below and in classes and subclasses as described herein.

In some embodiments the reaction further comprises a solvent. In some embodiments the solvent is acetonitrile. In some embodiments the reaction further comprises a solvent. In some embodiments the solvent is DMSO. In some embodiments the solvent is a mixture of water and acetonitrile.

In some embodiments, LG is a halogen. In some embodiments, LG is chlorine. In some embodiments, LG is a sulfonate. In some embodiments, a catalyst is added to promote the displacement. In some embodiments, the catalyst is generated from 3^{rd} Generation XPhos precatalyst. In some embodiments, the solvent is tert-butanol. In some embodiments, the solvent is a mixture of water and tert-butanol.

In certain embodiments, compounds of the present invention of formula I are generally prepared according to Scheme II set forth below:

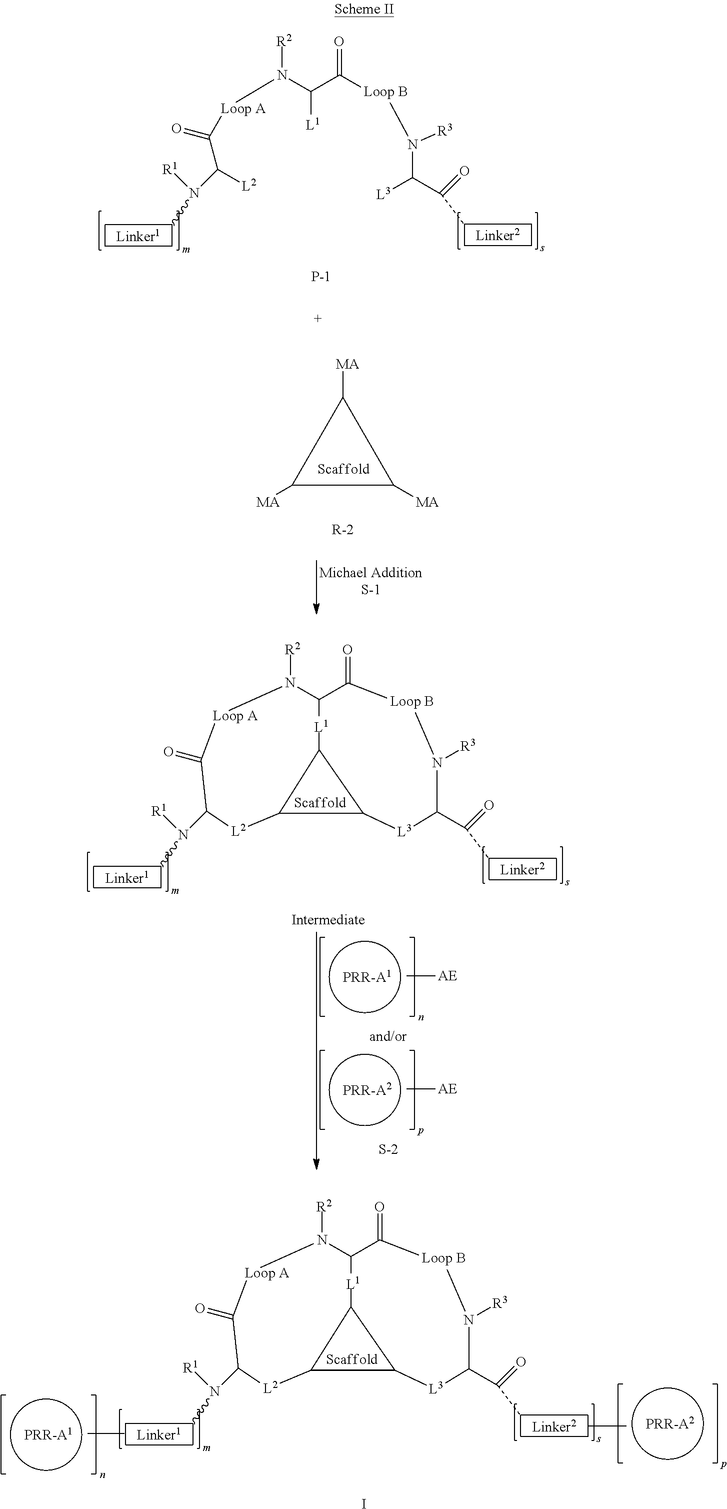

In Scheme II above, each of MA, L¹, L², L³, Scaffold, Linker¹, Linker², R¹, R², R³, Loop A, Loop B, PRR-A¹, PRR-A², AE, m, n, s, and p is as defined above and below and in classes and subclasses as described herein.

In one aspect, the present invention provides methods for preparing compounds of formula I according to the steps depicted in Scheme II, above. In some embodiments, step A-1 comprises contacting the scaffold reagent R-2 with a peptide P-1 to affect a Michael addition to MA, thereby forming a an intermediate which is further treated with an activated ester of PRR-A in step S-2 to afford a compound of formula I. In some embodiments, MA is an α,β-unsaturated amide. In some embodiments, MA is an α,β-unsaturated ketone. In some embodiments, MA is an α,β-unsaturated ester. In some embodiments, MA is an α,β-unsaturated sulfone. In some embodiments, MA is an α,β-unsaturated nitrile. In some embodiments, a base is added to promote the Michael addition. In some embodiments, AE is a N-succinimidyl ester. In some embodiments, the base is ammonium carbonate. In some embodiments, the base is an amine. In some embodiments, the base is N,N-diisopropylethylamine.

In certain embodiments, step A-1 comprises contacting a compound of formula P-1 with a compound of the formula

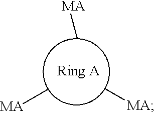

wherein
MA and Ring A are defined above and below and in classes and subclasses as described herein.

In some embodiments the reaction further comprises a solvent. In some embodiments the solvent is acetonitrile. In some embodiments the reaction further comprises a solvent. In some embodiments the solvent is DMSO. In some embodiments the solvent is a mixture of water and acetonitrile.

In some embodiments, MA is an α,β-unsaturated amide. In some embodiments, MA is an α,β-unsaturated ketone. In some embodiments, MA is an α,β-unsaturated ester. In some embodiments, MA is an α,β-unsaturated sulfone. In some embodiments, MA is an α,β-unsaturated nitrile. In some embodiments, a base is added to promote the Michael addition. In some embodiments, the base is ammonium carbonate. In some embodiments, the base is an amine. In some embodiments, the base is N,N-diisopropylethylamine.

### Scheme III.

In some embodiments, the present invention provides a method for synthesizing a compound of formula I by coupling a Bicycle peptide intermediate (“BPI”) to a PRR-A intermediate (“PLI”) via click chemistry. In some embodiments, a method for synthesizing a compound of formula I comprises coupling a Bicycle peptide intermediate having an alkyne group to a PRR-A intermediate having an azide group. In some embodiments, a method for synthesizing a compound of formula I comprises coupling a Bicycle peptide intermediate having an azide group to a PRR-A intermediate having an alkyne group. In some embodiments, each of a Bicycle peptide intermediate and a PRR-A¹ intermediate in a coupling reaction comprises part of Linker¹, wherein the coupling reaction forms Linker¹ between the Bicycle peptide moiety and the PRR-A¹ moiety, and Linker¹ comprises a 1,2,3-triazole moiety. In some embodiments, each of a Bicycle peptide intermediate and a PRR-A² intermediate in a coupling reaction comprises part of Linker², wherein the coupling reaction forms Linker² between the Bicycle peptide moiety and the PRR-A² moiety, and Linker² comprises a 1,2,3-triazole moiety.

In some embodiments, the present invention provides a method for synthesizing a compound of formula IV by click chemistry, as shown below is Scheme III.

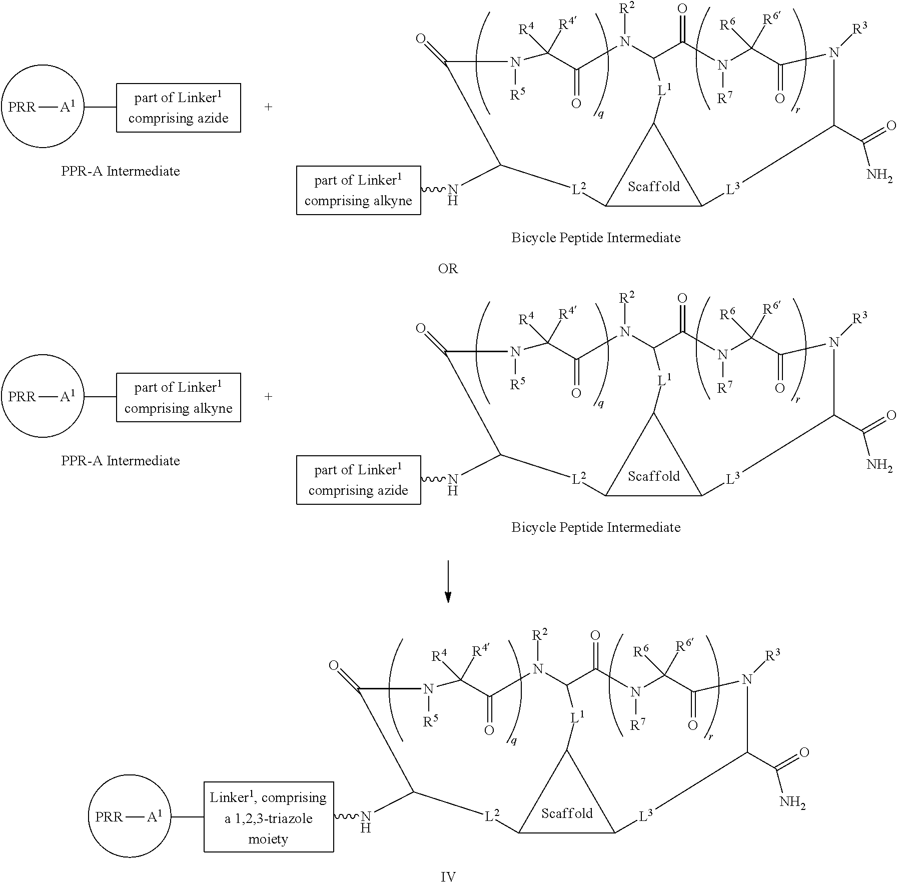

In Scheme III above, each of L¹, L², L³, Scaffold, R², R³, R⁴, R^{4′}, R⁵, R⁶, R^{6′}, R⁷, Linker¹, PRR-A¹, q and r is as defined above and below and in classes and subclasses as described herein.

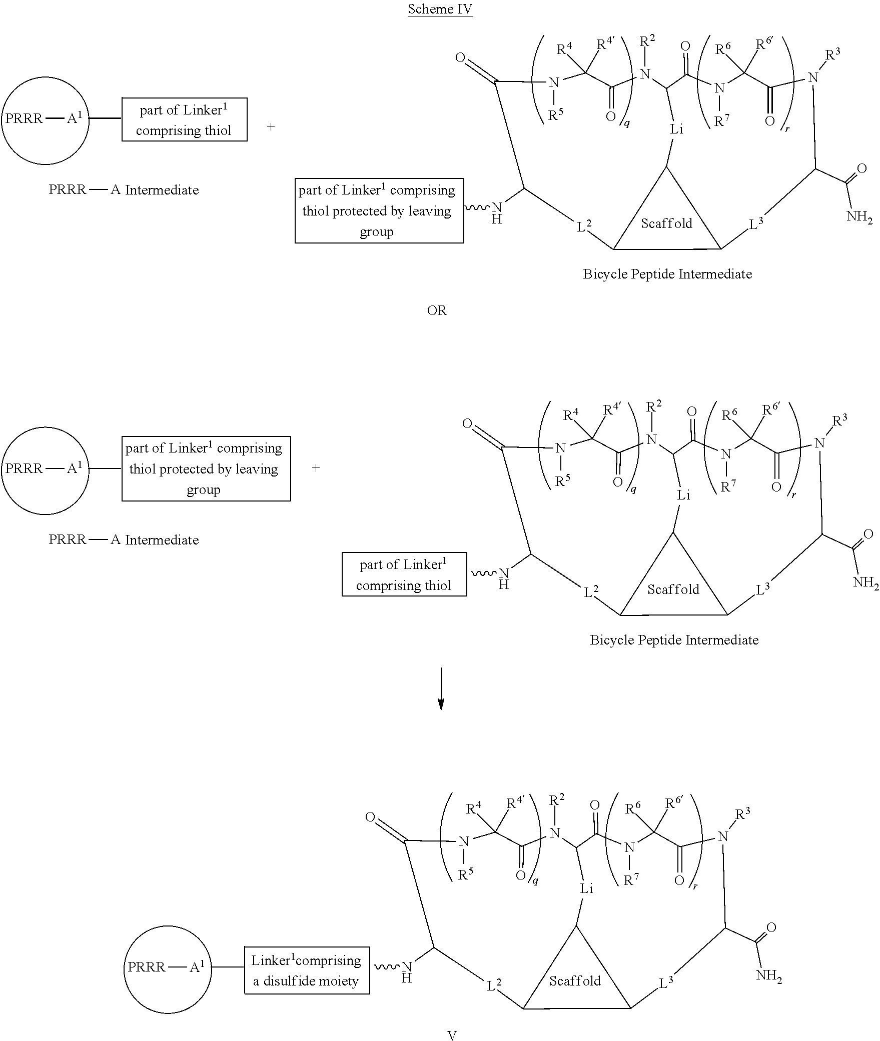

In some embodiments, the present invention provides a method for synthesizing a compound of formula I by coupling a Bicycle peptide intermediate (“BPI”) to a PRR-A intermediate (“PLI”) via disulfide chemistry. In some embodiments, a method for synthesizing a compound of formula I comprises coupling a Bicycle peptide intermediate having a thiol group to a PRR-A intermediate having a thiol group that is protected by a leaving group (for example, 2-mercaptopyridyl). In some embodiments, a method for synthesizing a compound of formula I comprises coupling a Bicycle peptide intermediate having a thiol group that is protected by a leaving group (for example, 2-mercaptopyridyl) to a PRR-A intermediate having a thiol group. In some embodiments, each of a Bicycle peptide intermediate and a PRR-A¹ intermediate in a coupling reaction comprises part of Linker¹, wherein the coupling reaction forms Linker¹ between the Bicycle peptide moiety and the PRR-A¹ moiety, and Linker¹ comprises a disulfide moiety. In some embodiments, each of a Bicycle peptide intermediate and a PRR-A² intermediate in a coupling reaction comprises part of Linker², wherein the coupling reaction forms Linker² between the Bicycle peptide moiety and the PRR-A² moiety, and Linker² comprises a disulfide moiety.

In some embodiments, the present invention provides a method for synthesizing a compound of formula V by disulfide chemistry, as shown below is Scheme IV.

In Scheme III above, each of L¹, L², L³, Scaffold, R², R³, R⁴, R^{4′}, R⁵, R⁶, R^{6′}, R⁷, Linker¹, PRR-A¹, q and r is as defined above and below and in classes and subclasses as described herein.

One of skill in the art will appreciate that compounds of formula I may contain one or more stereocenters, and may be present as an racemic or diastereomeric mixture. One of skill in the art will also appreciate that there are many methods known in the art for the separation of isomers to obtain stereoenriched or stereopure isomers of those compounds, including but not limited to HPLC, chiral HPLC, fractional crystallization of diastereomeric salts, kinetic enzymatic resolution (e.g. by fungal-, bacterial-, or animal-derived lipases or esterases), and formation of covalent diastereomeric derivatives using an enantioenriched reagent.

One of skill in the art will appreciate that various functional groups present in compounds of the invention such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. “March's Advanced Organic Chemistry”, 5^{th} Ed., Ed.: Smith, M. B. and March, J., John Wiley & Sons, New York: 2001, the entirety of which is incorporated herein by reference. Such interconversions may require one or more of the aforementioned techniques, and certain methods for synthesizing compounds of the invention are described below in the Exemplification.

In some embodiments, a PRR-A intermediate is selected from Table 2 below.

TABLE 2
Exemplary PRR-A intermediates.

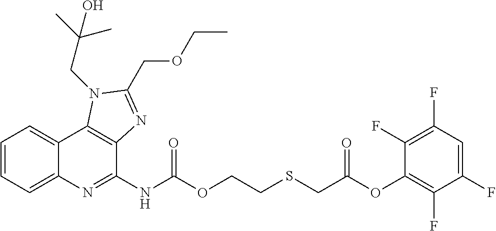 [SMILES: CCOCc1nc2c(NC(=O)OCCSCC(=O)Oc3c(F)c(F)cc(F)c3F)nc3ccccc3c2n1CC(C)(C)O]

PLI-1

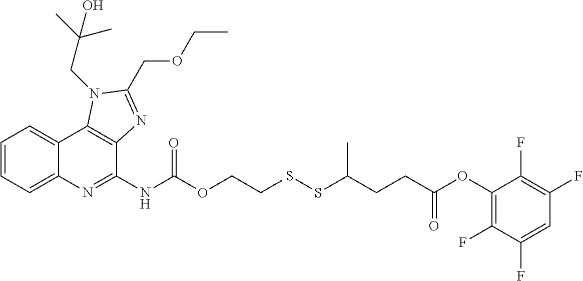 [SMILES: CCOCc1nc2c(NC(=O)OCCSSC(C)CCC(=O)Oc3c(F)c(F)cc(F)c3F)nc3ccccc3c2n1CC(C)(C)O]

PLI-2

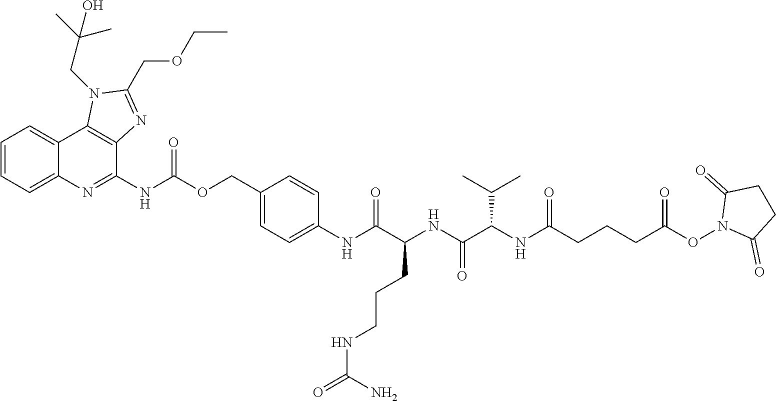 [SMILES: CCOCc1nc2c(NC(=O)OCc3ccc(NC(=O)[C@H](CCCNC(N)=O)NC(=O)[C@@H](NC(=O)CCCC(=O)ON4C(=O)CCC4=O)C(C)C)cc3)nc3ccccc3c2n1CC(C)(C)O]

PLI-3

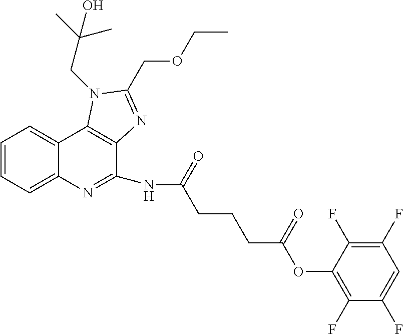 [SMILES: CCOCc1nc2c(NC(=O)CCCC(=O)Oc3c(F)c(F)cc(F)c3F)nc3ccccc3c2n1CC(C)(C)O]

PLI-4

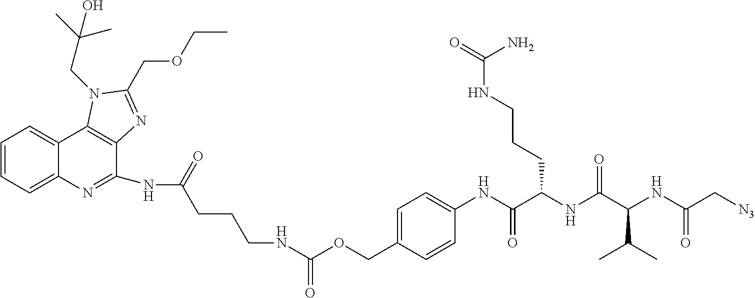 [SMILES: CCOCc1nc2c(NC(=O)CCCNC(=O)OCc3ccc(NC(=O)[C@H](CCCNC(N)=O)NC(=O)[C@@H](NC(=O)CN=[N+]=[N-])C(C)C)cc3)nc3ccccc3c2n1CC(C)(C)O]

PLI-5

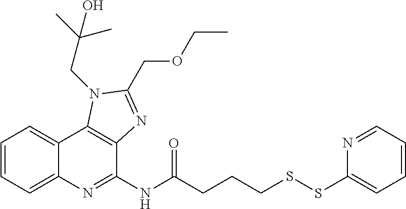 [SMILES: CCOCc1nc2c(NC(=O)CCCSSc3ccccn3)nc3ccccc3c2n1CC(C)(C)O]

PLI-6

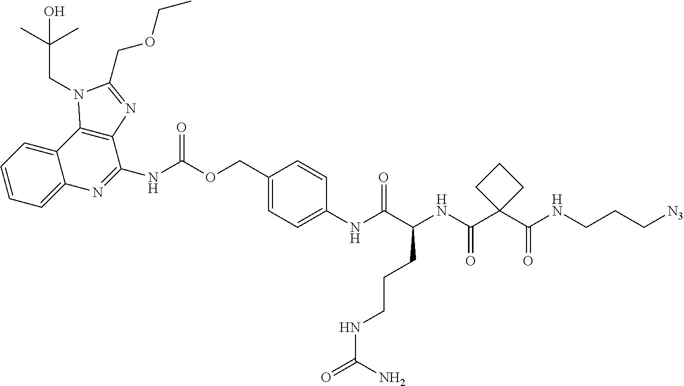 [SMILES: CCOCc1nc2c(NC(=O)OCc3ccc(NC(=O)[C@H](CCCNC(N)=O)NC(=O)C4(C(=O)NCCCN=[N+]=[N-])CCC4)cc3)nc3ccccc3c2n1CC(C)(C)O]

PLI-7

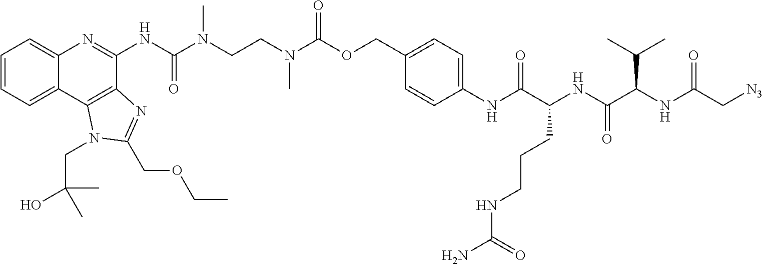 [SMILES: CCOCc1nc2c(NC(=O)N(C)CCN(C)C(=O)OCc3ccc(NC(=O)[C@@H](CCCNC(N)=O)NC(=O)[C@H](NC(=O)CN=[N+]=[N-])C(C)C)cc3)nc3ccccc3c2n1CC(C)(C)O]

PLI-8

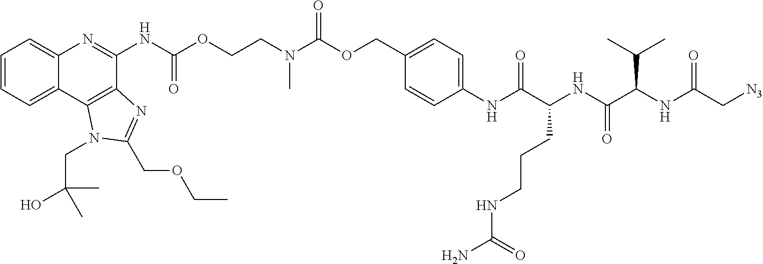 [SMILES: CCOCc1nc2c(NC(=O)OCCN(C)C(=O)OCc3ccc(NC(=O)[C@@H](CCCNC(N)=O)NC(=O)[C@H](NC(=O)CN=[N+]=[N-])C(C)C)cc3)nc3ccccc3c2n1CC(C)(C)O]

PLI-9

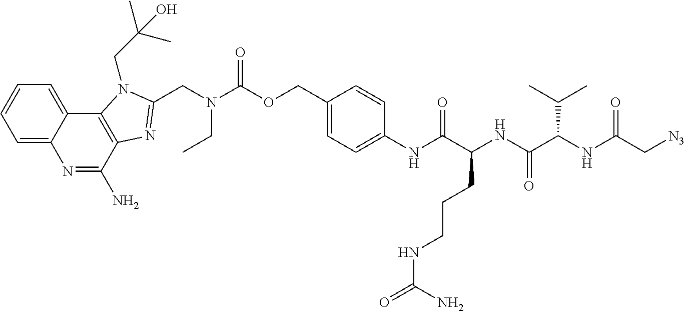 [SMILES: CCN(Cc1nc2c(N)nc3ccccc3c2n1CC(C)(C)O)C(=O)OCc1ccc(NC(=O)[C@H](CCCNC(N)=O)NC(=O)[C@@H](NC(=O)CN=[N+]=[N-])C(C)C)cc1]

PLI-10

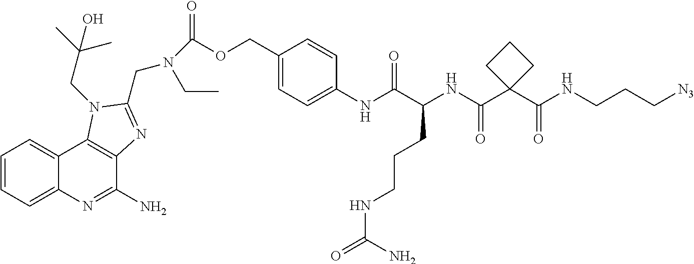 [SMILES: CCN(Cc1nc2c(N)nc3ccccc3c2n1CC(C)(C)O)C(=O)OCc1ccc(NC(=O)[C@H](CCCNC(N)=O)NC(=O)C2(C(=O)NCCCN=[N+]=[N-])CCC2)cc1]

PLI-11

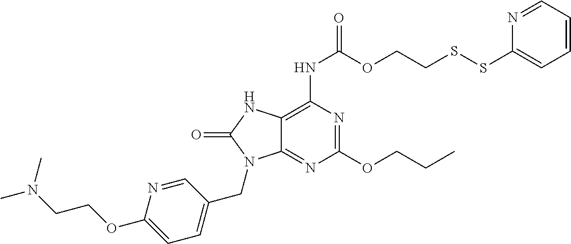 [SMILES: CCCOc1nc(NC(=O)OCCSSc2ccccn2)c2[nH]c(=O)n(Cc3ccc(OCCN(C)C)nc3)c2n1]

PLI-12

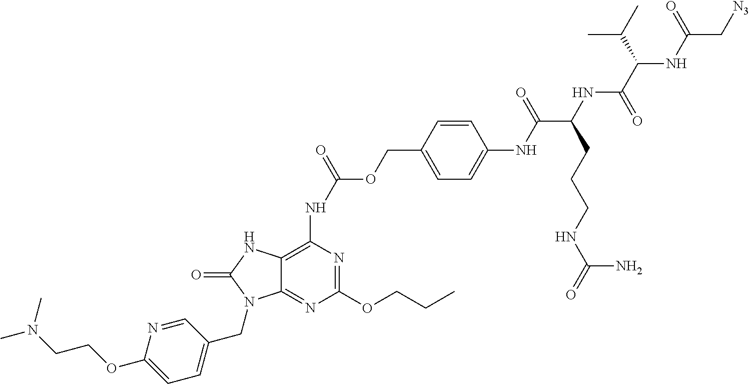 [SMILES: CCCOc1nc(NC(=O)OCc2ccc(NC(=O)[C@H](CCCNC(N)=O)NC(=O)[C@@H](NC(=O)CN=[N+]=[N-])C(C)C)cc2)c2[nH]c(=O)n(Cc3ccc(OCCN(C)C)nc3)c2n1]

PLI-13

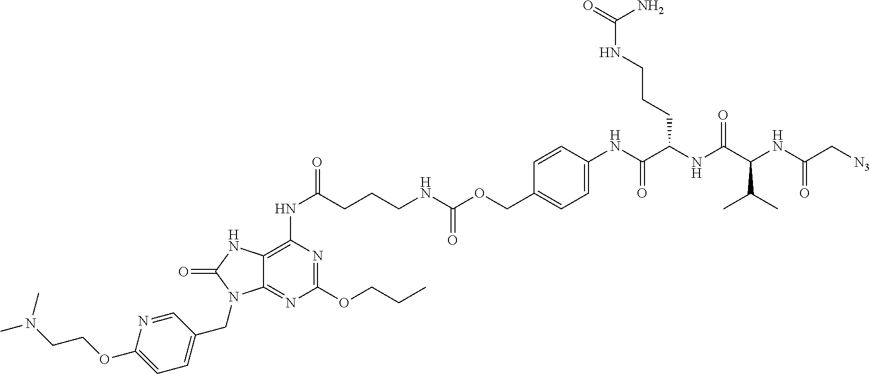 [SMILES: CCCOc1nc(NC(=O)CCCNC(=O)OCc2ccc(NC(=O)[C@H](CCCNC(N)=O)NC(=O)[C@@H](NC(=O)CN=[N+]=[N-])C(C)C)cc2)c2[nH]c(=O)n(Cc3ccc(OCCN(C)C)nc3)c2n1]

PLI-14

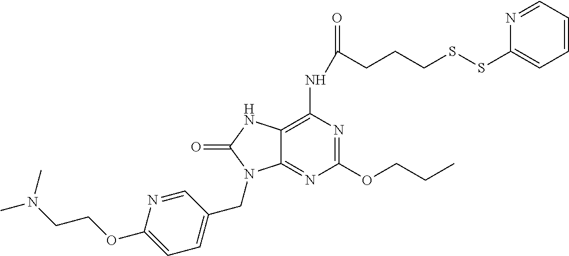 [SMILES: CCCOc1nc(NC(=O)CCCSSc2ccccn2)c2[nH]c(=O)n(Cc3ccc(OCCN(C)C)nc3)c2n1]

PLI-15

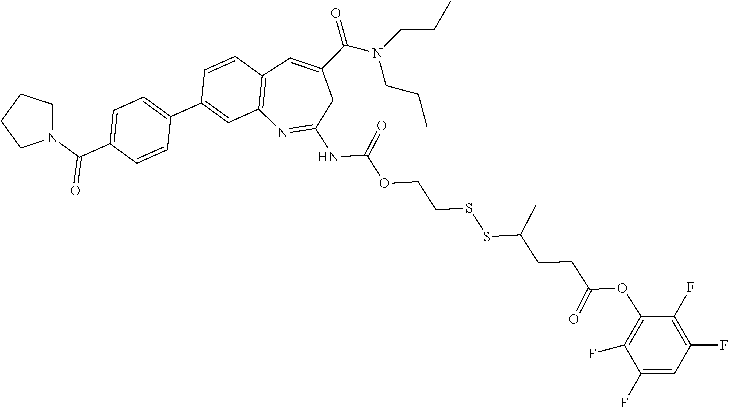 [SMILES: CCCN(CCC)C(=O)C1=Cc2ccc(-c3ccc(C(=O)N4CCCC4)cc3)cc2N=C(NC(=O)OCCSSC(C)CCC(=O)Oc2c(F)c(F)cc(F)c2F)C1]

PLI-16

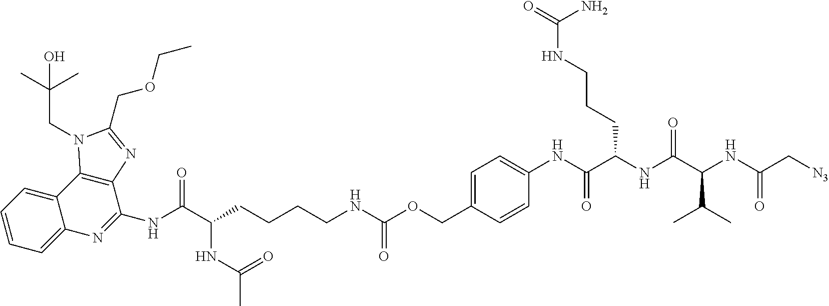 [SMILES: CCOCc1nc2c(NC(=O)[C@H](CCCCNC(=O)OCc3ccc(NC(=O)[C@H](CCCNC(N)=O)NC(=O)[C@@H](NC(=O)CN=[N+]=[N-])C(C)C)cc3)NC(C)=O)nc3ccccc3c2n1CC(C)(C)O]

PLI-17

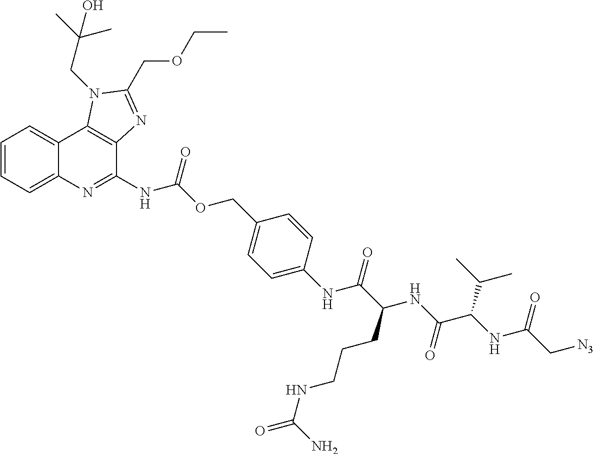 [SMILES: CCOCc1nc2c(NC(=O)OCc3ccc(NC(=O)[C@H](CCCNC(N)=O)NC(=O)[C@@H](NC(=O)CN=[N+]=[N-])C(C)C)cc3)nc3ccccc3c2n1CC(C)(C)O]

PLI-18

In some embodiments, a bicycle peptide intermediate is selected from Table 3 below.

TABLE 3
Exemplary bicycle peptide intermediates.

| | |
|---|---|
| 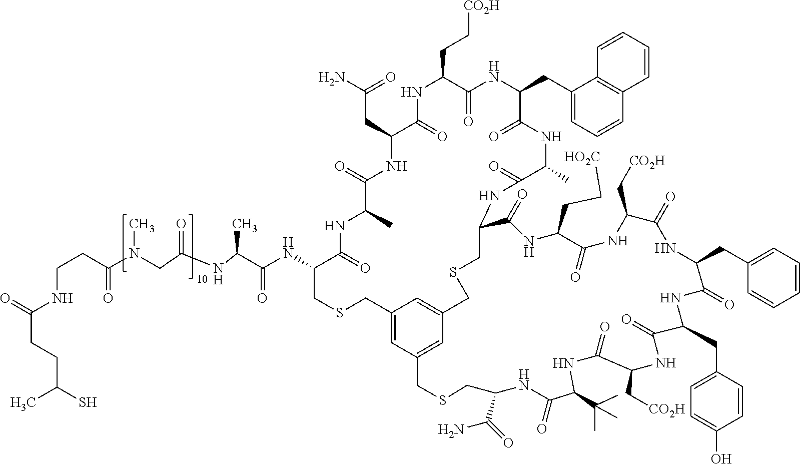 | BPI-1 |
| 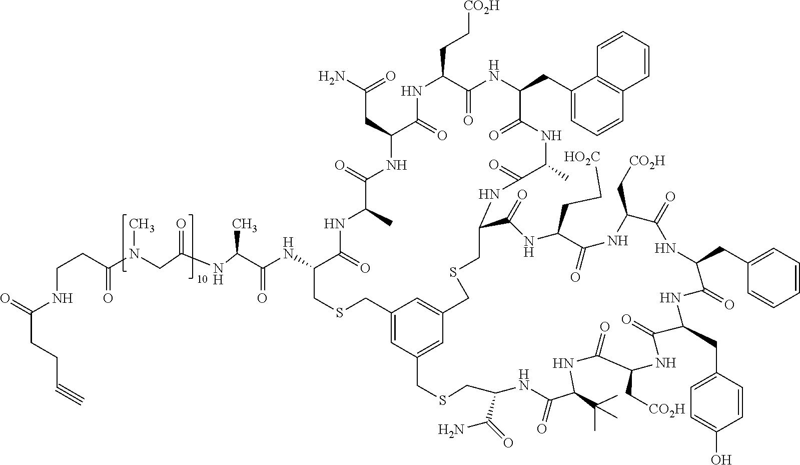 | BPI-2 |

### 5. Uses, Formulation and Administration

Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that induces an immune response in a biological sample or in a patient. In certain embodiments, the amount of compound in compositions of this invention is such that is effective to induce an immune response in a biological sample or in a patient. In certain embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

As used herein, the term “inhibitorily active metabolite or residue thereof” means that a metabolite or residue thereof is also an inhibitor of MT1-MMP, or a mutant thereof.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Uses of Compounds and Pharmaceutically Acceptable Compositions

In another aspect, certain bicyclic peptides of the invention have specific utility as high affinity binders of membrane type 1 metalloprotease (MT1-MMP, also known as MMP14). MT1-MMP is a transmembrane metalloprotease that plays a major role in the extracellular matrix remodeling, directly by degrading several of its components and indirectly by activating pro-MMP2. MT1-MMP is crucial for tumor angiogenesis (Sounni et al (2002) FASEB J. 16(6), 555-564) and is over-expressed on a variety of solid tumors, therefore the MT1-MMP-binding bicycle peptides of the present invention have particular utility in the targeted treatment of cancer, in particular solid tumors such as non-small cell lung carcinomas, via targeted delivery of a conjugated payload such as a PRR-A. In one embodiment, the bicyclic peptide of the invention is specific for human MT1-MMP. In a further embodiment, the bicyclic peptide of the invention is specific for mouse MT1-MMP. In a yet further embodiment, the bicyclic peptide of the invention is specific for human and mouse MT1-MMP. In a yet further embodiment, the bicyclic peptide of the invention is specific for human, mouse and dog MT1-MMP.

Compounds and compositions described herein are generally useful for the inhibition of metalloprotease activity of one or more enzymes.

Polypeptide ligands selected according to the method of the present invention may be employed in in vivo therapeutic and prophylactic applications, in vitro and in vivo diagnostic applications, in vitro assay and reagent applications, and the like. Ligands having selected levels of specificity are useful in applications which involve testing in non-human animals, where cross-reactivity is desirable, or in diagnostic applications, where cross-reactivity with homologues or paralogues needs to be carefully controlled. In some applications, such as vaccine applications, the ability to elicit an immune response to predetermined ranges of antigens can be exploited to tailor a vaccine to specific diseases and pathogens.

Substantially pure peptide ligands of at least 90 to 95% homogeneity are preferred for administration to a mammal, and 98 to 99% or more homogeneity is most preferred for pharmaceutical uses, especially when the mammal is a human. Once purified, partially or to homogeneity as desired, the selected polypeptides may be used diagnostically or therapeutically (including extracorporeally) or in developing and performing assay procedures, immunofluorescent stainings and the like (Lefkovite and Pernis, (1979 and 1981) Immunological Methods, Volumes I and II, Academic Press, NY).

The activity of a compound utilized in this invention as an inhibitor of MT1-MMP, or a mutant thereof, may be assayed in vitro, in vivo or in a cell line. Alternative in vitro assays quantitate the ability of the inhibitor to bind to MT1-MMP. Alternatively, inhibitor binding may be determined by running a competition experiment where new inhibitors are incubated with MT1-MMP bound to known radioligands. Representative in vitro and in vivo assays useful in assaying an MT1-MMP inhibitor include those described and disclosed in: Pietraszek et al., (2014) FEBS Letters 588(23), 4319-4324; Cheltsov et al., (2012) Cancer Res. 72(9), 2339-49; and WO 2009/098450, each of which is herein incorporated by reference in its entirety. Detailed conditions for assaying a compound utilized in this invention as an inhibitor of MT1-MMP, or a mutant thereof, are set forth in the Examples below.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Provided compounds are binders of MT1-MMP and are therefore useful for the targeted treatment of MT1-MMP expressing cancer cells. Thus, in certain embodiments, the present invention provides a method for the targeted treatment of a disorder comprising the step of administering to a patient in need thereof a compound of the present invention, or pharmaceutically acceptable composition thereof.

Examples of cancers (and their benign counterparts) which may be treated (or inhibited) include, but are not limited to tumors of epithelial origin (adenomas and carcinomas of various types including adenocarcinomas, squamous carcinomas, transitional cell carcinomas and other carcinomas) such as carcinomas of the bladder and urinary tract, breast, gastrointestinal tract (including the esophagus, stomach (gastric), small intestine, colon, rectum and anus), liver (hepatocellular carcinoma), gall bladder and biliary system, exocrine pancreas, kidney, lung (for example adenocarcinomas, small cell lung carcinomas, non-small cell lung carcinomas, bronchioalveolar carcinomas and mesotheliomas), head and neck (for example cancers of the tongue, buccal cavity, larynx, pharynx, nasopharynx, tonsil, salivary glands, nasal cavity and paranasal sinuses), ovary, fallopian tubes, peritoneum, vagina, vulva, penis, cervix, myometrium, endometrium, thyroid (for example thyroid follicular carcinoma), adrenal, prostate, skin and adnexae (for example melanoma, basal cell carcinoma, squamous cell carcinoma, keratoacanthoma, dysplastic naevus); hematological malignancies (i.e. leukemias, lymphomas) and premalignant hematological disorders and disorders of borderline malignancy including hematological malignancies and related conditions of lymphoid lineage (for example acute lymphocytic leukemia [ALL], chronic lymphocytic leukemia [CLL], B-cell lymphomas such as diffuse large B-cell lymphoma [DLBCL], follicular lymphoma, Burkitt's lymphoma, mantle cell lymphoma, T-cell lymphomas and leukemias, natural killer [NK] cell lymphomas, Hodgkin's lymphomas, hairy cell leukemia, monoclonal gammopathy of uncertain significance, plasmacytoma, multiple myeloma, and post-transplant lymphoproliferative disorders), and hematological malignancies and related conditions of myeloid lineage (for example acute myelogenousleukemia [AML], chronic myelogenousleukemia [CIVIL], chronic myelomonocyticleukemia [CMML], hypereosinophilic syndrome, myeloproliferative disorders such as polycythaemia vera, essential thrombocythaemia and primary myelofibrosis, myeloproliferative syndrome, myelodysplastic syndrome, and promyelocyticleukemia); tumors of mesenchymal origin, for example sarcomas of soft tissue, bone or cartilage such as osteosarcomas, fibrosarcomas, chondrosarcomas, rhabdomyosarcomas, leiomyosarcomas, liposarcomas, angiosarcomas, Kaposi's sarcoma, Ewing's sarcoma, synovial sarcomas, epithelioid sarcomas, gastrointestinal stromal tumors, benign and malignant histiocytomas, and dermatofibrosarcomaprotuberans; tumors of the central or peripheral nervous system (for example astrocytomas, gliomas and glioblastomas, meningiomas, ependymomas, pineal tumors and schwannomas); endocrine tumors (for example pituitary tumors, adrenal tumors, islet cell tumors, parathyroid tumors, carcinoid tumors and medullary carcinoma of the thyroid); ocular and adnexal tumors (for example retinoblastoma); germ cell and trophoblastic tumors (for example teratomas, seminomas, dysgerminomas, hydatidiform moles and choriocarcinomas); and pediatric and embryonal tumors (for example medulloblastoma, neuroblastoma, Wilms tumor, and primitive neuroectodermal tumors); or syndromes, congenital or otherwise, which leave the patient susceptible to malignancy (for example Xeroderma Pigmentosum).

In a further embodiment, the cancer is selected from cancer of the cervix, ovary, kidney, esophagus, lung, breast and brain.

References herein to the term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest.

Animal model systems which can be used to screen the effectiveness of the peptide ligands in protecting against or treating the disease are available. The use of animal model systems is facilitated by the present invention, which allows the development of polypeptide ligands which can cross react with human and animal targets, to allow the use of animal models.

Furthermore, the invention provides the use of a compound according to the definitions herein, or a pharmaceutically acceptable salt, or a hydrate or solvate thereof for the preparation of a medicament for the treatment of a proliferative disease.

Combination Therapies

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

In certain embodiments, combination therapies of the present invention, or a pharmaceutically acceptable composition thereof, are administered in combination with a monoclonal antibody or an siRNA therapeutic.

Those additional agents may be administered separately from a provided combination therapy, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a combination of the present invention may be administered with another therapeutic agent simultaneously or sequentially in separate unit dosage forms or together in a single unit dosage form.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

In one embodiment, the present invention provides a composition comprising a compound of formula I and one or more additional therapeutic agents. The therapeutic agent may be administered together with a compound of formula I, or may be administered prior to or following administration of a compound of formula I. Suitable therapeutic agents are described in further detail below. In certain embodiments, a compound of formula I may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a compound of formula I may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

In another embodiment, the present invention provides a method of treating a hematological malignancy comprising administering to a patient in need thereof a compound of formula I and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a TYK2 inhibitor, a PI3K inhibitor, a SYK inhibitor, and combinations thereof.

In another embodiment, the present invention provides a method of treating a solid tumor comprising administering to a patient in need thereof a compound of formula I and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a TYK2 inhibitor, a PI3K inhibitor, a SYK inhibitor, and combinations thereof.

In another embodiment, the present invention provides a method of treating a hematological malignancy comprising administering to a patient in need thereof a compound of formula I and a Hedgehog (Hh) signaling pathway inhibitor. In some embodiments, the hematological malignancy is DLBCL (Ramirez et al “Defining causative factors contributing in the activation of hedgehog signaling in diffuse large B-cell lymphoma” Leuk. Res. (2012), published online July 17, and incorporated herein by reference in its entirety).

In another embodiment, the present invention provides a method of treating diffuse large B-cell lymphoma (DLBCL) comprising administering to a patient in need thereof a compound of formula I and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, and combinations thereof.

In another embodiment, the present invention provides a method of treating multiple myeloma comprising administering to a patient in need thereof a compound of formula I and one or more additional therapeutic agents selected from bortezomib (Velcade®), and dexamethasone (Decadron®), a hedgehog signaling inhibitor, a BTK inhibitor, a JAK/pan-JAK inhibitor, a TYK2 inhibitor, a PI3K inhibitor, a SYK inhibitor in combination with lenalidomide (Revlimid®).

In another embodiment, the present invention provides a method of treating Waldenström's macroglobulinemia comprising administering to a patient in need thereof a compound of formula I and one or more additional therapeutic agents selected from chlorambucil (Leukeran®), cyclophosphamide (Cytoxan®. Neosar®), fludarabine (Fludara®), cladribine (Leustatin®), rituximab (Rituxan®), a hedgehog signaling inhibitor, a BTK inhibitor, a JAK/pan-JAK inhibitor, a TYK2 inhibitor, a PI3K inhibitor, and a SYK inhibitor.

In another embodiment, the present invention provides a method of treating or lessening the severity of a disease comprising administering to a patient in need thereof a compound of formula I and a BTK inhibitor, wherein the disease is selected from inflammatory bowel disease, arthritis, systemic lupus erythematosus (SLE), vasculitis, idiopathic thrombocytopenic purpura (ITP), rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, autoimmune thyroiditis, Sjogren's syndrome, multiple sclerosis, systemic sclerosis, Lyme neuroborreliosis, Guillain-Barre syndrome, acute disseminated encephalomyelitis, Addison's disease, opsoclonus-myoclonus syndrome, ankylosing spondylosis, antiphospholipid antibody syndrome, aplastic anemia, autoimmune hepatitis, autoimmune gastritis, pernicious anemia, celiac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, membranous glomerulonephropathy, endometriosis, interstitial cystitis, pemphigus vulgaris, bullous pemphigoid, neuromyotonia, scleroderma, vulvodynia, a hyperproliferative disease, rejection of transplanted organs or tissues, Acquired Immunodeficiency Syndrome (AIDS, also known as HIV), type 1 diabetes, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis, asthma, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis, B-cell proliferative disorder, e.g., diffuse large B cell lymphoma, follicular lymphoma, chronic lymphocytic lymphoma, chronic lymphocytic leukemia, acute lymphocytic leukemia, B-cell prolymphocytic leukemia, lymphoplasmacytic lymphoma/Waldenstrom macroglobulinemia, splenic marginal zone lymphoma, multiple myeloma (also known as plasma cell myeloma), non-Hodgkin's lymphoma, Hodgkin's lymphoma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukemia, or lymphomatoid granulomatosis, breast cancer, prostate cancer, or cancer of the mast cells (e.g., mastocytoma, mast cell leukemia, mast cell sarcoma, systemic mastocytosis), bone cancer, colorectal cancer, pancreatic cancer, diseases of the bone and joints including, without limitation, rheumatoid arthritis, seronegative spondyloarthropathies (including ankylosing spondylitis, psoriatic arthritis and Reiter's disease), Behcet's disease, Sjogren's syndrome, systemic sclerosis, osteoporosis, bone cancer, bone metastasis, a thromboembolic disorder, (e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, deep venous thrombosis), inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenic states, Goodpasture's syndrome, atherosclerosis, Addison's disease, Parkinson's disease, Alzheimer's disease, diabetes, septic shock, systemic lupus erythematosus (SLE), rheumatoid arthritis, psoriatic arthritis, juvenile arthritis, osteoarthritis, chronic idiopathic thrombocytopenic purpura, Waldenstrom macroglobulinemia, myasthenia gravis, Hashimoto's thyroiditis, atopic dermatitis, degenerative joint disease, vitiligo, autoimmune hypopituitarism, Guillain-Barre syndrome, Behcet's disease, scleroderma, mycosis fungoides, acute inflammatory responses (such as acute respiratory distress syndrome and ischemia/reperfusion injury), and Graves' disease.

In another embodiment, the present invention provides a method of treating or lessening the severity of a disease comprising administering to a patient in need thereof a compound of formula and a PI3K inhibitor, wherein the disease is selected from a cancer, a neurodegenerative disorder, an angiogenic disorder, a viral disease, an autoimmune disease, an inflammatory disorder, a hormone-related disease, conditions associated with organ transplantation, immunodeficiency disorders, a destructive bone disorder, a proliferative disorder, an infectious disease, a condition associated with cell death, thrombin-induced platelet aggregation, chronic myelogenous leukemia (CML), chronic lymphocytic leukemia (CLL), liver disease, pathologic immune conditions involving T cell activation, a cardiovascular disorder, and a CNS disorder.

In another embodiment, the present invention provides a method of treating or lessening the severity of a disease comprising administering to a patient in need thereof a compound of formula I and a PI3K inhibitor, wherein the disease is selected from benign or malignant tumor, carcinoma or solid tumor of the brain, kidney (e.g., renal cell carcinoma (RCC)), liver, adrenal gland, bladder, breast, stomach, gastric tumors, ovaries, colon, rectum, prostate, pancreas, lung, vagina, endometrium, cervix, testis, genitourinary tract, esophagus, larynx, skin, bone or thyroid, sarcoma, glioblastomas, neuroblastomas, multiple myeloma or gastrointestinal cancer, especially colon carcinoma or colorectal adenoma or a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, (including, for example, non-Hodgkin's Lymphoma (NHL) and Hodgkin's lymphoma (also termed Hodgkin's or Hodgkin's disease)), a mammary carcinoma, follicular carcinoma, undifferentiated carcinoma, papillary carcinoma, seminoma, melanoma, or a leukemia, diseases include Cowden syndrome, Lhermitte-Dudos disease and Bannayan-Zonana syndrome, or diseases in which the PI3K/PKB pathway is aberrantly activated, asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection, acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy, bronchitis of whatever type or genesis including, but not limited to, acute, arachidic, catarrhal, croupus, chronic or phthinoid bronchitis, pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis, Loffler's syndrome, eosinophilic, pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction, psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphigus, epidermolysis bullosa acquisita, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleroderma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary biliary cirrhosis, uveitis (anterior and posterior), keratoconjunctivitis sicca and vernal keratoconjunctivitis, interstitial lung fibrosis, psoriatic arthritis and glomerulonephritis (with and without nephrotic syndrome, e.g. including idiopathic nephrotic syndrome or minal change nephropathy, restenosis, cardiomegaly, atherosclerosis, myocardial infarction, ischemic stroke and congestive heart failure, Alzheimer's disease, Parkinson's disease, amyotrophic lateral sclerosis, Huntington's disease, and cerebral ischemia, and neurodegenerative disease caused by traumatic injury, glutamate neurotoxicity and hypoxia.

The compounds and compositions, according to the method of the present invention, may be administered using any amount and any route of administration effective for treating or lessening the severity of a cancer, an autoimmune disorder, a proliferative disorder, an inflammatory disorder, a neurodegenerative or neurological disorder, schizophrenia, a bone-related disorder, liver disease, or a cardiac disorder. The exact amount required will vary from subject to subject, depending on the species, age, and general condition of the subject, the severity of the infection, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

Pharmaceutically acceptable compositions of this invention can be administered to humans and other animals orally, rectally, parenterally, intracisternally, intravaginally, intraperitoneally, topically (as by powders, ointments, or drops), buccally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsulated matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar--, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to one embodiment, the invention relates to a method of inhibiting carbonic anhydrase activity in a biological sample comprising the step of contacting said biological sample with a compound of this invention, or a composition comprising said compound.

According to another embodiment, the invention relates to a method of inhibiting metalloprotease activity in a biological sample comprising the step of contacting said biological sample with a compound of this invention, or a composition comprising said compound.

According to another embodiment, the invention relates to a method of inhibiting integrin activity in a biological sample comprising the step of contacting said biological sample with a compound of this invention, or a composition comprising said compound.

According to another embodiment, the invention relates to a method of inhibiting MT1-MMP, or a mutant thereof, activity in a biological sample comprising the step of contacting said biological sample with a compound of this invention, or a composition comprising said compound.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition of MT1-MMP, or a mutant thereof, activity in a biological sample is useful for a variety of purposes that are known to one of skill in the art. Examples of such purposes include, but are not limited to, biological assays.

Another embodiment of the present invention relates to a method of inhibiting metalloprotease activity in a patient comprising the step of administering to said patient a compound of the present invention, or a composition comprising said compound.

According to another embodiment, the invention relates to a method of inhibiting MT1-MMP, or a mutant thereof, activity in a patient comprising the step of administering to said patient a compound of the present invention, or a composition comprising said compound.

Depending upon the particular condition, or disease, to be treated, additional therapeutic agents that are normally administered to treat that condition, may also be present in the compositions of this invention. As used herein, additional therapeutic agents that are normally administered to treat a particular disease, or condition, are known as “appropriate for the disease, or condition, being treated.”

A compound of the current invention may also be used to advantage in combination with other antiproliferative compounds. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide) (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZD6244 from AstraZeneca, PD181461 from Pfizer and leucovorin. The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™. Letrozole is marketed under the trade names Femara™ or Femar™ Aminoglutethimide is marketed under the trade name Orimeten™. A combination of the invention comprising a chemotherapeutic agent which is an aromatase inhibitor is particularly useful for the treatment of hormone receptor positive tumors, such as breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™. Raloxifene hydrochloride is marketed under the trade name Evista™. Fulvestrant can be administered under the trade name Faslodex™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (Casodex™). The term “gonadorelin agonist” as used herein includes, but is not limited to abarelix, goserelin and goserelin acetate. Goserelin can be administered under the trade name Zoladex™.

The term “topoisomerase I inhibitor” as used herein includes, but is not limited to topotecan, gimatecan, irinotecan, camptothecin and its analogues, 9-nitrocamptothecin and the macromolecular camptothecin conjugate PNU-166148. Irinotecan can be administered, e.g. in the form as it is marketed, e.g. under the trademark Camptosar™. Topotecan is marketed under the trade name Hycamptin™.

The term “topoisomerase II inhibitor” as used herein includes, but is not limited to the anthracyclines such as doxorubicin (including liposomal formulation, such as Caelyx™) daunorubicin, epirubicin, idarubicin and nemorubicin, the anthraquinones mitoxantrone and losoxantrone, and the podophillotoxines etoposide and teniposide. Etoposide is marketed under the trade name Etopophos™. Teniposide is marketed under the trade name VM 26-Bristol Doxorubicin is marketed under the trade name Acriblastin™ or Adriamycin™. Epirubicin is marketed under the trade name Farmorubicin™. Idarubicin is marketed. under the trade name Zavedos™. Mitoxantrone is marketed under the trade name Novantron.

The term “microtubule active agent” relates to microtubule stabilizing, microtubule destabilizing compounds and microtubulin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; colchicine and epothilones and derivatives thereof. Paclitaxel is marketed under the trade name Taxol™. Docetaxel is marketed under the trade name Taxotere™. Vinblastine sulfate is marketed under the trade name Vinblastin R.P™. Vincristine sulfate is marketed under the trade name Farmistin™.

The term “alkylating agent” as used herein includes, but is not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name Xeloda™. Gemcitabine is marketed under the trade name Gemzar™.

The term “platin compound” as used herein includes, but is not limited to, carboplatin, cis-platin, cisplatinum and oxaliplatin. Carboplatin can be administered, e.g., in the form as it is marketed, e.g. under the trademark Carboplat™. Oxaliplatin can be administered, e.g., in the form as it is marketed, e.g. under the trademark Eloxatin™.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the Axl receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, TYK2, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; Ilmofosine; RO 318220 and RO 320432; GO 6976; Isis 3521; LY333531/LY379196; isochinoline compounds; FTIs; PD184352 or QAN697 (a PI3K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR₁ ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, Cl-1033, EKB-569, GW-2016, E1.1, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib

Further examples of BTK inhibitory compounds, and conditions treatable by such compounds in combination with compounds of this invention can be found in WO2008039218 and WO2011090760, the entirety of which are incorporated herein by reference.

Further examples of SYK inhibitory compounds, and conditions treatable by such compounds in combination with compounds of this invention can be found in WO2003063794, WO2005007623, and WO2006078846, the entirety of which are incorporated herein by reference.

Further examples of PI3K inhibitory compounds, and conditions treatable by such compounds in combination with compounds of this invention can be found in WO2004019973, WO2004089925, WO2007016176, U.S. Pat. No. 8,138,347, WO2002088112, WO2007084786, WO2007129161, WO2006122806, WO2005113554, and WO2007044729 the entirety of which are incorporated herein by reference.

Further examples of JAK inhibitory compounds, and conditions treatable by such compounds in combination with compounds of this invention can be found in WO2009114512, WO2008109943, WO2007053452, WO2000142246, and WO2007070514, the entirety of which are incorporated herein by reference.

Further anti-angiogenic compounds include compounds having another mechanism for their activity, e.g. unrelated to protein or lipid kinase inhibition e.g. thalidomide (Thalomid™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), rofecoxib (Vioxx™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™. Tiludronic acid is marketed under the trade name Skelid™. Pamidronic acid is marketed under the trade name Aredia™. Alendronic acid is marketed under the trade name Fosamax™. Ibandronic acid is marketed under the trade name Bondranat™. Risedronic acid is marketed under the trade name Actonel™. Zoledronic acid is marketed under the trade name Zometa™. The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (Zarnestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (Velcade™) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; and ALK inhibitors, which are compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan®), PRO64553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

Other anti-leukemic compounds include, for example, Ara-C, a pyrimidine analog, which is the f-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; Angiostatin™; Endostatin™; anthranilic acid amides; ZD4190; ZD6474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; snRNA or siRNA; or miscellaneous compounds or compounds with other or unknown mechanism of action.

The structure of the active compounds identified by code numbers, generic or trade names may be taken from the actual edition of the standard compendium “The Merck Index” or from databases, e.g. Patents International (e.g. IMS World Publications).

A compound of the current invention may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of the current invention, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive compound can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

### EXEMPLIFICATION

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### List of Common Abbreviations Used in the Experimental Section

AcOH: acetic acid
ACN: acetonitrile
aq: aqueous
d: days
DCM: dichloromethane
DIPEA: N,N-diisopropylethylamine
DMA: N,N-dimethylacetamide
DMF: N,N-dimethylformamide
DMSO: dimethyl sulfoxide
EEDQ: Ethyl 2-ethoxy-1(2H)-quinolinecarboxylate
EDCI: 1-(3-dimethylaminopropyl)-3-ethylcarbodiimide hydrochloride
eq: equivalents
ESI: electrospray ionization
Et₃N: triethylamine
Et₂O: diethyl ether
EtOAc: ethyl acetate
EtOH: ethanol
hr: hours
HATU: N,N,N′,N′-tetramethyl-O-(7-azabenzotriazol-1-yl)uranium hexafluorophosphate
HOBt: Hydroxybenzotriazole
HPLC: high performance liquid chromatography
LC-MS: liquid chromatography-mass spectrometry
M: molar
MeCN: acetonitrile
MeOH: methanol
min: minutes
mL: milliliters
μL: microliters
mM: millimolar
mmol: millimoles
μmol: micromolar
MS: mass spectrometry
NMR: Nuclear Magnetic Resonance
° C.: degrees Celsius
PE: petroleum ether
prep-HPLC: preparative high performance liquid chromatography
PySSPy: 1,2-di(pyridin-2-yl)disulfane
rt: room temperature
TFA: trifluoracetic acid
TFP: 2,3,5,6-tetrafluorophenol
TLC: thin layer chromatography
THF: tetrahydrofuran

Preparative HPLC Method

Separation Condition: A phase: 0.075% TFA in H₂O, B phase: MeCN
Separation method: 18-48-55 min, RT=53.5 min
Separation column: Luna 200*25 mm 10 um, C18, 110A and Gemin150*30 mm, C18, 5 um, 110A, connection, 50° C.
Dissolve method: DMA
Separation purity: 95%

General Synthetic Methods

Peptide Synthesis—Molecular Scaffold Reagent with Leaving Groups

Peptide synthesis was based on Fmoc chemistry, using a Symphony peptide synthesizer manufactured by Peptide Instruments and a Syro II synthesizer by MultiSynTech. Standard Fmoc-amino acids were employed (Sigma, Merck), with appropriate side chain protecting groups: where applicable standard coupling conditions were used in each case, followed by deprotection using standard methodology. Peptides were purified using HPLC and following isolation they were modified with a molecular scaffold reagent with leaving groups. For this, linear peptide was diluted with H₂O up to ˜35 mL, ˜500 μL of 100 mM molecular scaffold reagent in acetonitrile was added, and the reaction was initiated with 5 mL of 1 M NH₄ HCO₃ in H₂O. The reaction was allowed to proceed for ˜30-60 min at RT, and lyophilized once the reaction had completed (as judged by MALDI). Following lyophilization, the reaction mixture was loaded onto a Gemini C18 column (Phenomenex). Solvents (H₂O, acetonitrile) were acidified with 0.1% trifluoroacetic acid. The gradient ranged from 30-70% acetonitrile in 15 minutes, at a flowrate of 15-20 mL/min, using a Gilson preparative HPLC system. Pure fractions containing the desired product were pooled, lyophilized and kept at −20° C. for storage.

### Peptide Synthesis—Molecular Scaffold Reagent Containing Michael Acceptors

Alternatively, peptides were purified using HPLC and following isolation they were modified with a molecular scaffold reagent containing Michael acceptors. For this, linear peptide was diluted with 50:50 MeCN:H₂O up to ˜35 mL, ˜500 μL of 100 mM molecular scaffold reagent containing Michael acceptors in acetonitrile was added, and the reaction was initiated with 5 mL of 1 M NH₄HCO₃ in H₂O. The reaction was allowed to proceed for ˜30-60 min at RT, and lyophilized once the reaction had completed (as judged by MALDI). Once completed, 1 mL of 1M L-Cysteine hydrochloride monohydrate (Sigma) in H₂O was added to the reaction for ˜60 min at RT to quench any excess molecular scaffold reagent containing Michael acceptors.

Following lyophilization, the modified peptide was purified as above, while replacing the Luna C8 with a Gemini C18 column (Phenomenex), and changing the acid to 0.1% trifluoroacetic acid. Pure fractions containing the correct desired product were pooled, lyophilized and kept at −20° C. for storage.

All amino acids, unless noted otherwise, were used in the L-configurations.

The preparation of Bicycle peptide (17-69-07-N241) is disclosed in WO 2016/067035, which is hereby incorporated by reference in its entirety.

Structure of 17-69-07-N241.

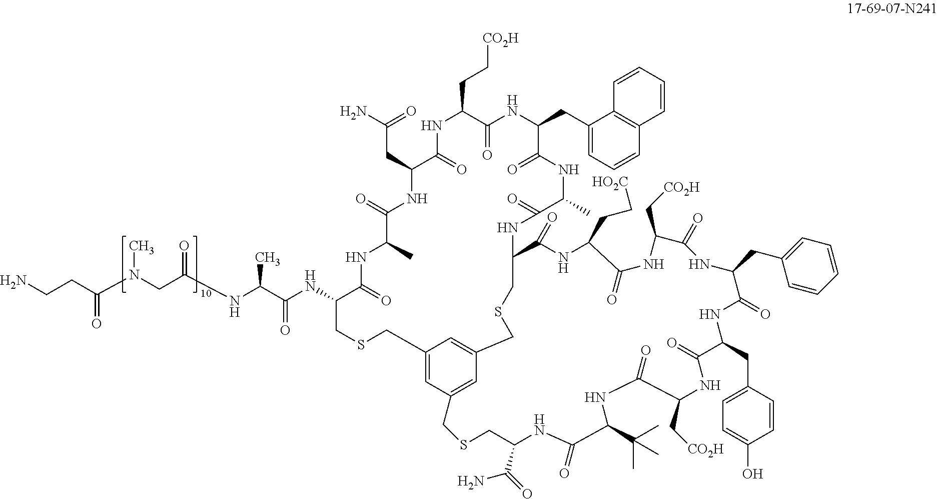

### Materials and Methods

### Example 1: Synthesis of I-7

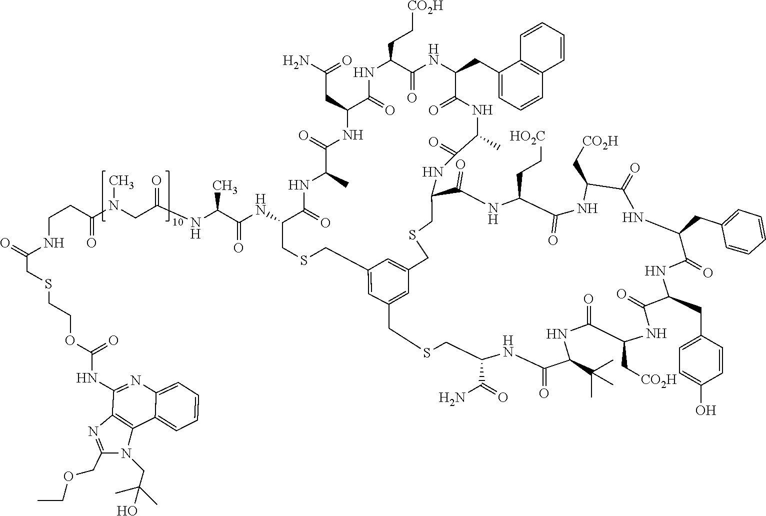

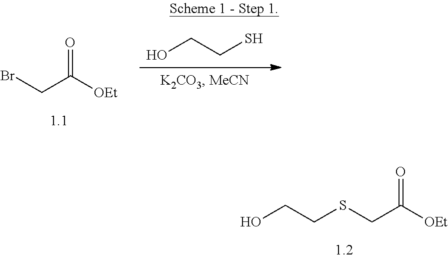

### Synthesis of Compound 1.2

To a solution of 2-sulfanylethanol (2.00 g, 25.6 mmol, 1.79 mL, 1 eq) and ethyl 2-bromoacetate (4.27 g, 25.6 mmol, 2.83 mL, 1 eq) in MeCN (50.0 mL) was added K₂CO₃ (5.31 g, 38.40 mmol, 1.5 eq). The mixture was stirred at 20° C. for 4 hr. TLC indicated Reactant 1.1 was consumed completely and one new spot formed. The reaction was clean according to TLC. The reaction mixture was quenched by addition saturated NH₄Cl 100 mL at 20° C., and then diluted with EtOAc 100 mL and extracted with EtOAc (100 mL×3). The combined organic layers were washed with brine (300 mL×2), dried over [Na₂SO₄], filtered and concentrated under reduced pressure to give a residue. The crude product compound 1.2 was obtained (3.3 g, crude) as a light yellow oil that was used into the next step without further purification.

¹H NMR: (400 MHz, CDCl₃) δ=4.21 (q, J=7.3 Hz, 2H), 3.78 (t, J=5.6 Hz, 2H), 3.26 (s, 2H), 2.85 (t, J=5.8 Hz, 2H), 1.30 (t, J=7.2 Hz, 3H).

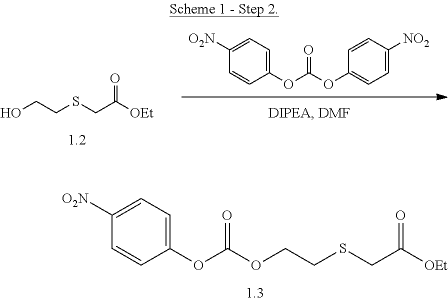 [SMILES: CCOC(=O)CSCCO ; O=C(Oc1ccc([N+](=O)[O-])cc1)Oc1ccc([N+](=O)[O-])cc1 ; CCOC(=O)CSCCOC(=O)Oc1ccc([N+](=O)[O-])cc1]

### Synthesis of Compound 1.3

To a solution of compound 1.2 (500.0 mg, 3.04 mmol, 1 eq) in THF (15 mL) was added bis(4-nitrophenyl) carbonate (1.85 g, 6.09 mmol, 2 eq) and DIPEA (787.0 mg, 6.09 mmol, 1.06 mL, 2 eq). The mixture was stirred at 20° C. for 1 hr. TLC indicated compound 1.2 was consumed completely and one new spot formed. The reaction was clean according to TLC. The reaction mixture was concentrated under reduced pressure to give a residue. The residue was purified by column chromatography (SiO₂, PE/EtOAc=9:1 to 4:1) Compound 1.3 (800.0 mg, 2.19 mmol, 71.8% yield) was obtained as a light yellow oil.

¹H NMR: (400 MHz, CDCl₃) δ=8.34-8.28 (m, 2H), 7.45-7.39 (m, 2H), 4.51 (t, J=6.5 Hz, 2H), 4.23 (q, J=7.0 Hz, 2H), 3.32 (s, 2H), 3.04 (t, J=6.5 Hz, 2H), 1.32 (t, J=7.2 Hz, 3H).

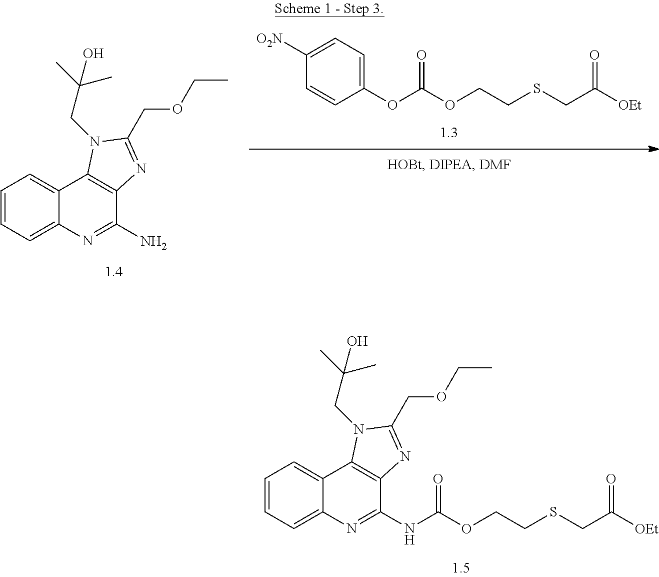 [SMILES: CCOCc1nc2c(N)nc3ccccc3c2n1CC(C)(C)O ; CCOC(=O)CSCCOC(=O)Oc1ccc([N+](=O)[O-])cc1 ; CCOCc1nc2c(NC(=O)OCCSCC(=O)OCC)nc3ccccc3c2n1CC(C)(C)O]

### Synthesis of Compound 1.5

To a solution of Resiquimod, 1.4, (100.0 mg, 318.1 μmol, 1 eq), HOBt (86.0 mg, 636.2 μmol, 2 eq) and DIPEA (123.3 mg, 954.3 μmol, 166.2 μL, 3.0 eq) in DMF (2.0 mL) was added compound 1.3 (209.5 mg, 636.2 μmol, 2 eq). The mixture was stirred at 30° C. for 12 hr. TLC indicated ˜5% of Resiquimod remained, and one major new spot with lower polarity was detected. The mixture was purified by column chromatography (SiO₂, DCM/MeOH=19/1). Compound 1.5 (134.0 mg, 256.0 μmol, 80.5% yield, 96.41% purity) was obtained as a light yellow solid.

¹H NMR: (400 MHz, DMSO-d₆) δ=9.85 (br s, 1H), 8.58 (d, J=8.3 Hz, 1H), 7.97 (d, J=8.3 Hz, 1H), 7.67 (t, J=7.3 Hz, 1H), 7.62-7.54 (m, 1H), 4.98 (s, 1H), 4.94-4.65 (m, 2H), 4.33 (t, J=6.5 Hz, 2H), 4.14 (q, J=7.2 Hz, 2H), 3.62-3.54 (m, 2H), 3.57 (q, J=6.9 Hz, 2H), 3.51 (s, 2H), 2.99-2.91 (m, 2H), 1.27-1.14 (m, 12H).

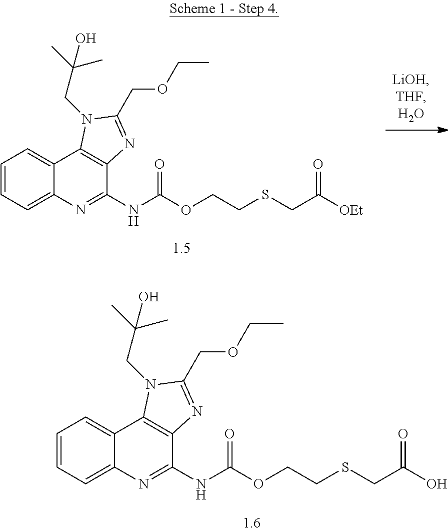 [SMILES: CCOCc1nc2c(NC(=O)OCCSCC(=O)OCC)nc3ccccc3c2n1CC(C)(C)O ; CCOCc1nc2c(NC(=O)OCCSCC(=O)O)nc3ccccc3c2n1CC(C)(C)O]

### Synthesis of Compound 1.6

To a solution of compound 1.5 (200.0 mg, 396.4 μmol, 1 eq) in THF (2.5 mL) was added LiOH.H₂O (33.3 mg, 792.7 μmol, 2 eq) in H₂O (0.5 mL). The mixture was stirred at 20° C. for 15 min. TLC indicated compound 1.5 was consumed completely and one new spot formed. The reaction was clean according to TLC. The reaction mixture was concentrated under reduced pressure to give a residue. The residue was purified by prep-HPLC (ACN/H₂O condition). Compound 1.6 (150.0 mg, 314.8 μmol, 79.4% yield) was obtained as a light yellow solid. MS (ESI) m/z: calcd. for [M+H]⁺, 477.17; found, 477.1 (M/1+H)⁺.

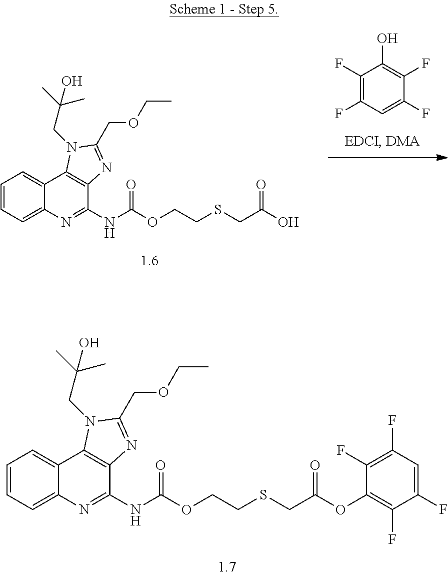 [SMILES: CCOCc1nc2c(NC(=O)OCCSCC(=O)O)nc3ccccc3c2n1CC(C)(C)O ; Oc1c(F)c(F)cc(F)c1F ; CCOCc1nc2c(NC(=O)OCCSCC(=O)Oc3c(F)c(F)cc(F)c3F)nc3ccccc3c2n1CC(C)(C)O]

### Synthesis of Compound 1.7

To a solution of compound 1.6 (150.0 mg, 314.8 μmol, 1 eq) and 2,3,5,6-tetrafluorophenol (156.8 mg, 944.3 μmol, 3 eq) in DMA (4.5 mL) and DCM (1.5 mL) was added EDCI (181.0 mg, 944.3 μmol, 3 eq). The mixture was stirred at 20° C. for 2 hr. LC-MS showed the desired compound was detected. The mixture was purified by prep-HPLC (ACN/H₂O condition). Compound 1.7 (140.0 mg, 224.1 μmol, 71.2% yield) was obtained as a white solid. MS (ESI) m/z: calcd. for [M+H]⁺, 625.17; found, 625.1 (M/1+H)⁺.

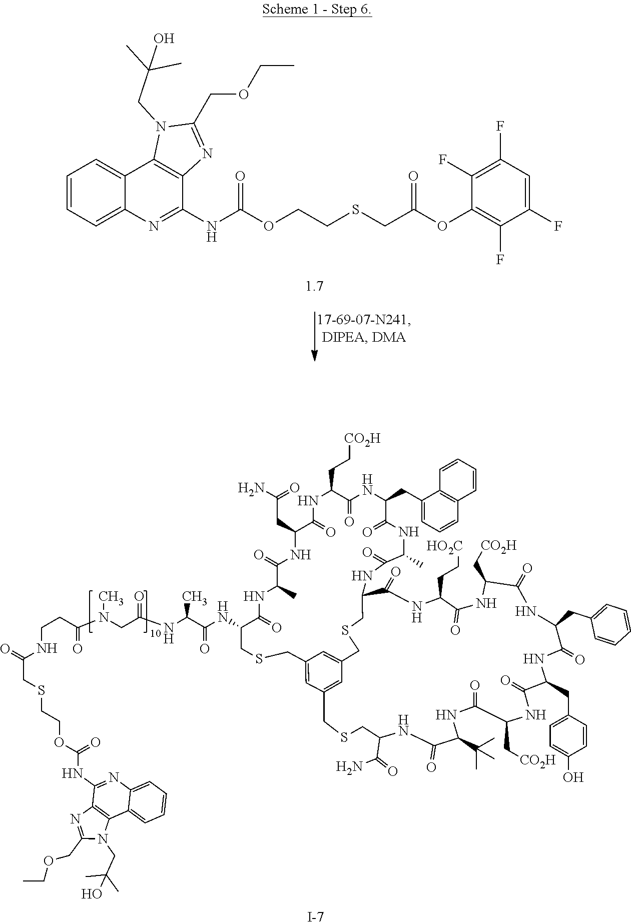

### Synthesis of I-7

To a solution of Bicycle peptide (17-69-07-N241) (170.3 mg, 64.0 μmol, 1 eq) in DMA (2 mL) was added DIPEA (24.8 mg, 192.1 μmol, 33.5 μL, 3 eq) and compound 1.7 (40.0 mg, 64.0 μmol, 1 eq). The mixture was stirred at 20° C. for 2 hr. LC-MS showed no compound 1.7 remained. Several new peaks were shown on LC-MS and −80% of desired compound was detected. The mixture was purified by prep-HPLC (TFA condition). Compound I-7 (80.2 mg, 25.7 μmol, 40.1% yield, 97.47% purity) was obtained as a white solid.

MS (ESI) m/z: calcd. for [M+H]⁺, 3118.1; found, 1039.6 (M/3+H)⁺.

### Example 2: Synthesis of I-8

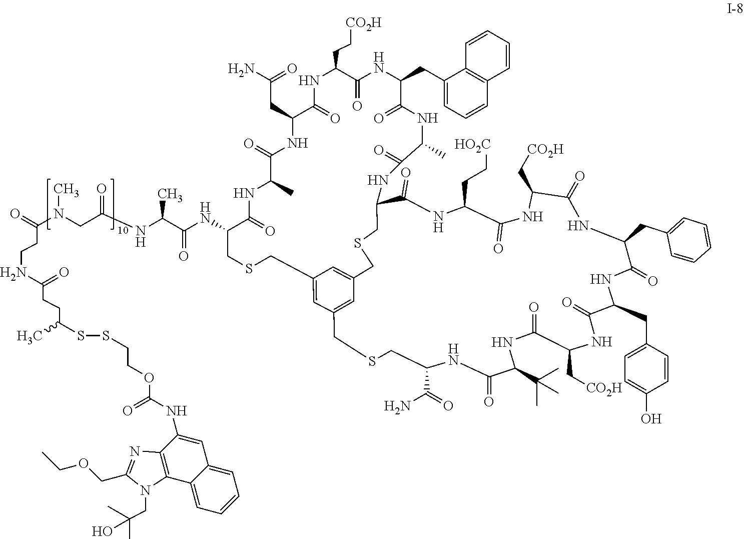

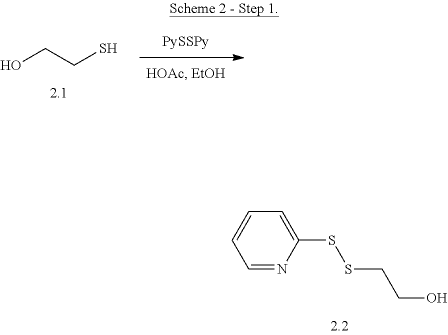

### Synthesis of Compound 2.2

To a solution of 2-sulfanylethanol (3.0 g, 38.40 mmol, 2.68 mL, 1 eq), PySSPy (12.7 g, 57.59 mmol, 1.5 eq) in MeOH (100 mL) was added AcOH (5.76 g, 95.99 mmol, 5.49 mL, 2.5 eq). The mixture was stirred at 25° C. for 16 hr under N2. TLC indicated 2-sulfanylethanol was consumed completely and one new spot formed. The reaction was clean according to TLC. The reaction mixture was concentrated under reduced pressure to remove solvent to give a residue. The residue was purified by flash silica gel chromatography (ISCO®; 120 g SepaFlash® Silica Flash Column, Eluent of 0˜40% EtOAc/PE gradient@80 mL/min). Compound 2.2 (5.5 g, 29.37 mmol, 76.5% yield) was obtained as a yellow oil. MS (ESI) m/z: calcd. for [M+H]⁺, 187.01; found, 188.1 (M/1+H)⁺.

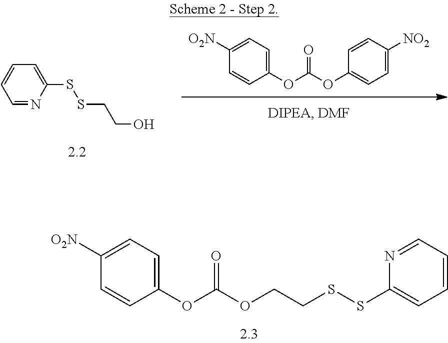 [SMILES: OCCSSc1ccccn1 ; O=C(Oc1ccc([N+](=O)[O-])cc1)Oc1ccc([N+](=O)[O-])cc1 ; O=C(OCCSSc1ccccn1)Oc1ccc([N+](=O)[O-])cc1]

### Synthesis of Compound 2.3

To a solution of compound 2.2 (0.50 g, 2.67 mmol, 1 eq) in THF (10 mL) was added DIPEA (1.04 g, 8.01 mmol, 1.40 mL, 3 eq) and bis(4-nitrophenyl) carbonate (1.62 g, 5.34 mmol, 2 eq). The mixture was stirred at 25° C. for 16 hr. TLC indicated compound 2.2 was consumed completely and two new spots formed. The reaction was clean according to TLC. The reaction mixture was concentrated under reduced pressure to remove solvent to give a residue. The residue was purified by flash silica gel chromatography (ISCO®; 40 g SepaFlash® Silica Flash Column, Eluent of 0-50% EtOAc/PE gradient@40 mL/min). Compound 2.3 (0.35 g, 993.23 μmol, 37.20% yield) was obtained as yellow oil. MS (ESI) m/z: calcd. for [M+H]⁺, 353.02; found, 353.0 (M/1+H)⁺.

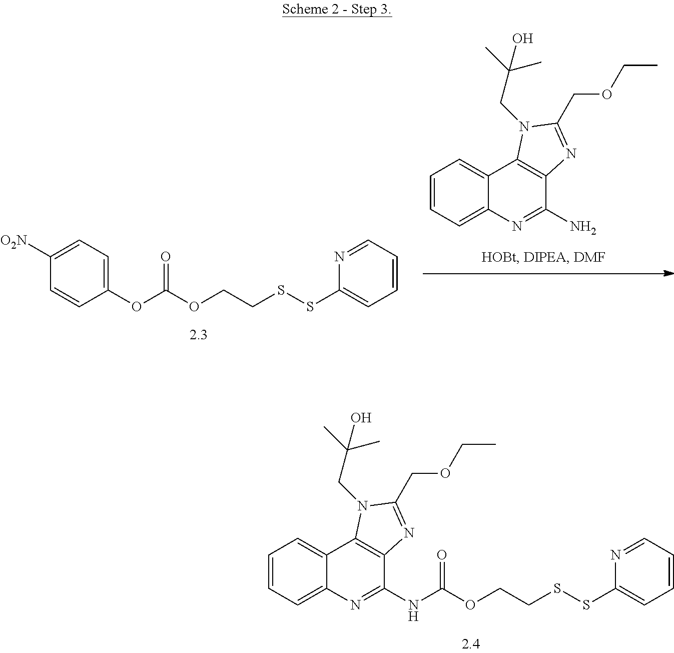 [SMILES: CCOCc1nc2c(N)nc3ccccc3c2n1CC(C)(C)O ; O=C(OCCSSc1ccccn1)Oc1ccc([N+](=O)[O-])cc1 ; CCOCc1nc2c(NC(=O)OCCSSc3ccccn3)nc3ccccc3c2n1CC(C)(C)O]

### Synthesis of Compound 2.4

To a solution of compound 2.3 (134.51 mg, 381.70 μmol, 1.2 eq) in DMF (2 mL) was added HOBt (51.58 mg, 381.70 μmol, 1.2 eq), DIPEA (123.33 mg, 954.26 μmol, 166.21 μL, 3 eq), and Resiquimod (0.1 g, 318.09 μmol, 1 eq). The mixture was stirred at 40° C. for 18 hr. LC-MS showed compound 2.3 was consumed completely and one main peak with desired MS was detected. The mixture was directly purified by prep-HPLC (ACN/H₂O condition). Compound 2.4 (0.05 g, 94.76 μmol, 29.79% yield) was obtained as a yellow solid. MS (ESI) m/z: calcd. for [M+H]⁺, 528.17; found, 528.1 (M/1+H)⁺.

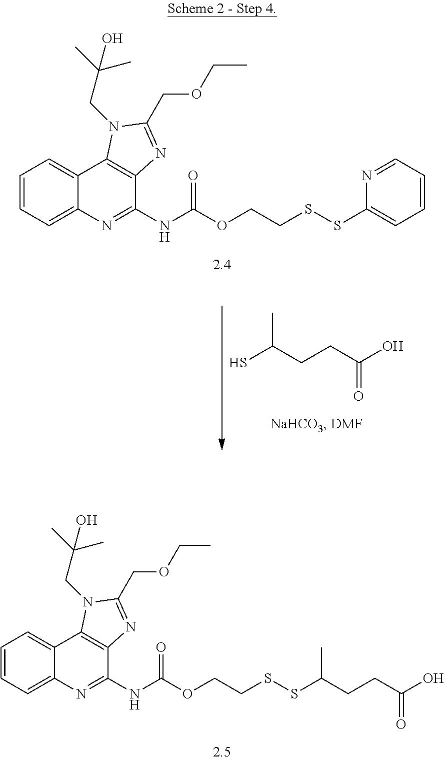

### Synthesis of Compound 2.5

To a solution of compound 2.4 (0.05 g, 94.76 μmol, 1 eq), 4-sulfanylpentanoic acid (25.4 mg, 189.52 μmol, 2 eq) in DMF (1 mL) was adjusted to pH 8 by the addition of aq NaHCO₃ solution. The mixture was stirred at 25° C. for 0.5 hr. LC-MS showed compound 2.4 was consumed completely and one main peak with desired MS was detected. The mixture was directly purified by prep-HPLC (ACN/H₂O condition). Compound 2.5 (0.04 g, 72.64 μmol, 76.6% yield) was obtained as a white solid. MS (ESI) m/z: calcd. for [M+H]⁺, 551.19; found, 551.1 (M/1+H)⁺.

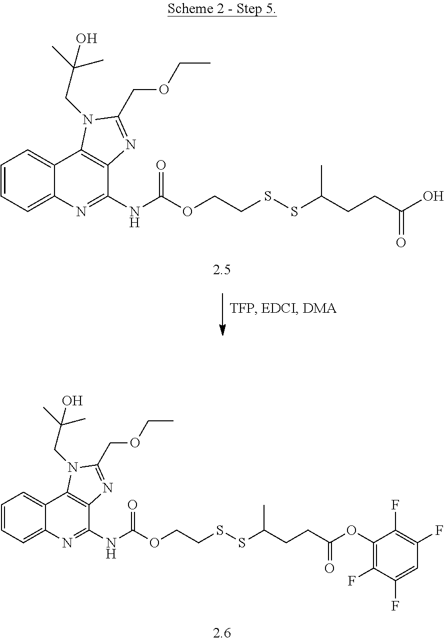 [SMILES: CCOCc1nc2c(NC(=O)OCCSSC(C)CCC(=O)O)nc3ccccc3c2n1CC(C)(C)O ; CCOCc1nc2c(NC(=O)OCCSSC(C)CCC(=O)Oc3c(F)c(F)cc(F)c3F)nc3ccccc3c2n1CC(C)(C)O]

### Synthesis of Compound 2.6

To a solution of compound 2.5 (0.04 g, 72.64 μmol, 1 eq), 2,3,5,6-tetrafluorophenol (36.2 mg, 217.91 μmol, 3 eq) in DMA (3 mL) and DCM (1 mL) was added EDCI (41.8 mg, 217.91 μmol, 3 eq). The mixture was stirred at 25° C. for 16 hr. LC-MS showed compound 5 was consumed completely and one main peak with desired MS was detected. The reaction was directly purified by prep-HPLC (ACN/H₂O condition). Compound 2.6 (0.035 g, 50.09 μmol, 68.9% yield) was obtained as a white solid. MS (ESI) m/z: calcd. for [M+H]⁺, 698.19; found, 699.0 (M/1+H)⁺.

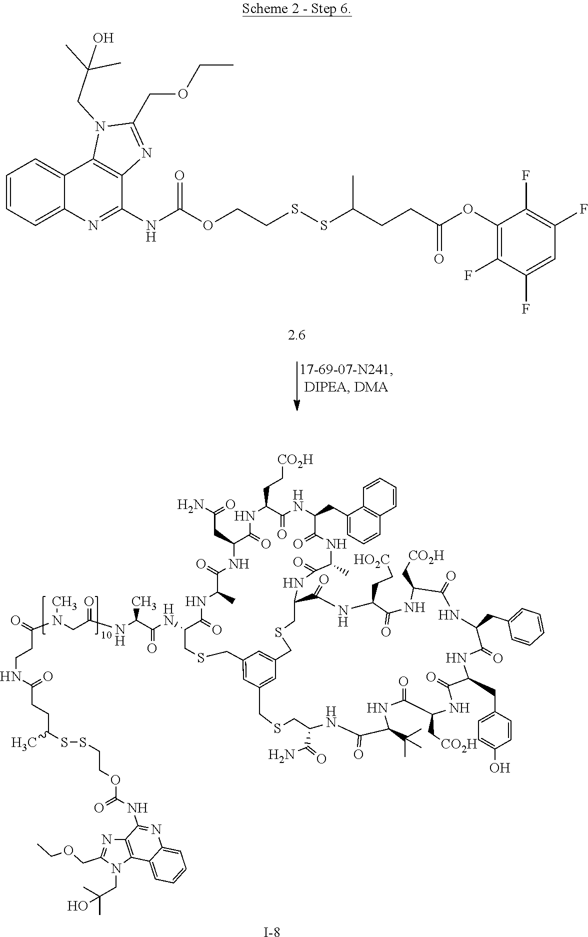

### Synthesis of I-8

To a solution of compound 2.6 (0.035 g, 50.09 μmol, 1 eq) in DMA (2 mL) was added DIPEA (19.4 mg, 150.3 μmol, 26.2 μL, 3 eq) and 17-69-07-N241 (146.5 mg, 55.1 μmol, 1.1 eq). The mixture was stirred at 25° C. for 16 hr. LC-MS showed compound 6 was consumed completely and one main peak with desired MS was detected. The reaction was directly purified by prep-HPLC (TFA condition). Compound I-8 (0.073 g, 22.8 μmol, 45.6% yield) was obtained as a white solid.

MS (ESI) m/z: calcd. for [M+H]⁺, 3191.1; found, 1064.5 (M/3+H)⁺.

### Example 3: Synthesis of I-9

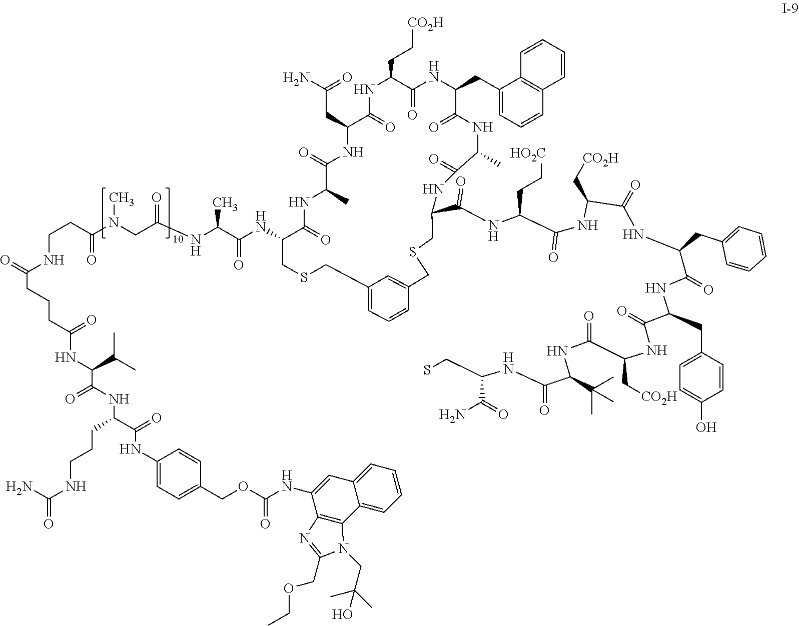

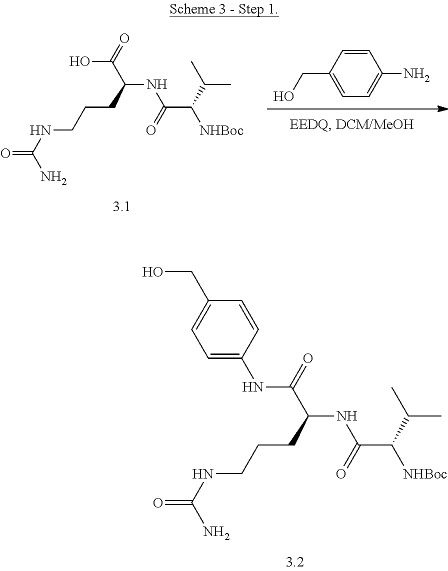 [SMILES: CC(C)[C@H](NC(=O)OC(C)(C)C)C(=O)N[C@@H](CCCNC(N)=O)C(=O)O ; Nc1ccc(CO)cc1 ; CC(C)[C@H](NC(=O)OC(C)(C)C)C(=O)N[C@@H](CCCNC(N)=O)C(=O)Nc1ccc(CO)cc1]

### Synthesis of Compound 3.2

To a solution of compound 3.1 (10.0 g, 26.71 mmol, 1 eq) in DCM (40 mL) and MeOH (20 mL) was added EEDQ (13.2 g, 53.41 mmol, 2 eq) and (4-aminophenyl)methanol (3.95 g, 32.05 mmol, 1.2 eq). The mixture was stirred at 25° C. for 12 hr. LC-MS showed compound 3.1 was consumed completely and one main peak with desired MS was detected. TLC indicated compound 3.1 was remained, and one major new spot with lower polarity was detected. The reaction mixture was concentrated under reduced pressure to remove solvent to give a residue. The residue was purified by flash silica gel chromatography (ISCO®; 330 g SepaFlash® Silica Flash Column, Eluent of 0-15% EtOAc/PE gradient@100 mL/min). Compound 3.2 (7.4 g, 15.43 mmol, 57.8% yield) was obtained as a white solid. MS (ESI) m/z: calcd. for [M+H]⁺, 480.27; found, 480.1 (M/1+H)⁺.

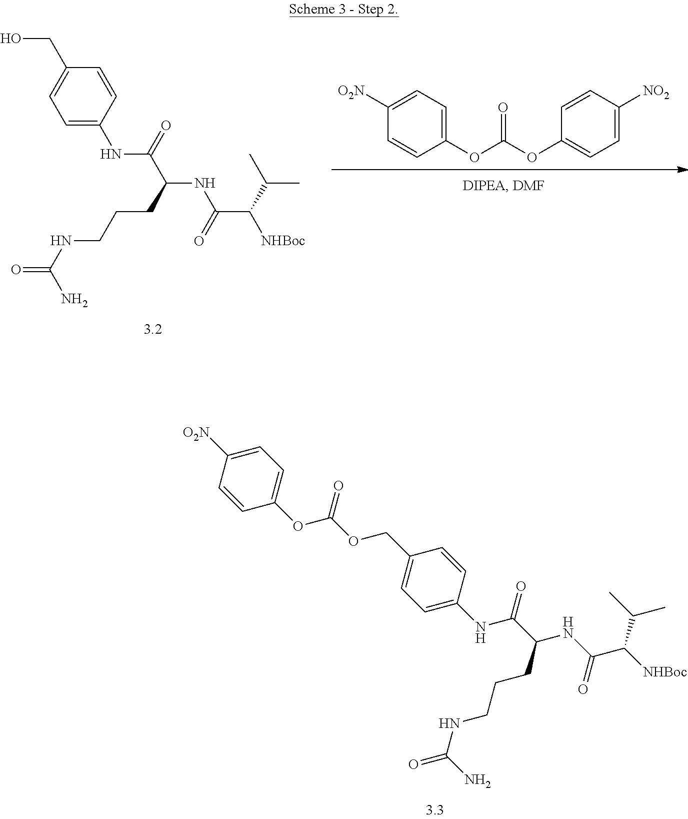 [SMILES: CC(C)[C@H](NC(=O)OC(C)(C)C)C(=O)N[C@@H](CCCNC(N)=O)C(=O)Nc1ccc(CO)cc1 ; O=C(Oc1ccc([N+](=O)[O-])cc1)Oc1ccc([N+](=O)[O-])cc1 ; CC(C)[C@H](NC(=O)OC(C)(C)C)C(=O)N[C@@H](CCCNC(N)=O)C(=O)Nc1ccc(COC(=O)Oc2ccc([N+](=O)[O-])cc2)cc1]

### Synthesis of Compound 3.3

To a solution of compound 3.2 (3.0 g, 6.26 mmol, 1 eq) in DMF (10 mL) was added DIPEA (2.4 g, 18.77 mmol, 3.3 mL, 3 eq) and bis(4-nitrophenyl) carbonate (3.8 g, 12.51 mmol, 2 eq). The mixture was stirred at 25° C. for 1 hr. LC-MS showed compound 3.2 was consumed completely and one main peak with desired MS was detected. The reaction was directly purified by prep-HPLC (ACN/H₂O condition). Compound 3.3 (2.6 g, 4.03 mmol, 64.47% yield) was obtained as a white solid. MS (ESI) m/z: calcd. for [M+H]⁺, 645.28; found, 645.1 (M/1+H)⁺.

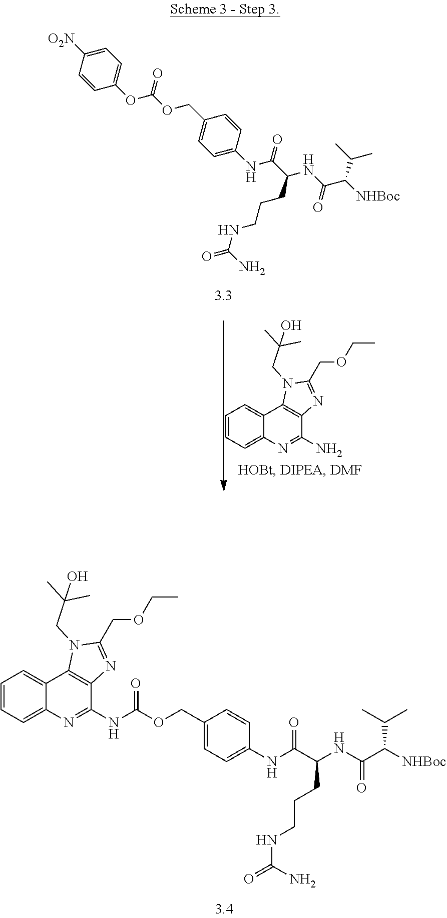 [SMILES: CC(C)[C@H](NC(=O)OC(C)(C)C)C(=O)N[C@@H](CCCNC(N)=O)C(=O)Nc1ccc(COC(=O)Oc2ccc([N+](=O)[O-])cc2)cc1 ; CCOCc1nc2c(N)nc3ccccc3c2n1CC(C)(C)O ; CCOCc1nc2c(NC(=O)OCc3ccc(NC(=O)[C@H](CCCNC(N)=O)NC(=O)[C@@H](NC(=O)OC(C)(C)C)C(C)C)cc3)nc3ccccc3c2n1CC(C)(C)O]

### Synthesis of Compound 3.4

To a solution of compound 3.3 (369.1 mg, 572.5 μmol, 1.5 eq) in DMF (4 mL) was added HOBt (61.9 mg, 458.04 μmol, 1.2 eq) and DIPEA (148.0 mg, 1.15 mmol, 199.5 μL, 3 eq), Resiquimod (0.12 g, 381.70 μmol, 1 eq). The mixture was stirred at 40° C. for 16 hr. LC-MS showed 50% Resiquimod remained. Several new peaks were shown on LC-MS and 30% of desired compound was detected. The reaction was directly purified by prep-HPLC (ACN/H₂O condition). Compound 3.4 (0.16 g, 195.14 μmol, 51.1% yield) was obtained as a white solid. MS (ESI) m/z: calcd. for [M+H]⁺, 820.43; found, 820.3 (M/1+H)⁺.

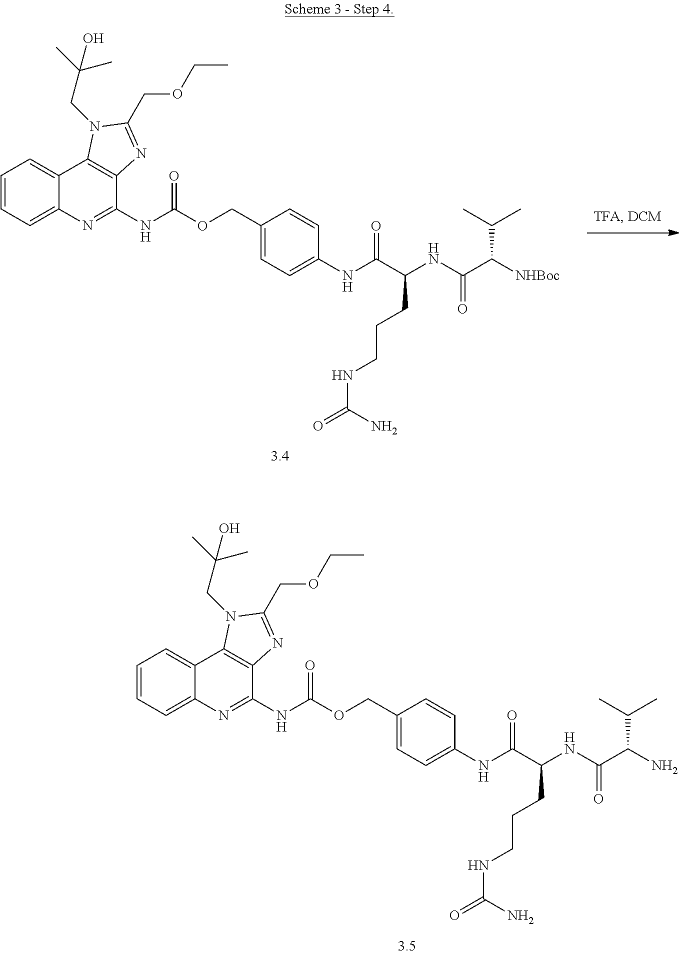 [SMILES: CCOCc1nc2c(NC(=O)OCc3ccc(NC(=O)[C@H](CCCNC(N)=O)NC(=O)[C@@H](NC(=O)OC(C)(C)C)C(C)C)cc3)nc3ccccc3c2n1CC(C)(C)O ; CCOCc1nc2c(NC(=O)OCc3ccc(NC(=O)[C@H](CCCNC(N)=O)NC(=O)[C@@H](N)C(C)C)cc3)nc3ccccc3c2n1CC(C)(C)O]

### Synthesis of Compound 3.5

To a solution of compound 3.4 (0.16 g, 195.14 μmol, 1 eq) in DCM (9 mL) was added TFA (1.54 g, 13.51 mmol, 1 mL, 69.21 eq). The mixture was stirred at 25° C. for 0.5 hr. LC-MS showed compound 3.4 was consumed completely and one main peak with desired MS was detected. The reaction mixture was concentrated under reduced pressure to remove solvent to give a residue. The residue was purified by prep-HPLC (ACN/H₂O condition). Compound 3.5 (0.08 g, 111.14 μmol, 56.9% yield) was obtained as a white solid. MS (ESI) m/z: calcd. for [M+H]⁺, 720.38 found, 720.3 (M/1+H)⁺.

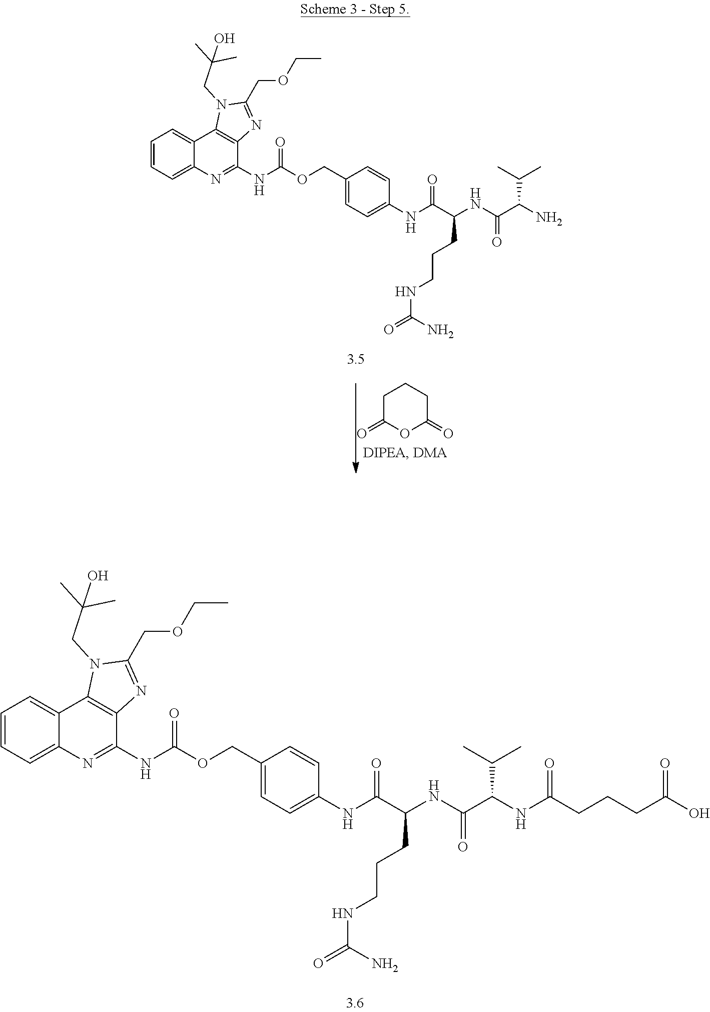 [SMILES: CCOCc1nc2c(NC(=O)OCc3ccc(NC(=O)[C@H](CCCNC(N)=O)NC(=O)[C@@H](N)C(C)C)cc3)nc3ccccc3c2n1CC(C)(C)O ; O=C1CCCC(=O)O1 ; CCOCc1nc2c(NC(=O)OCc3ccc(NC(=O)[C@H](CCCNC(N)=O)NC(=O)[C@@H](NC(=O)CCCC(=O)O)C(C)C)cc3)nc3ccccc3c2n1CC(C)(C)O]

### Synthesis of Compound 3.6

To a solution of compound 3.5 (0.08 g, 111.14 μmol, 1 eq) in DMA (3 mL) was added DIPEA (43.1 mg, 333.41 μmol, 58.1 μL, 3 eq) and tetrahydropyran-2,6-dione (38.0 mg, 333.41 μmol, 3 eq). The mixture was stirred at 25° C. for 1 hr. LC-MS showed compound 3.5 was consumed completely and one main peak with desired MS was detected. Reaction was directly purified by prep-HPLC (ACN/H₂O condition). Compound 3.6 (0.06 g, 68.35 μmol, 61.5% yield, 95% purity) was obtained as a white solid. MS (ESI) m/z: calcd. for [M+H]⁺, 834.41; found, 834.3 (M/1+H)⁺.

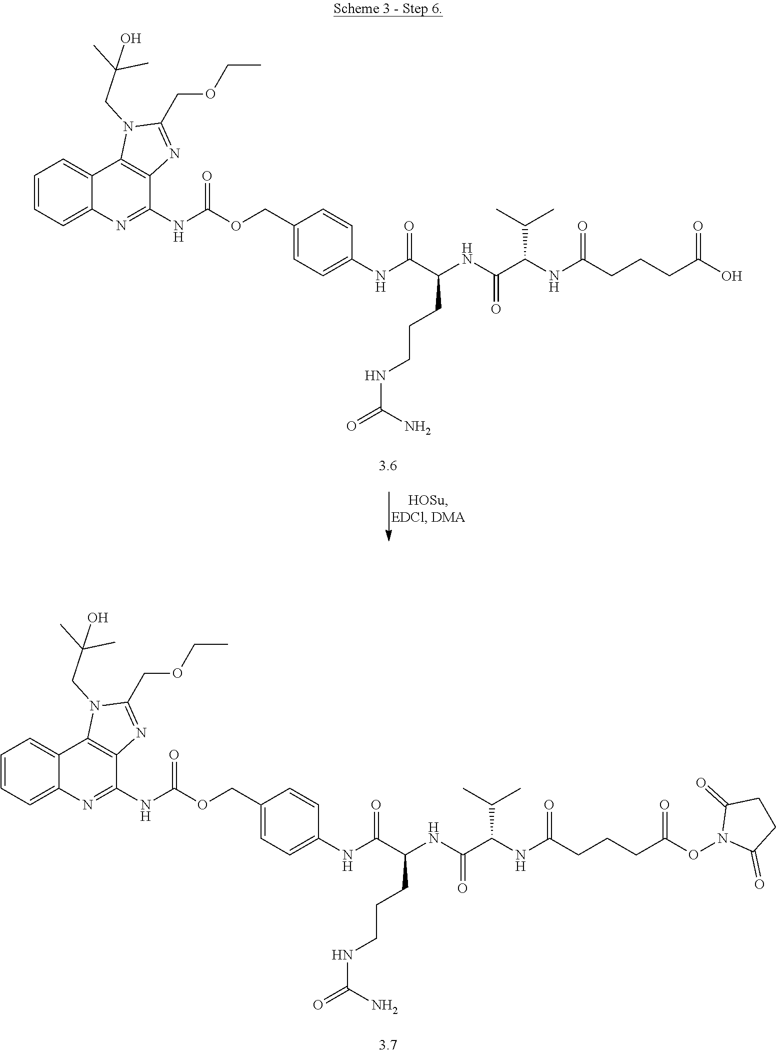 [SMILES: CCOCc1nc2c(NC(=O)OCc3ccc(NC(=O)[C@H](CCCNC(N)=O)NC(=O)[C@@H](NC(=O)CCCC(=O)O)C(C)C)cc3)nc3ccccc3c2n1CC(C)(C)O ; CCOCc1nc2c(NC(=O)OCc3ccc(NC(=O)[C@H](CCCNC(N)=O)NC(=O)[C@@H](NC(=O)CCCC(=O)ON4C(=O)CCC4=O)C(C)C)cc3)nc3ccccc3c2n1CC(C)(C)O]

### Synthesis of Compound 3.7

To a solution of compound 3.6 (0.04 g, 47.97 μmol, 1 eq), 1-hydroxypyrrolidine-2,5-dione (16.6 mg, 143.90 μmol, 3 eq) in DMA (3 mL) and DCM (1 mL) was added EDCI (27.6 mg, 143.90 μmol, 3 eq). The mixture was stirred at 25° C. for 16 hr. LC-MS showed compound 3.6 was consumed completely and one main peak with desired MS was detected. DCM was removed. Reaction was directly purified by prep-HPLC (ACN/H₂O condition). Compound 3.7 (0.035 g, 37.59 μmol, 78.4% yield) was obtained as a white solid. MS (ESI) m/z: calcd. for [M+H]⁺, 931.42; found, 931.3 (M/1+H)⁺.

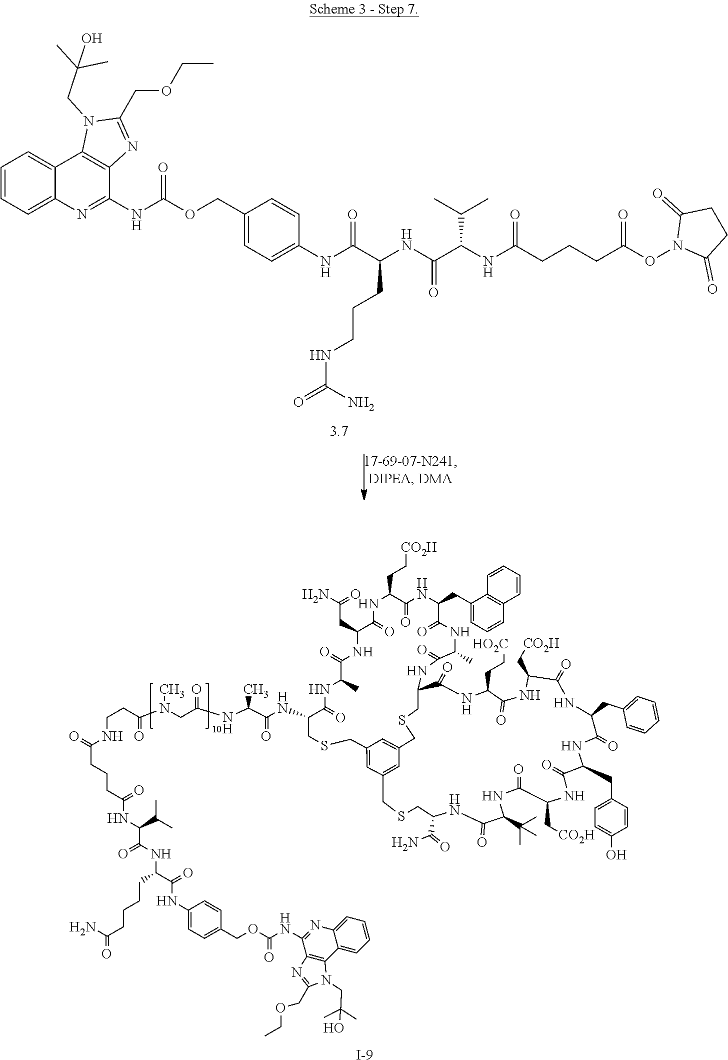

### Synthesis of I-9

To a solution of compound 3.7 (0.035 g, 37.59 μmol, 1 eq) in DMA (4 mL) was added DIPEA (14.6 mg, 112.78 μmol, 19.64 μL, 3 eq) and 17-69-07-N241 (94.6 mg, 37.59 μmol, 1 eq). The mixture was stirred at 25° C. for 12 hr. LC-MS showed compound 3.7 was consumed completely and one main peak with desired MS was detected. Reaction was directly purified by prep-HPLC (TFA condition), further purified by prep-HPLC (NH₄OAc condition). Compound I-9 (13.1 mg, 3.77 μmol, 10.0% yield) was obtained as a white solid.

MS (ESI) m/z: calcd. for [M+H]⁺, 3474.4; found, 1159.2 (M/3+H)⁺.

### Example 4: Dissociation Rate Constant Determination of Bicyclic Binders to MT1-MMP Direct Binding Fluorescence Polarization (Anisotropy) Assays

Direct Binding Fluorescence Polarization or Anisotropy Assays are performed by titrating a constant concentration of fluorescent tracer (here, the fluoresceinated bicyclic peptide to be studied) with its binding partner (here, the MT1-MMP hemopexin domain). As the concentration of binding partner increases during the titration, the polarization signal changes in proportion to the fraction of bound and unbound material. This allows determination of dissociation rates (K_{d}) quantitatively. Assay data can be fit using standard ligand binding equations.

Typically, concentrations of the tracer are ideally well below the K_{d} of the tracer:titrant pair, and concentrations chosen are usually at ˜1 nM or less. The titrant (binding partner) concentration is varied from 0.1 nM up to typically 5 μM. The range is chosen such that the maximum change in fluorescent polarization can be observed. Buffers employed are phosphate buffered saline in the presence of 0.01% Tween. Experiments were run in black 384 well low-bind/low volume plates (Corning 3820), and the fluorescent polarization signal was measured using a BMG Pherastar FS plate reader. Fluorescent tracers referred to in the text are bicyclic peptides that have been fluoresceinated using 5,6-carboxyfluorescein. Fluoresceination may be performed on the N-terminal amino group of the peptide, which is separated from the bicycle core sequence by a sarcosine spacer (usually Sar10). This can be done during Fmoc solid phase synthesis or post-synthetically (after cyclization with the molecular scaffold reagent and purification) if the N-terminal amino group is unique to the peptide. Fluoresceination can also be performed on the C-terminus, usually on a Lysine introduced as the first C-terminal residue, which is then separated from the bicycle core sequence by a sarcosine spacer (usually Sar6). Thus, N-terminal tracers can have a molecular format described as Fluo-Ala-Sar10-A(BicycleCoreSequence), and (BicycleCoreSequence)-A-Sar6-K(Fluo) for a C-terminally fluoresceinated construct.

Fluorescent tracers used in the Examples are A-(17-69)-A-Sar6-K(Fluo), A-(17-69-07)-A-Sar6-K(Fluo), and A-(17-69-12)-A-Sar6-K(Fluo). Due to the acidic nature of the 17-69 fluorescent peptides, they were typically prepared as concentrated DMSO stocks, from which dilution were prepared in 100 mM Tris pH 8 buffer.

### Example 5: Competition Assays Using Fluorescence Polarization (Anisotropy)

Due to their high affinities to the MT1-MMP Hemopexin domain (PEX), the fluoresceinated derivatives of 17-69-07 and 17-69-12 (denoted as 17-69-07-N040 and 17-69-12-N005, respectively) can be used for competition experiments (using FP for detection). Here, a preformed complex of PEX with the fluorescent PEX-binding tracer is titrated with free, non-fluoresceinated bicyclic peptide. Since all 17-69-based peptides are expected to bind at the same site, the titrant will displace the fluorescent tracer from PEX. Dissociation of the complex can be measured quantitatively, and the K_{d} of the competitor (titrant) to the target protein determined. The advantage of the competition method is that the affinities of non-fluoresceinated bicyclic peptides can be determined accurately and rapidly.

Concentrations of tracer are usually at the K_{d} or below (here, 1 nM), and the binding protein (here, hemopexin of MT1-MMP) is at a 15-fold excess such that >90% of the tracer is bound. Subsequently, the non-fluorescent competitor bicyclic peptide (usually just the bicycle core sequence) is titrated, such that it displaces the fluorescent tracer from the target protein. The displacement of the tracer is measured and associated with a drop in fluorescence polarization. The drop in fluorescence polarization is proportional to the fraction of target protein bound with the non-fluorescent titrant, and thus is a measure of the affinity of titrant to target protein.

The raw data is fit to the analytical solution of the cubic equation that describes the equilibria between fluorescent tracer, titrant, and binding protein. The fit requires the value of the affinity of fluorescent tracer to the target protein, which can be determined separately by direct binding FP experiments (see previous section). The curve fitting was performed using Sigmaplot 12.0 and used an adapted version of the equation described by Zhi-Xin Wang (FEBS Letters 360 (1995) 1 11-1 14).

### Example 6: In Vivo Efficacy of I-8 Alone or Combination with antiPD-1 mAb in Treatment of B16F10 Xenograft in C57BL/6J Mice

Experimental Methods and Procedures

B16F10 tumor cells were maintained in vitro as a monolayer culture in EMEM medium supplemented with 10% heat inactivated fetal bovine serum at 37° C. in an atmosphere of 5% CO₂ in air. The tumor cells were routinely subcultured twice weekly by trypsin-EDTA treatment. The cells growing in an exponential growth phase were harvested and counted for tumor inoculation.

6-8 week old female C57BL/6J mice were inoculated subcutaneously at the right flank with B16F10 tumor cells (5×10⁵) in 0.1 ml of PBS for tumor development. Animals were randomized when the average tumor volume reached 65 mm³.

Conjugates were formulated in 25 mM Histidine, 10% sucrose pH=7 buffer (vehicle) and administered intravenously. Anti-PD1 antibody (Wuxi AppTech, Shanghai, China) was formulated in aqueous buffer and administered intraperitoneally.

Tumor volume was measured three times weekly in two dimensions using a caliper, and the volume was expressed in mm³ using the formula: V=0.5 a×b² where a and b are the long and short diameters of the tumor, respectively. The tumor size was then used for calculations of T/C value. The T/C value (in percent) is an indication of antitumor effectiveness; T and C are the mean volumes of the treated and control groups, respectively, on a given day.

TGI was calculated for each group using the formula: TGI (%)=[1−(Tᵢ−T₀)/(Vᵢ−V₀)]×100; Tᵢ is the average tumor volume of a treatment group on a given day, T₀ is the average tumor volume of the treatment group on the day of treatment start, Vᵢ is the average tumor volume of the vehicle control group on the same day with Tᵢ, and V₀ is the average tumor volume of the vehicle group on the day of treatment start.

Summary statistics, including mean and the standard error of the mean (SEM), are provided for the tumor volume of each group at each time point.

Statistical analysis of difference in tumor volume among the groups was conducted on the data obtained at the best therapeutic time point after the final dose.

A one-way ANOVA was performed to compare tumor volume among groups, and when a significant F-statistics (a ratio of treatment variance to the error variance) was obtained, comparisons between groups were carried out with Games-Howell test. All data were analyzed using Prism. P<0.05 was considered to be statistically significant.

Experimental Design

Table 2 shows the experimental design for study 1.

TABLE 2
Experimental Design for Study 1.

| | | | | | Dose | | |
|---|---|---|---|---|---|---|---|
| | Treat- | | dose | Concen- | volume | Dose | dose |
| Gr | ment | n | (μg) | tration | (ul) | method | regime |
| 1 | Vehicle | 3 | | | 50 ul/ | intratumor | tiw |
| | | | | | mouse | injection | |
| 2 | I-8 | 3 | | 2 mg/ml | | iv | tiw |
| 3 | I-8 | 3 | | 6 mg/ml | | iv | tiw |

Table 3 shows the experimental design for study 2.

TABLE 3
Experimental Design for Study 2.

| | | | | Dosing | | | | Dosing |
|---|---|---|---|---|---|---|---|---|
| Gr | Treatment | Dose | Dose/wk | route | | Dose | Dose/wk | route |
| 1 | Vehicle1 | | | IV | Vehicle2 | | | IP |
| 2 | I-8 | 20 mpk | 3 | IV | Vehicle2 | | | IP |
| 3 | Vehicle2 | | | IV | aPD1 | 10 mpk | 1 | IP |
| 4 | I-8 | 20 mpk | 3 | IV | aPD1 | 10 mpk | 1 | IP |

Results

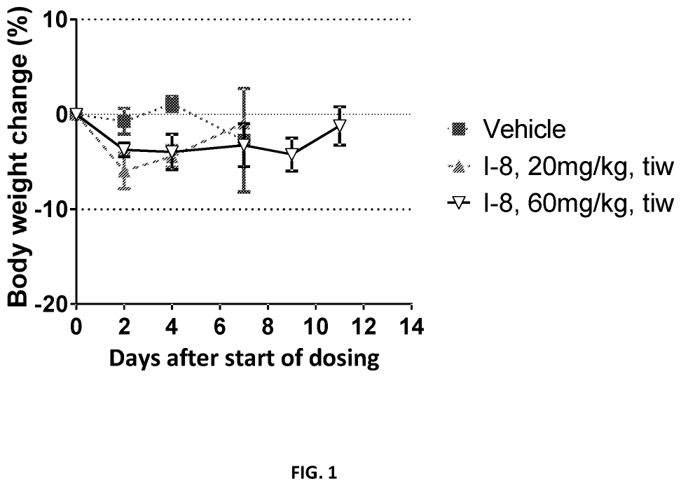

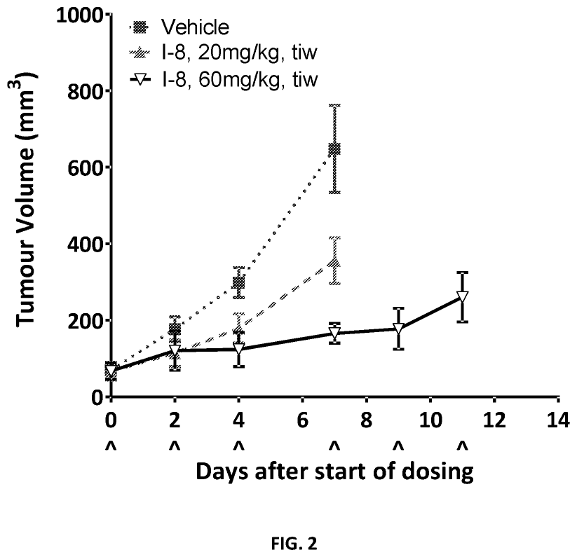

The results of study 1 are depicted in FIG. 1 and FIG. 2 which show the body weight changes and tumor volume trace after administering I-8 to female C57BL/6J mice bearing B16F10 xenograft.

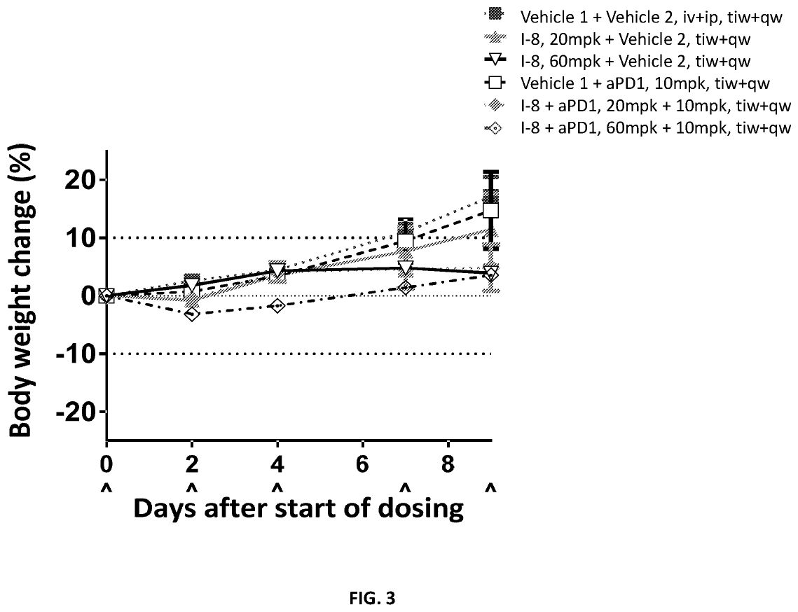

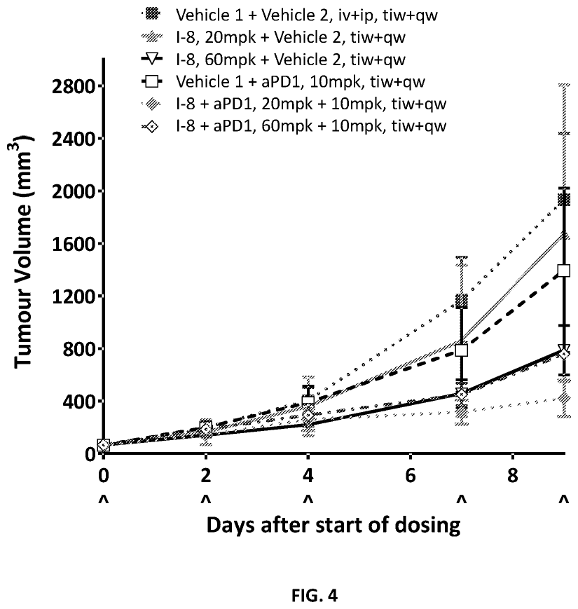

The results of study 2 are depicted in FIG. 3 and FIG. 4 which show the body weight changes and tumor volume trace after administering I-8 to female C57BL/6J mice bearing B16F10 xenograft.

Tumor Growth Inhibition Analysis

Tumor growth inhibition rate for I-8 alone or combination with aPD-1 in the B16F10 xenograft model was calculated based on tumor volume measurements at day 9 after the start of treatment.

Table 4 shows the tumor growth inhibition analysis for study 1.

TABLE 4
Tumor growth inhibition analysis for Study 1

| | | Tumor | | | |
|---|---|---|---|---|---|
| Gr | Treatment | Volume (mm³)^{a} | T/C^{b} (%) | TGI (%) | P value^{c} |
| 1 | Vehicle, tiw | 648 ± 114 | — | — | — |
| 2 | I-8, | 356 ± 60 | 54.9 | 50.0 | p > 0.05 |
| | 20 mpk, tiw | | | | |
| 3 | I-8, | 166 ± 26 | 25.6 | 82.9 | p < 0.05 |
| | 60 mpk, tiw | | | | |

^{a.}Mean ± SEM.
^{b.}Tumor Growth Inhibition is calculated by dividing the group average tumor volume for the treated group by the group average tumor volume for the control group (T/C).
^{c.}The p values of treatment groups using different chemicals were calculated respectively by one-way ANOVA or one-tailed t-test, compared with vehicle group.

Table 5 shows the tumor growth inhibition analysis for study 2.

TABLE 5
Tumor growth inhibition analysis Study 2

| | | Tumor | | | |
|---|---|---|---|---|---|
| Gr | Treatment | Volume (mm³)^{a} | T/C^{b} (%) | TGI (%) | P value^{c} |
| 1 | Vehicle1 + Vehicle2, | 1933 ± 506 | — | — | — |
| | Iv + Ip, | | | | |
| | tiw + qw | | | | |
| 2 | I-8 + Vehicle2, | 1678 ± 1129 | 86.8 | 13.7 | p > 0.05 |
| | 20 mpk(iv) + (ip), | | | | |
| | tiw + qw | | | | |
| 4 | Vehicle1 + aPD1, | 1395 ± 627 | 72.2 | 28.7 | p > 0.05 |
| | iv + 10 mpk(ip), | | | | |
| | tiw + qw | | | | |
| 5 | I-8 + aPD1, | 422 ± 138 | 21.8 | 80.9 | p > 0.05 |
| | 20 mpk(iv) + 10 mpk(ip), | | | | |
| | tiw + qw | | | | |

^{a.}Mean ± SEM.
^{b.}Tumor Growth Inhibition is calculated by dividing the group average tumor volume for the treated group by the group average tumor volume for the control group (T/C).
^{c.}The p values of treatment groups using different chemicals were calculated respectively by one-way ANOVA or one-tailed t-test, compared with vehicle group.

### Example 7: Human TLR7 and TLR8 Receptor Activation Using HEK293 Reporter Cell Lines

Compounds were tested for their activity to activate human TLR7 and TLR8 receptors using HEK293 reporter cell lines engineered to express respective receptors in accordance to manufacturer's instructions (InvivoGen). Reporter cells in HEK-Blue Detection medium (InvivoGen) were seeded to wells that contained test compounds of varied concentrations to achieve 10,000 cells/well in 50 uL volume. Incubation was for 16 hours at 37° C. and 5° C. CO₂. Absorption at 655 nm was read on CELARIOstar. Data was fitted using PRISM.

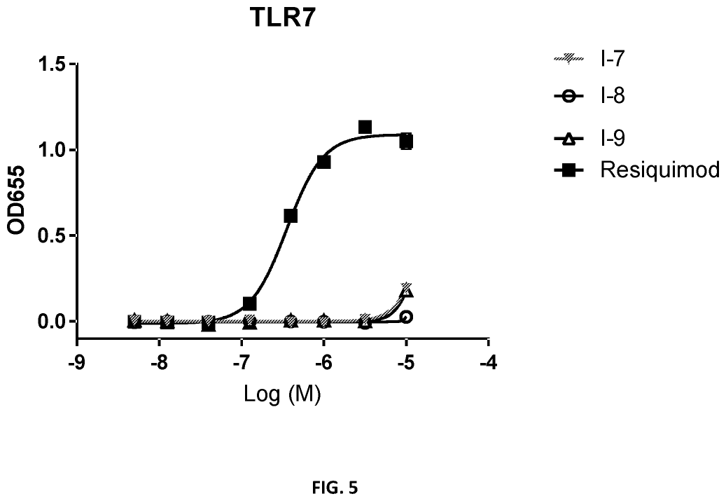

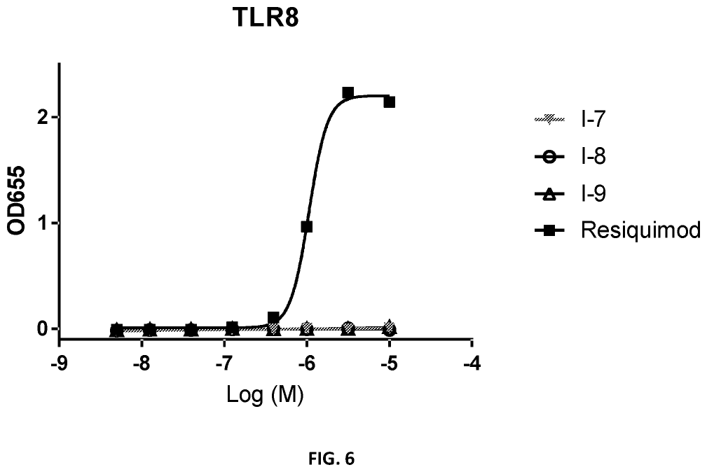

FIG. 5 depicts the assay results in the hTLR7 cell line and FIG. 6 depicts the assay results in the hTLR8 cell line. The results demonstrate that the resiquimod conjugates I-7, I-8, and I-9 demonstrate considerably reduced potency or inactivity as opposed to the naked payload resiquimod.

### Example 8. Synthesis of Compound I-25

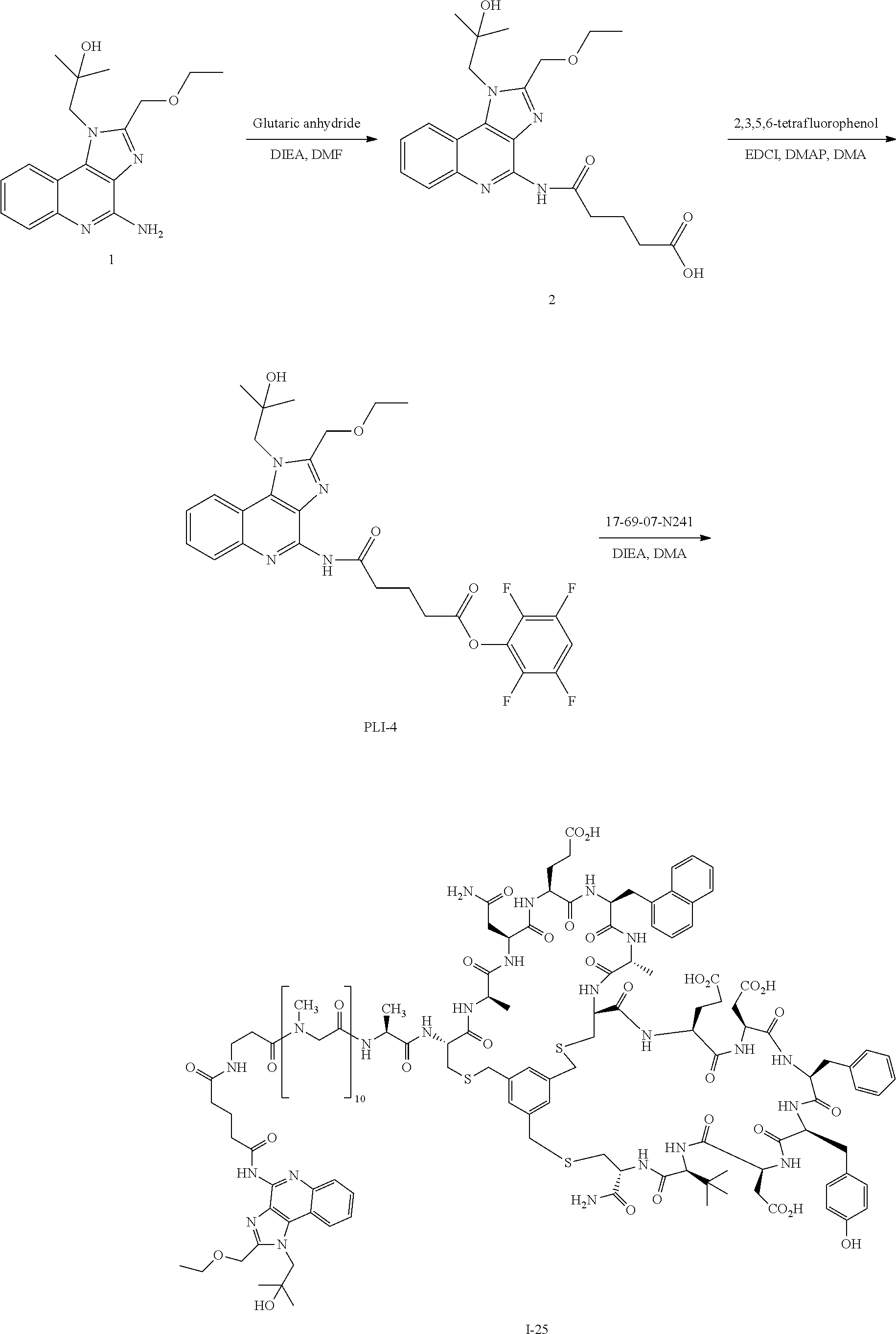

### Procedure for Preparation of Compound 2

To a solution of compound 1 (0.060 g, 190.85 umol, 1 eq) in DMF (1 mL) was added DIEA (74.00 mg, 572.55 umol, 99.73 uL, 3 eq) and glutaric anhydride (21.78 mg, 190.85 umol, 1 eq). The mixture was stirred at 25° C. for 1 hr. LC-MS showed compound 1 was consumed completely and a peak with desired mass (m/z: 429.0 ([M+H]⁺)) was detected. The reaction was filtered and concentrated under reduced pressure to give a residue. The residue was purified by prep-HPLC (A: H₂O, B: ACN). Compound 2 (0.040 g, 93.35 umol, 48.91% yield) was obtained as a white solid. Calculated MW: 428.4 observed m/z: 429.0 ([M+H]⁺)

### Procedure for Preparation of Compound PLI-4

To a solution of compound 2 (0.040 g, 93.35 umol, 1 eq) in DMA (5 mL) was added EDCI (53.69 mg, 280.06 umol, 3 eq), 2,3,5,6-tetrafluorophenol (46.51 mg, 280.06 umol, 3 eq) and DMAP (1.14 mg, 9.34 umol, 0.1 eq). The mixture was stirred at 25° C. for 2 hr. LC-MS showed compound 2 was consumed completely and a peak with desired mass (m/z: 577.0 ([M+H]⁺)) was detected. The reaction fixture was filtered and concentrated under reduced pressure to give a residue. The residue was purified by prep-HPLC (A: H₂O, B: ACN). Compound PLI-4 (0.030 g, 52.03 umol, 55.74% yield) was obtained as a white solid. Calculated MW: 576.5 observed m/z: 577.0 ([M+H]⁺)

### Procedure for Preparation of Compound I-25

To a solution of compound PLI-4 (0.030 g, 52.03 umol, 1 eq) in DMA (3 mL) was added DIEA (20.18 mg, 156.10 umol, 27.19 uL, 3 eq) and 17-69-07-N241 (152.19 mg, 57.24 umol, 1.1 eq). The mixture was stirred at 25° C. for 1 hr. LC-MS showed compound PLI-4 was consumed completely and a peak with desired m/z (m/z: 1023.5 ([M/3+H]⁺)) was detected. The reaction mixture was filtered and concentrated under reduced pressure to give a residue. The residue was purified by prep-HPLC (A: 0.075% TFA in H₂O, B: ACN). Compound I-25 (0.051 g, 15.83 umol, 30.43% yield, 95.3% purity) was obtained as a white solid. Calculated MW: 3069.4 observed m/z: 1023.5 ([M/3+H]⁺)

### Example 9. Synthesis of Compound I-27

### 9.1. Synthesis of Compound N₃-VC-Pab-PNP

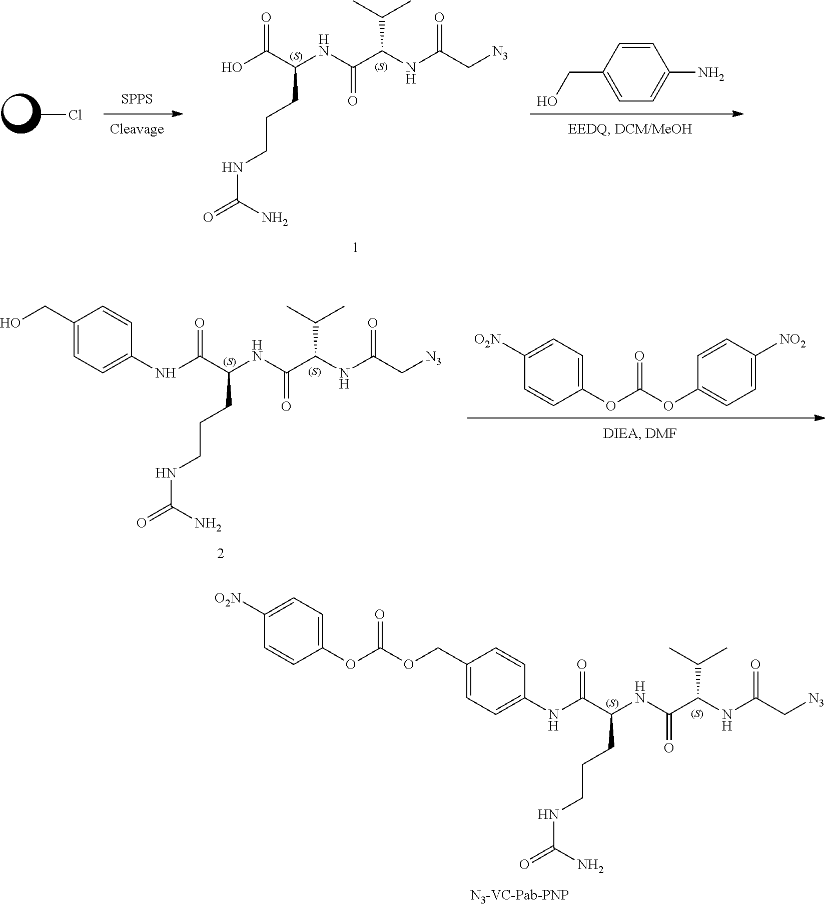

### General Procedure for Preparation of Compound 1

The peptide was synthesized using standard Fmoc chemistry.

DCM was added to the vessel containing Chlorotrityl resin (5 mmol, 4.3 g, 1.1 mmol/g) and then Fmoc-Cit-OH (1.98 g, 5 mmol, 1 eq) was added with N2 bubbling. DIEA (4.0 eq) was added dropwise and the mixture agitated for 2 hours. MeOH (5 mL) was then added and the mixture agitated for 30 min. The resin was then drained and washed with DMF 5 times. 20% piperidine/DMF was added to the resin and left to react for 30 min. The resin was then drained and washed with DMF 5 times. For subsequent couplings, Fmoc-amino acid solution in DMF was added to the resin and mixed for 30 seconds, then HBTU and DIPEA were added and the mixture agitated using nitrogen for 1 hour. Deprotection between couplings were carried out as described earlier.

| # | Materials | Coupling reagents |
|---|---|---|
| 1 | Fmoc-Cit-OH (1 eq) | DIEA (4.0 eq) |
| 2 | Fmoc-Val-OH (3 eq) | HBTU (2.85 eq) and DIEA (6.0 eq) |
| 3 | 2-azidoacetic acid (3 eq) | HBTU (2.85 eq) and DIEA (6.0 eq) |

After coupling of all listed amino acids, the resin was washed and dried. Cleavage from the resin was performed by addition of cleavage buffer (20% TFIP/80% DCM) to the flask containing the side chain protected peptide at room temperature and this was stirred for 1 hour. The solution was drained and the cleavage protocol repeated with fresh solution. The resin was filtered and the filtrate collected, the solvent was removed under reduced pressure and the crude peptide was lyophilized to give the final product (1.8 g, 90.2% purity, 45.8% yield).

### Procedure for Preparation of Compound 2

To a solution of compound 1 (0.50 g, 1.40 mmol, 1 eq.) in DCM (12.5 mL) and MeOH (6.25 mL) was added (4-aminophenyl)methanol (344.61 mg, 2.80 mmol, 2 eq.), then the reaction vessel was covered with aluminum foil. EEDQ (691.98 mg, 2.80 mmol, 2 eq.) was added and the mixture stirred at 25° C. in the dark for 3 h. LC-MS showed compound 1 was consumed completely and a peak with desired m/z (463.3 [M+H⁺]) was detected. The reaction mixture was directly purified by flash silica gel chromatography (ISCO®; 120 g SepaFlash® Silica Flash Column, Eluent of 0-30% MeOH/DCM gradient@40 mL/min) to give compound 2 (0.30 g, 648.65 umol, 46.36% yield) as a yellow solid. Calculated MW: 462.5 observed m/z: 463.3 ([M+H⁺]).

### Procedure for Preparation of Compound N3-VC-Pab-PNP

To a solution of compound 2 (0.30 g, 648.65 umol, 1 eq.) in DMF (3 mL) was added DIEA (503.00 mg, 3.89 mmol, 677.89 uL, 6 eq.) and bis(4-nitrophenyl) carbonate (789.3 mg, 2.59 mmol, 4 eq.). The reaction mixture was stirred at 25° C. for 2 h. LC-MS showed compound 2 was consumed completely and a peak with desired m/z (628.5 ([M+H⁺])) was detected. The reaction was directly purified by prep-HPLC (A: H₂O, B: ACN) to give N3-VC-Pab-PNP (0.25 g, 369.86 umol, 57.02% yield, 92.85% purity) as a white solid. Calculated MW: 627.6 observed m/z: 628.5 ([M+H⁺]).

### 9.2. Synthesis of Compound I-27

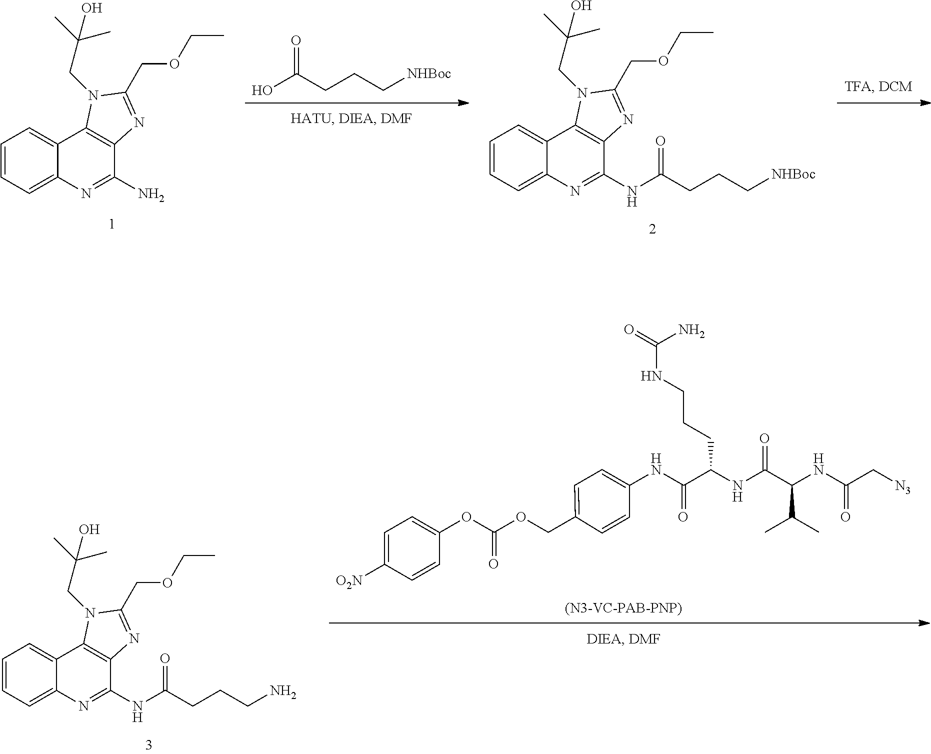 [SMILES: CCOCc1nc2c(N)nc3ccccc3c2n1CC(C)(C)O ; CC(C)(C)OC(=O)NCCCC(=O)O ; CCOCc1nc2c(NC(=O)CCCNC(=O)OC(C)(C)C)nc3ccccc3c2n1CC(C)(C)O ; CCOCc1nc2c(NC(=O)CCCN)nc3ccccc3c2n1CC(C)(C)O ; CC(C)[C@H](NC(=O)CN=[N+]=[N-])C(=O)N[C@@H](CCCNC(N)=O)C(=O)Nc1ccc(COC(=O)Oc2ccc([N+](=O)[O-])cc2)cc1 ; "(N3-VC-PAB-PNP)"]

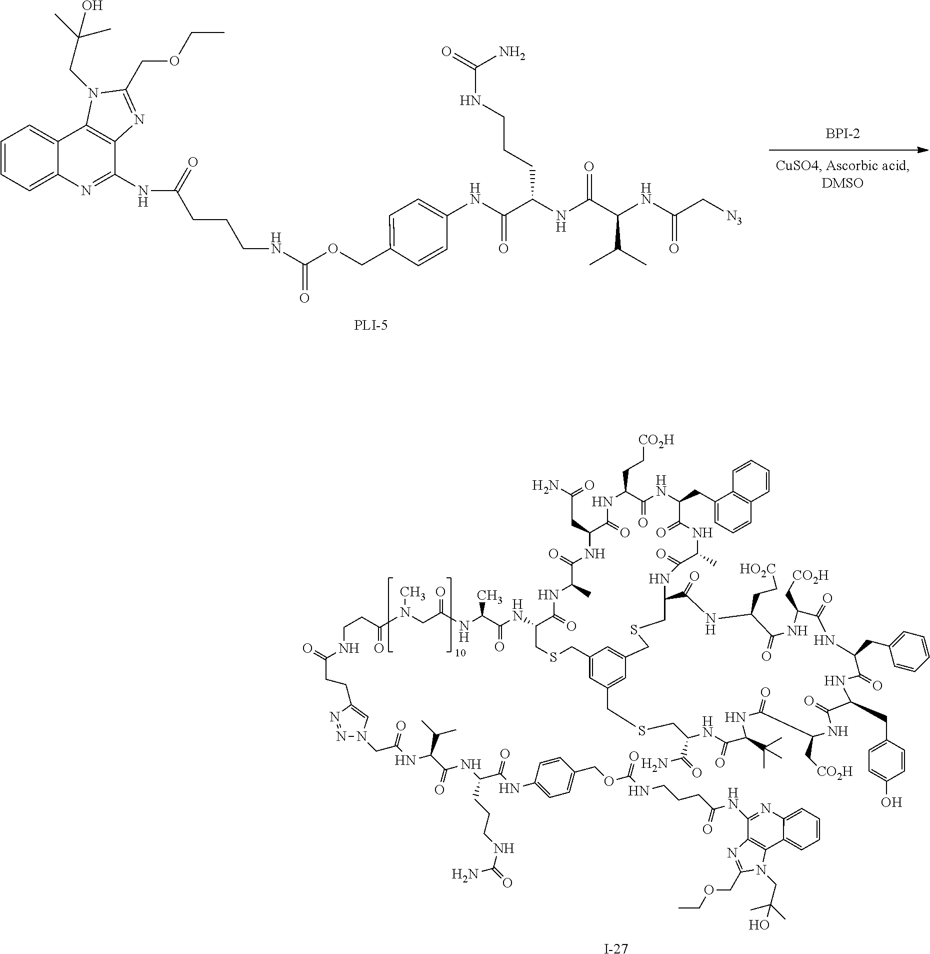

### Procedure for Preparation of Compound 2

To a solution of compound 1 (0.100 g, 318.08 umol, 1 eq) in DMF (5 mL) was added HATU (362.83 mg, 954.24 umol, 3 eq), 4-(tert-butoxycarbonylamino)butanoic acid (193.94 mg, 954.24 umol, 3 eq) and DIEA (411.10 mg, 3.18 mmol, 554.05 uL, 10 eq). The mixture was stirred at 25° C. for 2 hr. LC-MS showed compound 1 was consumed completely and a peak with desired mass (m/z: 500.0 ([M+H]⁺)) was detected. The reaction was concentrated under reduced pressure to give a residue. The residue was purified by prep-HPLC (A: H₂O, B: ACN). Compound 2 (0.085 g, 32.79 umol, 26.65% yield) was obtained as a white solid. Calculated MW: 499.6 observed m/z: 500.0 ([M+H]⁺)

### Procedure for Preparation of Compound 3

To a solution of compound 2 (0.085 g, 170.14 umol, 1 eq) in DCM (0.9 mL) was added TFA (2.62 g, 22.96 mmol, 1.70 mL, 134.95 eq). The mixture was stirred at 20° C. for 1 hr. LC-MS showed compound 2 was consumed completely and a peak with desired mass (m/z: 399.9 ([M+H]⁺)) was detected. The reaction mixture was concentrated under reduced pressure to give a residue. The residue was purified by prep-HPLC (A: H₂O, B: ACN). Compound 3 (0.045 g, 47.56 umol, 27.95% yield) was obtained as a white solid. Calculated MW: 399.4 observed m/z: 399.9 ([M+H]⁺)

### Procedure for Preparation of Compound PLI-5

To a solution of compound 3 (0.045 g, 112.64 umol, 1 eq) in DMF (2 mL) was added DIEA (14.56 mg, 112.64 umol, 19.62 uL, 1 eq) and N3-VC-PAB-PNP (70.70 mg, 112.64 umol, 1 eq). The mixture was stirred at 15° C. for 2 hr. LC-MS showed compound 3 was consumed completely and a peak with desired mass (m/z: 888.4 ([M+H]⁺)) was detected. The reaction mixture was concentrated under reduced pressure to give a residue. The residue was purified by prep-HPLC (A: H₂O, B: ACN). Compound PLI-5 (0.018 g, 20.27 umol, 18.00% yield) was obtained as a white solid. Calculated MW: 887.9 observed m/z: 888.4 ([M+H]⁺)

### Procedure for Preparation of Compound I-27

To a solution of compound PLI-5 (18.32 mg, 18.02 umol, 1 eq) in DMSO (4 mL) was added CuSO4 (0.8 M, 67.51 uL, 3 eq), Ascorbic acid (0.8 M, 135 uL, 6 eq) and compound BPI-2 (0.8 M, 225.06 uL, 10 eq). The mixture was stirred at 15° C. for 15 min. LC-MS showed compound PLI-5 was consumed completely and a peak with desired mass (observed m/z: 1209.2 ([M/3+H]⁺)) was detected. The reaction mixture was concentrated under reduced pressure to give a residue. The residue was purified by prep-HPLC (A: 0.075% NH₄OAc in H2O, B: ACN). Compound I-27 (0.0238 g, 6.27 umol, 64.82% yield, 95.6% purity) was obtained as a white solid. Calculated MW: 3627.0 observed m/z: 1209.2 ([M/3+H]⁺)

### Example 10. Synthesis of Compound I-28

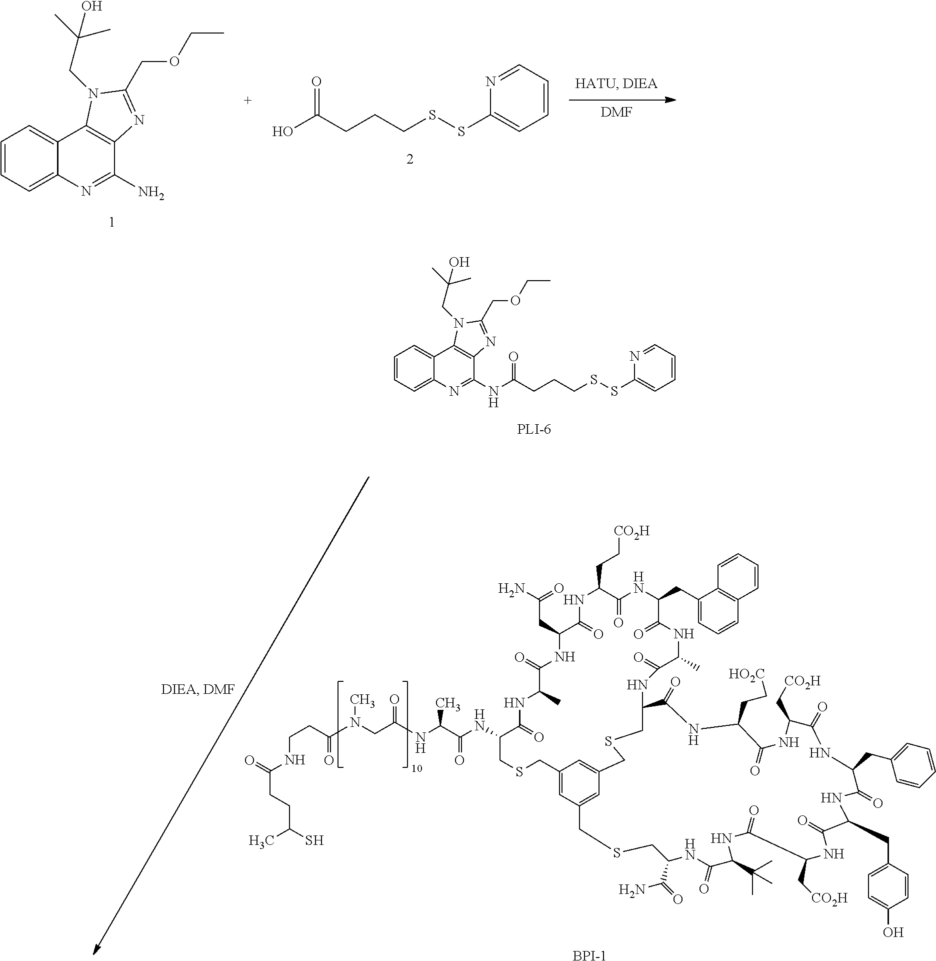

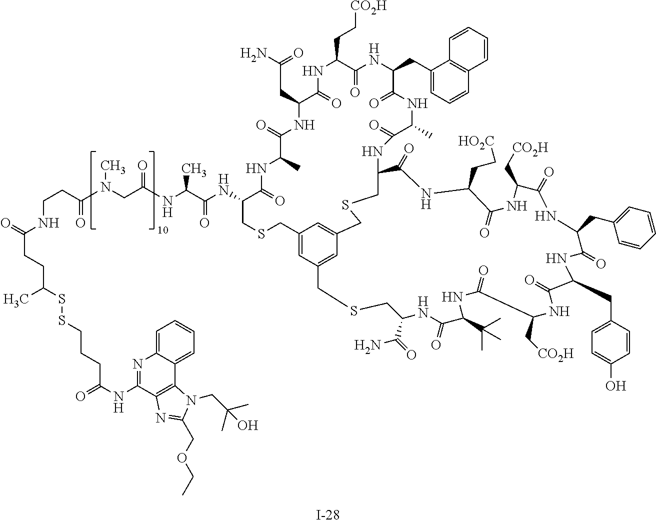

### Procedure for Preparation of Compound PLI-6

To a solution of compound 1 (0.100 g, 318.09 umol) in DMF (1 mL) was added DIEA (205.55 mg, 1.59 mmol, 277.02 uL), compound 2 (218.83 mg, 954.26 umol) and HATU (362.84 mg, 954.26 umol). The mixture was stirred at 25° C. for 2 hrs. LC-MS showed compound 1 was consumed completely and a peak with desired mass (m/z: 525.9 ([M+H]⁺)) was detected. The reaction mixture was concentrated under reduced pressure to remove solvent to give a residue. The crude compound PLI-6 (0.01 g, crude, TFA salt) (yellow oil) was used into the next step without further purification. Calculated MW: 525.69 observed m/z: 525.9 ([M+H]⁺)

### Procedure for Preparation of I-28

To a solution of compound PLI-6 (0.010 g, 19.02 umol) in DMF (1 mL) was added DIEA (24.59 mg, 190.23 umol, 33.13 uL) and compound BPI-1 (58.07 mg, 20.93 umol). The mixture was stirred at 20° C. for 1 hr. LC-MS showed compound PLI-6 was consumed completely and a peak with desired mass (m/z: 1063.6 (M/3+H)⁺) was detected. The reaction mixture was concentrated under reduced pressure to give a residue. The residue was purified by prep-HPLC (A: 0.075% TFA in H₂O, B: ACN). Compound I-28 (0.0081 g, 13.11% yield, 98.23% purity) was obtained as a white solid. Calculated MW: 3189.64, observed MS: 1063.6 (M/3+H)⁺

### Example 11. Synthesis of Compound I-29

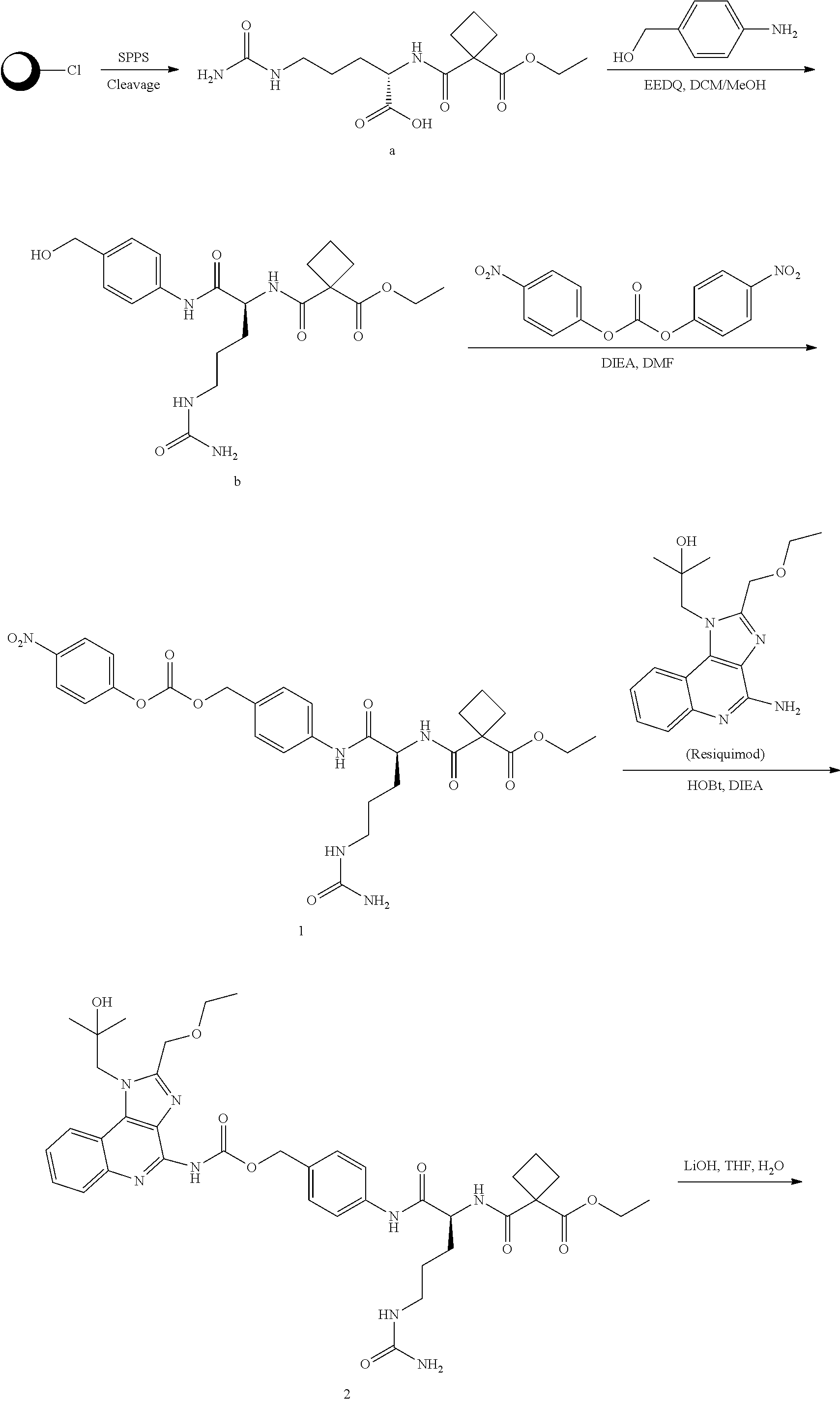

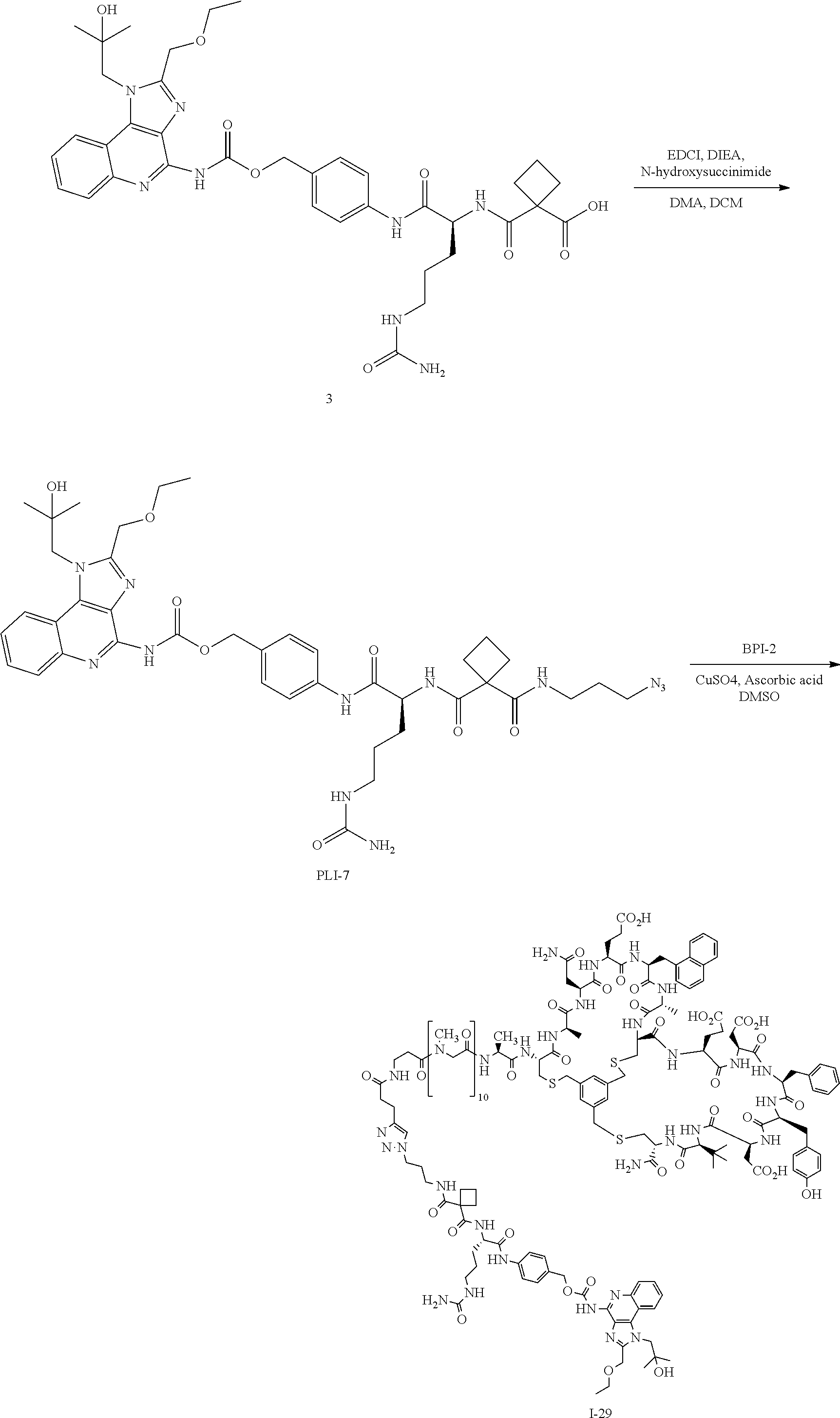

### General Procedure for Preparation of Compound a

Peptide Synthesis:

The peptide was synthesized using standard Fmoc chemistry.
1. Add DCM to the vessel containing CTC Resin (5 mmol, 4.3 g, 1.1 mmol/g) and Fmoc-Cit —OH (1 eq) (1.98 g, 5 mmol, 1 eq) with N2 bubbling.
2. Add DIEA (4.0 eq) dropwise and mix for 2 hours.
3. Add MeOH (3 mL) and mix for 30 min.
4. Drain and wash with DMF for 5 times.
5. Add 20% piperidine/DMF and react on 30 min.
6. Drain and wash with DMF for 5 times.
7. Add Fmoc-amino acid solution and mix 30 seconds, then add activation buffer, N2 bubbling for about 1 hour.
8. Repeat above step 4 to 7 for the coupling of following amino acids.

| # | Materials | Coupling reagents |
|---|---|---|
| 1 | Fmoc-Cit-OH (1 eq) | DIEA (4.0 eq) |
| 2 | (2S)-2-[(1- | HBTU (1.9 eq) and |
| | ethoxycarbonylcyclobutanecarbonyl)- | DIEA (4 eq) |
| | amino]-5- | |
| | ureido-pentanoic acid (2 eq) | |

20% piperidine in DMF was used for Fmoc deprotection for 30 min. The coupling reaction was monitored by ninhydrin test, and the resin was washed with DMF for 5 times.

Peptide Cleavage and Purification:
1. Add cleavage buffer (20% TFIP/80% DCM) to the flask containing the side chain protected peptide at room temperature and stir for 1 hours twice.
2. Filter and collect the filtrate.
3. Concentrate to remove the solvent.
4. The crude peptide was lyophilized to give the final product (1.7 g).

### Procedure for Preparation of Compound b

To a solution of compound a (1.65 g, 5.01 mmol, 1 eq) in DCM (10 mL) and MeOH (5 mL) was added (4-aminophenyl) methanol (740.37 mg, 6.01 mmol, 1.2 eq), then the reaction vessel was covered with aluminum foil. And then EEDQ (2.48 g, 10.02 mmol, 2 eq) was added to the reaction mixture, the reaction mixture was stirred at 25° C. in the dark for 16 h. TLC showed compound a was consumed completely. The reaction mixture was directly purified by flash silica gel chromatography (ISCO®; 80 SepaFlash® Silica Flash Column, Eluent of 0-15Ethyl DCM/MeOH gradient@60 L/min) to give compound b (1.3 g, 2.99 mmol, 59.72% yield) as a yellow solid.

### Procedure for Preparation of Compound 1 (EtO-cBut-Cit-Pab-PNP)

To a solution of compound b (1.3 g, 2.99 mmol, 1 eq) in IMF (10 mL) was added DIEA (2.32 g, 17.95 mmol, 3.13 mL, 6 eq) and bis(4-nitrophenyl) carbonate (3.64 g, 11.97 mmol, 4 eq), the reaction mixture was stirred at 15° C. for 1 h. LC-MS showed compound b was consumed completely and a peak with desired mass (m/z: 600.0 ([M+H⁺])) was detected. The reaction was directly purified by prep-HPLC (A: H₂O, B: ACN) to give compound 1 (EtO-cBut-Cit-Pab-PNP) (1.0 g, 1.67 mmol, 55.74% yield) as a yellow solid. (calculated MW: 599.59 observed m/z: 600.0 ([M+H⁺])).

### Procedure for Preparation of Compound 2

To a solution of compound 1 (0.250 g, 795.21 umol) in IMF (5 mL) was added HOBt (161.18 mg, 1.19 mmol), and DIEA (308.33 mg, 2.39 mmol, 415.54 uL) followed by Resiquimod (1.02 g, 1.19 mmol). The mixture was stirred at 15° C. for 16 hr. LC-MS showed compound 1 was consumed completely and a with desired mass (m/z: 775.1 ([M+H]⁺)) was detected. The reaction was directly purified by prep-HPLC (A: H2O, B: ACN) to give compound 2 (0.085 g, 109.70 umol, 13.79% yield) as a white solid. Calculated MW: 774.86 observed m/z: 775.1 ([M+H⁺])

### Procedure for Preparation of Compound 3

To a solution of compound 2 (0.055 g, 70.98 umol,) in THF (2 mL) was added LiOH (5.96 mg, 141.96 umol) and H₂O (2 mL). The mixture was stirred at 15° C. for 2 hr. LC-MS showed compound 2 was consumed completely and a peak with desired mass (m/z: 747.3 ([M+H⁺])) was detected. The reaction mixture was quenched by addition AcOH (1 mL) and used for next step directly without further purification. Compound 3 (0.030 g, 40.17 umol, 56.59% yield) was obtained as a white solid. Calculated MW: 746.81 observed m/z: 747.3 ([M+H⁺]).

### Procedure for Preparation of Compound PLI-7

To a solution of compound 3 (0.028 g, 37.49 umol,) in DMA (3 mL) was added EDCI (14.37 mg, 74.99 umol), DIEA (14.54 mg, 112.48 umol, 19.59 uL), 3-azidopropan-1-amine (18.77 mg, 187.46 umol), DCM (1 mL) and N-hydroxysuccinimide (8.63 mg, 74.99 umol). The mixture was stirred at 15° C. for 6 hr. LC-MS showed compound 3 was consumed completely and a peak with desired mass (m/z: 829.1 ([M+H⁺])) was detected. The reaction mixture was filtered and concentrated under reduced pressure to give a residue. It was purified by prep-HPLC (A: H2O, B: ACN) to give compound PLI-7 (0.030 g, 36.19 umol, 96.53% yield) as a white solid. Calculated MW: 828.91 observed m/z: 829.1 ([M+H⁺]).

### Procedure for Preparation of I-29

To a solution of compound PLI-7 (0.020 g, 24.13 umol, 1 eq) in DMSO (2 mL) was added CuSO₄ (0.8 M, 90.48 uL), Ascorbic acid (0.8 M, 301.60 uL) and compound BPI-2 (122.96 mg, 44.89 umol). The mixture was stirred at 15° C. for 15 min. LC-MS showed compound PLI-7 was consumed completely and a peak with desired mass (m/z: 1189.9 ([M+H⁺])) was detected. The reaction mixture was centrifuged and the solution phase was directly purified by prep-HPLC (A: 0.075% TFA in H2O, B: ACN). Compound I-29 (0.023 g, 5.54 umol, 22.98% yield, 86.1% purity) was obtained as a white solid. Calculated MW: 3567.93 observed m/z: 1189.9 ([M/3+H⁺]).

### Example 12. Synthesis of Compound I-31

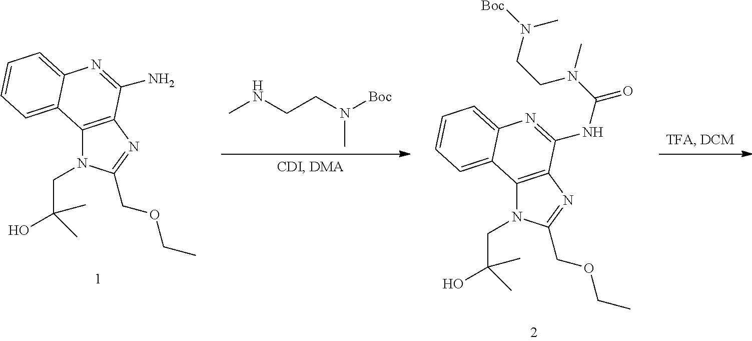

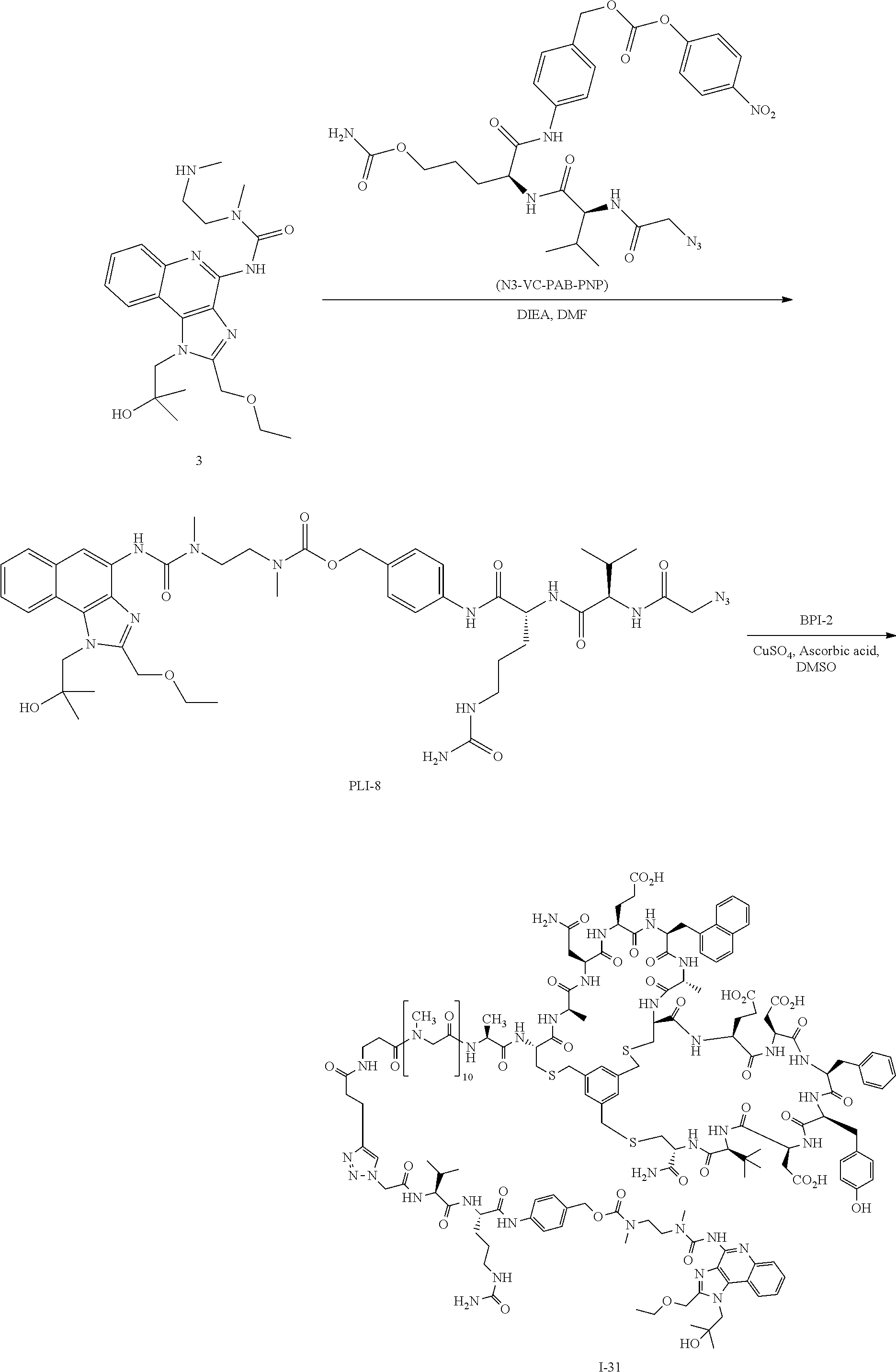

### Procedure for Preparation of Compound 2

To a solution of tert-butyl N-methyl-N-[2-(methylamino)ethyl]carbamate (89.83 mg, 477.13 umol, 3 eq) in DMA (1 mL) was added CDI (257.89 mg, 1.59 mmol, 10 eq) and compound 1 (Resiquimod, 0.050 g, 159.04 umol, 1 eq). The mixture was stirred at 15° C. for 2 hr. LC-MS showed compound 1 was consumed completely and a peak with desired mass (m/z: 529.2 ([M+H]⁺)) was detected. The residue was purified by prep-HPLC (A: H₂O, B: ACN). Compound 2 (0.060 g, 113.50 umol, 71.36% yield) was obtained as a white solid. Calculated MW: 528.6 observed m/z: 529.2 ([M+H]⁺)

### Procedure for Preparation of Compound 3

To a solution of Compound 2 (0.060 g, 113.50 umol, 1 eq) in DCM (9 mL) was added TFA (18.48 g, 162.07 mmol, 12.00 mL, 1427.97 eq). The mixture was stirred at 15° C. for 1 hr. LC-MS showed Compound 2 was consumed completely and a peak with desired mass (m/z: 429.3 ([M+H]⁺)) was detected. The residue was purified by prep-HPLC (A: H₂O, B: ACN). Compound 3 (0.045 g, 105.01 umol, 92.52% yield) was obtained as a white solid. Calculated MW: 428.5 observed m/z: 429.3 ([M+H]⁺)

### Procedure for Preparation of Compound PLI-8

To a solution of Compound 3 (0.045 g, 105.01 umol, 1 eq) in DMF (5 mL) was added DIEA (13.57 mg, 105.01 umol, 18.29 uL, 1 eq) and N₃—VC-PAB-PNP (98.86 mg, 157.52 umol, 1.5 eq). The mixture was stirred at 20° C. for 2 hr. TLC showed N₃-VC-PAB-PNP was consumed completely (DCM/MeOH=5:1) and a new spot was detected. The residue was purified by flash-chromatography (A: H₂O, B: ACN). Compound PLI-8 (0.060 g, 65.43 umol, 62.31% yield) was obtained as a white solid. Calculated MW: 917.0

### Procedure for Preparation of I-31

To a solution of Compound PLI-8 (0.030 g, 32.71 umol, 1 eq) in DMSO (4 mL) was added CuSO₄ (0.8 M, 122.68 uL, 3 eq), compound BPI-2 (80.65 mg, 29.44 umol, 0.9 eq) and Ascorbic acid (0.8 M, 408.93 uL, 10 eq). The mixture was stirred at 15° C. for 15 min. LC-MS showed Compound PLI-8 was consumed completely and a peak with desired mass (m/z: 1218.7 ([M/3+H]⁺)) was detected. The residue was purified by prep-HPLC (A: 0.075% NH₄OAc in H2O, B: ACN). Compound I-31 (0.030 g, 7.98 umol, 24.41% yield, 97.3% purity) was obtained as a white solid. Calculated MW: 3656.0 observed m/z: 1218.7 ([M/3+H]⁺)

### Example 13. Synthesis of Compound I-32

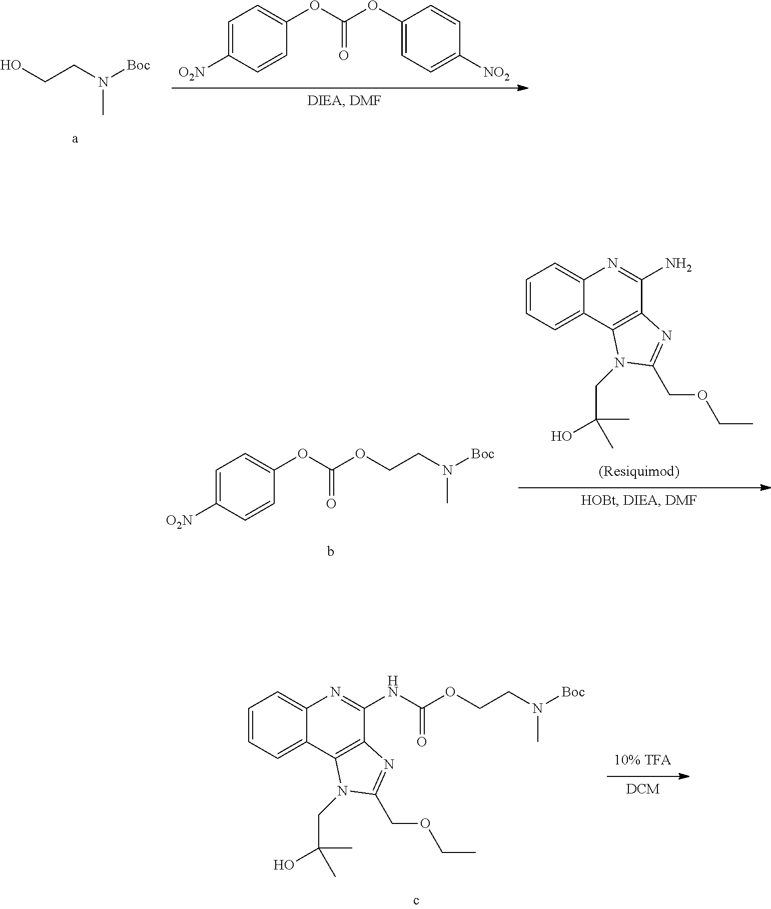

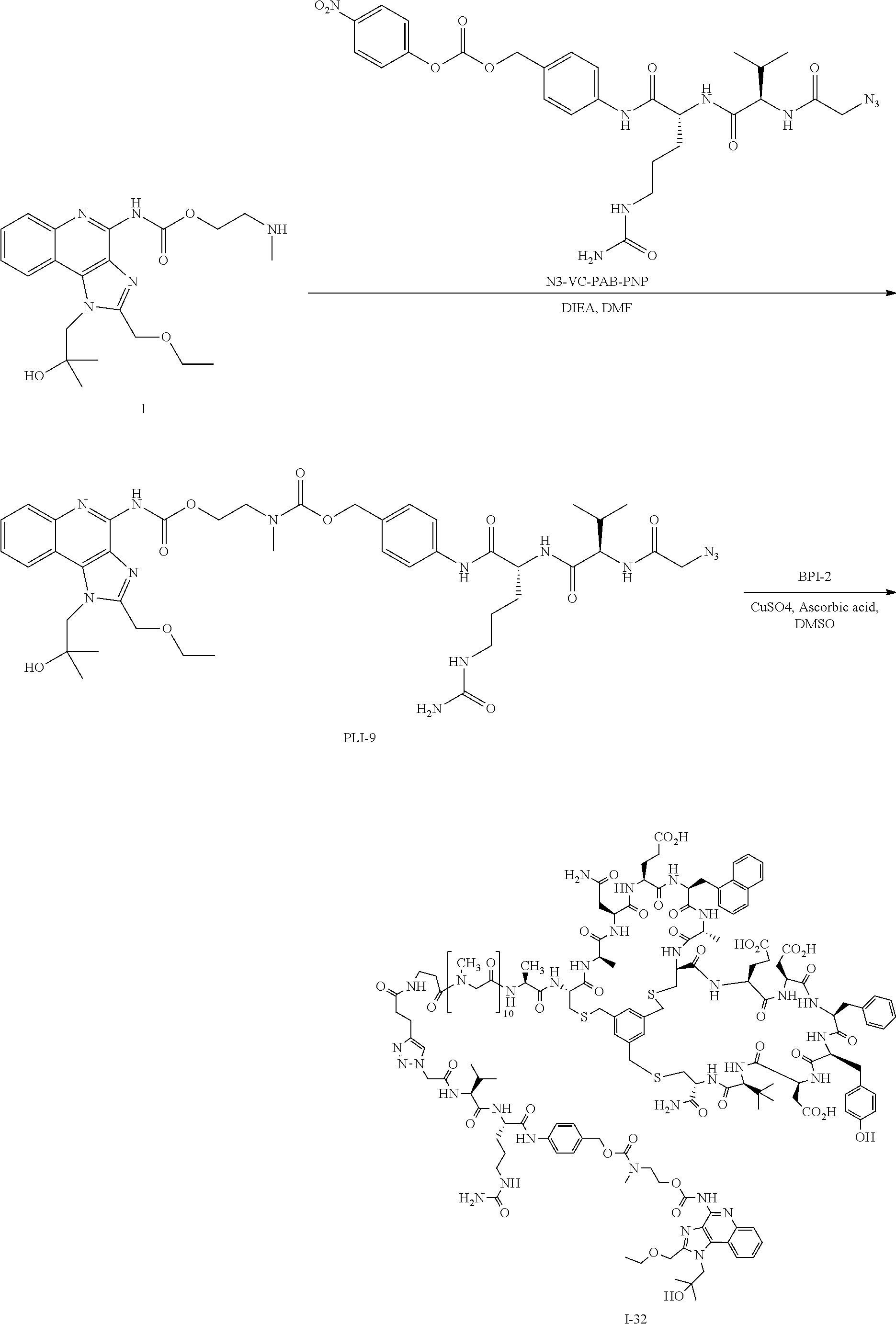

### Procedure for Preparation of Compound b

To a solution of compound a (2 g, 11.41 mmol, 1 eq) in DMF (1 mL) was added bis(4-nitrophenyl) carbonate (2.78 g, 9.13 mmol, 0.8 eq) and DIEA (4.43 g, 34.24 mmol, 5.96 mL, 3 eq). The mixture was stirred at 15° C. for 1 hr. LC-MS showed compound a was consumed completely and a peak with desired mass (m/z: 363.0 ([M+Na⁺])) was detected. The reaction mixture was directly purified by prep-HPLC (A: H2O, B: ACN) to give Compound b (1 g, 2.94 mmol, 25.74% yield) as a white solid. Calculated MW: 340.33 observed m/z: 363.0 ([M+Na]⁺)

### Procedure for Preparation of Compound c

To a solution of compound b (216.51 mg, 636.17 umol, 2 eq) in DMF (1 mL) was added HOBt (128.94 mg, 954.26 umol, 3 eq), resiquimod (0.100 g, 318.09 umol, 1 eq) and DIEA (205.55 mg, 1.59 mmol, 277.02 uL, 5 eq). The mixture was stirred at 15° C. for 2 hr. LC-MS showed compound b was consumed completely and a peak with desired mass (m/z: 538.3 ([M+Na]⁺)) was detected. The reaction was directly purified by prep-HPLC (A: H2O, B: ACN) to give compound c (0.070 g, 135.76 umol, 42.68% yield) as a white solid. Calculated MW: 515.60 observed m/z: 538.3 ([M+Na]⁺)

### Procedure for Preparation of Compound 1

To a solution of compound c (0.070 g, 135.73 umol, 1 eq) in DCM (5 mL) was added TFA (0.5 mL). The mixture was stirred at 15° C. for 1 hr. LC-MS showed compound c was consumed completely and a peak with desired mass (m/z: 416.2 ([M+H]⁺)) was detected. The reaction was directly purified by prep-HPLC (A: H2O, B: ACN) to give compound 1 (0.070 g, 168.47 umol crude) as a pale yellow oil (calculated MW: 415.48 observed m/z: 416.2 ([M+H]⁺)).

### Procedure for Preparation of Compound PLI-9

To a solution of compound 1 (0.075 g, 180.51 umol) in DMF (3 mL) was added DIEA (116.65 mg, 902.56 umol, 157.21 uL) and N₃—VC-PAB-PNP (147.28 mg, 234.67 umol). The mixture was stirred at 20° C. for 2 hr. LC-MS showed compound 1 was consumed completely and a peak with desired mass (m/z: 904.1 ([M+H⁺])) was detected. The reaction mixture was directly purified by prep-HPLC (A: H2O, B: ACN) to give Compound PLI-9 (0.050 g, 55.31 umol, 30.64% yield) as a white solid. Calculated MW: 903.98 observed m/z: 904.1 ([M+H⁺])

### Procedure for Preparation of I-32

To a solution of compound PLI-9 (0.025 g, 27.66 umol) in DMSO (4 mL) was added copper sulfate (13.24 mg, 82.98 umol, 12.73 uL), Ascorbic acid and compound BPI-2 (68.17 mg, 24.89 umol), The mixture was stirred at 15° C. for 15 min. LC-MS showed compound 2 was consumed completely and a with desired mass (m/z: 1214.5 ([M/3+H⁺])) was detected. The reaction was directly purified by prep-HPLC (A: H2O, B: ACN) to give Compound I-32 (0.0114 g, 3.00 umol, 10.86% yield, 96.6% purity) as a white solid. Calculated MW: 3643.00 observed m/z: 1214.7 ([M/3+H⁺])

### Example 14. Synthesis of Compound I-33

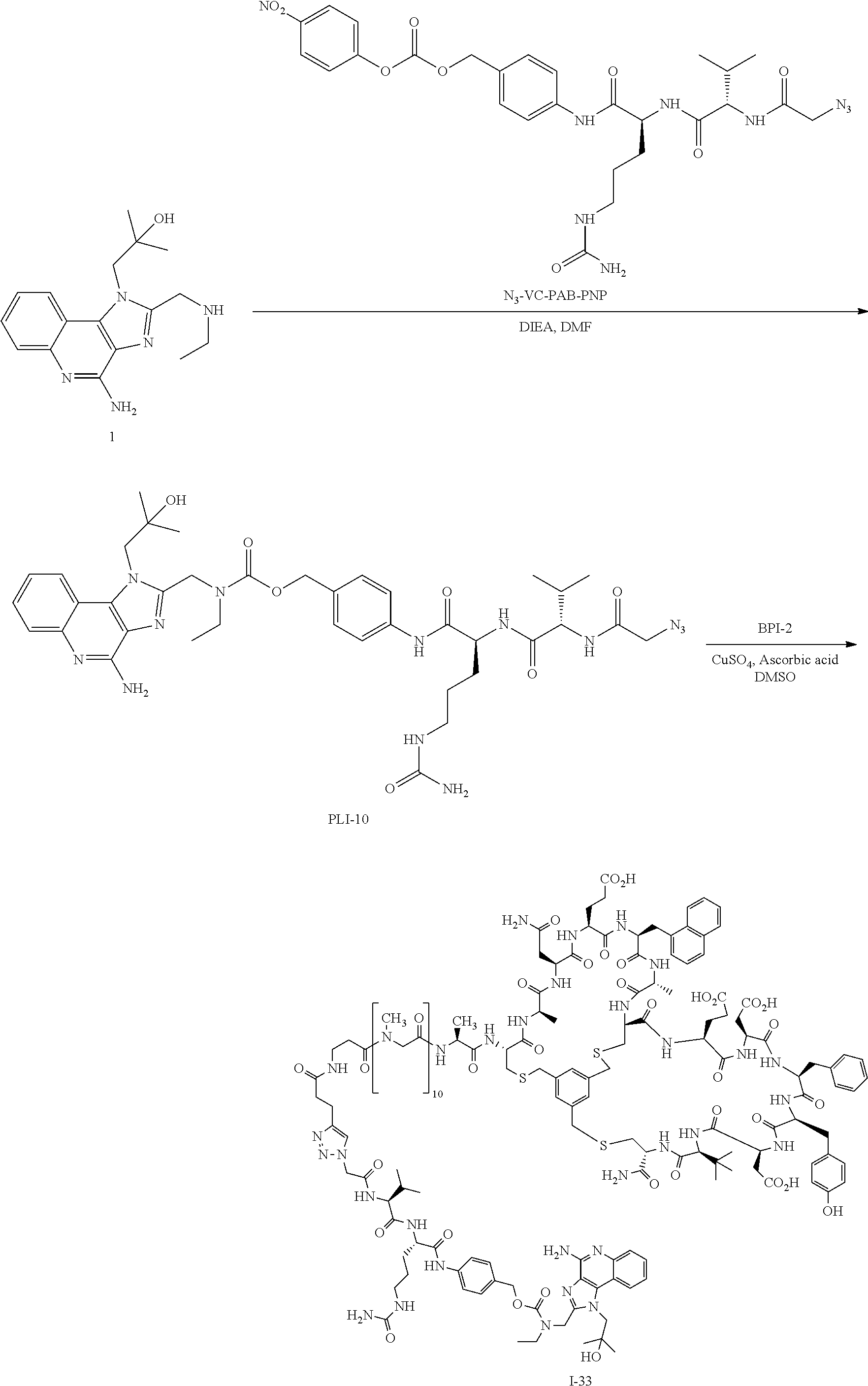

### Procedure for Preparation of Compound PLI-10

To a solution of N₃-VC-PAB-PNP (0.100 g, 159.34 umol) in DMF (1 mL) was added compound 1 (Gardiquimod, 60.39 mg, 111.54 umol, 2TFA Salt) and DIEA (61.78 mg, 478.01 umol, 83.26 uL). The mixture was stirred at 15° C. for 16 hr. LC-MS showed compound N₃-VC-PAB-PNP was consumed completely and a peak with mass (m/z: 801.7 ([M+H]⁺)) was detected. The reaction was purified by flash-HPLC (A: H₂O, B: ACN) to give compound PLI-10 (0.060 g, 74.82 umol, 46.96% yield) as a white solid. Calculated MW: 801.8 observed m/z: 801.7 ([M+H⁺])

### Procedure for Preparation of I-33

To a solution of compound PLI-10 (0.030 g, 37.41 umol) in DMSO (0.5 mL) was added copper sulfate (0.8 M, 140.29 uL), ascorbic acid (0.8 M, 467.64 uL) and BPI-2 (122.96 mg, 44.89 umol), The mixture was stirred at 15° C. for 15 min. LC-MS showed compound PLI-10 was consumed completely and a peak with desired mass (m/z: 1180.8 ([M/3+H⁺])) was detected. The reaction was purified by prep-HPLC (A: 0.075% NH₄OAc in H₂O, B: ACN) to give I-33 (0.0354 g, 10.00 umol, 26.72% yield, 95.1% purity) as a white solid. Calculated MW: 3540.9 observed m/z: 1180.8 ([M/3+H⁺])

### Example 15. Synthesis of Compound I-30

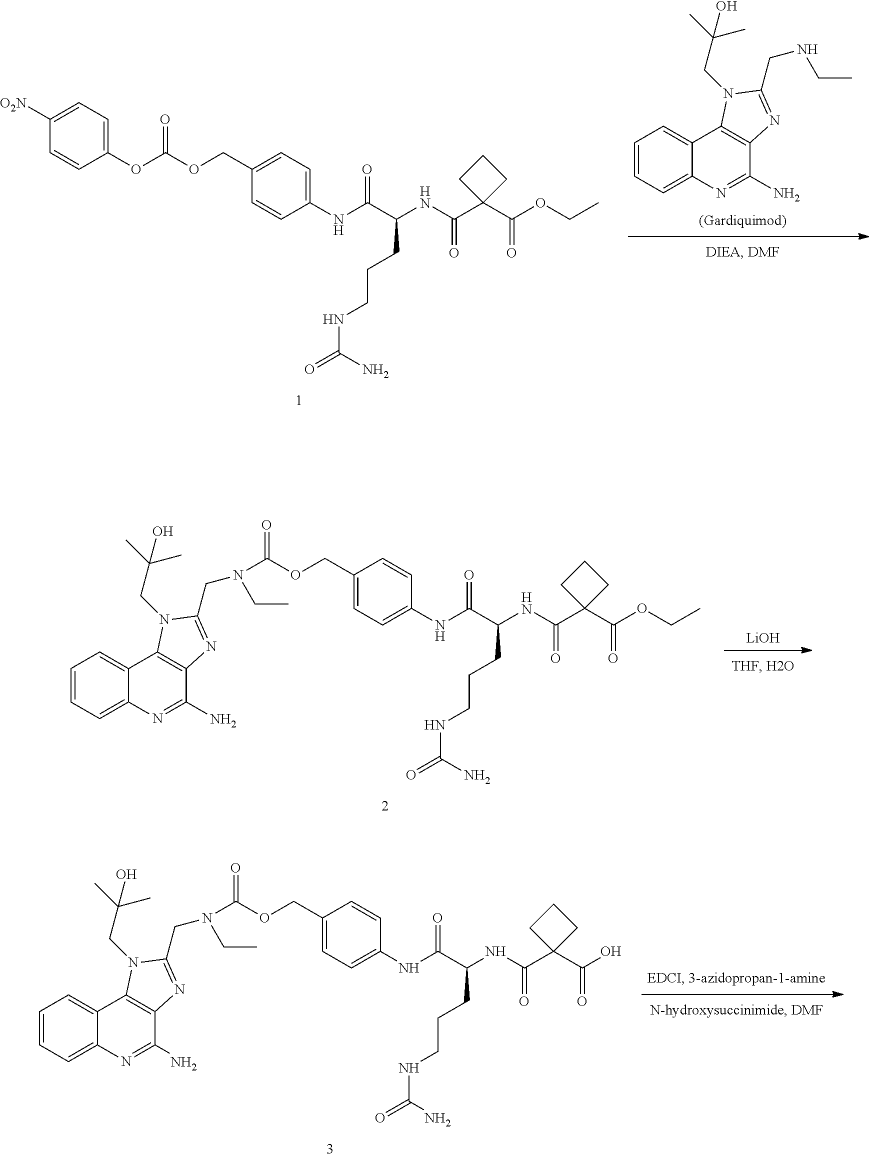

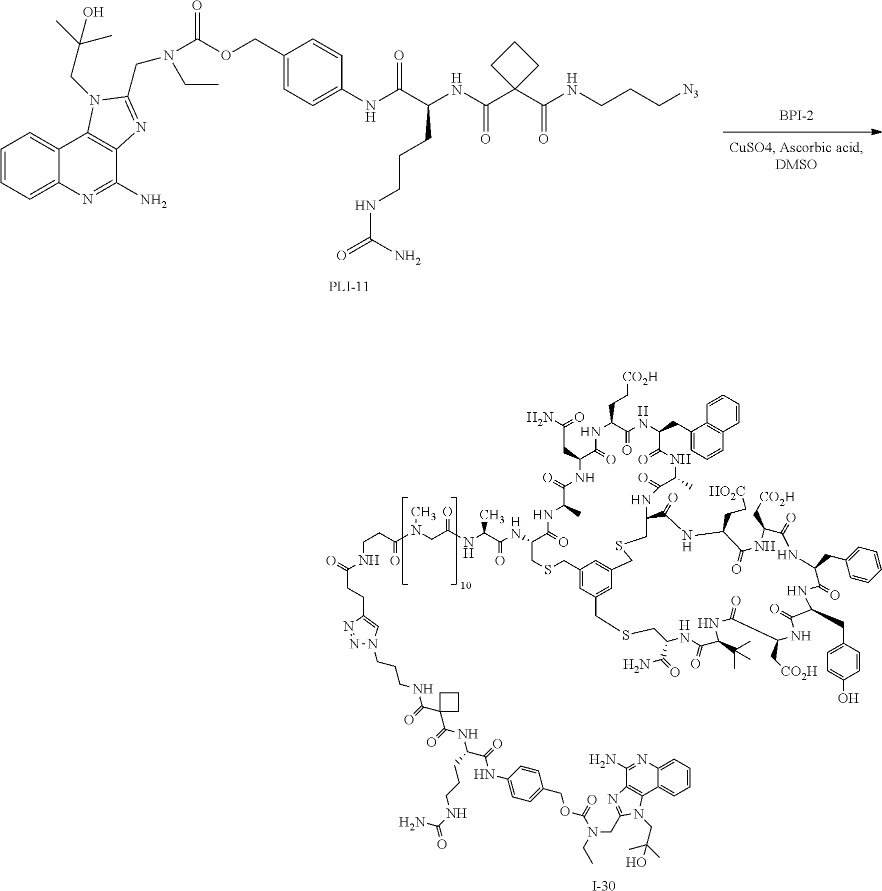

### Procedure for Preparation of Compound 2

Compound 1 was prepared as described in the synthesis of compound I-29 in example 11. To a solution of compound 1 (0.070 g, 116.75 umol) in DMF (1 mL) was added gardiquimod (44.25 mg, 81.72 umol, 0.7 eq, 2TFA) and DIEA (15.09 mg, 116.75 umol, 20.34 uL). The mixture was stirred at 15° C. for 1 hr. LC-MS showed compound 1 was consumed completely and a peak with desired mass (m/z: 774.1 ([M+H⁺])) was detected. The reaction mixture was directly purified by Prep-HPLC (A: H2O, B: ACN) to give compound 2 (0.050 g, 64.61 umol, 55.34% yield) as a white solid. Calculated MW: 773.8 observed m/z: 774.1 ([M+H⁺]))

### Procedure for Preparation of Compound 3

To a solution of compound 2 (0.050 g, 64.61 umol) in THF (2 mL) was added LiOH (3.09 mg, 129.22 umol) and H₂O (2 mL). The mixture was stirred at 15° C. for 2 hr. LC-MS showed compound 2 was consumed completely and a peak with desired mass (m/z: 746.4 ([M+H⁺])) was detected. The reaction mixture was directly purified by Prep-HPLC (A: H2O, B: ACN) to give compound 3 (0.045 g, 60.34 umol, 93.39% yield) as a white solid. Calculated MW: 745.8 observed m/z: 746.4 ([M+H⁺])

### Procedure for Preparation of Compound PLI-11

To a solution of N-hydroxysuccinimide (6.17 mg, 53.63 umol, 1 eq) in DMF (3 mL) was added EDCI (10.28 mg, 53.63 umol), compound 3 (0.040 g, 53.63 umol) and 3-azidopropan-1-amine (10.74 mg, 107.26 umol). The mixture was stirred at 15° C. for 2 hr. LC-MS showed compound 3 was consumed completely and a peak with desired mass (m/z: 828.1 ([M+H⁺])) was detected. The reaction mixture was directly purified by Prep-HPLC (A: H2O, B: ACN) to give compound PLI-11 (0.040 g, 48.31 umol, 90.08% yield) as a white solid. Calculated MW: 827.9 observed m/z: 828.1 ([M+H⁺]).

### Procedure for Preparation of I-30

To a solution of compound PLI-11 (0.025 g, 30.20 umol) in DMSO (4 mL) was added ascorbic acid (0.8 M, 377.45 uL) BPI-2 (74.44 mg, 27.18 umol) and CuSO4 (0.8 M, 113.23 uL). The mixture was stirred at 20° C. for 15 min. LC-MS showed compound PLI-11 was consumed completely and a peak with desired mass (calculated MW: 3566.9 observed m/z: 1190.3 ([M/3+H+])) was detected. The residue was directly purified by prep-HPLC (NH4OAc condition) to give I-30 (0.0149 g, 4.13 umol, 13.67% yield, 97.0% purity) as a white solid. Calculated MW: 3566.9 observed m/z: 1189.7 ([M/3+H+])

### Example 16: Synthesis of Compound I-17

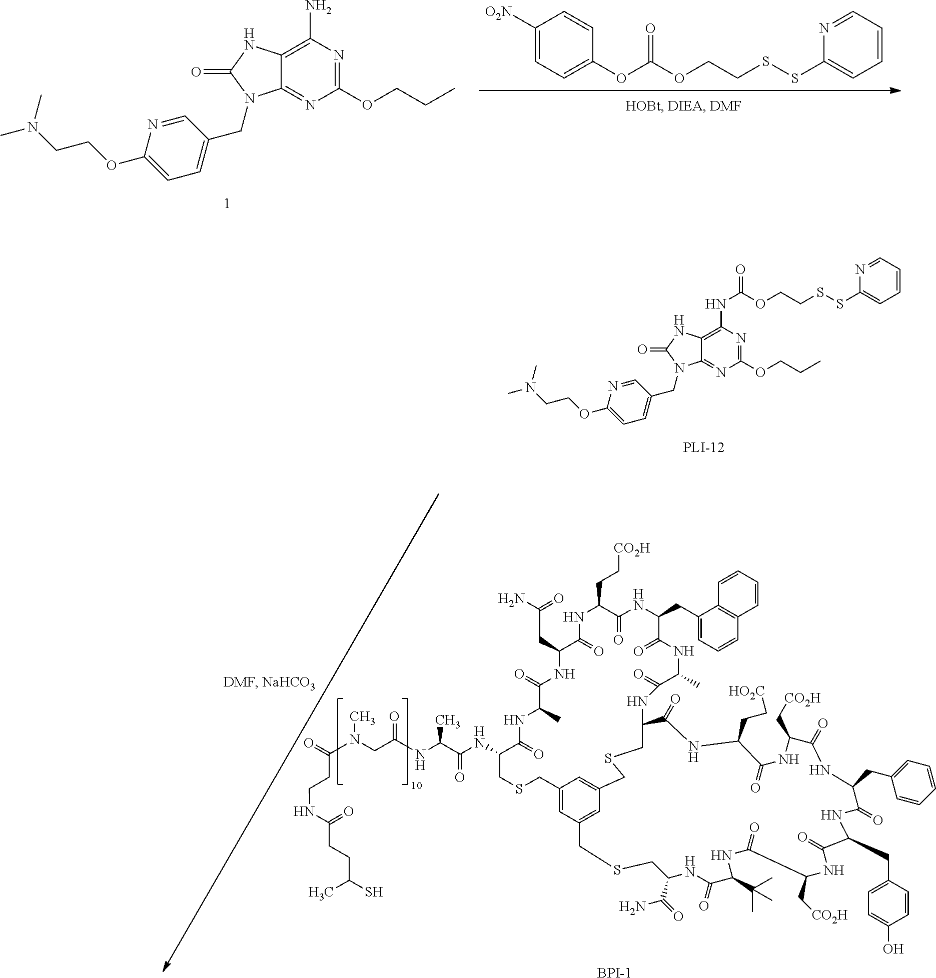

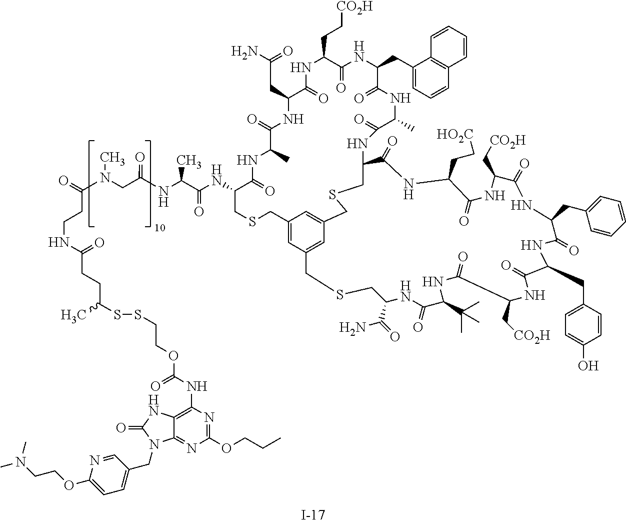

### Procedure for Preparation of Compound PLI-12

To a solution of compound 1 (0.02 g, 51.6 umol), (4-nitrophenyl) 2-(2-pyridyldisulfanyl)ethyl carbonate (21.8 mg, 62.0 umol) in DMF (2 mL) was added HOBt (10.5 mg, 77.4 umol) and DIEA (20.0 mg, 155 umol, 27.0 uL). The mixture was stirred at 25° C. for 16 hr. LC-MS showed most of compound 1 was consumed and a peak with desired m/z (601.0 ([M+H+])) was detected. The solvent was removed to give a residue, which was purified by flash silica gel chromatography (ISCO®; 20 g SepaFlash® Silica Flash Column, Eluent of 0-20% DCM/MeOH gradient@35 mL/min) to give compound PLI-12 (30.0 mg, crude) as a white solid. Calculated MW: 600.7 observed m/z: 601.0 ([M+H+])

### Procedure for Preparation of I-17

To a solution of compound PLI-12 (24.0 mg, 40.0 umol, 1 eq) and BPI-1 (111 mg, 40.0 umol) in DMF (2 mL) was added NaHCO₃ (1 mL) to adjust the PH to ˜8 and the mixture was stirred at 25° C. for 1 hr. LC-MS showed compound PLI-12 was consumed completely and a peak with desired m/z (1632.7 ([M/2+H+]), 1088.9 ([M/3+H+]), 816.5 ([M/4+H+])) was detected. The reaction mixture was purified by prep-HPLC (A: 0.075% TFA in H2O, B: ACN) to give compound I-17 (41.5 mg, 12.4 umol, 30.9% yield, 97.2% purity) as a white solid. Calculated MW: 3264.6 observed m/z: 1632.8 ([M/2+H+]), 1088.7 ([M/3+H+]), 816.7 ([M/4+H+])

### Example 17. Synthesis of Compound I-18

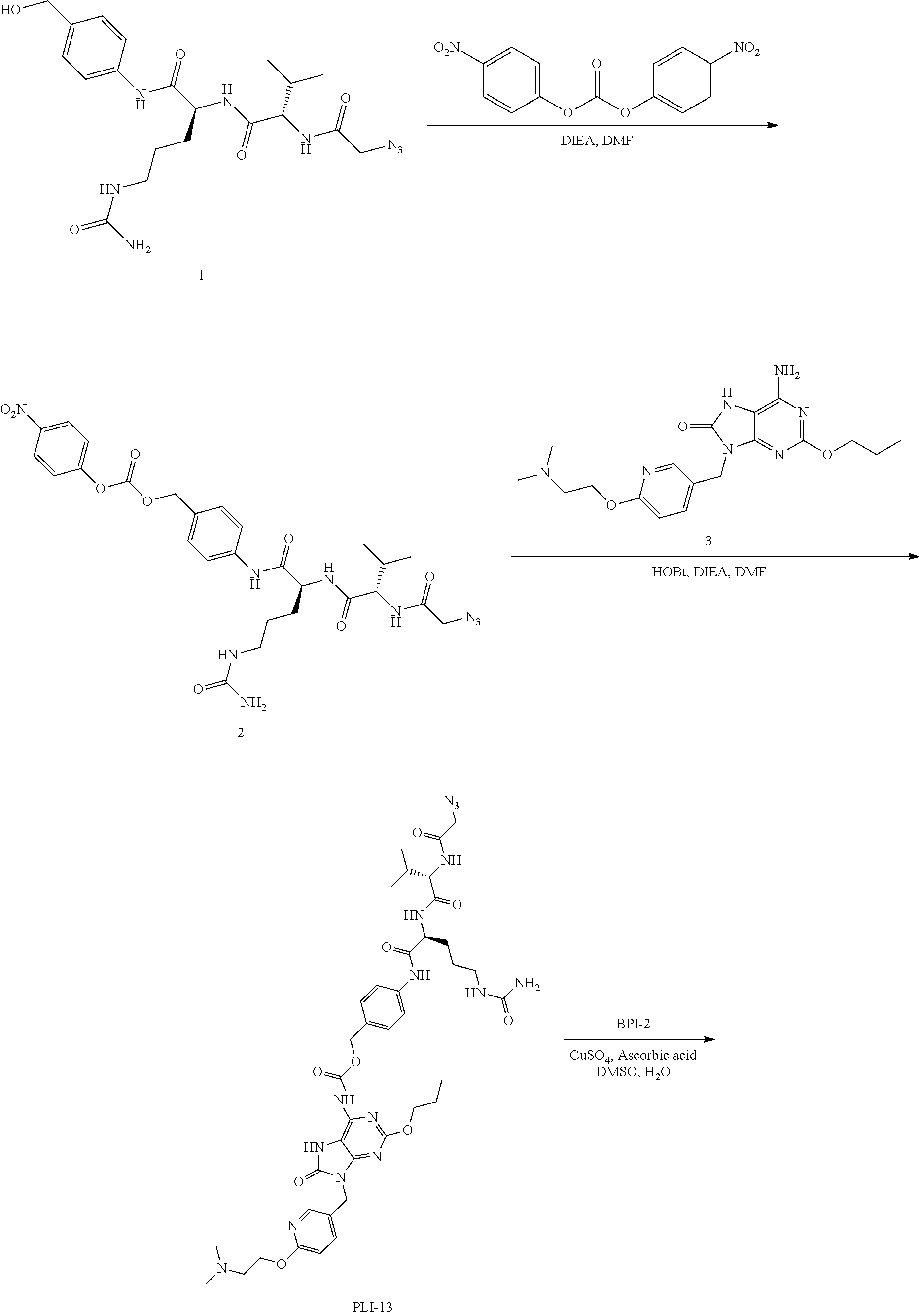 [SMILES: CC(C)[C@H](NC(=O)CN=[N+]=[N-])C(=O)N[C@@H](CCCNC(N)=O)C(=O)Nc1ccc(CO)cc1 ; O=C(Oc1ccc([N+](=O)[O-])cc1)Oc1ccc([N+](=O)[O-])cc1 ; CC(C)[C@H](NC(=O)CN=[N+]=[N-])C(=O)N[C@@H](CCCNC(N)=O)C(=O)Nc1ccc(COC(=O)Oc2ccc([N+](=O)[O-])cc2)cc1 ; CCCOc1nc(N)c2[nH]c(=O)n(Cc3ccc(OCCN(C)C)nc3)c2n1 ; CCCOc1nc(NC(=O)OCc2ccc(NC(=O)[C@H](CCCNC(N)=O)NC(=O)[C@@H](NC(=O)CN=[N+]=[N-])C(C)C)cc2)c2[nH]c(=O)n(Cc3ccc(OCCN(C)C)nc3)c2n1]

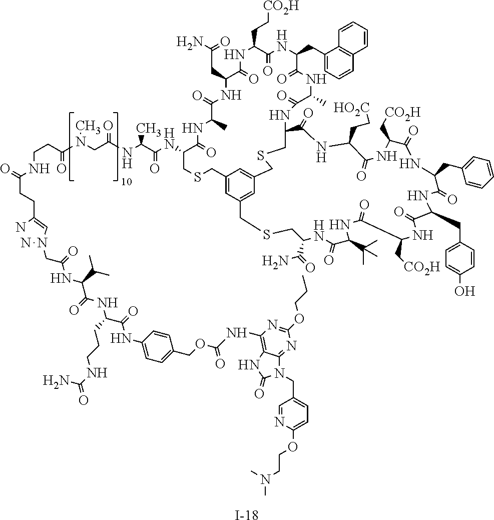

### Procedure for Preparation of Compound 2

To a solution of compound 1 (0.300 g, 648.65 umol, 1.0 eq) in DMF (10 mL) was added DIEA (83.83 mg, 648.65 umol, 112.98 uL, 1.0 eq) and bis (4-nitrophenyl) carbonate (789.30 mg, 2.59 mmol, 4.0 eq). The mixture was stirred at 25° C. for 1 hr. LC-MS showed compound 1 was consumed completely and a peak with desired m/z (628.0 ([M+H]⁺)) was detected. The residue was purified by prep-HPLC (A: H₂O, B: ACN). Compound 2 (0.110 g, 175.27 umol, 27.02% yield) was obtained as a white solid. Calculated MW: 627.6 observed m/z: 628.0 ([M+H]⁺)

### Procedure for Preparation of Compound PLI-13

To a solution of compound 3 (0.040 g, 103.24 umol, 1.0 eq), compound 2 (97.19 mg, 154.86 umol, 1.5 eq) in DMF (5 mL) was added HOBt (20.93 mg, 154.86 umol, 1.5 eq) and DIEA (40.03 mg, 309.73 umol, 53.95 uL, 3.0 eq). The mixture was stirred at 25° C. for 16 hr. LC-MS showed 20% of compound 2 remained and a peak with desired m/z (438.8 ([M/2+H]⁺) was detected. The residue was purified by prep-HPLC (A: 0.075% NH₄OAc in H2O, B: ACN). Compound PLI-13 (0.050 g, 57.08 umol, 55.29% yield) was obtained as a light yellow solid. Calculated MW: 875.9 observed m/z: 438.8 ([M/2+H]⁺

### Procedure for Preparation of I-18

To a solution of compound PLI-13 (0.050 g, 57.08 umol, 1.0 eq) in DMSO (1 mL) was added CuSO4 (0.8 M, 214.06 uL, 3.0 eq), BPI-2 (187.61 mg, 68.50 umol, 1.2 eq) and Ascorbic acid (0.8 M, 713.53 uL, 10.0 eq). The mixture was stirred at 25° C. for 15 min. LC-MS showed compound PLI-13 was consumed completely and a peak with desired m/z (1205.2 ([M/3+H]⁺)) was detected. The residue was purified by prep-HPLC (A: 0.075% TFA in H2O, B: ACN) and then prep-HPLC (A: 0.075% NH₄OAc in H2O, B: ACN). I-18 (0.0061 g, 1.6 umol, 88.1% Purity) was obtained as a white solid. Calculated MW: 3614.9 observed m/z: 1205.2 ([M/3+H]⁺)

Compound 3 above was prepared as follows.

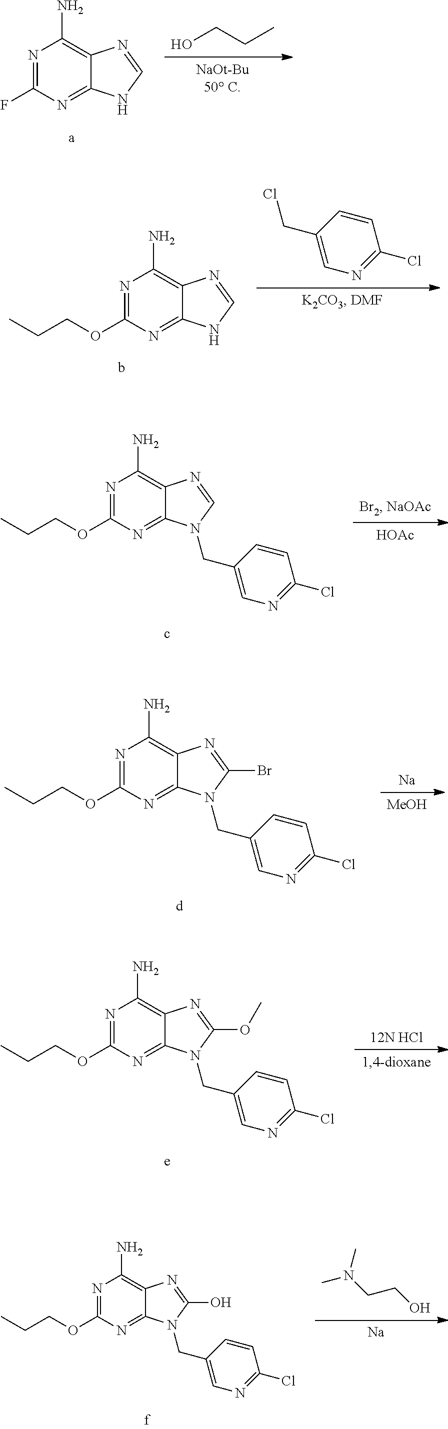

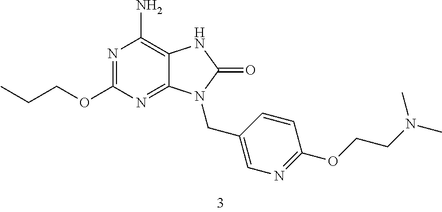 [SMILES: CCCOc1nc(N)c2[nH]c(=O)n(Cc3ccc(OCCN(C)C)nc3)c2n1]

### General Procedure for Preparation of Compound b

t-BuONa (15.69 g, 163.27 mmol, 5 eq) was added gradually to a stirred mixture of propan-1-ol (19.62 g, 326.55 mmol, 24.53 mL, 10 eq) and DME (50 mL). The resulting mixture was heated to 50° C. under an atmosphere of nitrogen and then 2-fluoro-9H-purin-6-amine (5 g, 32.65 mmol, 1 eq) was added. The mixture was maintained at 50° C. for 12 hours. LC-MS showed 2-fluoro-9H-purin-6-amine was consumed completely and one main peak with desired mass was detected. The reaction mixture was quenched by addition of H₂O (80 mL). The reaction mixture was filtered and the filter cake was dried to give Compound b (3.45 g, 17.86 mmol, 54.68% yield) as a yellow solid without further purification.

¹H NMR: (400 MHz DMSO-d6)

δ 7.88 (s, 1H), 7.06 (br s, 2H), 4.12 (t, J=6.6 Hz, 2H), 1.76-1.61 (m, 2H), 0.94 (t, J=7.4 Hz, 3H)

### General Procedure for Preparation of Compound c

To a solution of compound b (3.4 g, 17.60 mmol, 1 eq) in DMF (35 mL) was added K₂CO₃ (2.92 g, 21.12 mmol, 1.2 eq) and 2-chloro-5-(chloromethyl)pyridine (3.42 g, 21.12 mmol, 1.2 eq). The mixture was stirred at 80° C. for 2.5 hr. LC-MS showed compound b was consumed completely and one main peak with desired mass was detected. The reaction solution was concentrated under reduced pressure, water (100 mL) was added, and the resultant was neutralized with 1N hydrochloric acid (20 mL). The precipitated solid was collected by filtration to give the crude compound c (5.69 g, crude) as a yellow solid, which was used into the next step without further purification.

¹H NMR: (400 MHz, DMSO-d6)

δ 8.47 (br d, J=1.6 Hz, 1H), 8.07 (s, 1H), 7.80 (dd, J=2.0, 8.2 Hz, 1H), 7.49 (br d, J=8.3 Hz, 1H), 7.23 (br s, 2H), 5.31 (s, 2H), 4.14 (t, J=6.6 Hz, 2H), 1.75-1.59 (m, 2H), 0.93 (t, J=7.4 Hz, 3H).

### General Procedure for Preparation of Compound d

To a solution of compound c (5.59 g, 17.54 mmol, 1 eq) in HOAc (100 mL) was added NaOAc (5.75 g, 70.15 mmol, 4 eq) and molecular bromine (11.21 g, 70.15 mmol, 3.62 mL, 4 eq). The mixture was stirred at 25° C. for 1 hr. LC-MS showed compound c was consumed completely and one main peak with desired mass was detected. The reaction solution was evaporated under reduced pressure, water (200 mL) was added to the residue, and the resultant was neutralized with 5N sodium hydroxide under ice cooling. The precipitated crystal was collected by filtration and dried under reduced pressure to give the crude compound d (7.84 g, crude) as a yellow solid, which was used into the next step without further purification.

¹H NMR: (400 MHz, DMSO-d6)

δ=8.44 (br d, J=2.0 Hz, 1H), 7.74 (dd, J=2.2, 8.2 Hz, 1H), 7.52 (d, J=8.4 Hz, 1H), 5.34 (s, 2H), 4.25 (br t, J=6.5 Hz, 2H), 1.71 (sxt, J=7.1 Hz, 2H), 0.95 (t, J=7.4 Hz, 3H).

### General Procedure for Preparation of Compound e

To a solution of compound d (7.74 g, 19.46 mmol, 1 eq) in MeOH (100 mL) was added Na (2.24 g, 97.32 mmol, 5 eq), and the mixture was heated to 80° C. and stirred for 2 hours. LC-MS showed compound d was consumed completely and one main peak with desired mass was detected. The reaction solution was concentrated under reduced pressure to give a residue, which was poured into ice-water (100 mL), and the resultant was neutralized with concentrated hydrochloric acid under ice cooling until pH=8-9. The precipitated solid was collected by filtration and washed with water (20 mL). The solid was dried to give the crude compound e (4.8 g, crude) as a yellow gum, which was used into the next step without further purification. MS (ESI) m/z: calcd. for [M+H]⁺, 349.11; found, 349.1 (M/1+H)⁺

### General Procedure for Preparation of Compound f

A solution of compound e (4 g, 11.47 mmol, 1 eq) in 12 N HCl (45 mL) and dioxane (10 mL) was stirred at 100° C. for 1 hr. LC-MS showed compound e was consumed completely and one main peak with desired mass was detected. The reaction solution was concentrated under reduced pressure, water (100 mL) was added to the residue, and the resultant was neutralized with an aqueous solution of 5N sodium hydroxide under ice. The precipitated crystal was collected by filtration. The filtered cake was dried to give crude compound f (3 g, crude) as a white solid, which was used into the next step without further purification.

¹H NMR: (400 MHz, DMSO-d6)

δ 10.07 (br s, 1H), 8.40 (br s, 1H), 7.76 (br d, J=7.9 Hz, 1H), 7.48 (br d, J=8.1 Hz, 1H), 6.51 (br s, 2H), 4.90 (s, 2H), 4.09 (br t, J=6.5 Hz, 2H), 1.74-1.54 (m, 2H), 0.92 (br t, J=7.2 Hz, 2H), 0.99-0.82 (m, 1H).

### General Procedure for Preparation of Compound 3

To a solution of compound f (1.4 g, 4.18 mmol, 1 eq) in 2-(dimethylamino)ethanol (31.08 g, 348.68 mmol, 35.00 mL, 83 eq) was added Na (577 mg, 25 mmol, 6 eq) and the mixture was heated at 120° C. for 3 hours. LC-MS showed compound f was consumed completely and one main peak with desired mass was detected. The mixture was neutralized with 12N hydrochloric acid until pH=7 and extracted with ethyl acetate (60 mL*3). The combined organic layers were washed with brine (25 mL), dried over Na₂SO₄, filtered and concentrated under reduced pressure to give a residue. The residue was purified by prep-HPLC (neutral condition; Phenomenex Gemini C18 250*50 10u column; 12-42% acetonitrile in 10 mM NH₄HCO₃-ACN, 21 min gradient) to give compound 3 (406 mg, 1.03 mmol, 24.56% yield, 98.01% purity) as a white solid. MS (ESI) m/z: calcd. for [M+H]⁺, 388.20; found, 388.2 (M/1+H)⁺

¹H NMR: (400 MHz, DMSO-d6)

δ 9.96 (br s, 1H), 8.13 (s, 1H), 7.64 (br d, J=8.6 Hz, 1H), 6.76 (d, J=8.6 Hz, 1H), 6.46 (br s, 2H), 4.80 (s, 2H), 4.29 (t, J=5.8 Hz, 2H), 4.10 (t, J=6.6 Hz, 2H), 2.57 (t, J=5.8 Hz, 2H), 2.24-2.08 (m, 6H), 1.68-1.65 (m, 2H), 0.93 (t, J=7.4 Hz, 3H)

### Example 18: Synthesis of Compound I-26

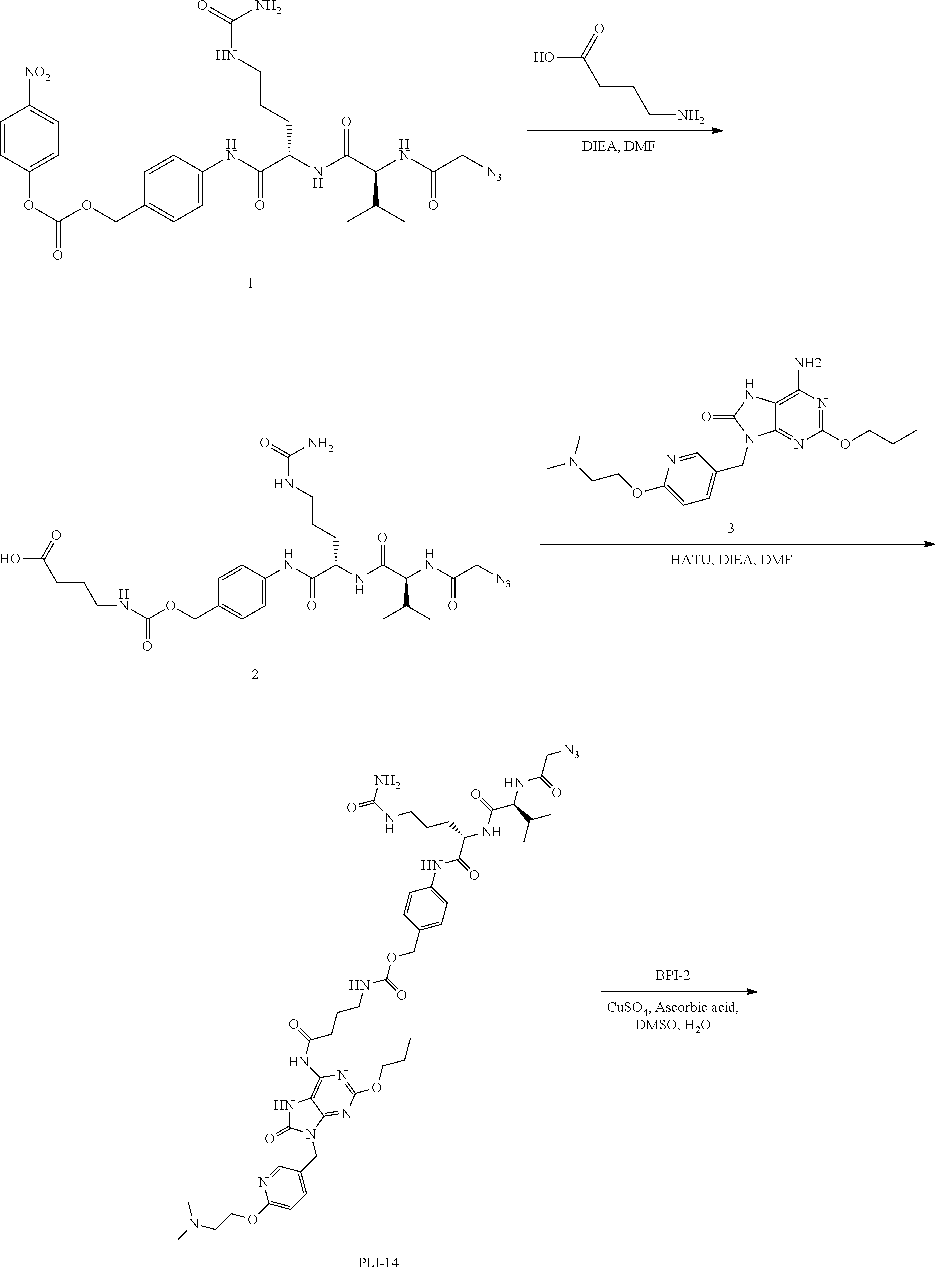 [SMILES: CC(C)[C@H](NC(=O)CN=[N+]=[N-])C(=O)N[C@@H](CCCNC(N)=O)C(=O)Nc1ccc(COC(=O)Oc2ccc([N+](=O)[O-])cc2)cc1 ; NCCCC(=O)O ; CC(C)[C@H](NC(=O)CN=[N+]=[N-])C(=O)N[C@@H](CCCNC(N)=O)C(=O)Nc1ccc(COC(=O)NCCCC(=O)O)cc1 ; CCCOc1nc(N)c2[nH]c(=O)n(Cc3ccc(OCCN(C)C)nc3)c2n1 ; CCCOc1nc(NC(=O)CCCNC(=O)OCc2ccc(NC(=O)[C@H](CCCNC(N)=O)NC(=O)[C@@H](NC(=O)CN=[N+]=[N-])C(C)C)cc2)c2[nH]c(=O)n(Cc3ccc(OCCN(C)C)nc3)c2n1]

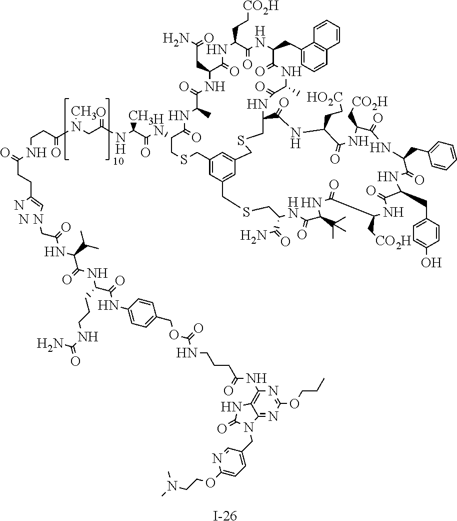

### Procedure for Preparation of Compound 2

To a solution of compound 1 (20.0 mg, 319 umol) in DMF (5 mL) was added DIEA (124 mg, 956 umol, 167 uL) and 4-aminobutanoic acid (49.3 mg, 478 umol). The mixture was stirred at 15° C. for 1 hr. LC-MS showed compound 1 was consumed completely and a peak with desired m/z (m/z: 592.1 ([M+H⁺])) was detected. The reaction mixture was filtered and concentrated under reduced pressure to give a residue. The residue was purified by prep-HPLC (A: H2O, B: ACN) to give compound 2 (13.0 mg, 219.74 umol, 68.95% yield) as a white solid. Calculated MW: 591.6 observed m/z: 592.1 ([M+H⁺])

### Procedure for Preparation of Compound PLI-14

To a solution of compound 2 (30.0 mg, 50.7 umol) in DMF (3 mL) was added HATU (19.3 mg, 50.7 umol), Compound 3 (synthesis described in Example 17) (14.7 mg, 38.0 umol) and DIEA (32.8 mg, 254 umol, 44.2 uL). The mixture was stirred at 15° C. for 2 hr. LC-MS showed compound 2 was consumed completely and a peak with desired mass (m/z: 481.6 (M/2+H⁺)) was detected. The reaction mixture was filtered and concentrated under reduced pressure to give a residue. The residue was purified by prep-HPLC (A: H2O, B: ACN) to give compound PLI-14 (14.0 g, 14.6 umol, 57.5% yield) as a white solid. Calculated MW: 961.0 observed m/z: 481.6 ([M/2+H⁺])

### Procedure for Preparation of I-26

To a solution of compound PLI-14 (12.0 mg, 12.5 umol) in DMSO (5 mL) was added CuSO4 (0.4 M, 93.7 uL), BPI-2 (4.2 mg, 12.5 umol, 1 eq) and ascorbic acid (0.4 M, 312 uL). The mixture was stirred at 15° C. for 1 hr. LC-MS showed compound PLI-14 was consumed completely and one main peak with desired m/z (calculated MW: 3700.06 observed m/z: 1233.8 ([M/3+H⁺], 925.5 [M/4+H]) was detected. The reaction mixture was filtered and concentrated under reduced pressure to give a residue. The residue was purified by prep-HPLC (A: 0.075% NH₄OAc in H2O, B: ACN) to give I-26 (19.1 mg, 4.85 umol, 38.9% yield, 94.0% purity) as a white solid. Calculated MW: 3700.06 observed m/z: 1233.9 ([M/3+H⁺], 925.5 [M/4+H⁺])

### Example 19. Synthesis of Compound I-23

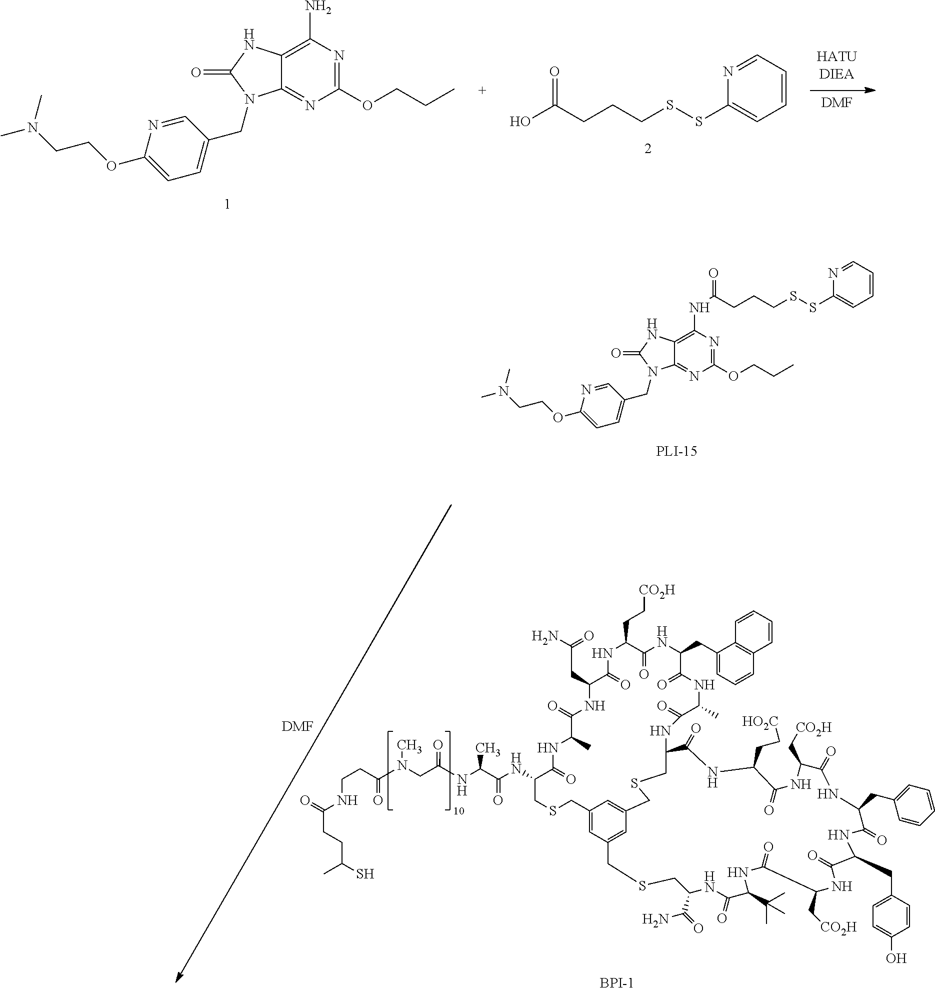

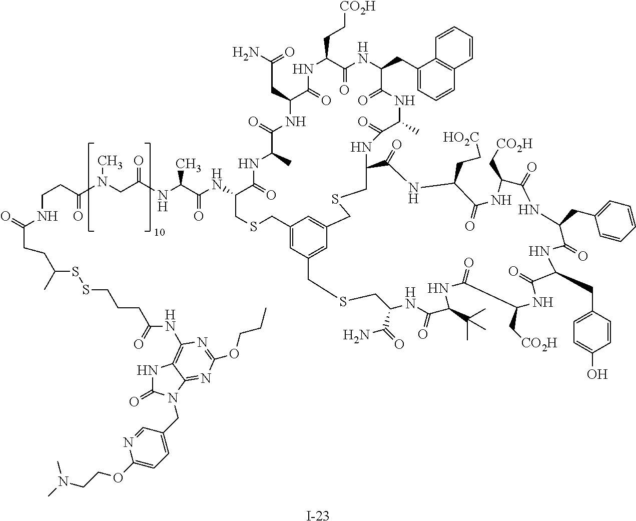

### Procedure for Preparation of Compound PLI-15

To a solution of compound 1 (0.020 g, 51.62 umol) in DMF (2 mL) was added HATU (29.44 mg, 77.43 umol), compound 2 (23.68 mg, 103.24 umol) and DIEA (20.02 mg, 154.86 umol, 26.97 uL). The mixture was stirred at 25° C. for 2 hrs. TLC (DCM/MeOH=10:1) showed compound 1 was consumed. The reaction mixture was filtered and concentrated under reduced pressure to give a residue. The residue was purified by prep-HPLC (A: H2O, B: ACN)). Compound PLI-15 (0.004 g, 6.68 umol, 12.94% yield) was obtained as a white solid. Calculated MW: 598.7

### Procedure for Preparation of I-23

To a solution of compound PLI-15 (0.005 g, 8.35 umol) in DMF (1 mL) was added compound BPI-1 (30.13 mg, 10.86 umol). The mixture was stirred at 20° C. for 1 hr. LC-MS showed compound PLI-15 was consumed completely and a peak with desired m/z (1087.9 ([M/3+H]⁺)) was detected. The mixture was filtered and concentrated under reduced pressure to give a residue. The residue was purified by prep-HPLC (A: 0.075% TFA in H2O, B: ACN)). Compound I-23 (0.0141 g, 48.52% yield, 93.76% purity) was obtained as a white solid. Calculated MW: 3262.6, observed MS: 1088.0 ([M/3+H]⁺).

### Example 20. Synthesis of Compound I-2

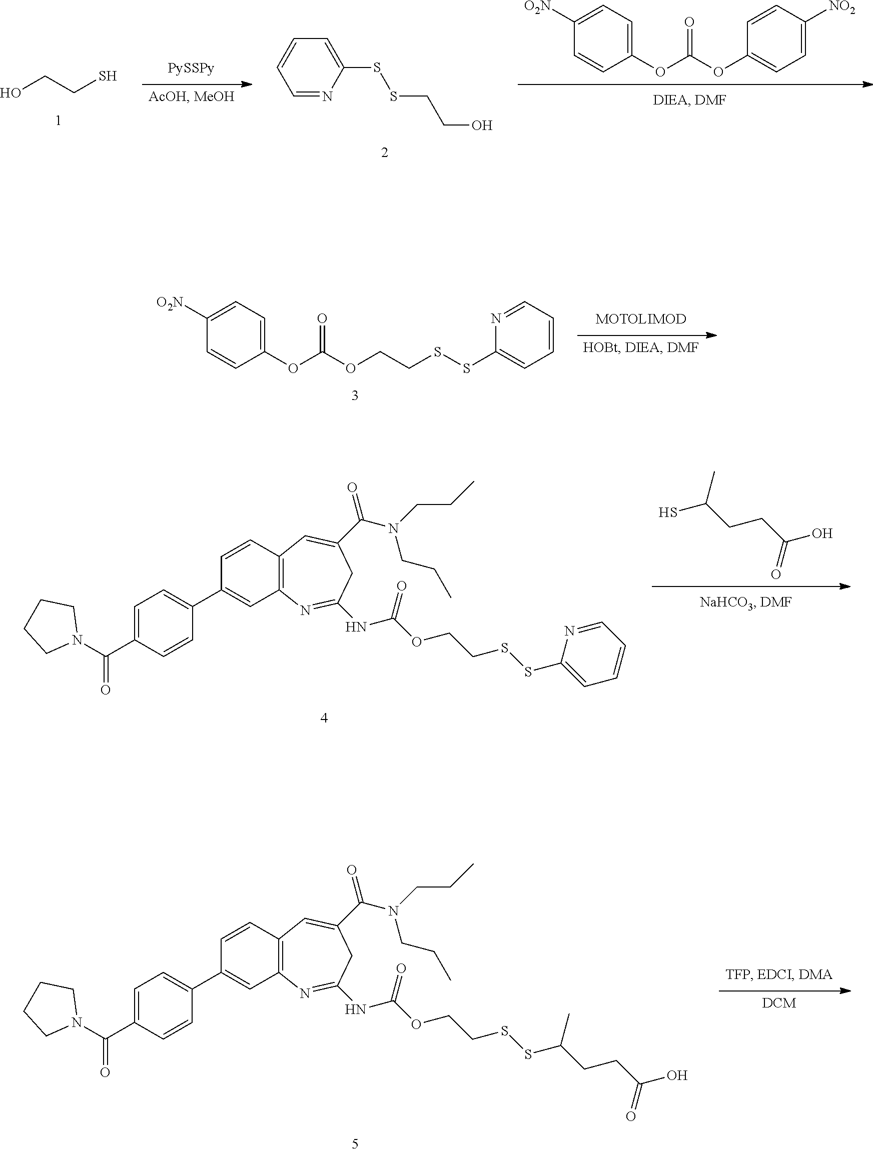

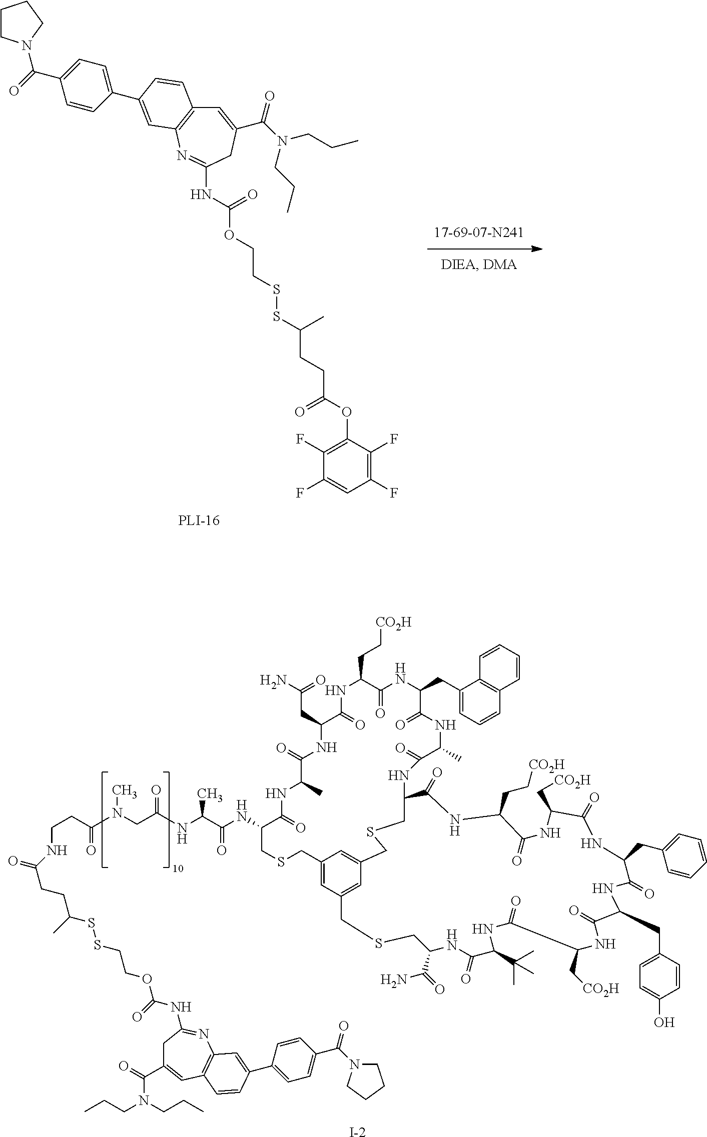

### Procedure for Preparation of Compound 2

To a solution of compound 1 (3 g, 38.40 mmol, 2.68 mL), 2-(2-pyridyldisulfanyl) pyridine (12.69 g, 57.59 mmol) in MeOH (100 mL) was added AcOH (5.76 g, 95.99 mmol, 5.49 mL). The mixture was stirred at 25° C. for 16 hr under N2 atmosphere. LC-MS indicated compound 1 was consumed completely and a peak with desired mass (m/z: 188.1 ([M+H⁺])) was detected. The reaction mixture was concentrated under reduced pressure to remove MeOH to give a residue. The residue was purified by flash silica gel chromatography (ISCO®; 120 g SepaFlash® Silica Flash Column, Eluent of 0-40% Ethylacetate/Petroleum ether gradient@80 mL/min). Compound 2 (5.5 g, 29.37 mmol, 76.49% yield) was obtained as a yellow oil. Calculated MW: 187.2 observed m/z: 188.1 ([M+H⁺])

### Procedure for Preparation of Compound 3

To a solution of compound 2 (2 g, 10.68 mmol) in THF (10 mL) was added DIEA (4.14 g, 32.04 mmol, 5.58 mL) and bis(4-nitrophenyl) carbonate (6.50 g, 21.36 mmol). The mixture was stirred at 25° C. for 16 hr. LC-MS showed compound 2 was consumed completely and a peak with desired mass (m/z: 353.1 ([M+H⁺])) was detected. The reaction mixture was concentrated under reduced pressure to remove THF to give a residue and the residue was purified by flash silica gel chromatography (ISCO®; 80 g SepaFlash® Silica Flash Column, Eluent of 0-50% Ethylacetate/Petroleum ether gradient@60 mL/min). Compound 2 (4.2 g, crude) was obtained as a yellow solid with traces of bis (4-nitrophenyl) carbonate in the product. Calculated MW: 352.3 observed m/z: 353.1 ([M+H⁺])

### Procedure for Preparation of Compound 4

To a solution of compound 3 (230.52 mg, 654.17 umol) in DMF (2 mL) was added Motolimod (0.15 g, 327.09 umol), DIEA (126.82 mg, 981.26 umol, 170.92 uL) and HOBt (53.04 mg, 392.50 umol). The mixture was stirred at 25° C. for 16 hr. LC-MS indicated compound 3 was consumed completely and a peak with desired mass (m/z: 672.1 ([M+H⁺])) was detected. The reaction mixture was purified by prep-HPLC (A: H2O, B: ACN). Compound 4 (0.11 g, 163.72 umol, 50.05% yield) was obtained as a white solid. Calculated MW: 671.8 observed m/z: 672.1 ([M+H⁺])

### Procedure for Preparation of Compound 5

To a solution of compound 4 (0.1 g, 148.84 umol), was added 4-sulfanylpentanoic acid (29.96 mg, 223.26 umol, 1.5 eq) in DMF (4 mL) and pH was adjusted to 8 using NaHCO₃ solution. The mixture was stirred at 25° C. for 0.5 hr. LC-MS showed compound 4 was consumed completely and a peak with desired MS (m/z: 695.2 ([M+H⁺])) was detected. The reaction mixture was purified by prep-HPLC (A: H2O, B: ACN) to give Compound 5 (0.08 g, 115.12 umol, 77.35% yield) as a white solid. Calculated MW: 694.9 observed m/z: 695.2 ([M+H⁺]).

### Procedure for Preparation of Compound PLI-16

To a solution of compound 5 (0.08 g, 115.12 umol), 2,3,5,6-tetrafluorophenol (57.36 mg, 345.37 umol) in DMA (3 mL) and DCM (1 mL) was added EDCI (66.21 mg, 345.37 umol). The mixture was stirred at 25° C. for 4 hr. LC-MS showed compound 5 was consumed completely and a peak with desired mass (m/z: 843.1 ([M+H⁺])) was detected. The reaction mixture was directly purified by prep-HPLC (A: H2O, B: ACN) to give Compound PLI-16 (0.06 g, 64.06 umol, 55.64% yield) as a yellow solid. (Calculated MW: 842.9 observed m/z: 843.1 ([M+H⁺]))

### Procedure for Preparation of I-2

To a solution of compound PLI-16 (20 mg, 23.73 umol) in DMA was added 17-69-07-N241 (69.39 mg, 26.10 umol) and DIEA (9.20 mg, 71.18 umol, 12.40 uL). The mixture was stirred at 25° C. for 16 hr. LC-MS showed compound PLI-16 was consumed completely and one main peak with desired MS (calculated MW: 3335.8 observed m/z: 1112.9 ([M/3+H⁺])) was detected. The reaction mixture was directly purified by prep-HPLC (A: H2O, B: ACN) to give I-2 (MOT2, 20.1 mg, 5.54 umol, 23.36% yield, 92.1% purity) as a white solid. Calculated MW: 3335.8 observed m/z: 1112.9 ([M/3+H⁺])

### Example 21. Synthesis of Compound I-24

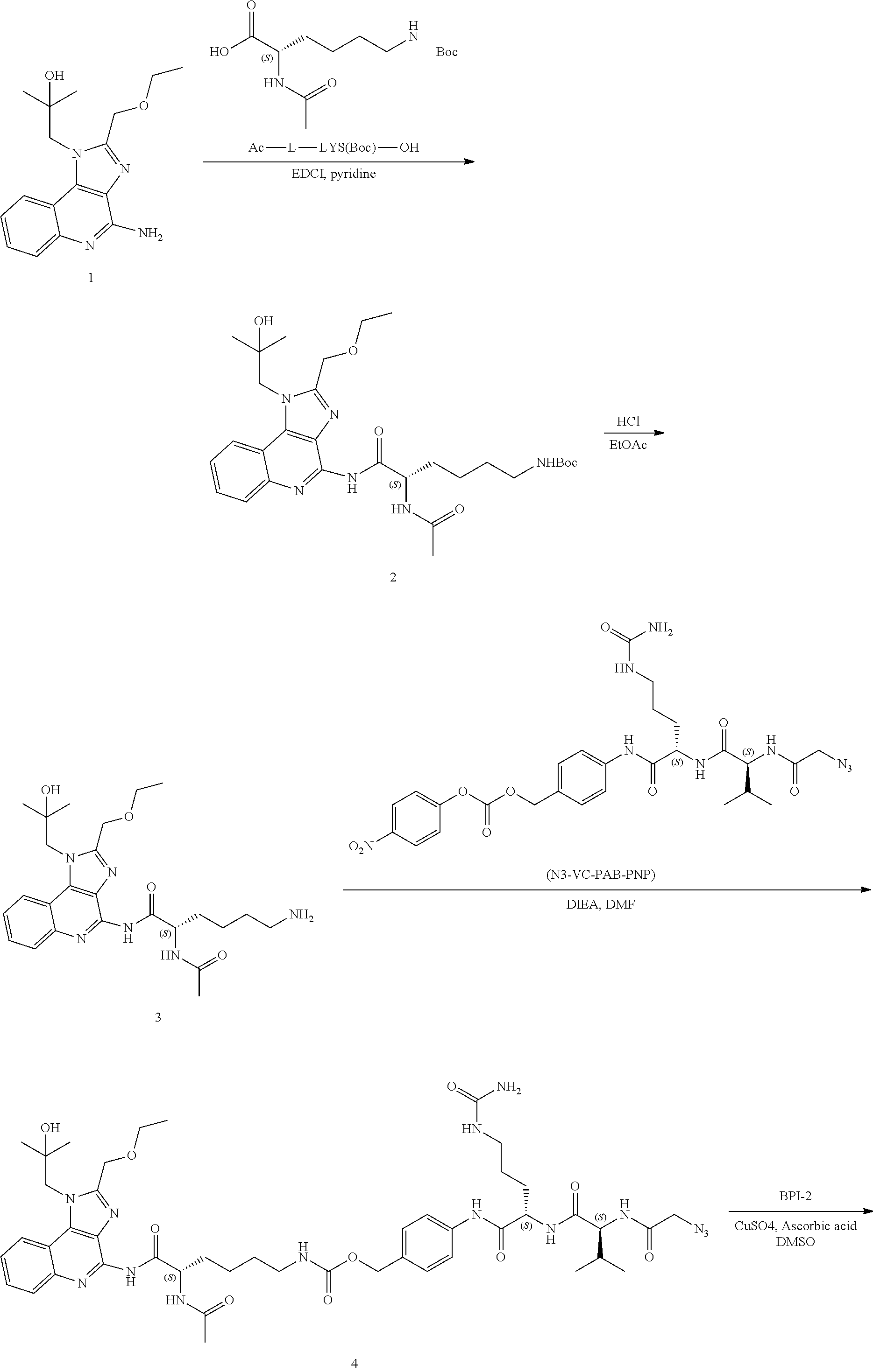

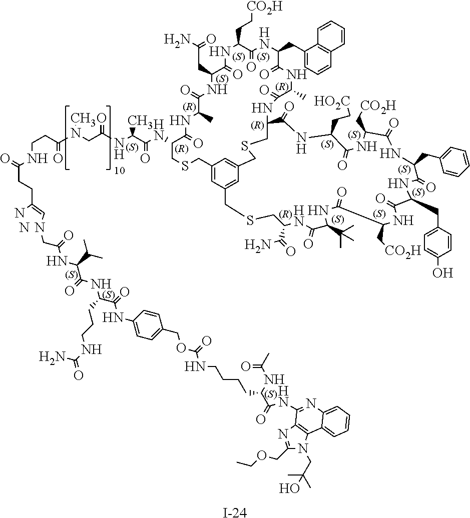

### Procedure for Preparation of Compound 2

To a solution of compound 1 (0.100 g, 318.09 umol, 1 eq) in pyridine (1 mL) was added EDCI (152.44 mg, 795.21 umol, 2.5 eq) and Ac-L-Lys(Boc)-OH (119.23 mg, 413.51 umol, 1.3 eq). The mixture was stirred at 25° C. for 1 hr. LC-MS showed 50% of Compound 1 remained and a peak with desired m/z (m/z: 586.1 ([M+H]⁺)) was detected. The residue was purified by prep-HPLC (A: H₂O, B: ACN). Compound 2 (0.120 g, 205.23 umol, 64.52% yield) was obtained as a white solid. Calculated MW: 584.7 observed m/z: 586.1 ([M+H]⁺)

### Procedure for Preparation of Compound 3

To a solution of compound 2 (0.090 g, 153.92 umol, 1 eq) was added 4 M HCl in EtOAc (3 mL). The mixture was stirred at 25° C. for 1 hr. LC-MS showed Compound 2 was consumed completely and a peak with desired m/z (m/z: 485.0 ([M+H]⁺)) was detected. The reaction mixture was filtered and concentrated under reduced pressure to give a residue. The residue was purified by prep-HPLC (A: 0.075% TFA in H₂O, B: ACN). Compound 3 (0.070 g, 144.45 umol, 93.85% yield) was obtained as a white solid. Calculated MW: 484.5 observed m/z: 485.0 ([M+H]⁺)

### Procedure for Preparation of Compound PLI-17

To a solution of compound 3 (0.050 g, 103.18 umol, 1 eq) in DMF (2 mL) was added DIEA (13.34 mg, 103.18 umol, 17.97 uL, 1 eq) and N3-VC-PAB-PNP (64.76 mg, 103.18 umol, 1 eq). The mixture was stirred at 25° C. for 2 hr. LC-MS showed compound 3 was consumed completely and a peak with desired m/z (m/z: 973.6 ([M+H]⁺)) was detected. The reaction mixture was filtered and concentrated under reduced pressure to give a residue. The residue was purified by prep-HPLC (A: H₂O, B: ACN). Compound PLI-17 (0.013 g, 13.36 umol, 12.95% yield) was obtained as a white solid. Calculated MW: 973.0 observed m/z: 973.6 ([M+H]⁺)

### Procedure for Preparation of I-24

To a solution of compound PLI-17 (0.012 g, 12.33 umol, 1 eq) in DMSO (4 mL) was added CuSO4 (0.8 M, 46.24 uL, 3 eq), BPI-2 (0.8 M, 154.15 uL, 10 eq) and ascorbic acid (21.7 mg, 123.3 umol, 10 equiv). The mixture was stirred at 15° C. for 15 min. LC-MS showed Compound PLI-17 was consumed completely and one main peak with desired m/z (calculated MW: 3712.1 observed m/z: 1237.9 ([M/3+H]⁺)) was detected. Filtered and concentrated under reduced pressure to give a residue. The residue was purified by prep-HPLC (A: 0.075% TFA in H₂O, B: ACN). I-24 (0.0119 g, 3.05 umol, 24.75% yield, 95.2% purity) was obtained as a white solid. Calculated MW: 3712.1 observed m/z: 1237.9 ([M/3+H]⁺)

### Example 22. Synthesis of Compound I-22

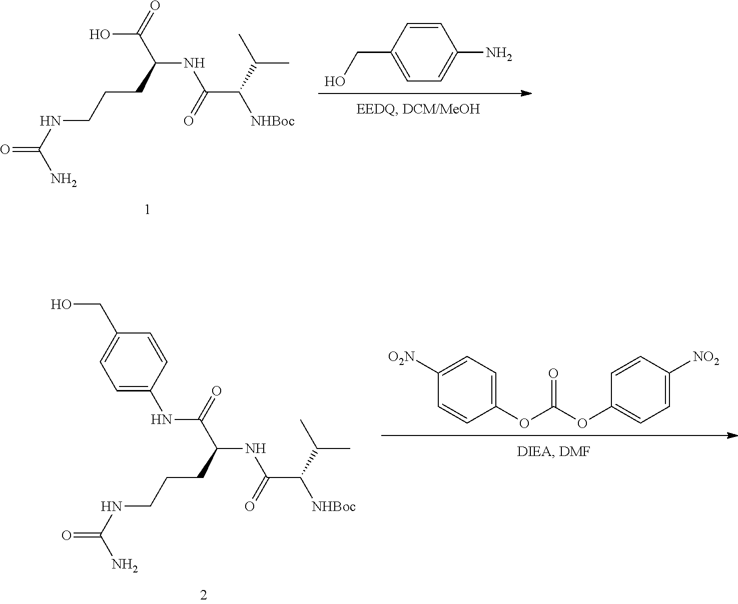 [SMILES: CC(C)[C@H](NC(=O)OC(C)(C)C)C(=O)N[C@@H](CCCNC(N)=O)C(=O)O ; Nc1ccc(CO)cc1 ; CC(C)[C@H](NC(=O)OC(C)(C)C)C(=O)N[C@@H](CCCNC(N)=O)C(=O)Nc1ccc(CO)cc1 ; O=C(Oc1ccc([N+](=O)[O-])cc1)Oc1ccc([N+](=O)[O-])cc1]

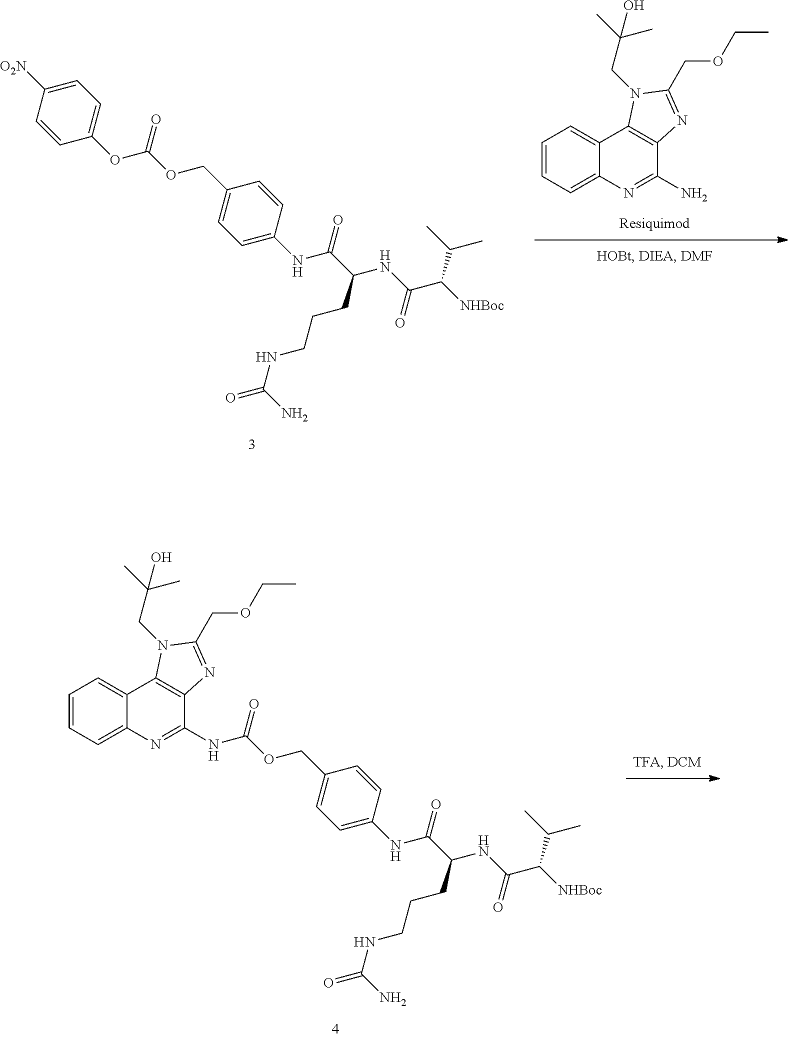 [SMILES: CC(C)[C@H](NC(=O)OC(C)(C)C)C(=O)N[C@@H](CCCNC(N)=O)C(=O)Nc1ccc(COC(=O)Oc2ccc([N+](=O)[O-])cc2)cc1 ; CCOCc1nc2c(N)nc3ccccc3c2n1CC(C)(C)O ; CCOCc1nc2c(NC(=O)OCc3ccc(NC(=O)[C@H](CCCNC(N)=O)NC(=O)[C@@H](NC(=O)OC(C)(C)C)C(C)C)cc3)nc3ccccc3c2n1CC(C)(C)O]

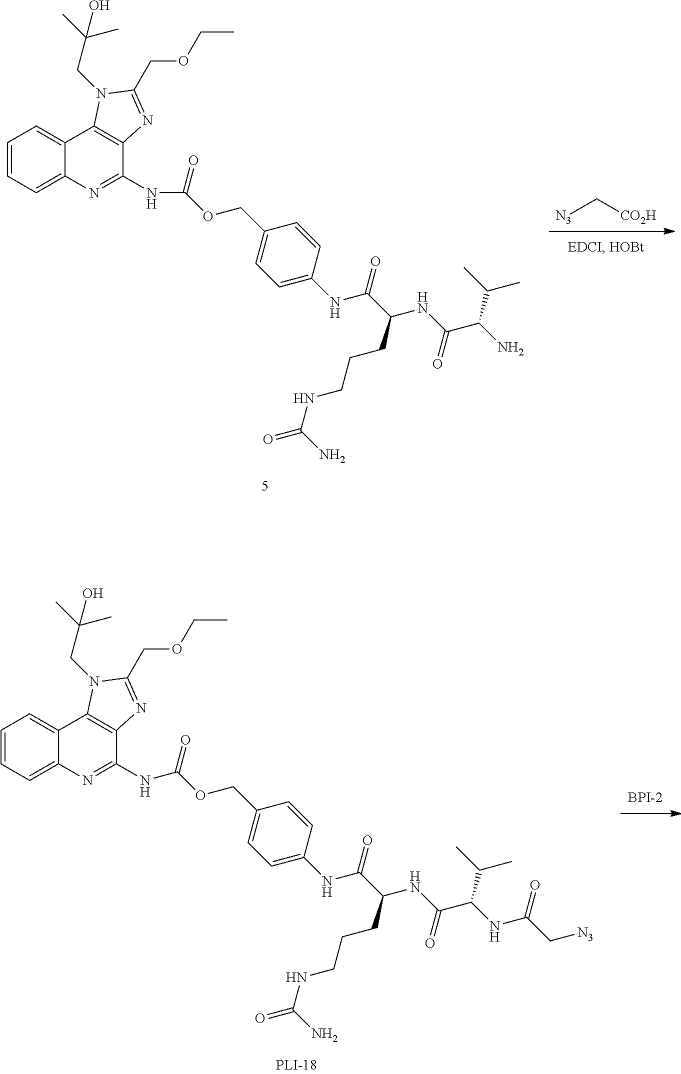 [SMILES: CCOCc1nc2c(NC(=O)OCc3ccc(NC(=O)[C@H](CCCNC(N)=O)NC(=O)[C@@H](N)C(C)C)cc3)nc3ccccc3c2n1CC(C)(C)O ; [N-]=[N+]=NCC(=O)O ; CCOCc1nc2c(NC(=O)OCc3ccc(NC(=O)[C@H](CCCNC(N)=O)NC(=O)[C@@H](NC(=O)CN=[N+]=[N-])C(C)C)cc3)nc3ccccc3c2n1CC(C)(C)O]

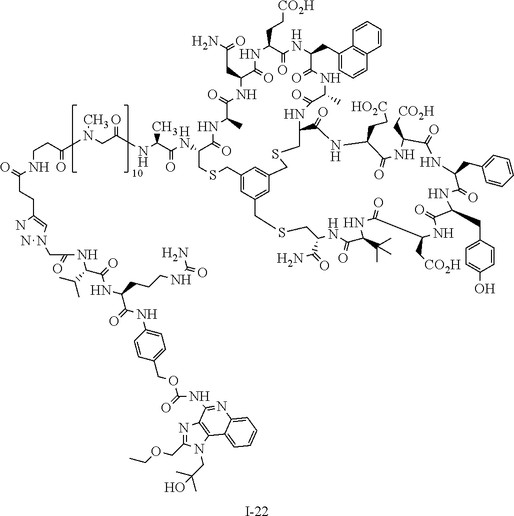

### General Procedure for Preparation of Compound 2

To a solution of compound 1 (10.0 g, 26.71 mmol, 1 eq) in DCM (40 mL) and MeOH (20 mL) was added EEDQ (13.2 g, 53.41 mmol, 2 eq) and (4-aminophenyl)methanol (3.95 g, 32.05 mmol, 1.2 eq). The mixture was stirred at 25° C. for 12 hr. LC-MS showed compound 1 was consumed completely and a peak with desired mass was detected (m/z: 480.1 ([M+H]⁺)). The reaction mixture was concentrated under reduced pressure to remove solvent to give a residue. The residue was purified by flash silica gel chromatography (ISCO®; 330 g SepaFlash® Silica Flash Column, Eluent of 0-15% Ethylacetate/Petroleum ether gradient@100 mL/min). Compound 2 (7.4 g, 15.43 mmol, 57.8% yield) was obtained as a white solid. Calculated MW: 480.27; observed m/z: 480.1 ([M+H]⁺).

### General Procedure for Preparation of Compound 3

To a solution of compound 2 (3.0 g, 6.26 mmol, 1 eq) in DMF (10 mL) was added DIEA (2.4 g, 18.77 mmol, 3.3 mL, 3 eq) and bis(4-nitrophenyl) carbonate (3.8 g, 12.51 mmol, 2 eq). The mixture was stirred at 25° C. for 1 hr. LC-MS showed compound 2 was consumed completely and a peak with desired mass (m/z: 645.1 ([M/1+H]⁺)) was detected. The reaction was directly purified by prep-HPLC (ACN/H₂O condition). Compound 3 (2.6 g, 4.03 mmol, 64.47% yield) was obtained as a white solid. Calculated MW: 645.28; observed m/z: 645.1 ([M/1+H]⁺).

### General Procedure for Preparation of Compound 4

To a solution of compound 3 (1.33 g, 2.07 mmol, 1.3 eq) in DMF (4 mL) was added HOBt (257.88 mg, 1.91 mmol, 1.2 eq), DIEA (616.65 mg, 4.77 mmol, 831.07 uL, 3 eq) and Resiquimod (0.500 g, 1.59 mmol, 1 eq). The mixture was stirred at 40° C. for 16 hr. LC-MS showed 50% of Resiquimod remained and a peak with the desired mass (m/z: 820.3 ([M/1+H]⁺)) was detected. The reaction was directly purified by prep-HPLC (ACN/H₂O condition). Compound 4 (0.820 g, 1.00 mmol, 62.88% yield) was obtained as a white solid. Calculated MW: 820.43; observed m/z: 820.3 ([M/1+H]⁺).

### General Procedure for Preparation of Compound 5

To a solution of compound 4 (0.820 g, 1.00 mmol, 1 eq) in DCM (9 mL) was added TFA (1.54 g, 13.51 mmol, 1 mL, 13.51 eq). The mixture was stirred at 25° C. for 0.5 hr. LC-MS showed compound 4 was consumed completely and a peak with desired mass was detected (m/z: 720.3 ([M/1+H]⁺)). The reaction mixture was concentrated under reduced pressure to remove solvent to give a residue. The residue was purified by prep-HPLC (ACN/H₂O condition). Compound 5 (0.300 g, 416.77 umol, 41.67% yield) was obtained as a white solid. Calculated MW: 720.38; observed m/z: 720.3 ([M/1+H]⁺).

### General Procedure for Preparation of Compound PLI-18

To a solution of compound 5 (0.300 g, 416.77 umol, 1 eq), 2-azidoacetic acid (84.24 mg, 833.53 umol, 2 eq), HOBt (61.95 mg, 458.44 umol, 1.1 eq), DIEA (107.73 mg, 833.53 umol, 145.19 uL, 2 eq) in DMF (10 mL) was added EDCI (159.79 mg, 833.53 umol, 2 eq). The mixture was stirred at 25° C. for 12 hr. LC-MS showed compound 5 was consumed completely and a peak with desired mass (m/z: 803.3 ([M/1+H]⁺)) was detected. The reaction mixture was directly purified by prep-HPLC (ACN/H₂O condition). Compound PLI-18 (0.105 g, 130.78 umol, 31.38% yield) was obtained as a white solid. Calculated MW: 802.88; observed m/z: 803.3 ([M/1+H]⁺).

### General Procedure for Preparation of I-22

To a solution of compound PLI-18 (0.050 g, 62.28 umol, 1 eq) in DMF (1 mL) was added CuSO4 (0.8 M, 233.54 uL, 3 eq), (2R)-2-[(1S)-1,2-dihydroxyethyl]-3,4-dihydroxy-2H-furan-5-one (0.8 M, 778.45 uL, 10 eq) and BPI-2 (170.00 mg, 981.47 umol, 15.76 eq), The mixture was stirred at 25° C. for 15 min. LC-MS showed compound PLI-18 was consumed completely and one main peak with desired m/z (Calculated MW: 3541.90 observed m/z: 1181.3 ([M/3+H])) was detected. The reaction was directly purified by prep-HPLC (A: H2O, B: ACN) to give I-22 (0.098 g, 27.60 umol) as a white solid. Calculated MW: 3541.90 observed m/z: 1181.2 ([M/3+H⁺])

### Example 23. Synthesis of Bicycle Peptide Intermediates

### 23.1. Synthesis of Compound BPI-1

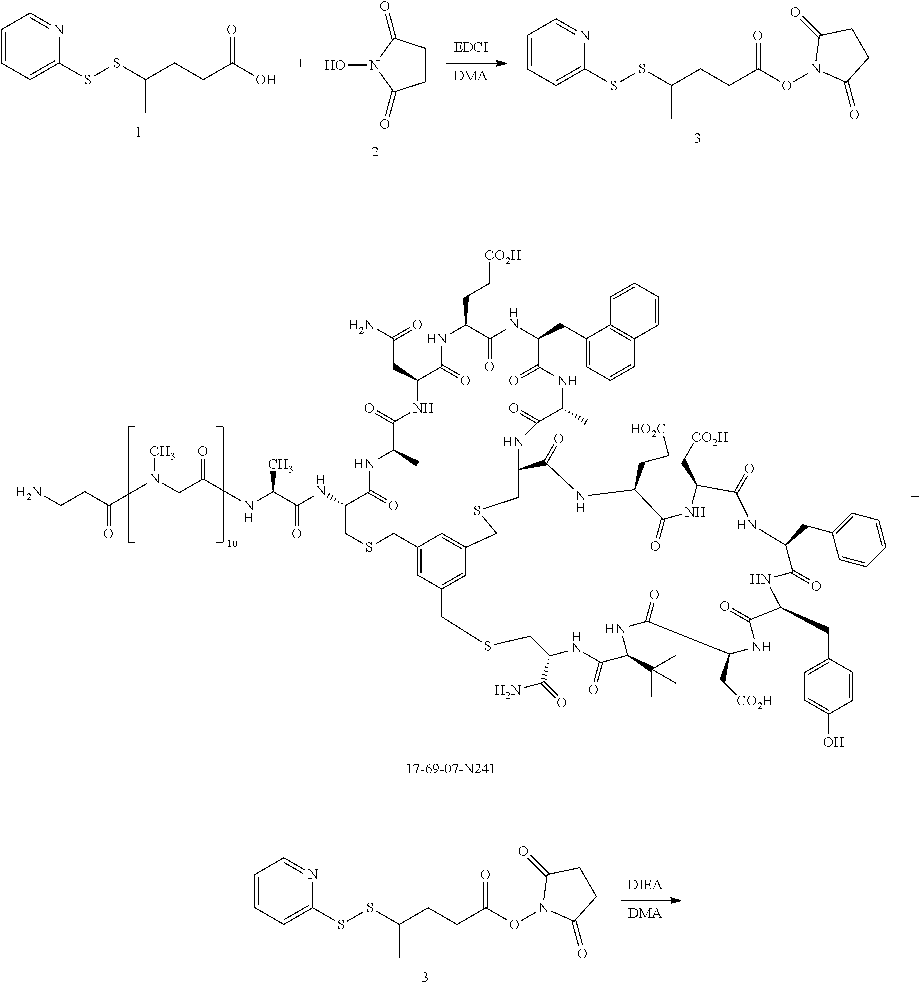

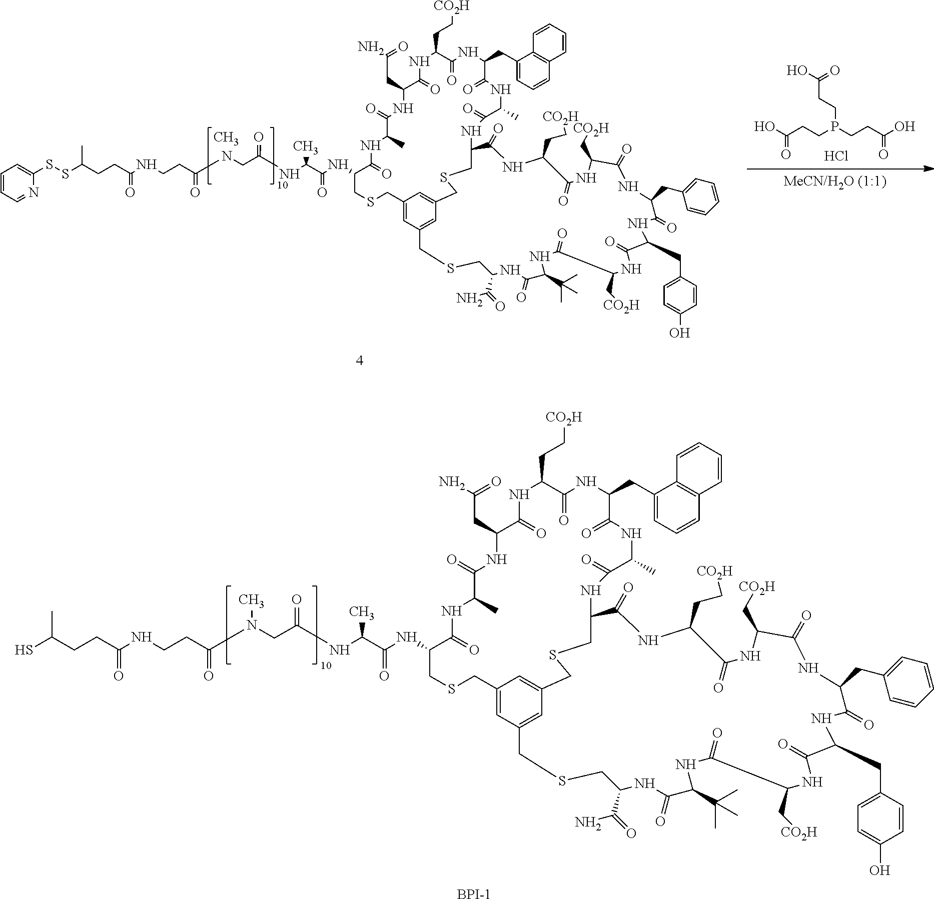

### Procedure for Preparation of Compound 3

To a solution of compound 1 (0.025 g, 102.73 umol, 1 eq.) in DMA (1 mL) was added EDCI (21.66 mg, 113.01 umol, 1.1 eq.) and compound 2 (13.01 mg, 113.01 umol, 1.1 eq.). The reaction mixture was stirred at 25° C. for 1 h. LC-MS showed a peak with desired m/z (341 [M+H⁺]) and the crude product was used directly in the next step without further purification.

### Procedure for Preparation of Compound 4

To a solution of compound 3 (0.025 g, 73.44 umol, 1 eq.) in DMA (1 mL) was added DIEA (28.47 mg, 220.32 umol, 3 eq.) and 17-69-07-N241 (0.195 g, 73.44 umol, 1 eq.). The reaction mixture was stirred at 25° C. for 4 h. LC-MS showed compound 3 was consumed completely and a peak with desired m/z (1442.6 [M/2+H⁺]) was detected. The reaction mixture was directly purified by prep-HPLC (A: H₂O, B: ACN) to give compound 4 (0.125 g, crude) as a white solid. Calculated MW: 2884.27 observed m/z: 1442.6 ([M/2+H⁺]).

### Procedure for Preparation of BPI-1

To a solution of compound 4 (0.125 g, 43.34 umol, 1 eq.) in MeCN (3 mL) and H₂O (3 mL) was added TCEP HCl (168.77 mg, 588.78 umol, 1.5 eq). The reaction mixture was stirred at 25° C. for 1 h. LC-MS showed compound 4 was consumed completely and a peak with desired m/z (1387.8 [M/2+H⁺])) was detected. The reaction was directly purified by prep-HPLC (A: H₂O, B: ACN) to give BPI-1 (0.30 g, crude) as a white solid. Calculated MW: 2775.12 observed m/z: 1387.8 ([M/2+H⁺]).

### 23.2. Synthesis of Compound BPI-2

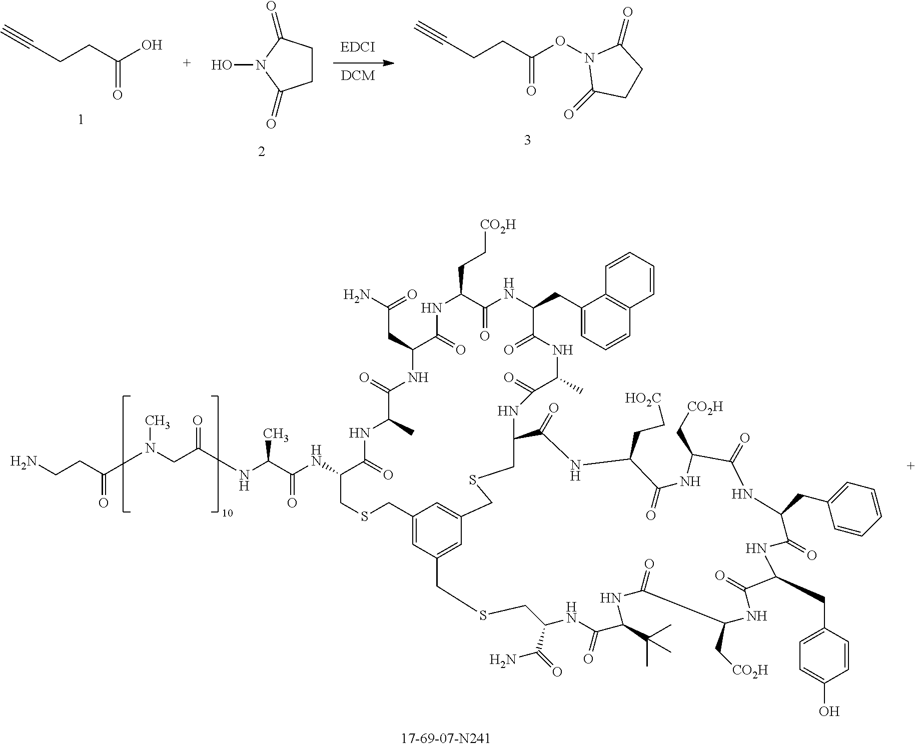

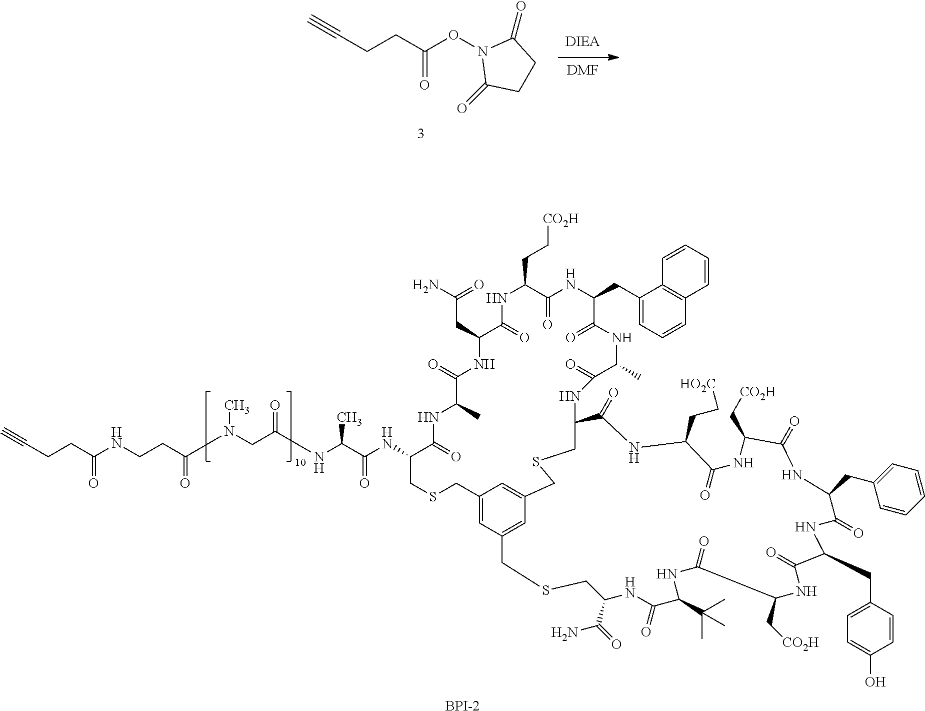

### Procedure for Preparation of Compound 3

To a solution of compound 1 (1 g, 10.19 mmol, 1 eq.) in DCM (50 mL) was added EDCI (3.91 g, 20.39 mmol, 2 eq.) and compound 2 (1.29 g, 11.21 mmol, 1.1 eq.). The reaction mixture was stirred at 25° C. for 1 h. TLC indicated compound 1 was consumed completely and one new spot was formed. The reaction mixture was directly purified by flash silica gel chromatography (ISCO®; 40 g SepaFlash® Silica Flash Column, Eluent of 0-50% Ethyl acetate/Petroleum ether gradient@40 mL/min) to give compound 3 (1.29 g, 6.28 umol, 61.6% yield) as a colorless solid.

### Procedure for Preparation of BPI-2

To a solution of 17-69-07-N241 (0.40 g, 150.44 umol, 1 eq.) in DIVIF (4 mL) was added DIEA (58.33 mg, 451.31 umol, 78.61 uL, 3 eq.) and compound 3 (58.72 mg, 300.87 umol, 2 eq.), the reaction mixture was stirred at 25° C. for 3 h. LC-MS showed 17-69-07-N241 was consumed completely and a peak with desired m/z (1370 [M/2+H⁺]) was detected. The reaction was directly purified by prep-HPLC (A: H₂O, B: ACN) to give BPI-2 (0.385 g, 140.56 umol, 93.44% yield, 81.49% purity) as a white solid. Calculated MW: 2739.02 observed m/z: 1370 ([M/2+H⁺]).

### Example 24. Plasma Stability

The pooled frozen mouse, rat, dog, monkey or human plasma is thawed in a water bath at 37° C. prior to experiment. Plasma is centrifuged at 4000 rpm for 5 min and the clots are removed if any. The pH is adjusted to 7.4±0.1 if required. 5 mM intermediate solutions of test compounds are prepared with DMSO. 1 mM intermediate solution of positive control Propantheline is prepared by diluting 5 μL of the stock solution with 45 μL ultra pure water. 1 mM intermediate solution of positive control Enalaprit is prepared by diluting 5 μL of the stock solution with 45 μL DMSO. 100 μM dosing solution is prepared by diluting 20 μL of the intermediate solution (1 mM) with 180 μL DMSO. For positive controls, 100 μM intermediate solution is prepared by diluting 20 μL of the stock solution with 180 μL 45% MeOH/H2O. For test compounds I-7 to I-9, I-17, I-18, and I-22 I-33, 100 μM working solution is prepared by diluting 10 μL of the intermediate solution with 490 μL DMSO. 196 μL of blank plasma is spiked with 4 μL of dosing solution (100 pA4) to achieve 2 μM of the final concentration in duplicate and samples are incubated at 37° C. in a water bath. For compounds I-7 to I-9, I-17, I-18, I-22, and I-24 to I-33, at each time point (0, 1, 2, 4, 6 and 24 hr), reactions were stopped by removing the plates from water bath and 200 μL 20 mM NH4OAC mix well first and then adding 800 μL of ice-cold stop solution (200 ng/mL Labetalol, 200 ng/mL Tolbutamide, 500 ng/mL N6-Ben and 40 mM DBAA in 100% Methanol). For compound I-23, propantheline and enalapril, at each time point (0, 1, 2, 4, 6 and 24 hr), reactions were stopped by removing the plates from water bath and adding 800 μL of ice-cold stop solution (200 ng/mL Tolbutamide and 200 ng/mL Labetalol in 0.1% FA in 100% Acetonitril or 200 ng/mL Tolbutamide and 200 ng/mL Labetalol in 50% Methanol/Acetonitril). Sample plates were vortexed immediately on a plate shaker at 800 rpm for 10 min and then centrifuged at 4,000 rpm for 10 min. An aliquot of supernatant (200 μL) is transferred from each well before submitting to LC-MS/MS analysis.

The percentage of test compound remaining at the individual time points relative to the 0 hour sample is calculated using following equation: Percent Remaining=100×(PAR at appointed incubation time/PAR at T₀ time) where PAR is the peak area ratio of analyte versus internal standard (IS).

The conjugates showed large differences in stability in plasma from various species (Table 6). The data show that the linker structure has a dramatic impact on the stability of the conjugates. A: T_{1/2}>24 hours; B: 6 hours<T_{1/2}<24 hours; C: 2 hours<T_{1/2}<6 hours; D: T_{1/2}<2 hours.

TABLE 6
Plasma stability.

| Compound # | T_{1/2} (human) | T_{1/2} (cyno) | T_{1/2} (rat) | T_{1/2} (mouse) | T_{1/2} (dog) |
|---|---|---|---|---|---|
| I-8 | C | N/A | D | D | N/A |
| I-7 | A | A | B | B | A |
| I-22 | A | A | C | C | A |
| I-9 | N/A | N/A | N/A | N/A | N/A |
| I-25 | A | A | A | A | N/A |
| I-24 | A | A | B | B | N/A |
| I-27 | A | A | A | D | N/A |
| I-28 | D | C | D | D | N/A |
| I-29 | A | A | B | A | N/A |
| I-31 | A | A | A | A | N/A |
| I-32 | A | A | D | D | N/A |
| I-33 | A | A | A | B | N/A |
| I-30 | A | A | A | A | N/A |
| I-17 | D | D | D | D | D |
| I-18 | D | D | D | D | D |
| I-26 | D | D | D | D | N/A |
| I-23 | D | D | D | D | N/A |

### Example 25. Blood Stability

CD-1 mouse blood (n≥2, male, fasted overnight) is collected from animals housed at an internal facility. (EDTA-K2 is used as anticoagulant)

The test compounds and positive control stock solutions are prepared to 10 mM in DMSO or ultrapure water. Control compounds bisacodyl, enalapril and propantheline known to be metabolised by blood are also incubated alongside each batch of test compounds I-7, I-22, I-24, I-25, I-27, and I-29 to I-33. The dosing solutions of test compounds and controls are prepared by diluting the stock solution with 45% MeOH/H₂O to achieve 100 uM. Test compound solutions (final incubation concentration=2 μM) are incubated with blood (final DMSO concentration≤1%) at six different time points (0, 1, 2, 4, 6 and 24 hours) in duplicate (n=2). The reaction is terminated by adding 100 uL ultra-pure water, mixing well, then adding 800 uL of 100% methanol containing 200 ng/mL tolbutamide and 200 ng/mL labetalol as internal standards. Samples are then vortexed and centrifuged at 3200×g for 20 minutes. Following centrifugation, the concentration of test compound in the supernatant is determined semi-quantitatively by LC-MS/MS. The percentage of test compound remaining at the individual time points relative to the 0 hour sample is then reported. The percent remaining of test compound after incubation in blood is calculated using following equation: Percent Remaining=100×(PAR at appointed incubation time/PAR at T₀ time) where PAR is the peak area ratio of analyte versus internal standard (IS).

The different TLR conjugates showed large differences in stability in mouse blood (Table 7). The data show that the linker structure has a dramatic impact on the stability of the conjugates. A: T_{1/2}>24 hours; B: 6 hours<T_{1/2}<24 hours; C: 2 hours<T_{1/2}<6 hours; D: T_{1/2}<2 hours.

TABLE 7
Blood stability

| Compound # | T_{1/2} (mouse) |
|---|---|
| I-7 | D |
| I-22 | D |
| I-25 | A |
| I-24 | B |
| I-27 | C |
| I-29 | D |
| 1-31 | B |
| I-32 | D |
| I-33 | B |
| I-30 | B |

### Example 26. Plasma Pharmacokinetics of Bicycle Conjugates and Released Payloads in CD-1 Mice

Male CD-1 mice were dosed with 3 mg/kg of Bicycle Conjugate formulated in 25 mM Histidine HCl, 10% sucrose pH 7 via tail vein injection. Serial bleeding (about 80 μL blood/time point) was performed via submadibular or saphenous vein at each time point. All blood samples were immediately transferred into prechilled microcentrifuge tubes containing 2 μL K2-EDTA (0.5M) as anti-coagulant and placed on wet ice. Blood samples were immediately processed for plasma by centrifugation at approximately +4° C., 3000 g. The precipitant including internal standard (350 uL) was immediately added into the 35 uL plasma sample, mixed well and centrifuged at 3220 g, +4° C. for 15 minutes. The supernatant was transferred into pre-labelled polypropylene microcentrifuge tubes, and then quick-frozen over dry ice. The samples were stored at −70° C. or below as needed until analysis. Supernatant samples were mixed with 50 uL water, vortexed well and centrifuged at 3220 g, +4° C. for 15 minutes. A sample of the supernatant was injected for LC-MS/MS analysis using an Acquity UPLC with AB Sciex 6500+Triple Quad MS in positive ion mode to determine the concentrations of Bicycle conjugate and released payload. Plasma concentration versus time data were analyzed by non-compartmental approaches using the Phoenix WinNonlin 6.3 software program. C₀, Cl, Vdₛₛ, T_{1/2}, AUC₍₀₋ₗₐₛₜ₎, AUC_{(0-inf)}, MRT₍₀₋ₗₐₛₜ₎, MRT_{(0-inf)} and graphs of plasma concentration versus time profile were reported.

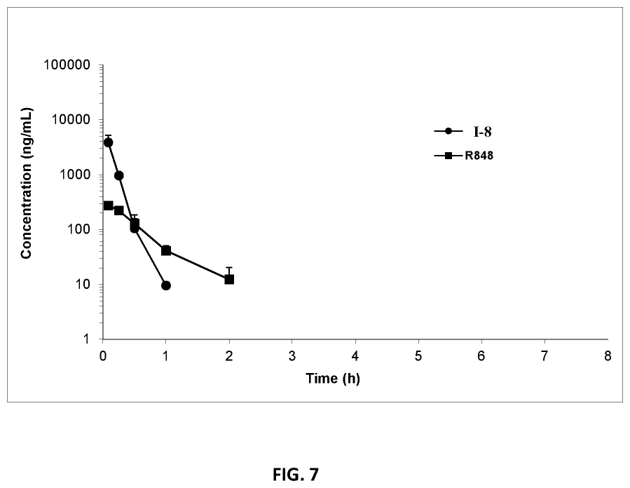

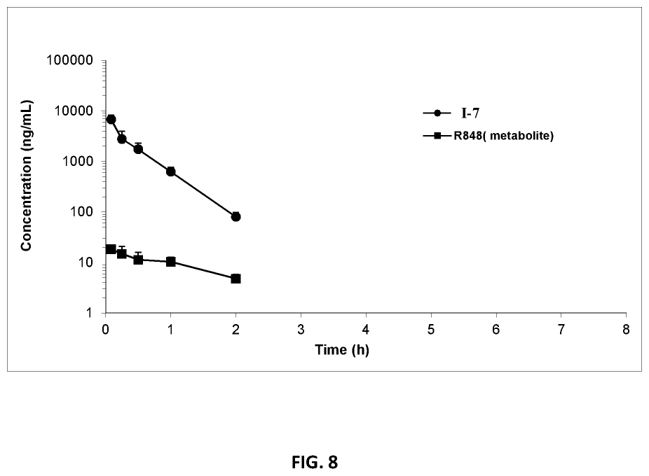

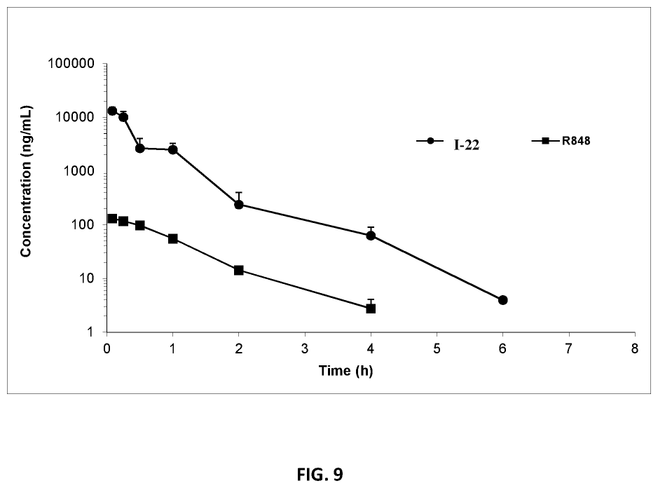

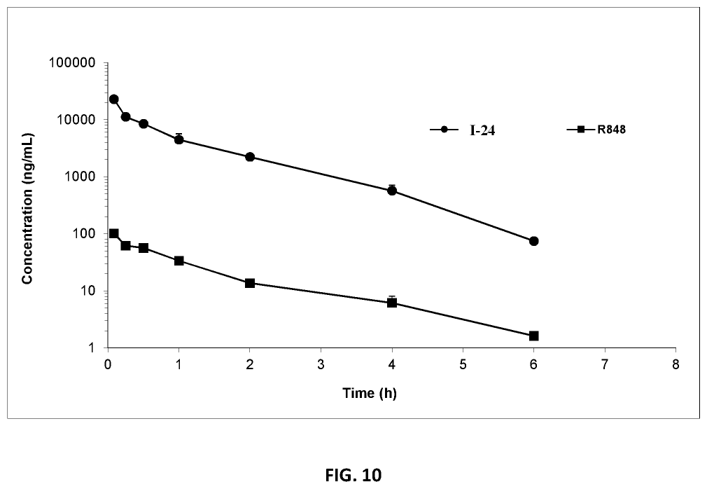

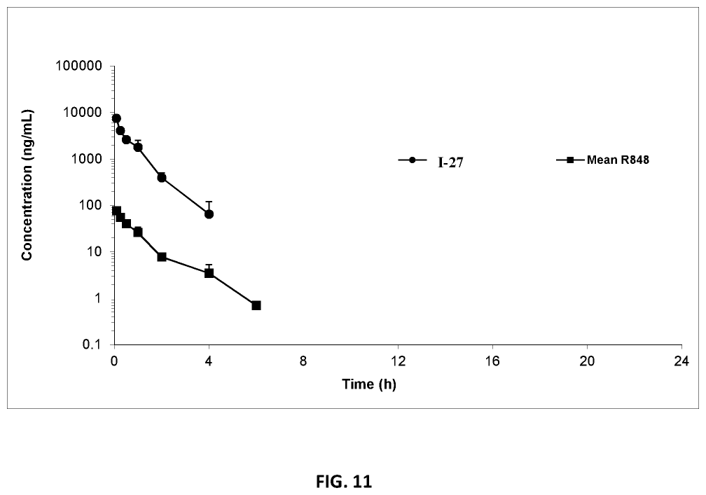

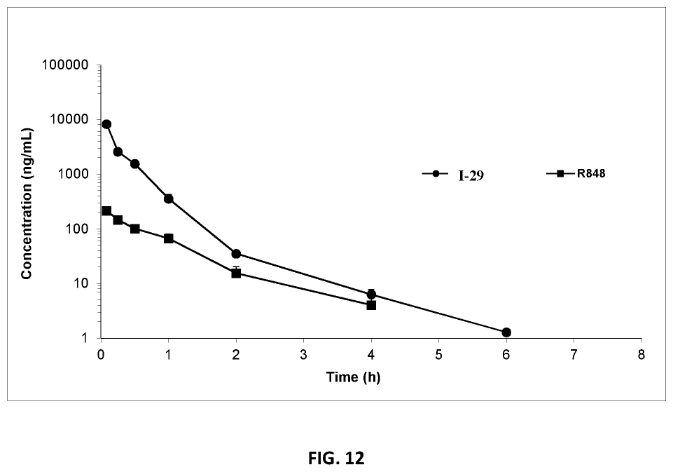

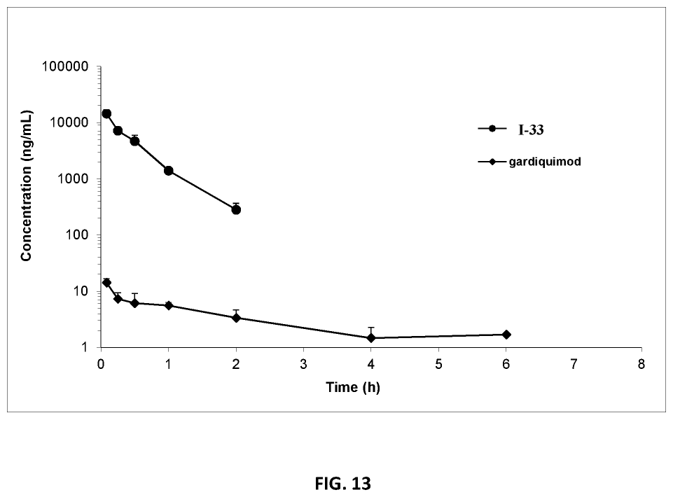

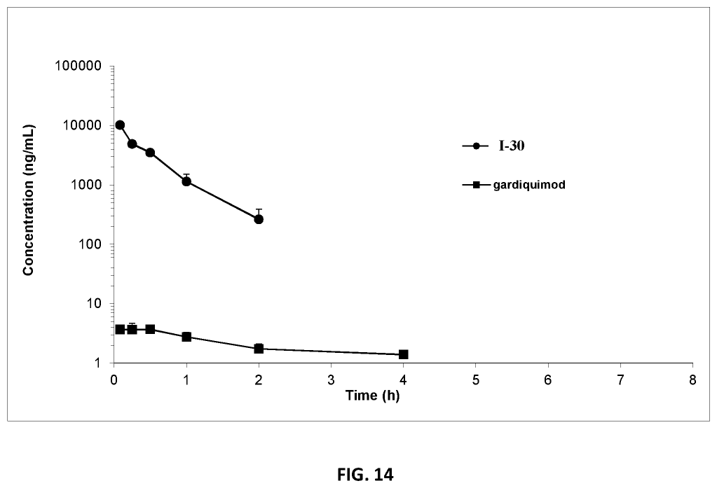

The results of the plasma concentration analysis in male CD-1 mice is shown in FIGS. 7-14, where it can be seen that the pharmacokinetic data show that the bicycle conjugates (in particular compounds I-7, I-8, I-22, I-24, I-27, I-29, I-30, and I-33) retain the property of rapid systemic elimination characteristic of bicyclic peptides and bicyclic peptide drug conjugates (BDCs).

### Example 27. Plasma Pharmacokinetics of Bicycle Conjugates and Released Payload in Cynomolgus Monkey

Male cynomolgus monkey (non-naïve) was dosed with 1 mg/kg of compound I-7 or I-22 formulated in 50 mM Acetate, 10% Sucrose (pH5) via intravenous infusion injection into the cephalic vein over 30 minutes. Serial bleeding (about 0.5 mL blood/time point) was performed via peripheral vein at each time point. All blood samples were collected in (K₂) EDTA*2H₂O (0.85-1.15 mg) containing blood collection tubes and placed on wet ice. Blood samples were immediately processed for plasma by centrifugation at approximately +4° C., 3000 g. The precipitant including internal standard was immediately added into the plasma sample, mixed well and centrifuged at 3220 g, +4° C. for 15 minutes. The supernatant was transferred into pre-labelled polypropylene microcentrifuge tubes, and then quick-frozen over dry ice. The samples were stored at −70° C. or below as needed until analysis. A sample was injected for LC-MS/MS analysis using an Acquity UPLC with AB Sciex 6500+ Triple Quad MS in positive ion mode to determine the concentrations of Bicycle conjugate and released payload. Plasma concentration versus time data were analyzed by non-compartmental approaches using the Phoenix WinNonlin 6.3 software program. C₀, Cl, Vdₛₛ, T_{1/2}, AUC₍₀₋ₗₐₛₜ₎, AUC_{(0-inf)}, MRT₍₀₋ₗₐₛₜ₎, MRT_{(0-inf)} and graphs of plasma concentration versus time profile were reported.

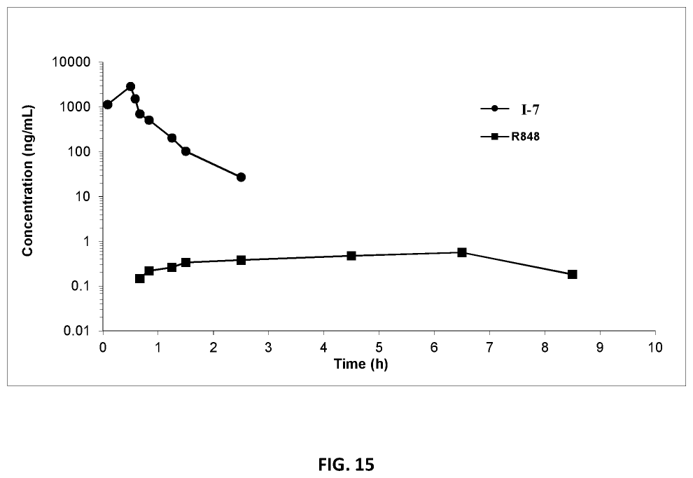

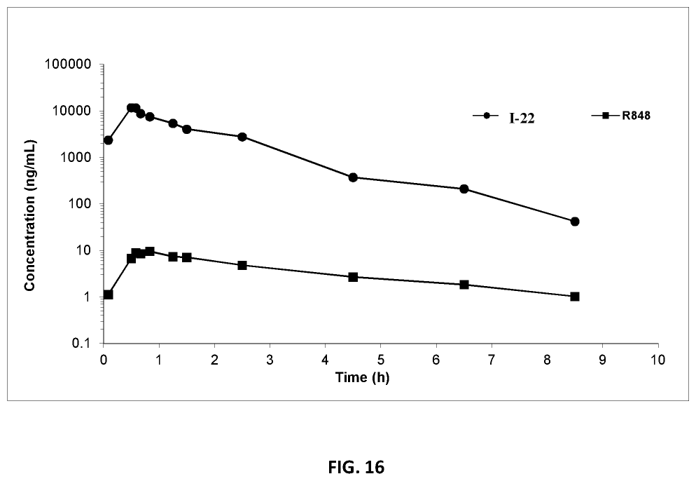

The results of the plasma concentration analysis in male cynomolgus monkey are shown in FIGS. 15-16, where it can be seen that the pharmacokinetic data show that the bicycle conjugates (in particular I-7 and I-22) retain the property of systemic elimination characteristic of bicyclic peptides and bicyclic peptide drug conjugates (BDCs).

### Example 28. Tumor Cytokine Pharmacodynamic Response of Bicycle Conjugates and Free Payloads in B16F10 Tumor Bearing C57BL/6 Mice after IT Dosing

Female C57BL/6 mice were implanted with 1×10⁶ B16F10 cells subcutaneously to induce tumor development. Tumor bearing mice were dosed intratumorally with vehicle, 1 mg of Bicycle conjugates or 0.1 mg of payloads formulated in 25 mM Histidine HCl, 10% sucrose, pH 7, when the average tumor volume was around 500 mm³ (I-7, I-22, n=3/group) or 460 mm³ (I-24, I-29, I-31 and I-33, n=4/group). Tumor, spleen and blood (by cardiac puncture) were harvested at 1 hour post dosing. Blood samples were immediately transferred into tubes containing EDTA as anti-coagulant for plasma preparation. Spleen, tumor and plasma were flash frozen and stored at −80° C. until homogenate preparation/analysis. Spleen and tumor samples for the cytokine analysis were prepared by homogenization in PBS with TissueLyser LT and centrifugation. Tissue lysates were stored at −80° C. until analysis.

TNF alpha, IFN beta, IL-6, IFN gamma and IL-12 p70 cytokine concentrations (for I-7 and I-22) or TNF alpha, IFN beta and IL-6 cytokine concentrations (for I-24, I-29, I-31, and I-33), were determined from the tumor, spleen and plasma for each mouse individually using LEGENDplex™ capture beads for murine TNF alpha, IFN beta, IL-6, IFN gamma and IL-12 p70, LEGENDplex™ Buffer Set I, LEGENDplex™ Mouse Anti-Virus Response Panel Standard and LEGENDplex™ Mouse Anti-Virus Response Panel Detection Antibodies (Biolegend). Analysis was performed using BD FACS Canto Plus Flow Cytometer.

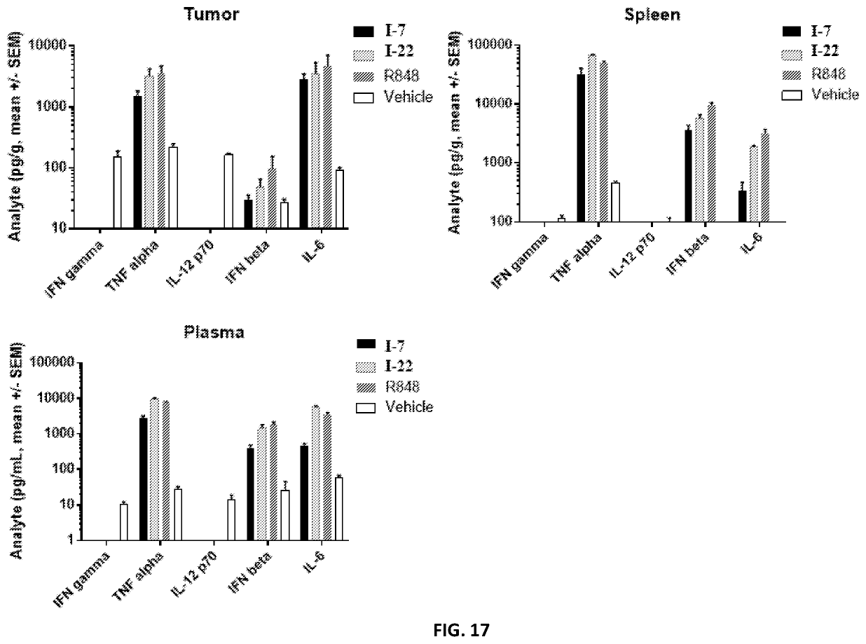

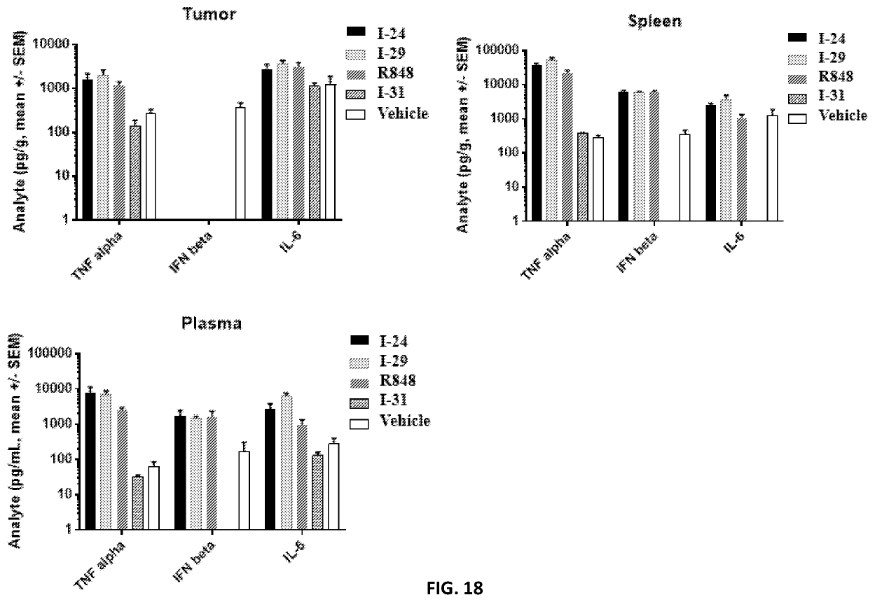

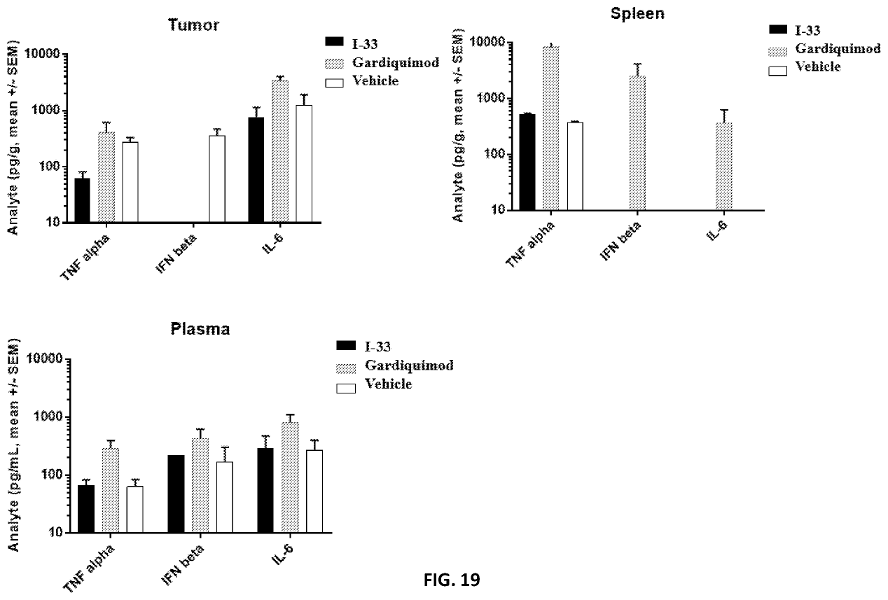

The results of the cytokine analysis from plasma, spleen and tumor are shown in FIGS. 17-19 where it can be seen that the Bicycle conjugates induce different levels of inflammatory cytokines in all tissues analyzed reflecting the particular linker and payload chemical structures.

### Example 29. Pharmacodynamic Response of Bicycle Conjugates and Free Payloads in C57BL/6 Mice after IV Dosing

Serum Cytokine Pharmacodynamic Response of Bicycle Conjugates and Free Payloads in C57BL/6 Mice after IV Dosing

Female C57BL/6 mice (n=4/group) were dosed with 20 mg/kg of Bicycle conjugates or 2 mg/kg of payloads (R848 or Gardiquimod) formulated in 25 mM Histidine HCl, 10% sucrose, pH 7, intravenously. Blood was collected by retro-orbital bleeding at 4 hours post dosing. Blood samples were immediately transferred into tubes containing EDTA as anti-coagulant for plasma preparation or into Gel Clot Activator tubes for serum separation. Blood samples were processed for serum and plasma which were stored at −80° C. until analysis.

IL-6 and TNF alpha cytokine concentration in serum were determined from each mouse individually using Becton Dickinson CBA analysis kits for murine IL-6 and TNF alpha according to manufacturer's instructions. IFN beta concentration was be determined for each mouse individually using Verikine ELISA for murine IFN beta manufacturer's instructions.

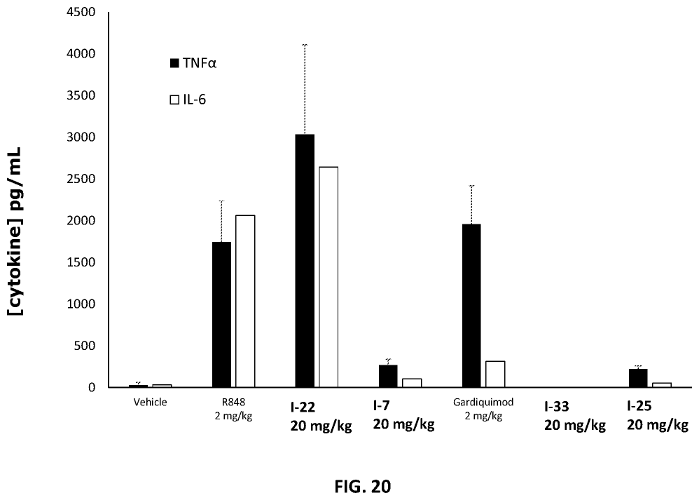

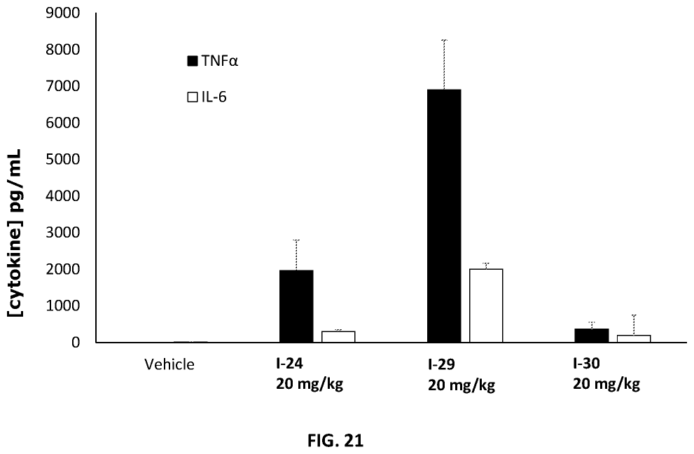

The results of the serum concentration analysis of cytokines are shown in FIGS. 20-21 where it can be seen that the bicycle conjugates induce differential levels of inflammatory cytokines reflecting the particular linker and payload chemical structures.

Tumor, Spleen and Plasma Cytokine Pharmacodynamic Response of Bicycle Conjugates and Free Payloads in B16F10 Tumor Bearing C57BL/6 Mice after IV Dosing

Female C57BL/6 mice were implanted with 1×10⁶ B16F10 cells subcutaneously to induce tumor development. Tumor bearing mice (n=3/group) were dosed intravenously with 20 mg/kg of I-7 or I-22, or 2 mg/kg of payload (R848) formulated in 25 mM Histidine HCl, 10% sucrose, pH 7, when the average tumor volume was around 590 mm³. Tumor, spleen and blood (by cardiac puncture) were harvested at 1 hour post dosing. Blood samples were immediately transferred into tubes containing EDTA as anti-coagulant for plasma preparation. Spleen, tumor and plasma were flash frozen and stored at −80° C. until homogenate preparation/analysis. Spleen and tumor samples for the cytokine analysis were prepared by homogenization in PBS with TissueLyser LT and centrifugation. Tissue lysates were stored at −80° C. until analysis.

TNF alpha, IFN beta, IL-6, IFN gamma and IL-12 p70 cytokine concentrations were determined for the tumor, spleen and plasma for each mouse individually using LEGENDplex™ capture beads for murine TNF alpha, IFN beta, IL-6, IFN gamma and IL-12 p′70, LEGENDplex™ Buffer Set I, LEGENDplex™ Mouse Anti-Virus Response Panel Standard and LEGENDplex™ Mouse Anti-Virus Response Panel Detection Antibodies (Biolegend). Analysis was performed using BD FACS Canto Plus Flow Cytometer.

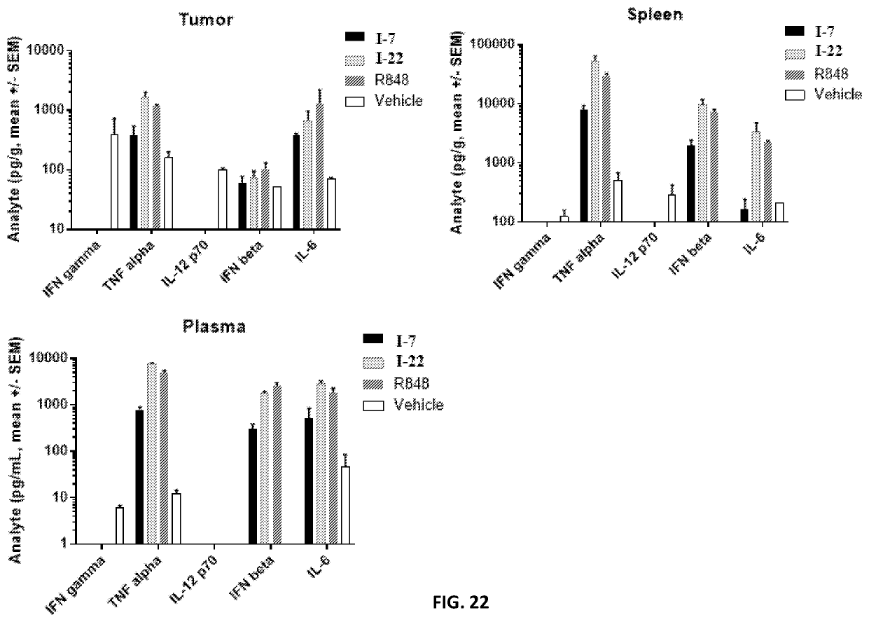

The results of the cytokine analysis from plasma, spleen and tumor are shown in FIG. 22 where it can be seen that the Bicycle conjugates induce different levels of inflammatory cytokines in all tissues analyzed reflecting the particular linker and payload chemical structures.

### Example 30. The Effect of IV Dosing in Mice

### The Effect of IV Dosing of Bicycle Conjugates on Tumor Growth in B16F10, MC38 and CT26 Tumor Bearing C57BL/6 Mice

Female C57BL/6 mice were implanted either with 1×10⁶ B16F10 cells, 3×10⁶ MC38 cells or 1×10⁶ CT26 cells subcutaneously to induce tumor development. B16F10, MC38 and CT26 tumor bearing mice were dosed three times a week intravenously with vehicle or 20 mg/kg of Bicycle conjugates (I-7, I-22, n=6/group) formulated in 25 mM Histidine HCl, 10% sucrose, pH 7. In addition, B16F10 tumor bearing mice were dosed three times a week (tiw) intravenously with 60 mg/kg of Bicycle conjugates. Treatment was initiated when the average tumor volume had reached 75 mm³ (B16F10), 68 mm³ (MC38) or 88 mm³ (CT26). Tumor volumes and mouse body weights were monitored 2-3 times a week.

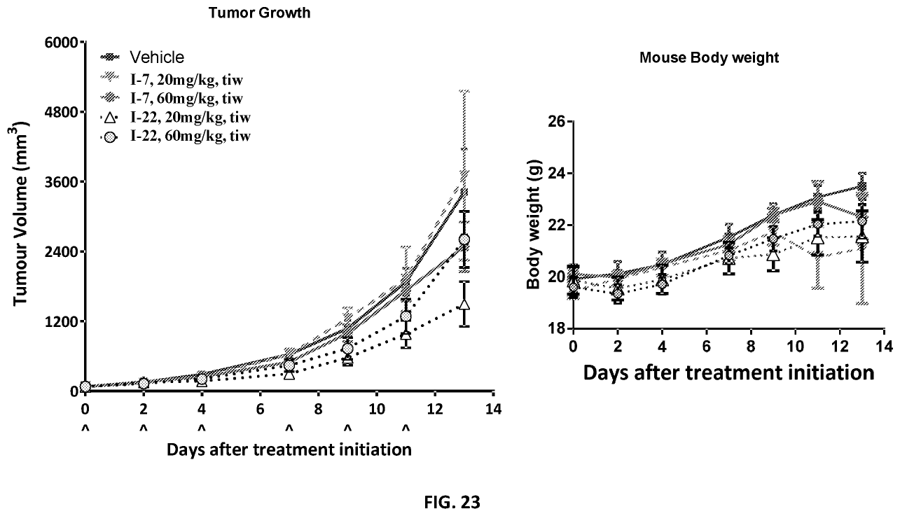

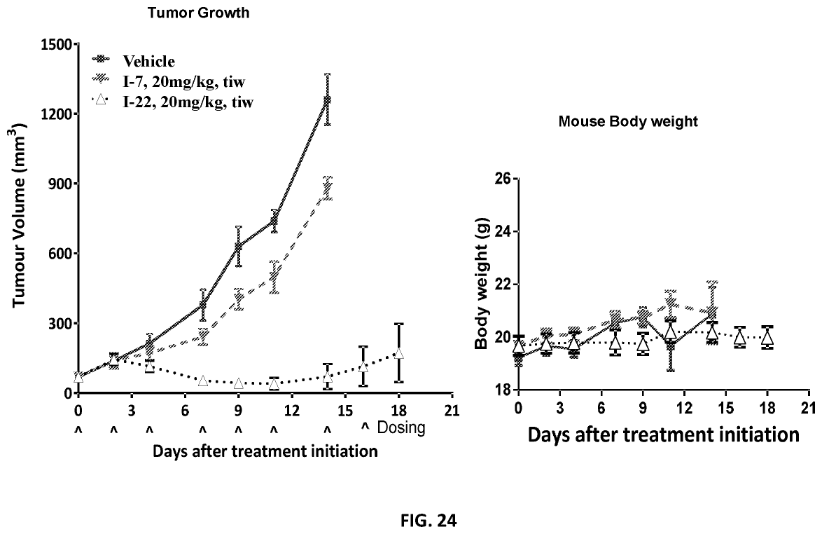

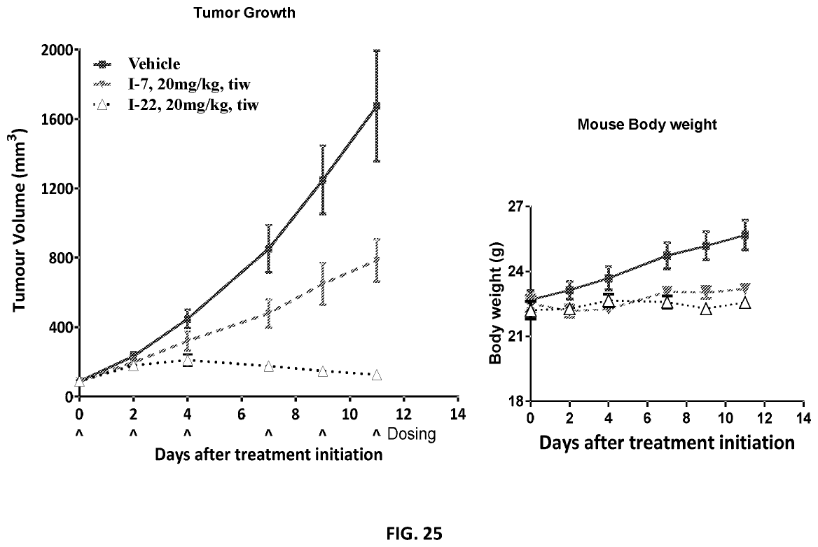

The results of the efficacy experiments in B16F10 (FIG. 23), MC38 (FIG. 24) and CT26 (FIG. 25) tumor bearing mice demonstrate that the Bicycle conjugates induce different levels of anti-tumor activity reflecting the particular linker and payload chemical structures. Treatments were well tolerated without significant mouse body weight loss.

The Effect of IV Dosing of Bicycle Conjugates in Combination with Anti-PD-1 Antibody on Tumor Growth in CT26 Tumor Bearing C57BL/6 Mice

Female C57BL/6 mice were implanted either with 1×10⁶ CT26 cells subcutaneously to induce tumor development. When the average tumor volume reached 67 mm³, mice (n=6/group) started receiving vehicle or 20 mg/kg of Bicycle conjugates (I-7, I-22) three times a week (tiw) intravenously or vehicle or 10 mg/kg anti-PD1 antibody intraperitoneally twice a week (biw) or a combination of I-7 (IV, tiw) and anti-PD1 antibody (IP, biw) or a combination of I-22 (IV, tiw) and anti-PD1 antibody (IP, biw). Tumor volumes and mouse body weights were monitored 2-3 times a week.

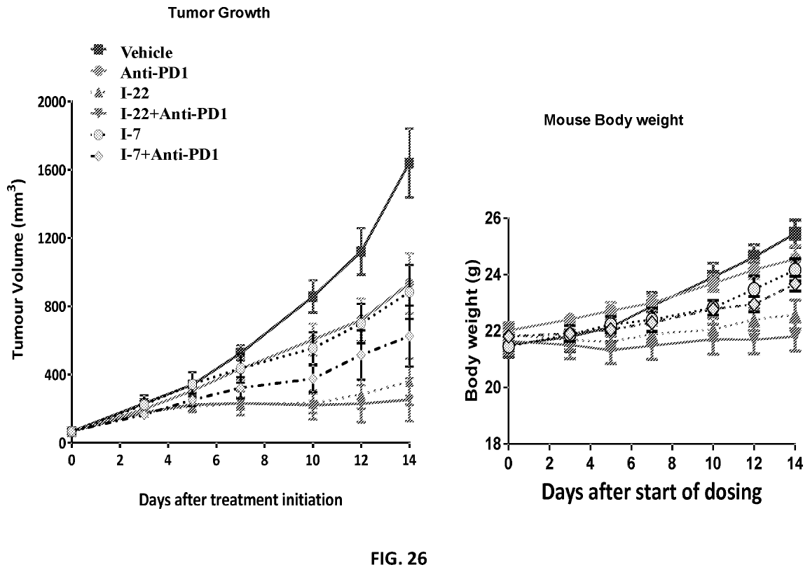

The results of the efficacy experiment in CT26 tumor bearing mice (FIG. 26) demonstrate that the Bicycle conjugates induce different levels of anti-tumor activity reflecting the particular linker and payload chemical structures. In addition, Bicycle conjugates induced different levels of combination effect with the anti-PD1 antibody treatment reflecting the particular linker and payload chemical structures of the Bicycle conjugates. Treatments were well tolerated without significant mouse body weight loss.

1. A compound of formula I: 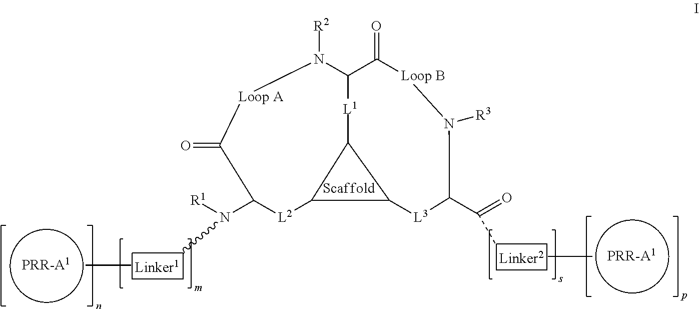
or a pharmaceutically acceptable salt thereof, wherein:
each of L¹, L², and L³ is independently a covalent bond or a C₁₋₈ bivalent hydrocarbon chain wherein one, two or three methylene units of the chain are optionally and independently replaced by —S—, —N(R)—, —O—, —C(O)—, —OC(O)—, —C(O)O—, —C(O)N(R)—, —N(R)C(O)—, —S(O)—, —S(O)₂— or —N(R)CH₂C(O)—;
each of R is independently hydrogen or C₁₋₄ alkyl;
each of m and s is independently 0 or 1;
n is 0, and p is 1; or n is 1, and p is 0; or n is 1, and p is 1;
each of q and r is independently 1, 2, 3, 4, 5, 6, 7, 8, 9, 10, 11, 12, 13, 14 or 15;
R¹ is R or —C(O)R;
each of R⁴ and R⁶ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
each of R^{4′} and R^{6′} is independently hydrogen or methyl;
each of R², R³, R⁵, and R⁷ is independently hydrogen, or C₁₋₄ aliphatic, or:
an R⁵ group and its adjacent R⁴ group are optionally taken together with their intervening atoms to form a 4-8 membered saturated or partially unsaturated monocyclic heterocyclic ring having 1-2 heteroatoms independently selected from nitrogen, oxygen, or sulfur; or
an R⁷ group and its adjacent R⁶ group are optionally taken together with their intervening atoms to form a 4-8 membered saturated or partially unsaturated monocyclic heterocyclic ring having 1-2 heteroatoms independently selected from nitrogen, oxygen, or sulfur;
Scaffold is a trivalent group that connects and orients a cyclic peptide;
Loop A is a bivalent natural or unnatural amino acid residue or peptide attached to the amino acid residue linked to L² and the amino acid residue linked to L¹, wherein Loop A comprises 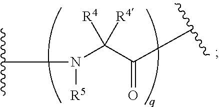
Loop B is a bivalent natural or unnatural amino acid residue or peptide attached to the amino acid residue linked to L¹ and the amino acid residue linked to L³, wherein Loop B comprises 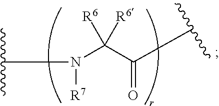 [symbol]
indicates the site of attachment to the N-terminus of the Bicycle; [symbol]
indicates the site of attachment to the C-terminus of the Bicycle;
PRR-A¹ is a pattern recognition receptor agonist;
PRR-A² is a pattern recognition receptor agonist;
Linker¹ is hydrogen or a bivalent moiety that connects the N-terminus of the Bicycle with PRR-A¹, wherein when n is 0, Linker¹ is hydrogen;
Linker² is —NH₂ or a bivalent moiety that connects the C-terminus of the Bicycle with PRR-A², wherein when p is 0, Linker² is —NH₂.

2. The compound of claim 1, wherein each of L¹, L², and L³ is a C₁₋₈ bivalent hydrocarbon chain wherein one, two or three methylene units of the chain are optionally and independently replaced by —S—, —N(R)—, —O—, —C(O)—, —OC(O)—, —C(O)O—, —C(O)N(R)—, —N(R)C(O)—, —S(O)—, —S(O)₂— or —N(R)CH₂C(O)—.

3. The compound of claim 1, wherein R′ is hydrogen or —C(O)CH₃.

4. The compound of claim 1, wherein Linker¹ is a covalent bond,

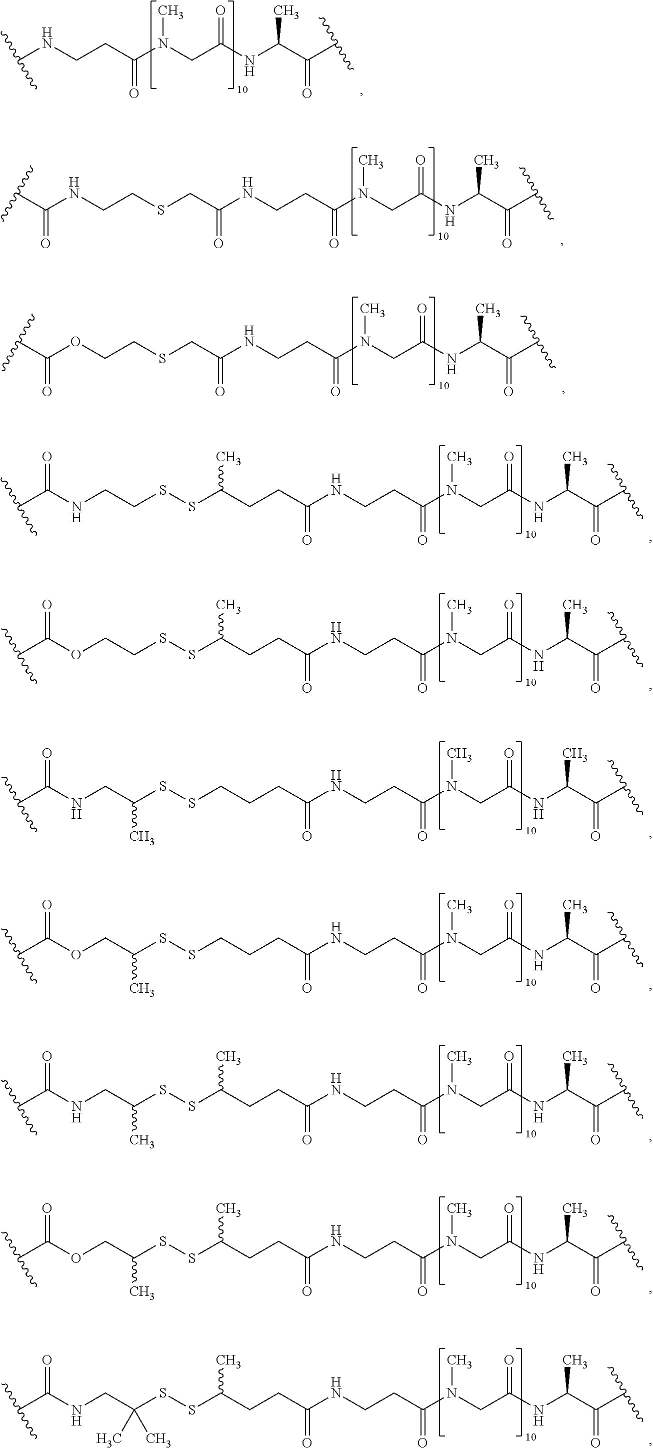

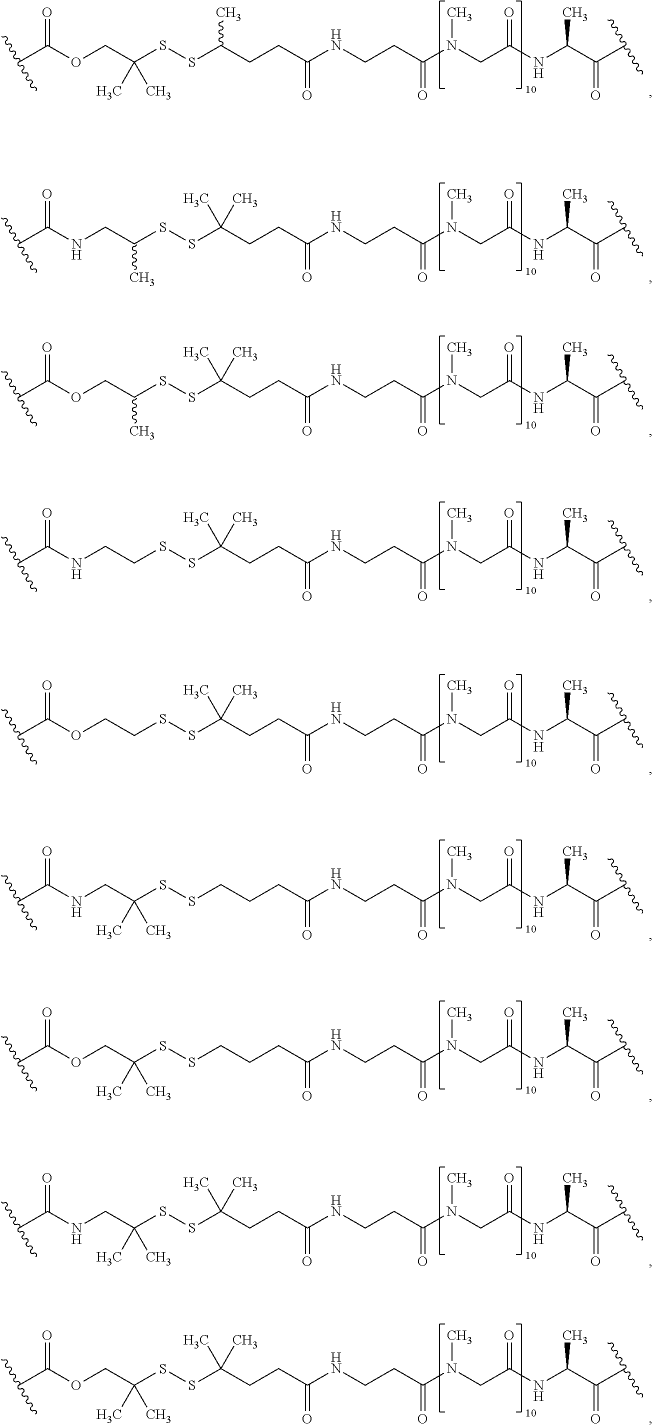

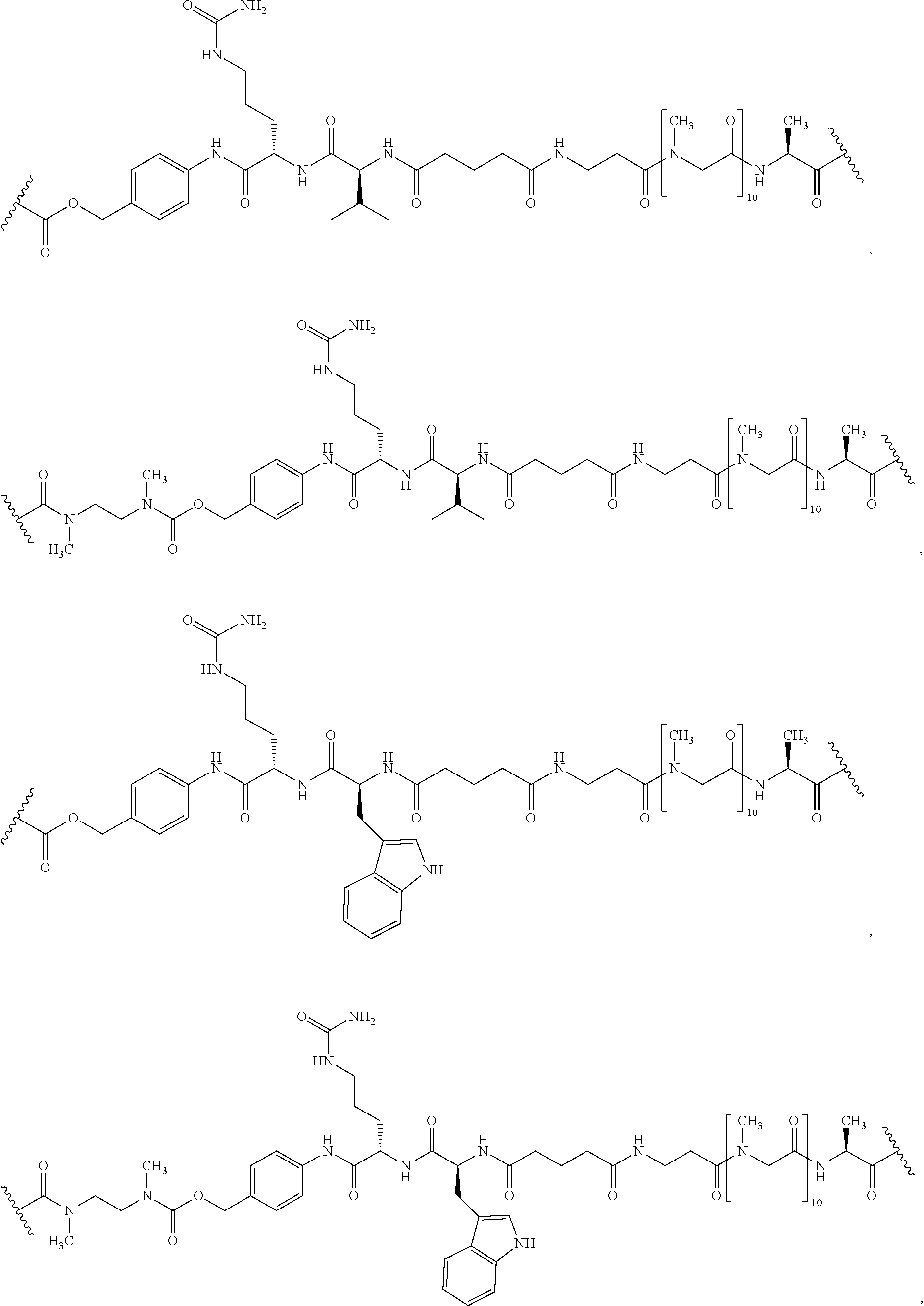

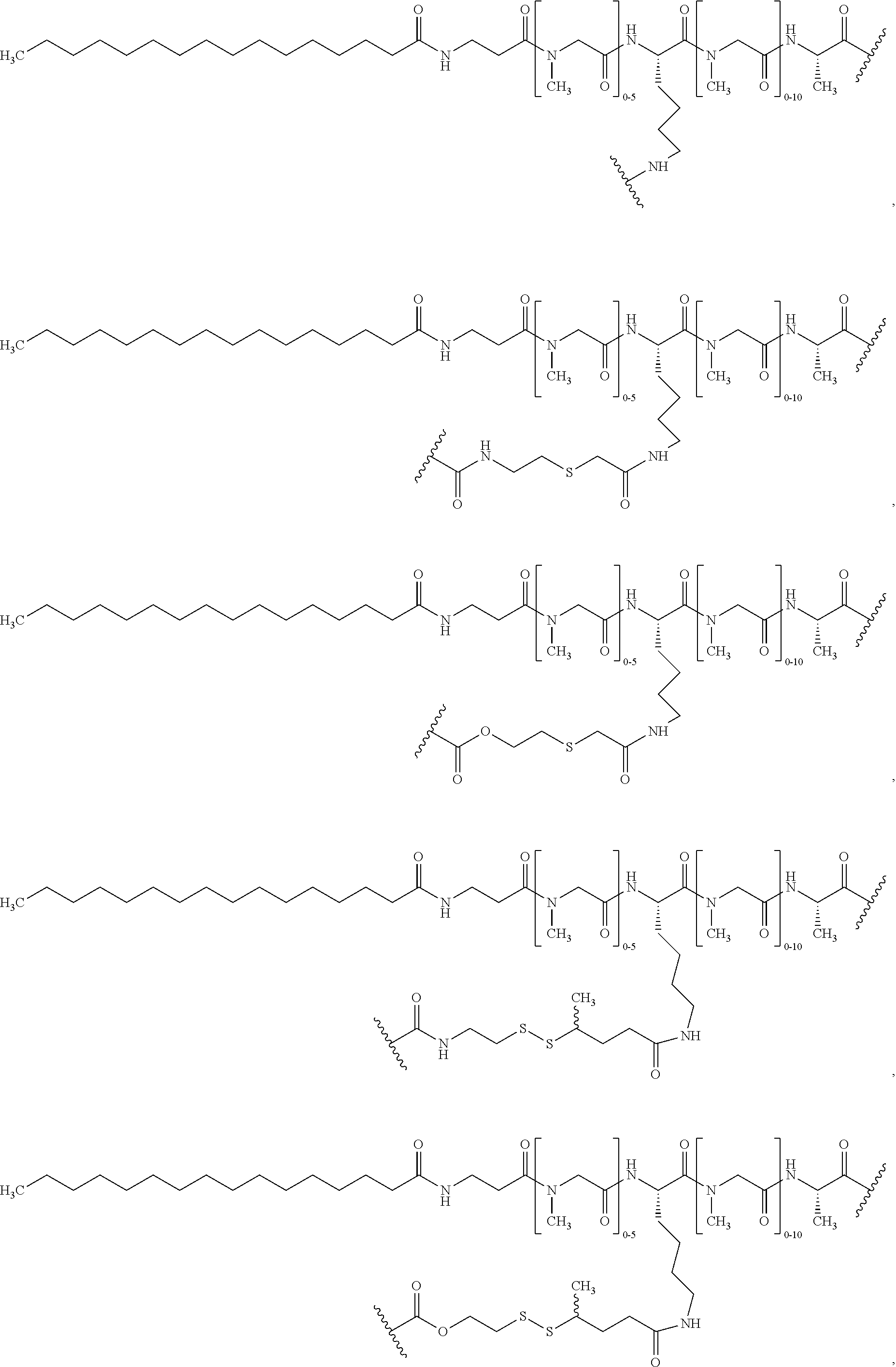

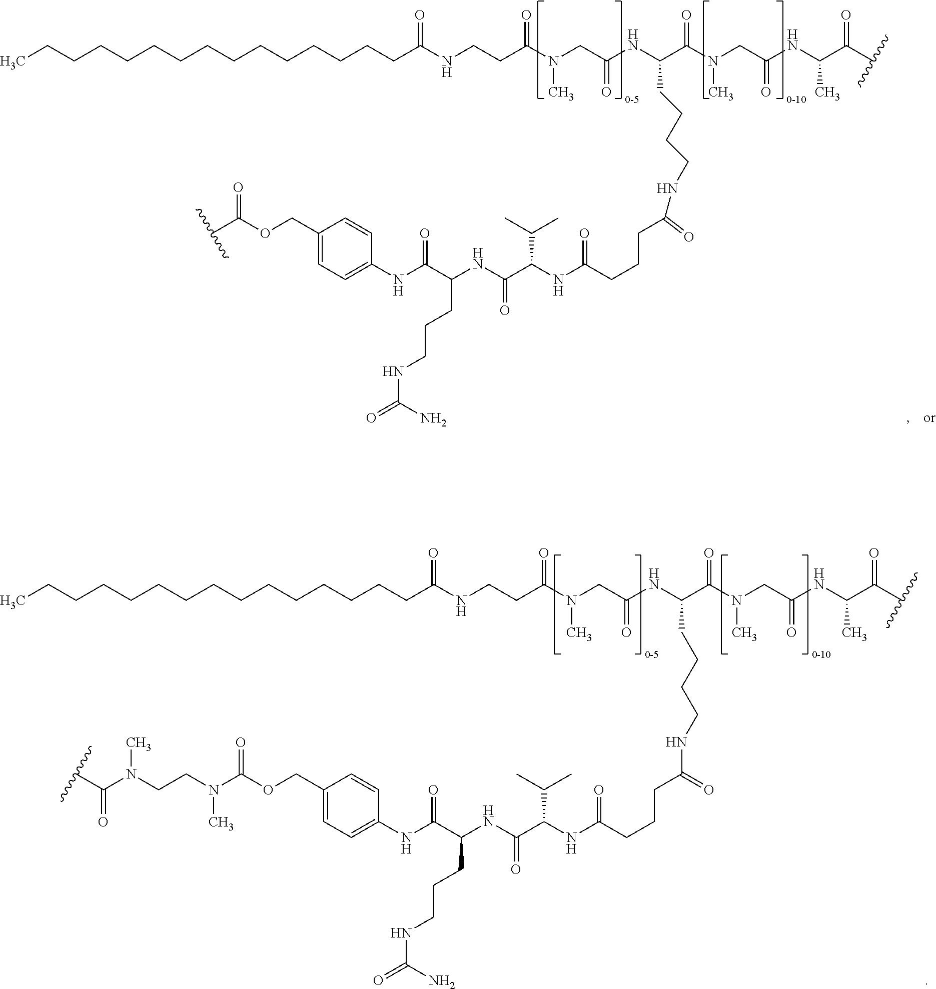

5. The compound of claim 1, wherein p is 0 and Linker² is —NH₂.

6. The compound of claim 1, wherein Scaffold is

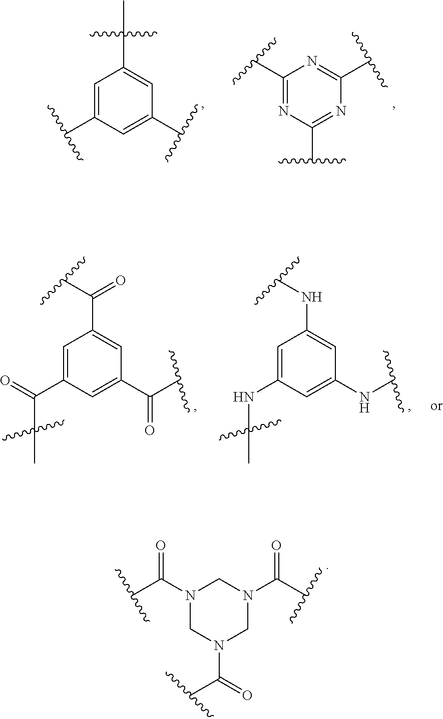 [SMILES: *c1cc(*)cc(*)c1 ; *c1nc(*)nc(*)n1 ; *C(=O)c1cc(C(*)=O)cc(C(*)=O)c1 ; *Nc1cc(N*)cc(N*)c1 ; *C(=O)N1CN(C(*)=O)CN(C(*)=O)C1]

7. The compound of claim 1, wherein PRR-A¹ is a toll-like receptor (TLR) agonist, a NOD-like receptor pyrin domain containing 3 (NLRP3) agonist, or both a TLR and NLRP3 agonist.

8. The compound of claim 1, wherein PRR-A¹ is

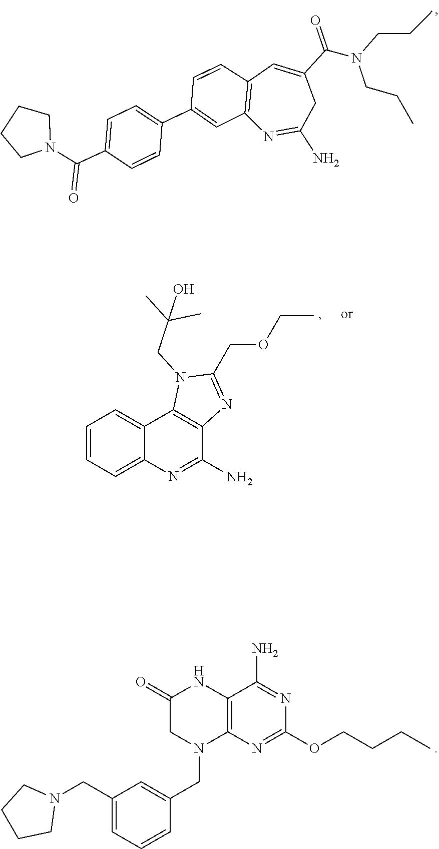 [SMILES: CCCN(CCC)C(=O)C1=Cc2ccc(-c3ccc(C(=O)N4CCCC4)cc3)cc2N=C(N)C1 ; CCOCc1nc2c(N)nc3ccccc3c2n1CC(C)(C)O ; CCCCOc1nc(N)c2c(n1)N(Cc1cccc(CN3CCCC3)c1)CC(=O)N2]

9. The compound of claim 1, wherein PRR-A² is a toll-like receptor (TLR) agonist, a NOD-like receptor pyrin domain containing 3 (NLRP3) agonist, or both a TLR and NLRP3 agonist.

10. The compound of claim 1, wherein PRR-A² is

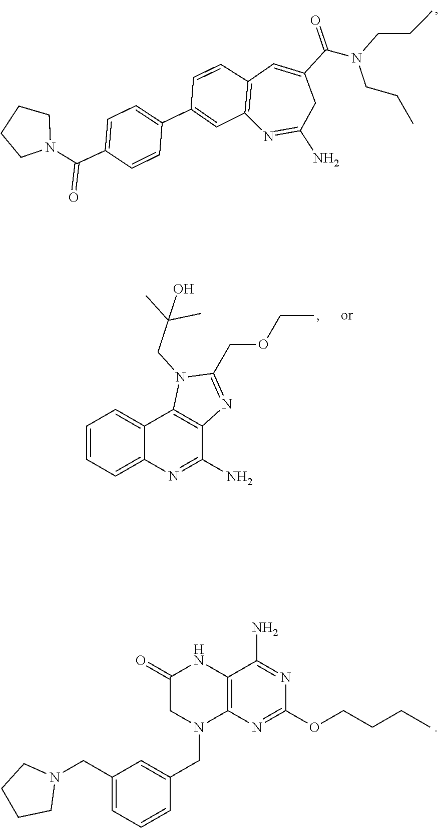 [SMILES: CCCN(CCC)C(=O)C1=Cc2ccc(-c3ccc(C(=O)N4CCCC4)cc3)cc2N=C(N)C1 ; CCOCc1nc2c(N)nc3ccccc3c2n1CC(C)(C)O ; CCCCOc1nc(N)c2c(n1)N(Cc1cccc(CN3CCCC3)c1)CC(=O)N2]

11. The compound of claim 1, wherein Loop A is

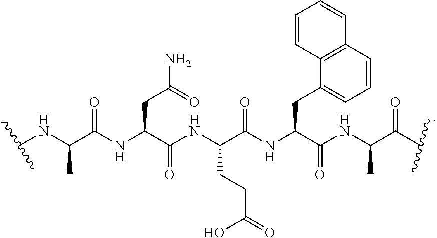 [SMILES: *N[C@H](C)C(=O)N[C@@H](CC(N)=O)C(=O)N[C@@H](CCC(=O)O)C(=O)N[C@@H](Cc1cccc2ccccc12)C(=O)N[C@H](C)C(*)=O]

12. The compound of claim 1, wherein Loop B is

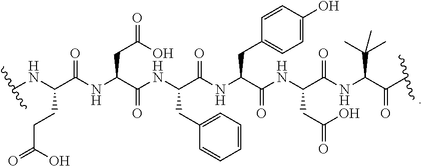 [SMILES: *N[C@@H](CCC(=O)O)C(=O)N[C@@H](CC(=O)O)C(=O)N[C@@H](Cc1ccccc1)C(=O)N[C@@H](Cc1ccc(O)cc1)C(=O)N[C@@H](CC(=O)O)C(=O)N[C@H](C(*)=O)C(C)(C)C]

13. The compound of claim 1, n is 0 and Linker¹ is hydrogen.

14. The compound of claim 1, wherein the compound is selected from

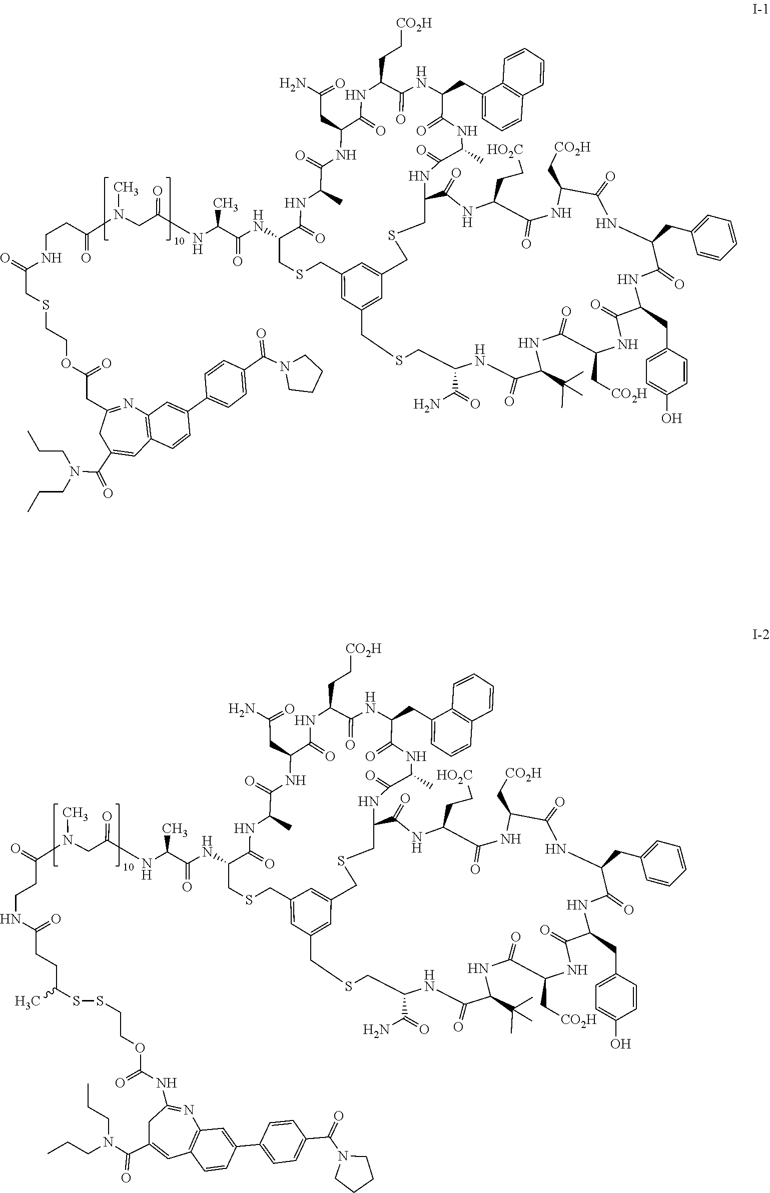

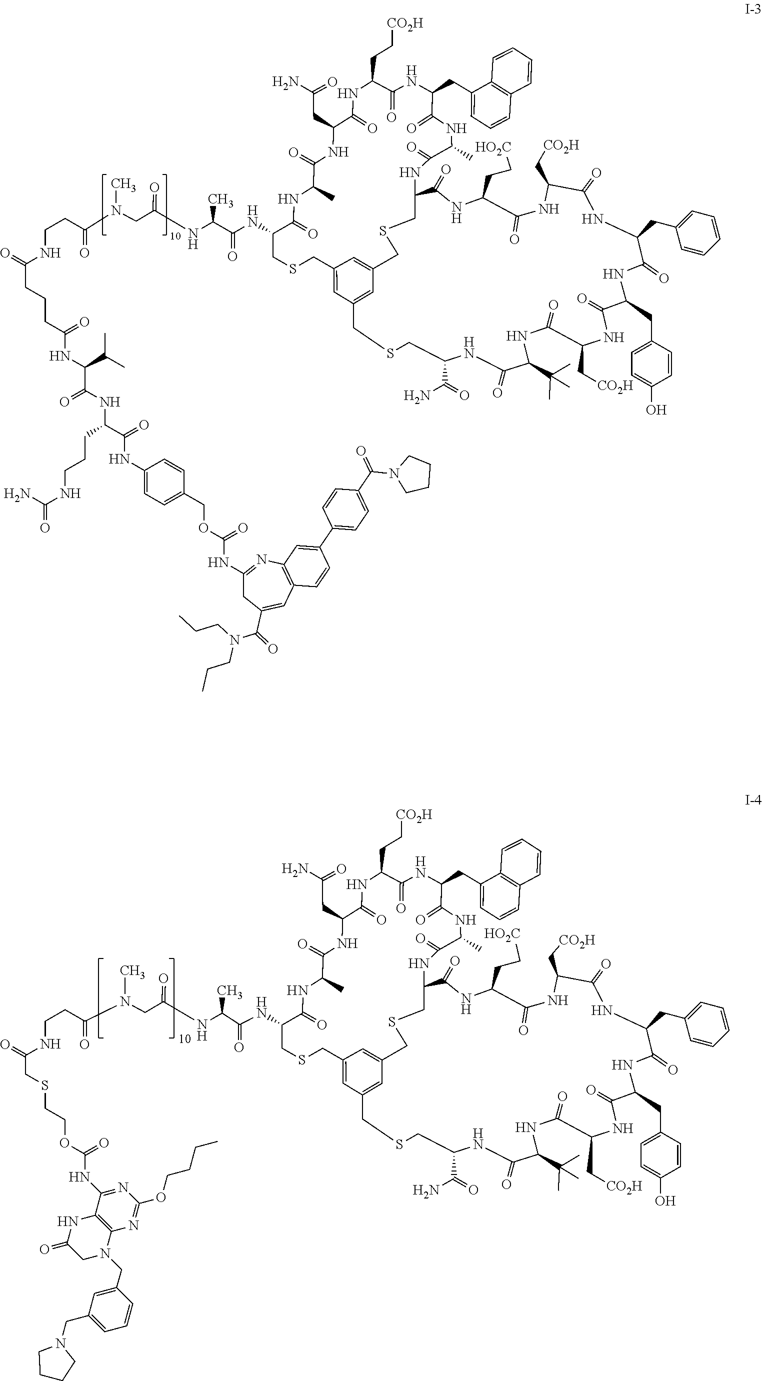

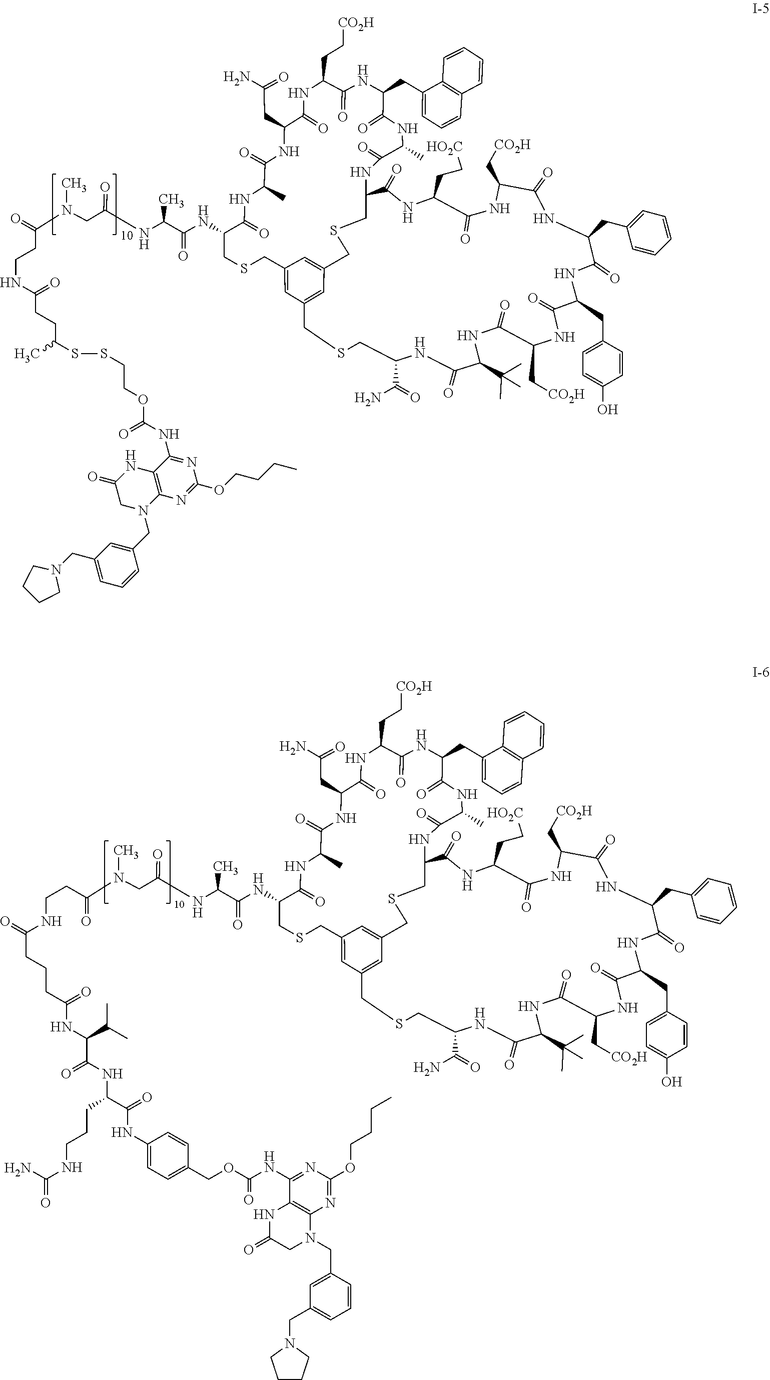

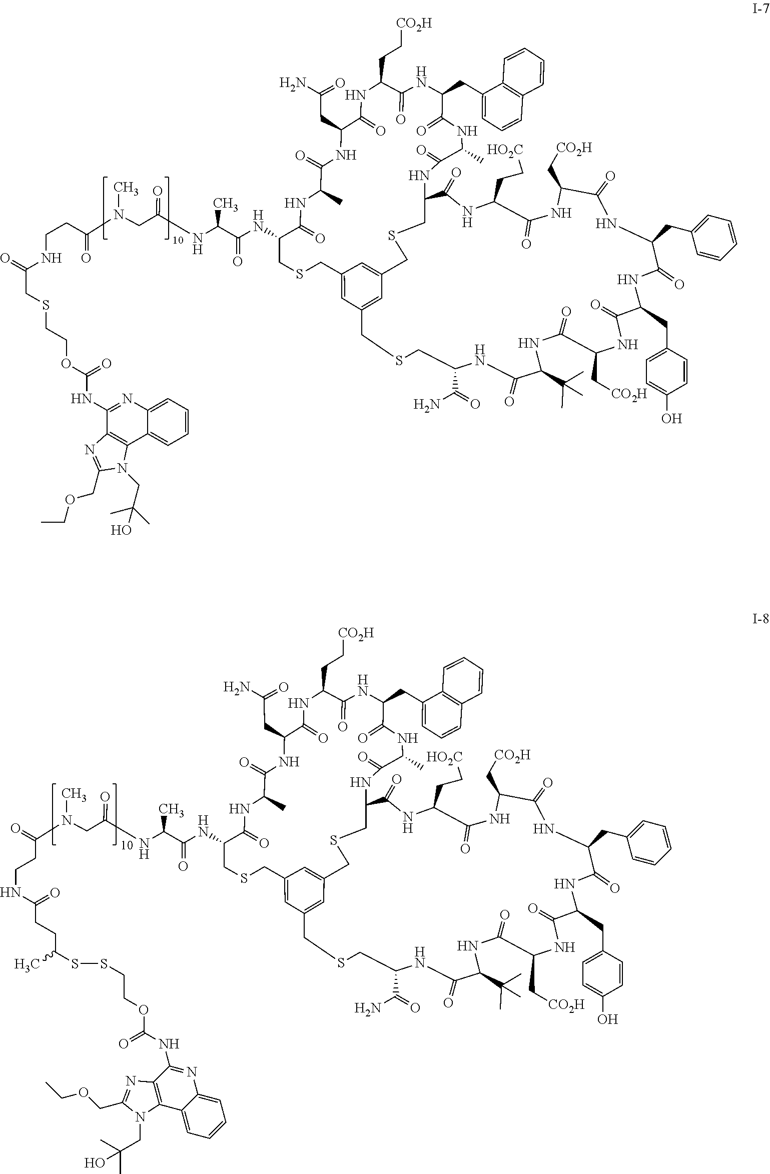

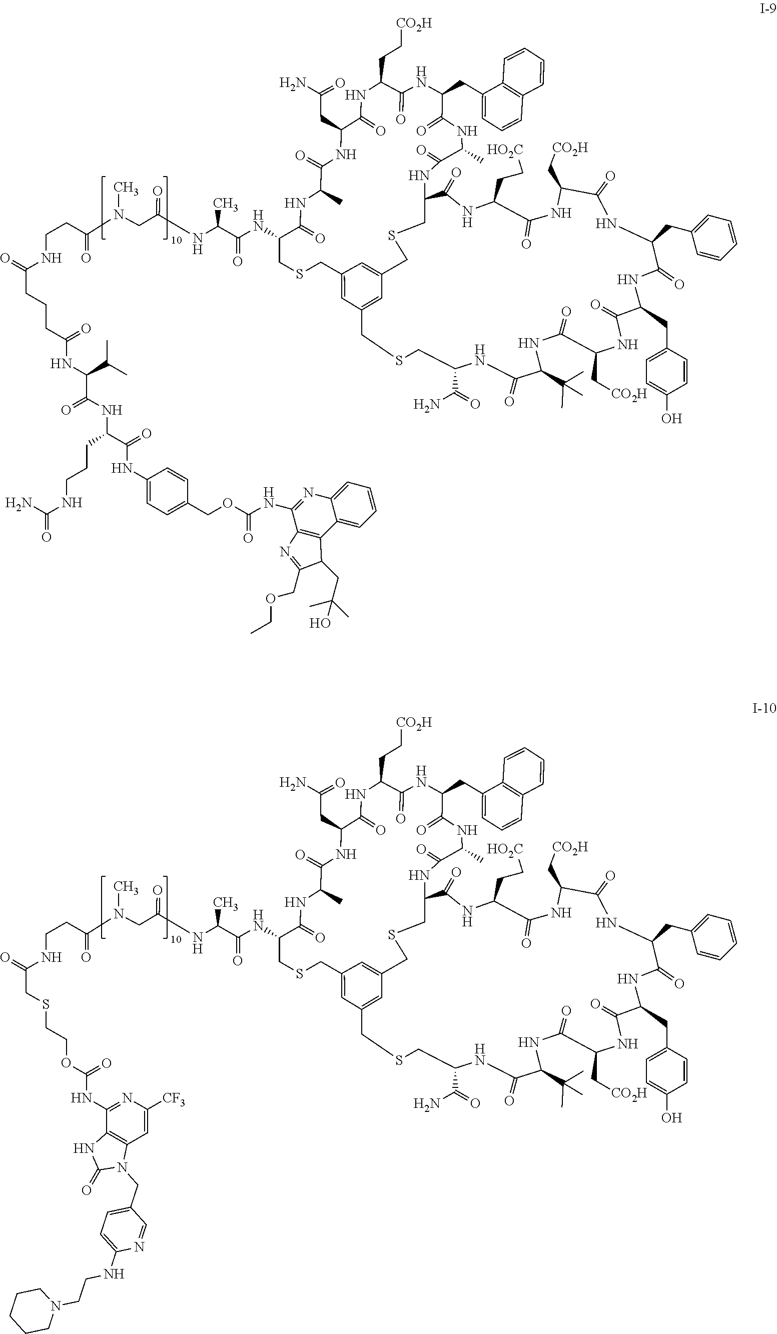

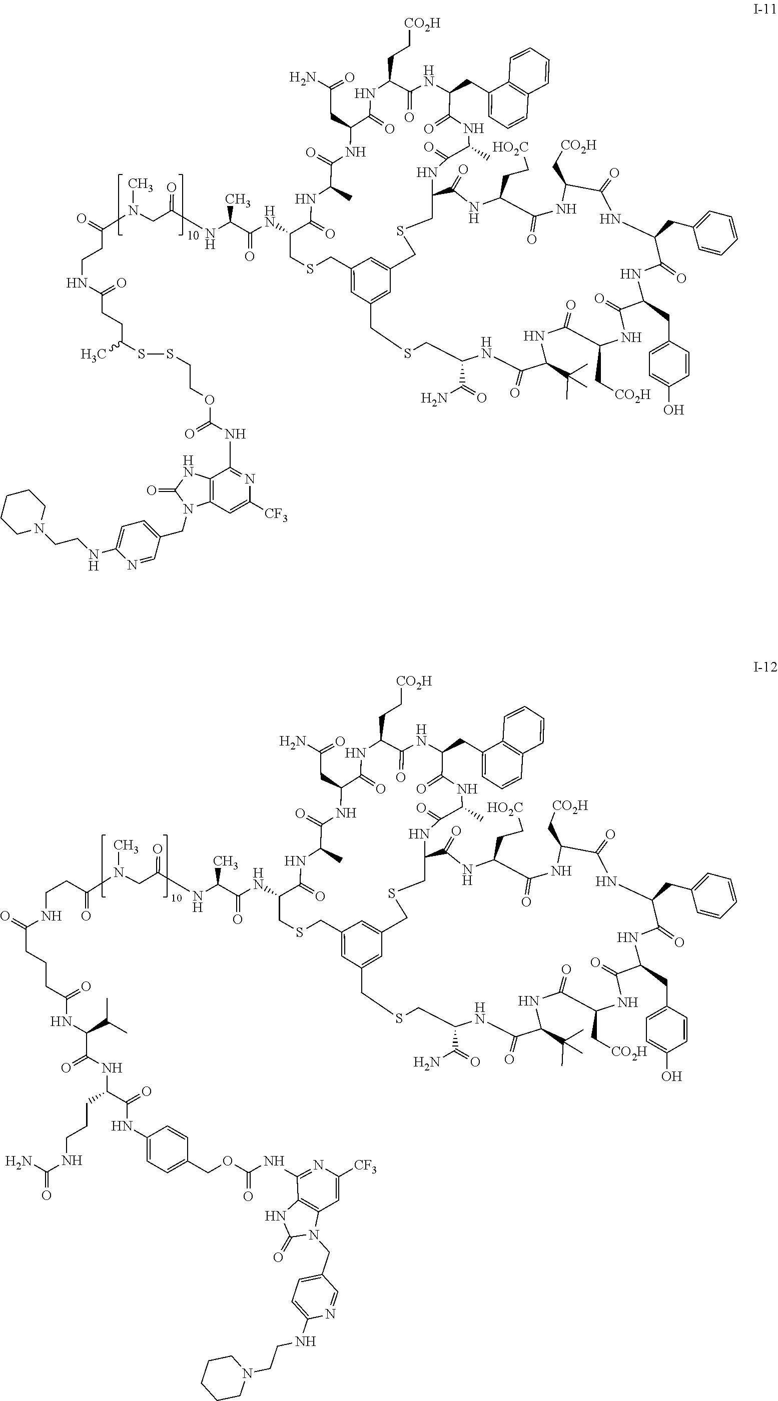

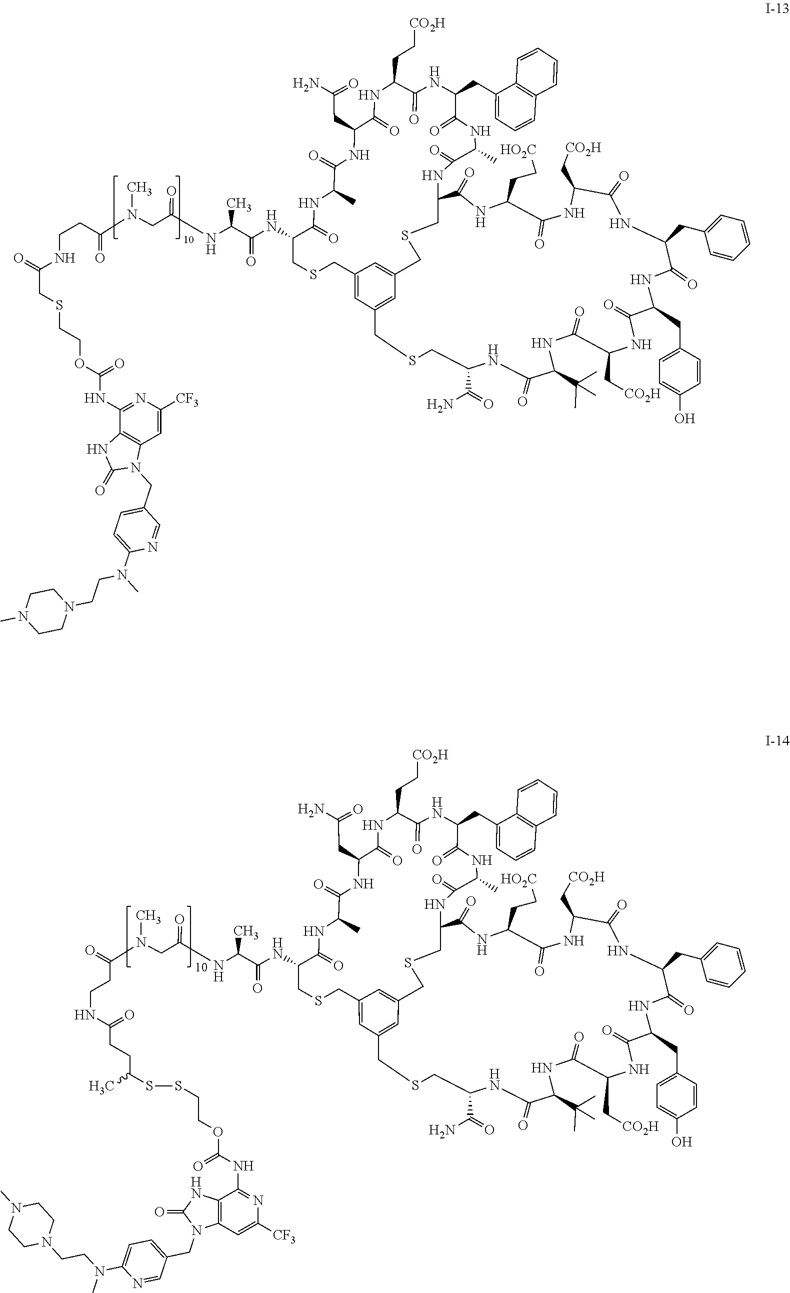

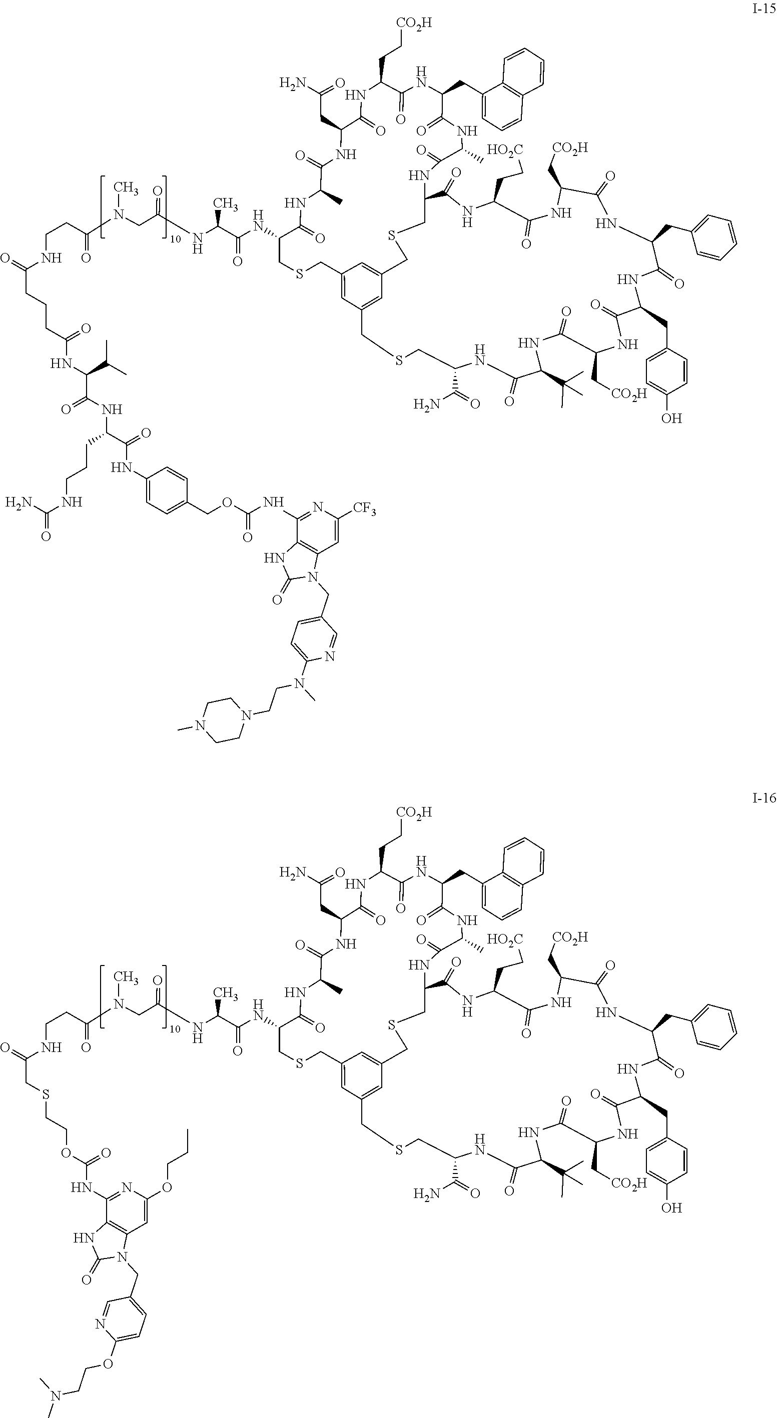

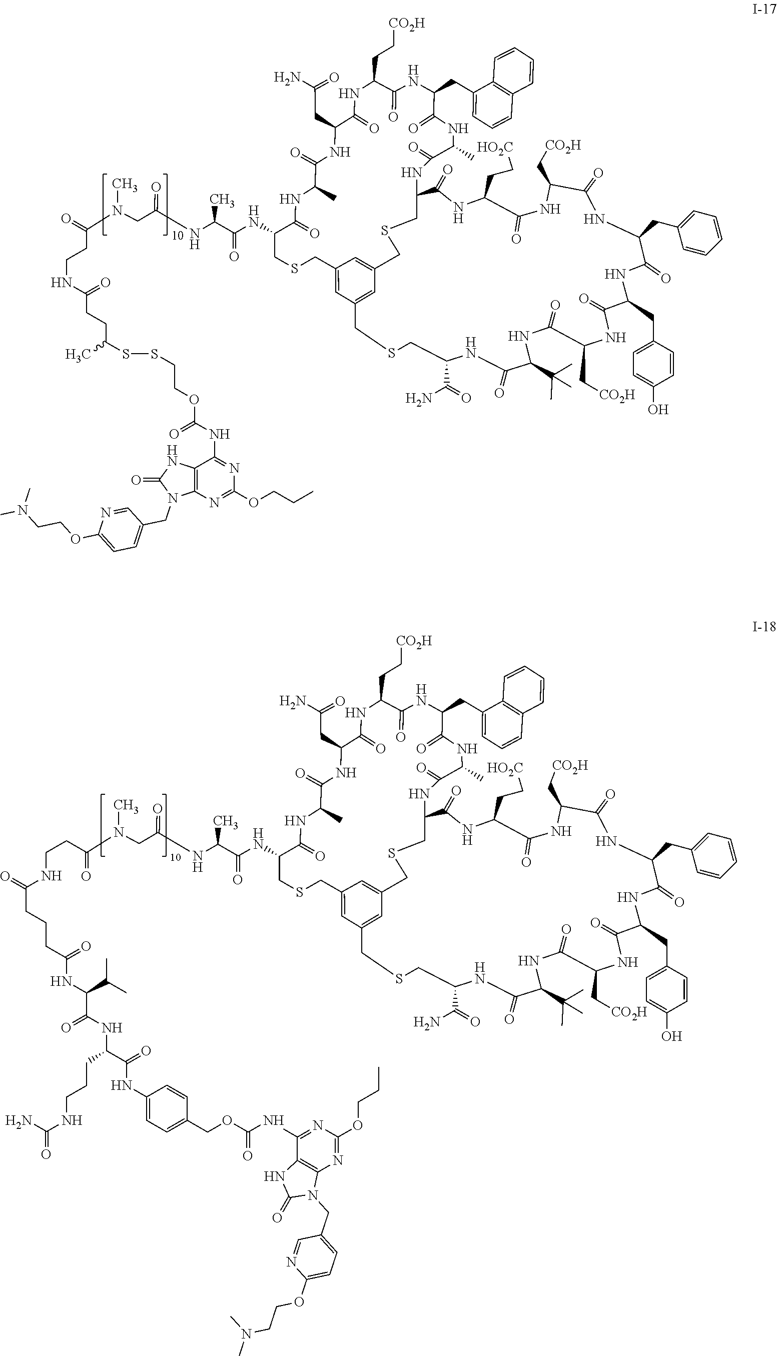

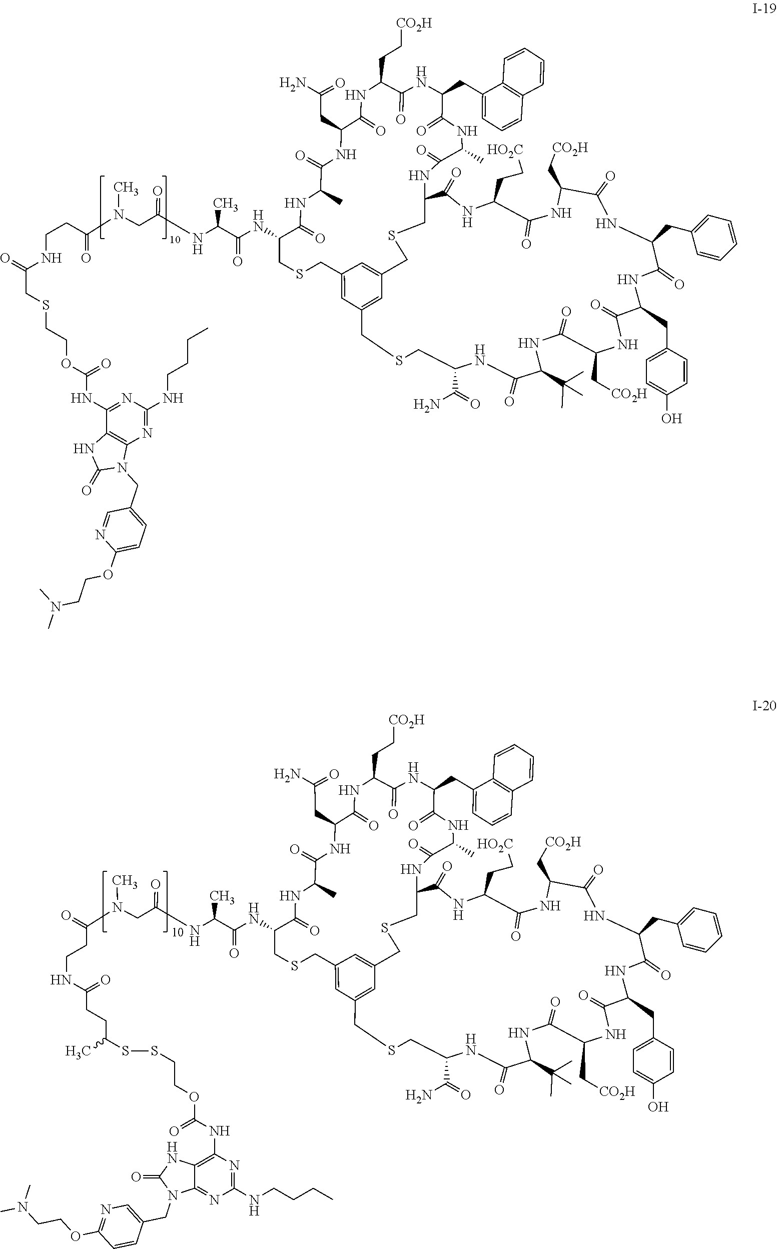

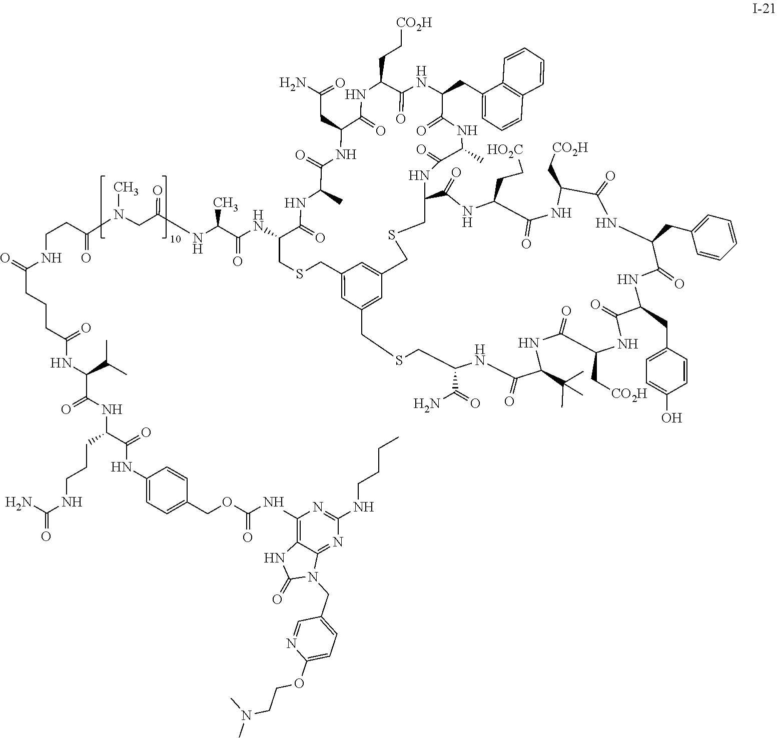

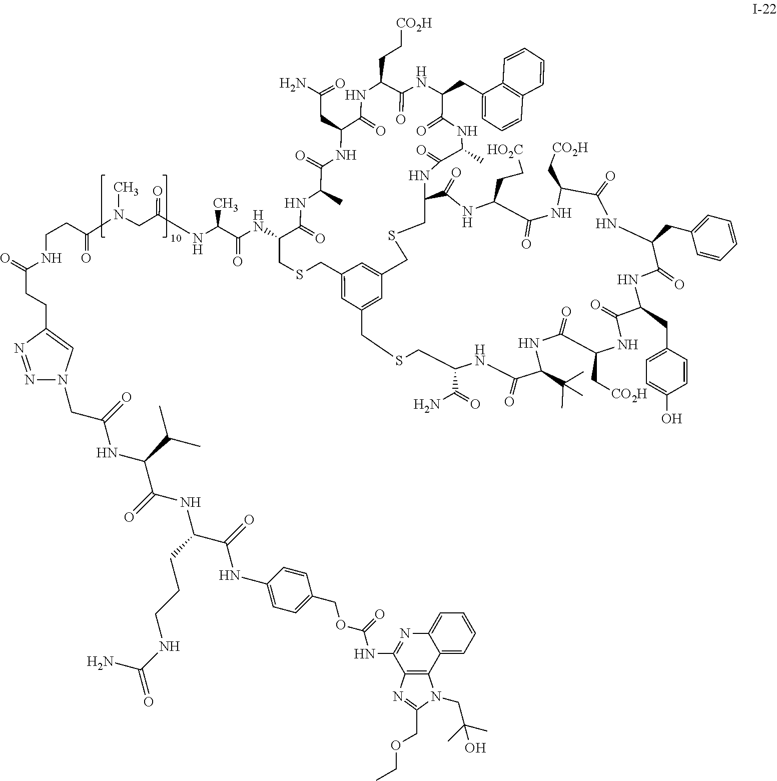

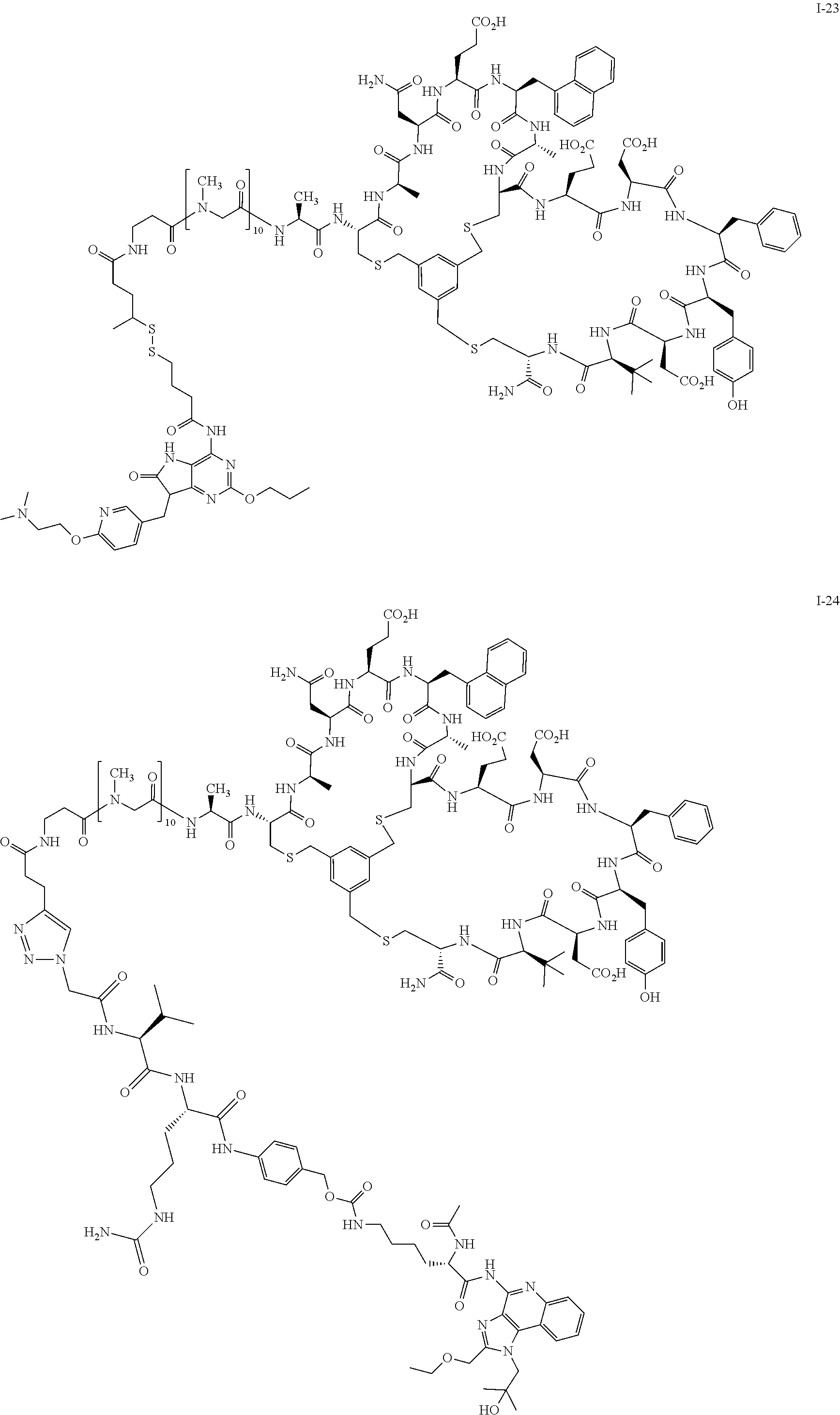

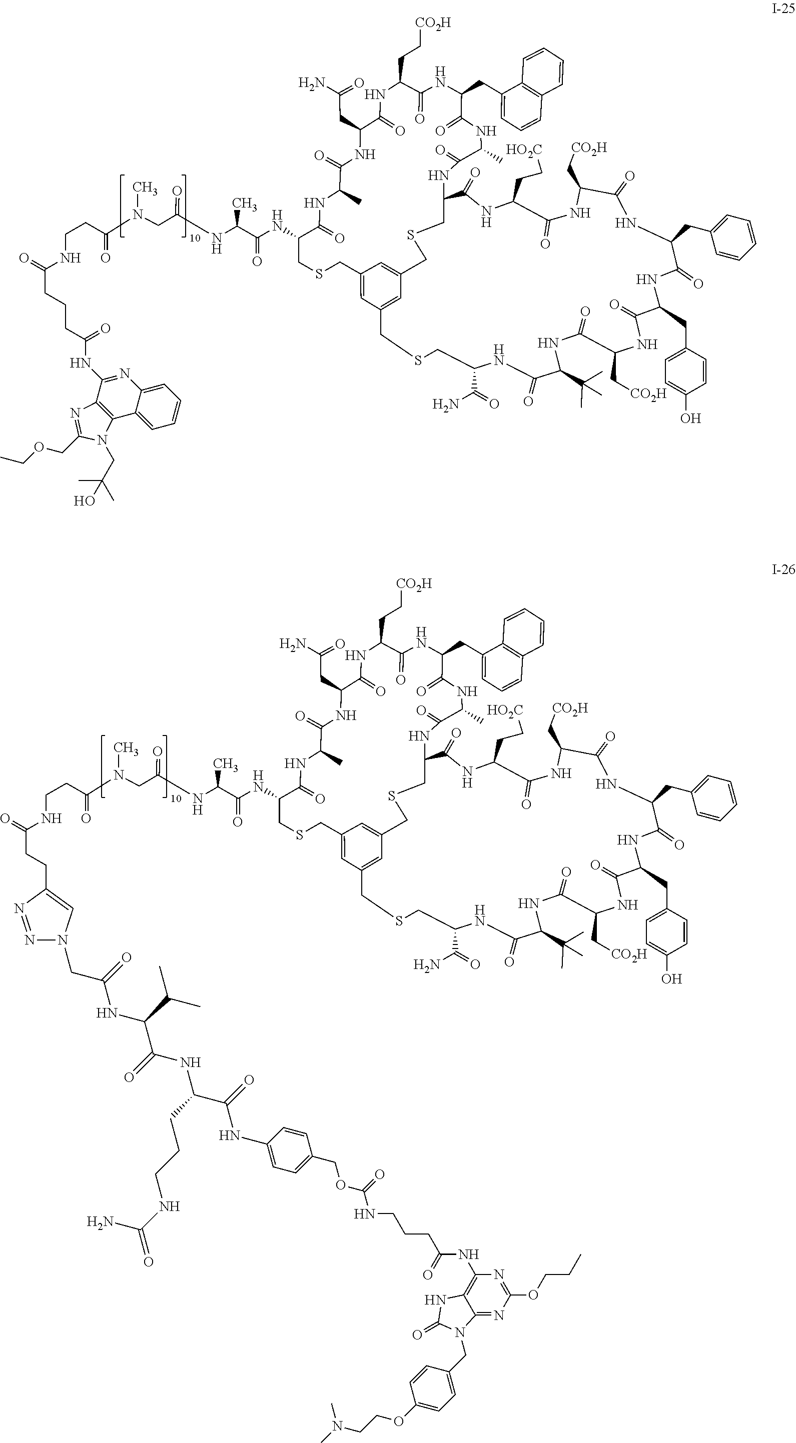

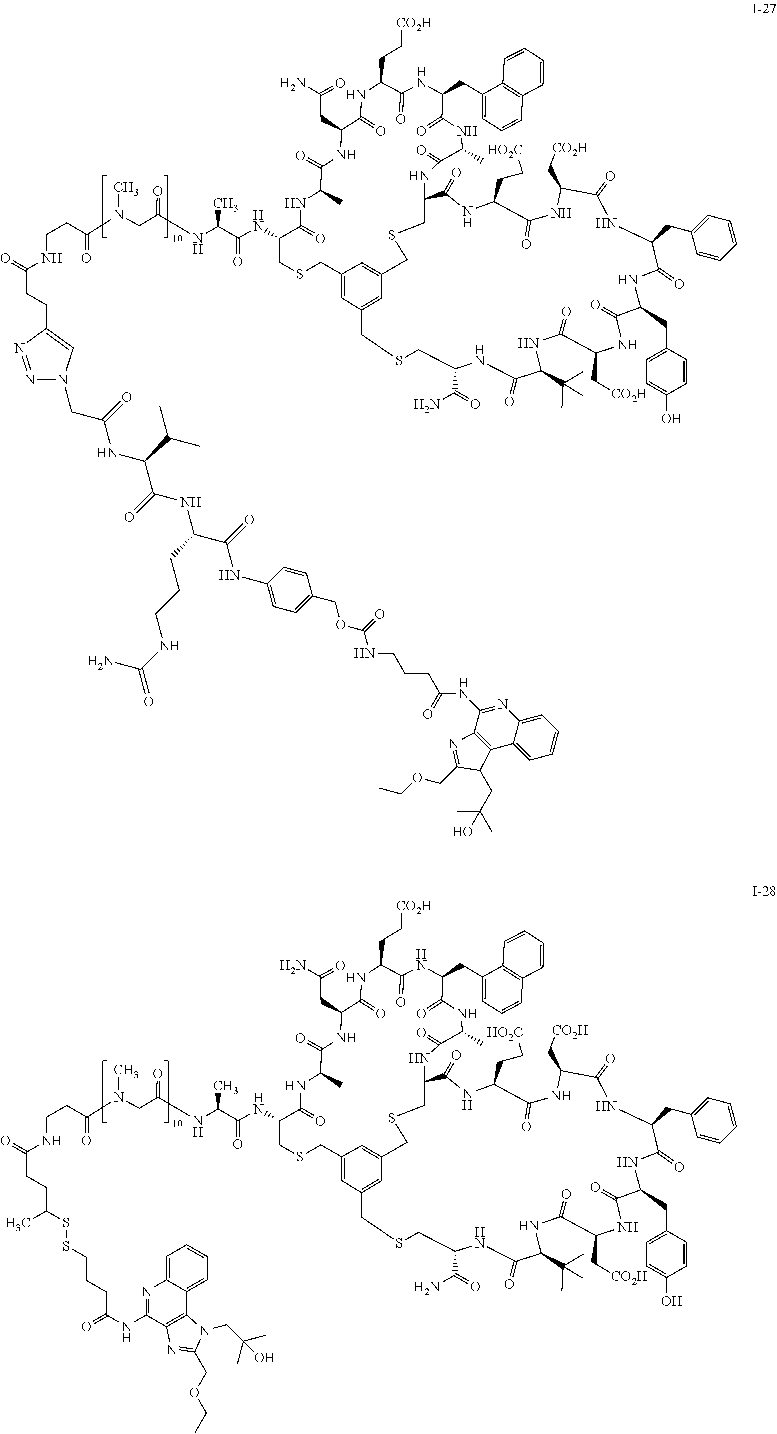

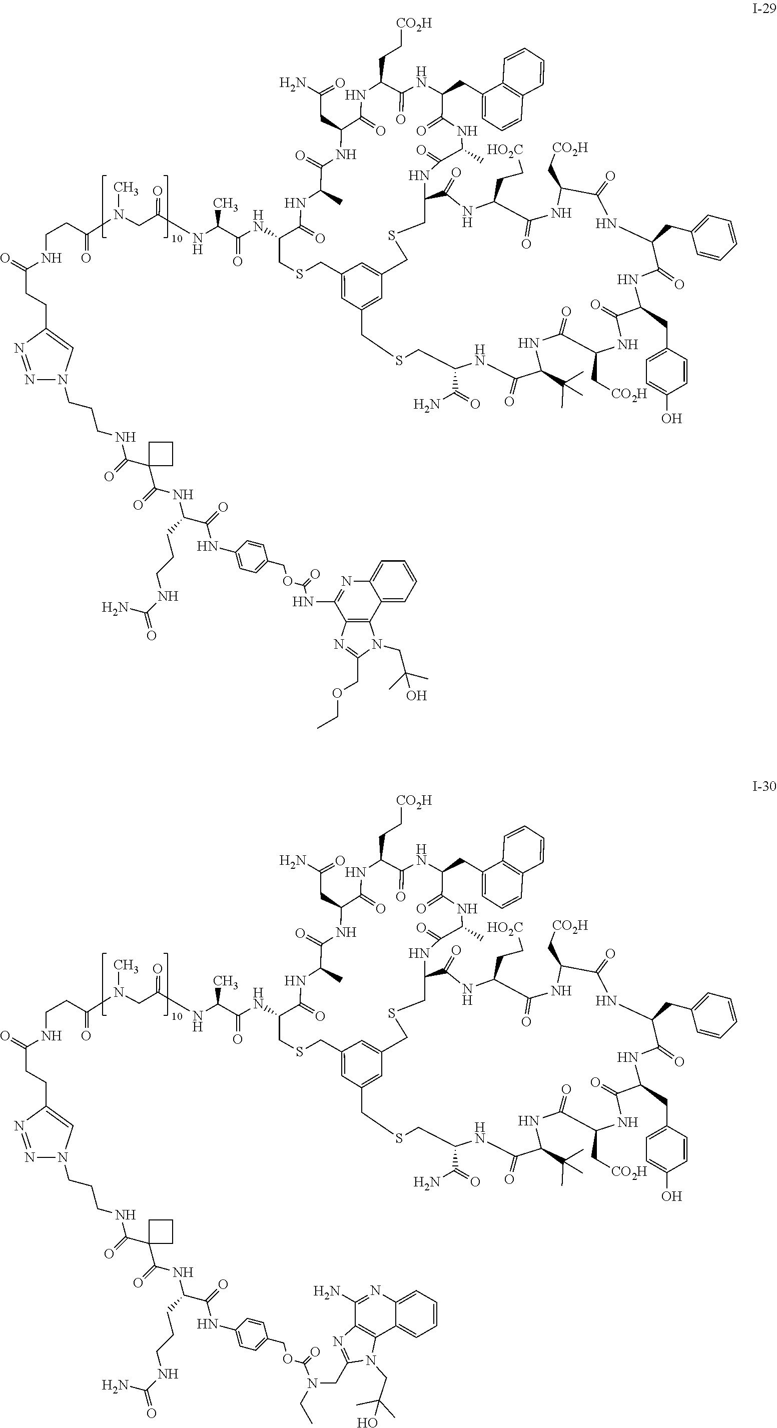

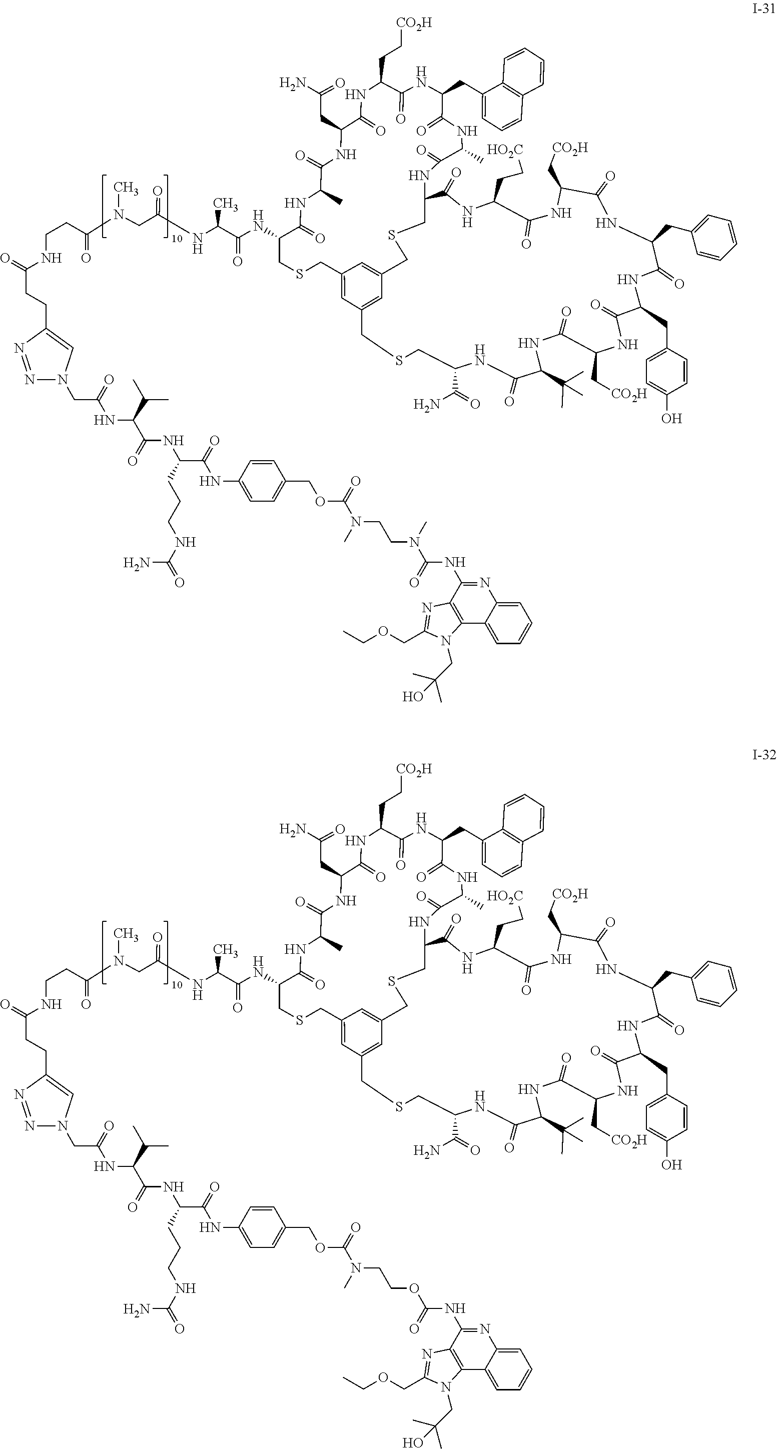

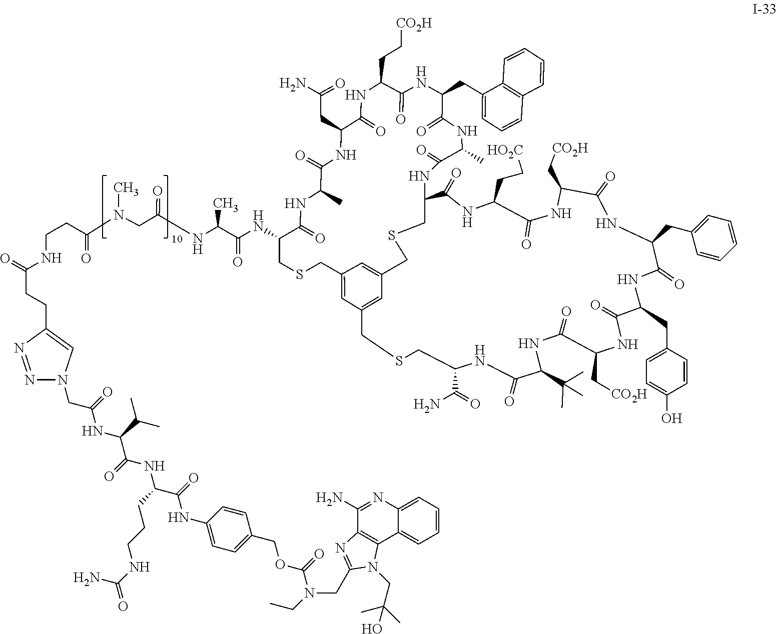

15. A pharmaceutical composition comprising a compound according to claim 1, or a pharmaceutically acceptable salt thereof, and a pharmaceutically acceptable carrier, adjuvant, or vehicle.

16. A method of inducing an immune response in a patient or biological sample comprising administering to said patient, or contacting said biological sample with a compound according to claim 1, or a pharmaceutically acceptable salt thereof.

17. A method of inducing a PRR-A-mediated immune response in a patient or biological sample comprising administering to said patient, or contacting said biological sample with a compound according to claim 1, or a pharmaceutically acceptable salt thereof.

18. A method of treating a disorder, disease, or condition in a patient comprising administering to said patient a compound according to claim 1, or a pharmaceutically acceptable salt thereof.

19. The method of claim 18, wherein the disorder, disease or condition is selected from the group consisting of a cancer and a proliferative disorder.

20. The method of claim 19, wherein the cancer or proliferative disorder is selected from the group consisting of tumors of epithelial origin (adenomas and carcinomas of various types including adenocarcinomas, squamous carcinomas, transitional cell carcinomas and other carcinomas) such as carcinomas of the bladder and urinary tract, breast, gastrointestinal tract (including the esophagus, stomach (gastric), small intestine, colon, rectum and anus), liver (hepatocellular carcinoma), gall bladder and biliary system, exocrine pancreas, kidney, lung (for example adenocarcinomas, small cell lung carcinomas, non-small cell lung carcinomas, bronchioalveolar carcinomas and mesotheliomas), head and neck (for example cancers of the tongue, buccal cavity, larynx, pharynx, nasopharynx, tonsil, salivary glands, nasal cavity and paranasal sinuses), ovary, fallopian tubes, peritoneum, vagina, vulva, penis, cervix, myometrium, endometrium, thyroid (for example thyroid follicular carcinoma), adrenal, prostate, skin and adnexae (for example melanoma, basal cell carcinoma, squamous cell carcinoma, keratoacanthoma, dysplastic naevus); hematological malignancies (i.e. leukemias, lymphomas) and premalignant hematological disorders and disorders of borderline malignancy including hematological malignancies and related conditions of lymphoid lineage (for example acute lymphocytic leukemia [ALL], chronic lymphocytic leukemia [CLL], B-cell lymphomas such as diffuse large B-cell lymphoma [DLBCL], follicular lymphoma, Burkitt's lymphoma, mantle cell lymphoma, T-cell lymphomas and leukemias, natural killer [NK] cell lymphomas, Hodgkin's lymphomas, hairy cell leukemia, monoclonal gammopathy of uncertain significance, plasmacytoma, multiple myeloma, and post-transplant lymphoproliferative disorders), and hematological malignancies and related conditions of myeloid lineage (for example acute myelogenousleukemia [AML], chronic myelogenousleukemia [CML], chronic myelomonocyticleukemia [CMML], hypereosinophilic syndrome, myeloproliferative disorders such as polycythaemia vera, essential thrombocythaemia and primary myelofibrosis, myeloproliferative syndrome, myelodysplastic syndrome, and promyelocyticleukemia); tumors of mesenchymal origin, for example sarcomas of soft tissue, bone or cartilage such as osteosarcomas, fibrosarcomas, chondrosarcomas, rhabdomyosarcomas, leiomyosarcomas, liposarcomas, angiosarcomas, Kaposi's sarcoma, Ewing's sarcoma, synovial sarcomas, epithelioid sarcomas, gastrointestinal stromal tumors, benign and malignant histiocytomas, and dermatofibrosarcomaprotuberans; tumors of the central or peripheral nervous system (for example astrocytomas, gliomas and glioblastomas, meningiomas, ependymomas, pineal tumors and schwannomas); endocrine tumors (for example pituitary tumors, adrenal tumors, islet cell tumors, parathyroid tumors, carcinoid tumors and medullary carcinoma of the thyroid); ocular and adnexal tumors (for example retinoblastoma); germ cell and trophoblastic tumors (for example teratomas, seminomas, dysgerminomas, hydatidiform moles and choriocarcinomas); and pediatric and embryonal tumors (for example medulloblastoma, neuroblastoma, Wilms tumor, and primitive neuroectodermal tumors); or syndromes, congenital or otherwise, which leave the patient susceptible to malignancy (for example Xeroderma Pigmentosum).
